(19) United States
(12) Patent Application Publication
(10) Pub. No.: US 2026/0358315 A1
(43) Pub. Date: Nov. 12, 2026

(54) ANALYSIS OF HYDROGEN AND RELATED SUBSTANCES IN MATERIALS

(71) Applicant: Michael P. Smith, Tulsa, OK (US)

(72) Inventor: Michael P. Smith, Tulsa, OK (US)

(21) Appl. No.: 19/713,781

(22) Filed: Jun. 19, 2026

Related U.S. Application Data
(63) Continuation of application No. PCT/US2024/061798, filed on Dec. 23, 2024.
(60) Provisional application No. 63/613,751, filed on Dec. 21, 2023.

(51) Int. Cl. G01N 33/00

(52) U.S. Cl. CPC G01N 33/005

(57) ABSTRACT

Methods for quantifying hydrogen and evaluating hydrogen purity in materials, such as in geologic materials of a geologic unit, are provided, as well as devices for performing such analyses, and other related methods or other embodiments. One exemplary aspect is a method for measuring the amount of hydrogen in a material comprising obtaining an analyzable amount of a material as a sample, subjecting the sample to one or more gentle vacuum equivalent forces that extract one or more easily released volatile substances from the sample, if present, the one or more easily released volatile substances comprising at least one hydrogen proxy, collecting at least a portion of the extracted easily released volatile substances, and measuring the amount of the at least one hydrogen proxy in the collected portion of the easily released volatile substances and using the measurement in a determination of the amount of hydrogen in the material.

### RELATED APPLICATIONS/PRIORITY

This Application is a continuation of PCT Application PCT/US2024/061798, filed Dec. 23, 2024, entitled, ANALYSIS OF HYDROGEN AND RELATED OR OTHER SUBSTANCES FROM MATERIALS, INCLUDING GEOLOGIC MATERIALS; RELATED METHODS AND DEVICES; AND NEW APPLICATIONS OF HYDROGEN AND OTHER SUBSTANCES, which claimed priority to U.S. Provisional Patent Application No. 63/613,751 filed Dec. 21, 2023, entitled ANALYSIS OF HYDROGEN AND RELATED OR OTHER SUBSTANCES FROM MATERIALS, INCLUDING GEOLOGIC MATERIALS; RELATED DEVICES, COMPOSITIONS, AND METHODS; AND NEW APPLICATIONS OF HYDROGEN AND OTHER SUBSTANCES. This application claims the benefit of priority to the above-referenced application.

### FIELD OF THE INVENTION

This disclosure primarily relates to method(s) of analyzing (e.g., quantifying or measuring), compound(s) in or associated with, e.g., extracted from, material(s), such as geologic material(s), wherein such compound(s) include, e.g., hydrogen, helium, or both. This disclosure further relates to system(s), including, e.g., related computer system(s), device(s), and associated method(s), for obtaining and using hydrogen and helium data from material samples as well as to new applications of such data.

### BACKGROUND OF THE INVENTION

Earlier patents belonging to the inventor of the present Application disclose methods and related devices for the analysis of materials, such as geologic materials, including volatile substances associated with such materials, e.g., that can be analyzed from samples associated with petroleum operations, such as, e.g., drill cuttings or cores. This prior work is collectively referred to as the “Prior Smith Patents,” examples of which are provided in the “Terms and Principles Specific to the Technology” section of this Application.

These documents are disclosed herein by reference, and methods, materials, systems, etc., of any one, some, most, or all of such disclosures can be combined with aspects of this disclosure, uncontradicted. However, given the differences between the methods, devices, compositions, etc., of this disclosure, any element of the disclosure of any one of, some, or all of the Prior Smith Patents also can be, uncontradicted, excluded from any aspect of this disclosure.

The method(s) and device(s) of the Prior Smith Patents have primarily focused on the analysis of hydrocarbons or other compounds (e.g., organic acids) for the identification or characterization of petroleum production properties associated with geologic materials and related sites and, more recently, to the analysis of carbon sequestration capability or performance.

Hydrogen is an attractive potential fuel source and resource. However, there are a number of impediments and challenges to identifying useful sources of fuel grade hydrogen and obtaining fuel grade hydrogen from geologic or other materials. For example, there currently are a lack of methods, systems, and devices that can analyze subsurface hydrogen efficiently or effectively, and it is the common view of the scientific community that hydrogen is normally presented in mixed composition, e.g., less than about 99.95%, less than about 99.99%, or less than about 99.995% pure hydrogen, with other elements (e.g., helium), rendering identified sources as unsuitable (accurately or not). Additionally, there remains a lack of compositions, e.g., materials, pipes, seals, valves and other equipment associated with the extraction and production of gases and liquids, that are stable and robust in the presence of hydrogen. These facts remain true even though hydrogen exploration and production are key areas of focus for institutions and governments as humankind seeks to find better alternative fuel sources. In view of the failure to develop such effective methods, devices, compositions, and systems, despite significant attention of the scientific community to such problems, it is clear that the development of effective methods for hydrogen identification, analysis, quantification, production, and the development of devices, systems, compositions, and methods related thereto, requires the application of inventive ingenuity.

### SUMMARY OF THE INVENTION

This document includes a section entitled “CONSTRUCTION PRINCIPLES AND DESCRIPTION OF SELECT TERMS” that readers are encouraged to consult to help properly interpret the disclosure provided in this section and elsewhere here. That section includes a list of acronyms frequently used in this disclosure.

This “Summary of the Invention” section (“Summary”) briefly describes the elements and characteristics of selected illustrative embodiment(s) of the invention. The brief summaries of such embodiments provided here are primarily intended to illustrate the nature of the invention and, accordingly, the content of this Summary is not intended to be all-inclusive, and the scope of the invention is not limited to, or by, the exemplary aspects of the invention provided in this section. Any of the aspects of the invention described in this section can be combined with any other aspect described in this or any other aspect of this disclosure.

In aspects, the technology(ies) herein provide method(s) for measuring the amount of hydrogen in a material. In aspects, such method(s) comprise obtaining an analyzable amount of a material as a sample. In aspects, such methods(s) comprise subjecting the sample to one or more gentle vacuum equivalent forces that extract one or more easily released volatile substances from the sample, if present, the one or more easily released volatile substances comprising at least one hydrogen proxy. In aspects, such method(s) comprise collecting at least a portion of the extracted easily released volatile substances. In aspects, such methods comprise measuring the amount of the at least one hydrogen proxy in the collected portion of the easily released volatile substances and using the measurement in a determination of the amount of hydrogen in the material.

In other respects, the technology(ies) herein provide method(s) for identifying conditions for enhanced production of highly purified hydrogen from a material. In aspects, the method(s) comprise obtaining at least one sample comprising an analyzable amount of a material comprising hydrogen and helium. In aspects, such method(s) comprise subjecting the sample to one or more gentle vacuum-equivalent forces to extract a plurality of extracted gas aliquots from the sample, each aliquot comprising a plurality of easily released volatile substances if such easily released volatile substances are present in the sample. In aspects, the plurality of extracted easily released volatile substances comprise (i) hydrogen, a hydrogen proxy, or both hydrogen and a hydrogen proxy, and (ii) helium, and, e.g., either (1) the at least one sample comprises multiple samples, wherein a relatively higher amount of hydrogen than helium is extracted from at least one sample of the multiple samples than at least one other sample of the multiple samples, (2) the method comprises subjecting the at least one sample to at least two different gentle vacuum equivalent forces comprising a first gentle vacuum equivalent force that extracts a relatively higher amount of hydrogen than helium from the at least one sample than at least a second gentle vacuum equivalent force of the at least two different gentle vacuum equivalent forces, or (3) the at least one sample comprises multiple samples, wherein a relatively higher amount of hydrogen than helium is extracted from at least one sample of the multiple samples than at least one other sample of the multiple samples and the method comprises subjecting the at least one sample to at least two different gentle vacuum equivalent forces comprising a first gentle vacuum equivalent force that extracts a relatively higher amount of hydrogen than helium from the at least one sample than at least a second gentle vacuum force of the at least two different gentle vacuum forces. In aspects, the method(s) comprise collecting at least a portion of each of the plurality of extracted gas aliquots. In aspects, the method(s) comprise obtaining measurements of (i) hydrogen, the hydrogen proxy, or both hydrogen and the hydrogen proxy, and (ii) helium, in each of the collected portions of extracted gas aliquots. In aspects, the method(s) comprise using the measurements from the collected portions of the extracted gas aliquots to identify (a) a gentle vacuum equivalent force that selectively extracts hydrogen from the material, (b) the at least one sample that is associated with the relatively higher amount of hydrogen extraction than helium extraction, or (c) both (a) and (b).

In still further respects, disclosed herein are method(s) for evaluating the hydrogen generation capacity of a material comprising obtaining a solid or semisolid mineral aggregate material. In aspects, method(s) comprise contacting the material with an aqueous liquid under conditions that will result in hydrogen generation if the material is an effective hydrogen-generating material, to form a water-treated material. In aspects, method(s) comprise subjecting the water-treated material to one or more gentle vacuum equivalent forces that extract one or more easily released volatile substances from the sample, if present, the one or more easily released volatile substances comprising hydrogen, at least one hydrogen proxy, or both. In aspects, method(s) comprise collecting at least a portion of the extracted easily released volatile substances. In aspects, method(s) comprise measuring the hydrogen content, hydrogen proxy content, or both the hydrogen and hydrogen proxy content of the portion/in the portion. In aspects, method(s) comprise evaluating the ability of the material to generate hydrogen from the measurement or measurements obtained by the method.

In a further aspect, technology(ies) herein provide a device for the rapid analysis of hydrogen in a solid or semisolid mineral aggregate material. In aspects, the device comprises a movable container comprising a plurality of compartments, each compartment occupying a position among a plurality of positions, each compartment (a) comprising a separate inlet, a separate outlet, or both, and (b) being configured to receive an analyzable sample of a solid or semisolid mineral aggregate material at a first position among the plurality of positions and to deliver the sample to one or more other positions of the plurality of positions. In aspects, the device comprises an extraction component positioned in effective proximity to at least one of the plurality of positions, and that is configured to selectively apply one or more gentle vacuum equivalent extraction forces to extract one or more aliquots of easily extractable volatile substances from the sample of solid or semisolid mineral aggregate material received by the movable container. In aspects, the device comprises a container movement component that causes the movable container to move and thereby causes the different compartments of the plurality of the compartments to be located at the plurality of positions at different times during operation of the device, the plurality of positions comprising a first position and a second position, wherein a first compartment when located in the first position is configured to receive a first sample delivered to the device at the same time that a second compartment in the second position is oriented to expose a second sample contained therein to the extraction component. In aspects, the device comprises a trap component that selectively removes water from at least a first portion of each of the one or more aliquots. In aspects, the device comprises a collection component that selectively collects a second portion of each of the one or more aliquots. In aspects, the device comprises an analytical component that analyzes the easily released volatile content of each collected second portion of the one or more aliquots and measures the amount of the at least one hydrogen proxy therein. In aspects, the device comprises an output component for relaying the analysis of the analytical component to a user, a different system, or both. In aspects, the device is configured to cause the container movement component to move the movable container to cause the first compartment to be in the first position and the second compartment to be in the second position and to thereafter move each of the first compartment and second compartment to different positions.

### BRIEF DESCRIPTION OF THE DRAWINGS/FIGURES

The drawings/figures provided here and the associated following brief description of figures are intended to exemplify certain aspects and principles of the invention without limiting its scope.

FIG. 1 illustrates an embodiment of the technology(ies) disclosed herein, wherein the measurement of hydrogen, ammonia produced from hydrogen (and/or the consumption of nitrogen in the production of ammonia), and water produced from hydrogen (and/or the consumption of oxygen in the production of water), with calculations applied as applicable, is are combined to estimate the amount of hydrogen present at the time the sample was first collected (e.g., at the time a drill cutting reached the surface of the Earth before reacting with air).

FIG. 2 illustrates an embodiment of the technology(ies) disclosed herein, wherein distinct helium-rich versus hydrogen-rich zones are identified across differing strata of a geologic location, e.g., a well or borehole.

FIG. 3 illustrates an embodiment of the technology(ies) disclosed herein, demonstrating that the application of at least two different force(s) to a single material sample, such as differing vacuum forces, can cause the detectable or significant release of one of hydrogen or helium but not the other from the material.

FIGS. 4-8 illustrate an analyzer/device (analysis system) for collecting and analyzing the amount of particular gas(es) (volatile(s)) in sample(s).

FIGS. 9A-9D illustrate exemplary concepts related to multi-aliquot rock volatile stratigraphy methods.

FIG. 10A illustrates a first interpretation of a pattern of data obtained from the application of multi-aliquot rock volatile stratigraphy methods.

FIG. 10B illustrates a second interpretation of a pattern of data obtained from the application of multi-aliquot rock volatile stratigraphy methods.

FIG. 11 provides an exemplary method for determining the amount of hydrogen present in a material sample.

FIG. 12 provides an exemplary method for determining the relative purity of hydrogen, helium, or both, in a material sample compared to a standard or to at least one other second material sample.

FIGS. 13A-13D provide exemplary method(s) of the technology(ies) herein directed to the capture, analysis, and application of multi-aliquot rock volatile stratigraphy method(s) and the interpretation of data resulting therefrom, wherein FIGS. 13C and 13D provide more specific examples of the method(s) exemplified in FIGS. 13A and 13B, respectively.

FIG. 14 provides an exemplary method of the technology(ies) herein for identifying differentiated storage of helium and hydrogen within the same material.

FIG. 15 provides an exemplary method of the technology(ies) herein directed to the generation of a reference for use in, or directed directly to, the determination of optimized condition(s) for synthetic hydrogen generation.

FIG. 16 provides an exemplary method of the technology(ies) herein directed to the generation of synthetic hydrogen.

FIGS. 17-20 provide exemplary method(s) of the technology(ies) herein directed to the determination of the amount of hydrogen present in a material sample, wherein FIG. 18 is a subprocess which can be conducted as a component of the method illustrated in FIG. 17;

FIG. 19 is a subprocess which can be conducted as a component of the method illustrated in FIG. 17 and related figures; and FIG. 20 is a subprocess which can be conducted as a component of the method illustrated in FIG. 17 and related figures),

FIG. 21 provides an exemplary method of the technology(ies) herein directed to protecting a hydrogen-sensitive material from exposure to hydrogen using an inert gas such as helium.

### LISTED EXEMPLARY ASPECTS

Disclosed here are several different but related exemplary aspects (variations) of the technology. The following is a non-limiting list of exemplary aspects of the technology, which illustrates embodiments of the technology in a summary form to aid readers in quickly understanding the overall scope of the technology. Similar to patent claims, the listed aspects described in the paragraphs of this section may refer to (depend on/from) one or more other paragraphs. Readers will understand that such references mean that the features/characteristics or steps of such referenced aspects are incorporated into/combined with the referring aspect. E.g., if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of a composition, method, system, device, etc., including the features of both aspect 1 and aspect 2).

Lists of aspects describing specific exemplary embodiments of the technology are sometimes employed to aid the reader in understanding the technology. Such aspects can, within them, reference other exemplary aspects, either individually or as groups of aspects (e.g., via reference to a range within a list of numbered aspects when such aspects are provided as a numbered list). Reference to ranges of aspects should be interpreted as referencing all such aspects individually, each as a unique embodiment of the technology, and in combination with one another as unique embodiment(s) of the technology, according to the presentation provided of such aspects, unless such an aspect within such a referenced range is either contradictory or nonsensical. If contradicted, reference to the contradictory aspect should be excluded. The term “preceding applicable aspects” used in one aspect to refer to all the lower-numbered aspects that precede it in the list means all those preceding aspects that can be sensibly combined with the content of the applicable referring aspect. In this respect, the phrase “applicable aspects” works in a manner similar to the phrase “uncontradicted,” discussed below.

It is intended that these listed exemplary aspects begin with the first listed aspect (ASPECT 1) and thereafter be numbered sequentially and incrementally by the inclusion of a reference (ASPECT 2, ASPECT 3, etc.). Readers will recognize that in a complicated listing of aspects, however, errors can sometimes arise. In case of a missing aspect reference or repeated aspect reference, the order of placement of the actual recited aspect in the list that is associated with the repeated aspect reference or missing aspect reference will control (e.g., if there is an unlabeled aspect located between a first aspect labeled ASPECT 1 and a third aspect labeled aspect referenced as ASPECT 2, the unlabeled aspect should be treated as ASPECT 2, and the aspect labeled as ASPECT 2 treated as ASPECT 3, etc.), and all numbering in the list (including all references to aspects in the list) be interpreted as accordingly modified (e.g., if the fourth aspect in such list was labeled as ASPECT 3, it should be interpreted as being labeled as ASPECT 4, and if such aspect referred to “any one or both of aspect 1 or aspect 2,” it should be read as referring to “any one or more of aspects 1-3”). Similarly, if an aspect is misnumbered (e.g., by a number in the sequence being skipped or otherwise missing), readers will construe this list of aspects according to the order of placement of the recited aspects, over the numerical references. Further, if one or more of the listed exemplary aspects of the technology in this section fails to reference any other aspects of the technology, such aspect, uncontradicted, should be interpreted as applying to or as capable of being incorporated into any one or more other exemplary aspect(s) provided in this section.

These listed aspects can be combined with, or further modified by, any of the other specific disclosures provided elsewhere in this document. The inclusion of headers herein is for the convenience of the reader only (e.g., in identifying aspects relating to particular applications, embodiments, etc.), and, uncontradicted, do not limit the applicability of aspects. With this in mind, the specific listed exemplary aspects of the technology are—

### Aspect 1

A method for measuring the amount of hydrogen in a material comprising
(1) obtaining an analyzable amount of a material as a sample,
(2) subjecting the sample to one or more gentle vacuum equivalent forces that extract one or more easily released volatile substances from the sample, if present, the one or more easily released volatile substances comprising at least one hydrogen proxy,
(3) collecting at least a portion of the extracted easily released volatile substances, and
(4) measuring the amount of the at least one hydrogen proxy in the collected portion of the easily released volatile substances and using the measurement in a determination of the amount of hydrogen in the material.

### Aspect 2

The method of ASPECT 1, wherein the method comprises isolating the sample by placing the sample in an enclosed environment prior to subjecting the sample to the one or more gentle vacuum equivalent forces.

### Aspect 3

The method of any one or more of the preceding applicable aspects, wherein the sample is isolated in the enclosed environment with a gas.

### Aspect 4

The method of any one or more of Aspect 2 and Aspect 3, wherein the enclosed environment is a container.

### Aspect 5

The method of any one or more of the preceding applicable aspects wherein the gas comprises, mostly comprises, generally consists of, substantially consists of, consists essentially of, or is air.

### Aspect 6

The method of any one or more of the preceding applicable aspects wherein the gas comprises, mostly comprises, generally consists of, substantially consists of, consists essentially of, a noble gas, such as argon, or a mixture of noble gases.

### Aspect 7

The method of any one or more of the preceding applicable aspects, wherein the one or more gentle vacuum equivalent forces comprises at least one application of a gentle vacuum pressure.

### Aspect 8

The method of ASPECT 8, wherein the enclosed environment is a container that comprises a needle-puncturable seal component.

### Aspect 9

The method of any one or more of the preceding applicable aspects, wherein the method comprises analyzing the amount of hydrogen in the sample and using both the amount of hydrogen and the amount of the at least one hydrogen proxy to quantify the hydrogen in the material.

### Aspect 10

The method of any one or more of the preceding applicable aspects wherein the at least one hydrogen proxy comprises ammonia.

### Aspect 11

The method of any one or more of the preceding applicable aspects, wherein the sample is isolated in the enclosed environment with a gas, the gas is mostly, generally, substantially, essentially, or entirely air, and the method comprises measuring the amount of argon in the air to determine the amount of air in the enclosed environment.

### Aspect 12

The method of any one or more of the preceding applicable aspects, wherein the at least one hydrogen proxy comprises an ammonia proxy.

### Aspect 13

The method of ASPECT 12, wherein the ammonia proxy comprises nitrogen, and wherein the method comprises using the quantity of air in the enclosed environment to determine the amount of ammonia produced from hydrogen in the sample prior to isolating the sample.

### Aspect 14

The method of any one or more of the preceding applicable aspects, wherein the at least one hydrogen proxy comprises a water proxy.

### Aspect 15

The method of ASPECT 14, wherein the sample is isolated in the enclosed environment with a gas, the gas is air, and the method comprises measuring the amount of argon in the air to determine the amount of air in the enclosed environment; the water proxy is oxygen; and wherein the method comprises using the quantity of air in the enclosed environment to determine the amount of water produced from hydrogen in the sample prior to isolating the sample.

### Aspect 16

The method of any one or more of the preceding applicable aspects, wherein the at least one hydrogen proxy comprises ammonia or an ammonia proxy and water or a water proxy, and the quantity of hydrogen in the material is determined by adding an amount of hydrogen lost to ammonia production as determined at least in part by the measurement of the ammonia or the ammonia proxy; an amount of hydrogen lost to water production as determined at least in part by the measurement of the water or the water proxy; and the hydrogen directly measured in the material.

### Aspect 17

The method of any one or more of the preceding applicable aspects wherein the sample is isolated in the enclosed environment with a gas, the gas is air, and the method comprises measuring the amount of argon in the air to determine the amount of air in the enclosed environment.

### Aspect 18

The method of any one or more of the preceding applicable aspects, wherein the method comprises removing water from the sample, the easily released volatile substances, or both, prior to or concurrently with collecting the portion of the extracted easily released volatile substances comprising the at least one hydrogen proxy.

### Aspect 19

The method of ASPECT 18, wherein the removal of water from the sample, the easily released volatile substances, or both, is performed by, i.a., subjecting the easily released volatile substances to a media that selectively traps a portion of the easily released volatile substances that (1) comprises water, (2) does not comprise all of the very small volatile substances in the easily released volatile substances, and (3) does not comprise the at least one hydrogen proxy.

### Aspect 20

The method of ASPECT 19, wherein the removal of water from the sample, the easily released volatile substances, or both, is performed by contacting the easily released volatile substances with a cryogenic trap such that the trapped portion of the easily released volatile substances condenses to the cryogenic trap and the collected portion of the easily released volatile substances comprising the at least one hydrogen proxy does not condense to the cryogenic trap.

### Aspect 21

The method of ASPECT 18, wherein the removal of water from the sample is performed by contacting the sample, the easily released volatile substances, or both, with a desiccant (e.g., calcium, gypsum, or a combination thereof) or with an equivalently or better means for water removal.

### Aspect 22

The method of any one or more of the preceding applicable aspects, wherein the determination of the amount of hydrogen comprises using an atom compound presence factor to assess the amount of hydrogen originally contained in the sample.

### Aspect 23

The method of any one or more of the preceding applicable aspects, wherein the method comprises collecting multiple samples from different parts of a geologic unit and separately subjecting each of the samples to the other steps of the method to generate a map of hydrogen amounts present in the different parts of the geologic unit.

### Aspect 24

The method of ASPECT 23, wherein the multiple samples comprise a plurality of one or more drill cuttings, mud sample(s), core sample(s), or a combination of any or all thereof.

### Aspect 25

The method of any one of any one or more of the preceding applicable aspects, wherein the method comprises comparing the quantity of hydrogen measured in the one or more easily released volatile substances to the quantity of helium measured in the one or more easily released volatile substances.

### Aspect 26

The method of ASPECT 25, wherein the method comprises analyzing a plurality of samples from different parts of a geologic unit, determining the amount of hydrogen and the amount of helium in the plurality of samples, and identifying one or more areas of high hydrogen content; one or more areas of high helium content; one or more areas of high hydrogen content and low helium content; one or more areas of high helium content and low hydrogen content; or any combination thereof in the geologic unit.

### Aspect 27

The method of any one or more of the preceding applicable aspects, wherein the method comprises subjecting one or more samples to different volatile extraction conditions, wherein one of the different volatile extraction conditions results in the extraction of a significantly greater amount of hydrogen than helium and a different one of the different volatile extraction conditions results in the extraction of a significantly greater amount of helium than hydrogen.

### Aspect 28

The method of any one or more of the preceding applicable aspects, wherein the application of one or more gentle vacuum equivalent forces comprises subjecting the sample to at least two separate applications of gentle vacuum pressure, the at least two separate applications of gentle vacuum pressure comprising a first vacuum having a first pressure applied for a first time period and a second vacuum having either the first pressure or a pressure that is substantially the same pressure as the first pressure applied for a second period, wherein the second period is substantially longer than the first period, and, wherein the first vacuum and the second vacuum extract different amounts of hydrogen from the sample.

### Aspect 29

The method of ASPECT 28, wherein the at least two separate applications of gentle vacuum pressure further comprise a third vacuum having a second pressure applied for a third period and a fourth vacuum having either the second pressure or a that is substantially the same pressure as the second pressure applied for a fourth period, wherein the fourth period is substantially longer than the third period, and, wherein at least one of the first vacuum, second vacuum, third vacuum, and fourth vacuum extracts different amounts of hydrogen from the sample.

### Aspect 30

A method for identifying conditions for enhanced production of highly purified hydrogen from a material comprising
(1) obtaining at least one sample comprising an analyzable amount of a material comprising hydrogen and helium,
(2) subjecting the sample to one or more gentle vacuum equivalent forces to extract a plurality of extracted gas aliquots from the sample, each aliquot comprising a plurality of easily released volatile substances if such easily released volatile substances are present in the sample, wherein
(3) the plurality of extracted easily released volatile substances comprise (i) hydrogen, a hydrogen proxy, or both hydrogen and a hydrogen proxy, and (ii) helium, and
(4) either
   (a) the at least one sample comprises multiple samples, wherein a relatively higher amount of hydrogen than helium is extracted from at least one sample of the multiple samples than at least one other sample of the multiple samples,
   (b) the method comprises subjecting the at least one sample to at least two different gentle vacuum equivalent forces comprising a first gentle vacuum equivalent force that extracts a relatively higher amount of hydrogen than helium from the at least one sample than at least a second gentle vacuum equivalent force of the at least two different gentle vacuum equivalent forces, or
   (c) the at least one sample comprises multiple samples, wherein a relatively higher amount of hydrogen than helium is extracted from at least one sample of the multiple samples than at least one other sample of the multiple samples and the method comprises subjecting the at least one sample to at least two different gentle vacuum equivalent forces comprising a first gentle vacuum equivalent force that extracts a relatively higher amount of hydrogen than helium from the at least one sample than at least a second gentle vacuum force of the at least two different gentle vacuum forces,
(5) collecting at least a portion of each of the plurality of extracted gas aliquots, and
(6) obtaining measurements of (i) hydrogen, the hydrogen proxy, or both hydrogen and the hydrogen proxy, and (ii) helium, in each of the collected portions of extracted gas aliquots, and
(7) using the measurements from the collected portions of the extracted gas aliquots to identify (a) a gentle vacuum equivalent force that selectively extracts hydrogen from the material, (b) the at least one sample that is associated with the relatively higher amount of hydrogen extraction than helium extraction, or (c) both (a) and (b).

### Aspect 31

The method of ASPECT 30, wherein the at least one sample comprises multiple samples, wherein a relatively higher amount of hydrogen than helium is extracted from at least one sample of the multiple samples than at least one other sample of the multiple samples, and the measurements are used to identify the at least one sample that is associated with the relatively higher amount of hydrogen than helium.

### Aspect 32

The method of ASPECT 29 or Aspect 31, wherein the samples are obtained from a geologic unit, and the method comprises identifying separated areas of relatively high helium concentration and relatively high hydrogen concentration in the geologic unit.

### Aspect 33

The method of any one or more of the preceding applicable aspects, wherein the method comprises subjecting the at least one sample to at least two different gentle vacuum equivalent forces comprising a first gentle vacuum equivalent force that extracts a relatively higher amount of hydrogen than helium from the at least one sample than at least a second gentle vacuum force of the at least two different gentle vacuum forces and the measurements are used to identify a gentle vacuum equivalent force that selectively extracts hydrogen from the material.

### Aspect 34

The method of any one or more of the preceding applicable aspects, wherein the first gentle vacuum equivalent force and the second gentle vacuum equivalent force each comprise gentle vacuum pressures.

### Aspect 35

The method of any one or more of the preceding applicable aspects, wherein the first gentle vacuum equivalent force and the second gentle vacuum equivalent force differ in terms of the duration of the gentle vacuum force application to the sample.

### Aspect 36

The method of any one or more of the preceding applicable aspects, wherein the first gentle vacuum equivalent force and the second gentle vacuum equivalent forces differ according to the strength of the vacuum pressure applied to the sample.

### Aspect 37

The method of any one or more of the preceding applicable aspects, wherein the material comprises a sample (i.e., one or more samples) of the material (uncontradicted, in this and other aspects a sample may include one or several separate pieces of material—e.g., rocks or cuttings).

### Aspect 38

The method of any one or more of the preceding applicable aspects, wherein the method comprises isolating the sample by placing the sample in an enclosed environment prior to subjecting the sample to the one or more gentle vacuum equivalent forces.

### Aspect 39

The method of any one or more of the preceding applicable aspects, wherein the sample is isolated in the enclosed environment with a gas.

### Aspect 40

The method of any one or more of Aspect 38 and Aspect 39, wherein the enclosed environment that the material/sample is placed in before extracting the easily released volatile substances is a container.

### Aspect 41

The method of any one or more of the preceding applicable aspects wherein the gas in contact with the sample/material, if applicable, comprises, mostly comprises, generally consists of, substantially consists of, consists essentially of, or is air.

### Aspect 42

The method of any one or more of the preceding applicable aspects wherein the gas in contact with the sample/material, if applicable, comprises, mostly comprises, generally consists of, substantially consists of, consists essentially of, a noble gas, such as argon, or a mixture of noble gases.

### Aspect 43

The method of any one or more of the preceding applicable aspects, wherein the one or more gentle vacuum equivalent forces comprises at least one application of a gentle vacuum pressure.

### Aspect 44

The method of ASPECT 43, wherein the enclosed environment that the easily released volatile substances are extracted from the material/sample in is a container that comprises a needle-puncturable seal component, e.g., where a needle of a device can puncture the container and extract easily released volatile substances in the container after the application of a gentle vacuum equivalent force on the material/sample in the container.

### Aspect 45

The method of any one or more of the preceding applicable aspects, wherein the method comprises analyzing the amount of hydrogen in the material by measuring the amount of at least one hydrogen proxy in the material/easily released volatile substances.

### Aspect 46

The method of any one or more of the preceding applicable aspects, wherein the method comprises analyzing the amount of hydrogen in the sample and using both the amount of hydrogen and the amount of at least one hydrogen proxy to quantify the hydrogen in the material.

### Aspect 47

The method of any one or more of the preceding applicable aspects wherein at least one hydrogen proxy used to measure the amount of hydrogen in the material comprises ammonia.

### Aspect 48

The method of any one or more of the preceding applicable aspects, wherein the sample is isolated in the enclosed environment with a gas, the gas is mostly, generally, substantially, essentially, or entirely air, and the method comprises measuring the amount of argon in the air to determine the amount of air in the enclosed environment.

### Aspect 49

The method of any one or more of the preceding applicable aspects, wherein the at least one hydrogen proxy comprises an ammonia proxy.

### Aspect 50

The method of ASPECT 49, wherein the ammonia proxy comprises nitrogen, and wherein the method comprises using the quantity of air in the enclosed environment to determine the amount of ammonia produced from hydrogen in the sample prior to isolating the sample.

### Aspect 51

The method of any one or more of the preceding applicable aspects, wherein the at least one hydrogen proxy comprises a water proxy.

### Aspect 52

The method of ASPECT 51, wherein the sample is isolated in the enclosed environment with a gas, the gas is air, and the method comprises measuring the amount of argon in the air to determine the amount of air in the enclosed environment; the water proxy is oxygen; and wherein the method comprises using the quantity of air in the enclosed environment to determine the amount of water produced from hydrogen in the sample prior to isolating the sample.

### Aspect 53

The method of any one or more of the preceding applicable aspects, wherein the at least one hydrogen proxy comprises ammonia or an ammonia proxy and water or a water proxy, and the quantity of hydrogen in the material is determined by adding an amount of hydrogen lost to ammonia production as determined at least in part by the measurement of the ammonia or the ammonia proxy; an amount of hydrogen lost to water production as determined at least in part by the measurement of the water or the water proxy; and the hydrogen directly measured in the material.

### Aspect 54

The method of any one or more of the preceding applicable aspects wherein the sample is isolated in the enclosed environment with a gas, the gas is air, and the method comprises measuring the amount of argon in the air to determine the amount of air in the enclosed environment.

### Aspect 55

The method of any one or more of the preceding applicable aspects, wherein the method comprises removing water from the material/sample, the easily released volatile substances, or both, prior to or concurrently with collecting the portion of the extracted easily released volatile substances.

### Aspect 56

The method of ASPECT 55, wherein the removal of water from the sample, the easily released volatile substances, or both, is performed by subjecting the easily released volatile substances to a media that selectively traps a portion of the easily released volatile substances that (1) comprises water, (2) does not comprise all of the very small volatile substances in the easily released volatile substances, and (3) does not comprise at least one hydrogen proxy.

### Aspect 57

The method of ASPECT 55 or Aspect 56, wherein the removal of water from the sample is performed by contacting the easily released volatile substances with a cryogenic trap such that the trapped portion of the easily released volatile substances condenses to the cryogenic trap and the collected portion of the easily released volatile substances comprising the at least one hydrogen proxy does not condense to the cryogenic trap.

### Aspect 58

The method of ASPECT 55, wherein the removal of water from the sample is performed by contacting the sample, the easily released volatile substances, or both, with a desiccant (e.g., calcium, gypsum, or a combination thereof) or with an equivalently or better means for water removal.

### Aspect 59

The method of any one or more of the preceding applicable aspects, wherein the determination of the amount of hydrogen comprises using an atom compound presence factor to assess the amount of hydrogen originally contained in the sample.

### Aspect 60

The method of any one or more of the preceding applicable aspects, wherein the method comprises collecting/obtaining (uncontradicted, such terms/elements being SWOSB in this or any other aspect of the technology described herein) multiple samples from different parts of a geologic unit and separately subjecting each of the samples to the other steps of the method to generate a map of hydrogen amounts present in the different parts of the geologic unit. Here, the acronym “ARTA” means “also referred to as,” and the acronym “SWOSB” means “substitutable with or supplemented by.”

### Aspect 61

The method of ASPECT 23, wherein the multiple samples comprise a plurality of one or more drill cuttings, mud sample(s), core sample(s), or a combination of any or all thereof.

### Aspect 62

The method of any one of any one or more of the preceding applicable aspects, wherein the method comprises comparing the quantity of hydrogen measured in the one or more easily released volatile substances to the quantity of helium measured in the one or more easily released volatile substances.

### Aspect 63

The method of any one or more of the preceding applicable aspects, wherein the method comprises analyzing a plurality of samples from different parts of a geologic unit, determining the amount of hydrogen and the amount of helium in the plurality of samples, and identifying one or more areas of high hydrogen content; one or more areas of high helium content; one or more areas of high hydrogen content and low helium content; one or more areas of high helium content and low hydrogen content; or any combination thereof in the geologic unit.

### Aspect 64

The method of any one or more of the preceding applicable aspects, wherein the method comprises subjecting one or more samples/material to different volatile extraction conditions, wherein one of the different volatile extraction conditions results in the extraction of a significantly greater amount of hydrogen than helium and a different one of the different volatile extraction conditions results in extraction of a detectably, sizably, or significantly greater amount of helium than hydrogen (uncontradicted, each such term in any aspect here is SWOSB the other terms (e.g., detectably is SWOSB significantly and vice versa)).

### Aspect 65

The method of any one or more of the preceding applicable aspects, wherein the application of one or more gentle vacuum equivalent forces comprises subjecting the sample to at least two separate applications of gentle vacuum pressure, the at least two separate applications of gentle vacuum pressure comprising a first vacuum having a first pressure applied for a first time period and a second vacuum having either the first pressure or a pressure that is substantially the same pressure as the first pressure applied for a second period, wherein the second period is substantially longer than the first period, and, wherein the first vacuum and the second vacuum extract different amounts of hydrogen from the sample.

### Aspect 66

The method of ASPECT 65, wherein the at least two separate applications of gentle vacuum pressure further comprise a third vacuum having a second pressure applied for a third period and a fourth vacuum having either the second pressure or a that is substantially the same pressure as the second pressure applied for a fourth period, wherein the fourth period is substantially longer than the third period, and, wherein at least one of the first vacuum, second vacuum, third vacuum, and fourth vacuum extracts different amounts of hydrogen from the sample.

### Aspect 67

A method for evaluating the hydrogen generation capacity of a material comprising
(1) obtaining a solid or semisolid mineral aggregate material,
(2) contacting the material with an aqueous liquid under conditions that will result in hydrogen generation if the material is an effective hydrogen-generating material, to form a water-treated material,
(3) subjecting the water-treated material to one or more gentle vacuum equivalent forces that extract one or more easily released volatile substances from the sample, if present, the one or more easily released volatile substances comprising hydrogen, at least one hydrogen proxy, or both,
(4) collecting at least a portion of the extracted easily released volatile substances,
(5) measuring the hydrogen content, hydrogen proxy content, or both the hydrogen and hydrogen proxy content of the portion in the portion, and
(6) evaluating the ability of the material to generate hydrogen from the measurement or measurements obtained from step (5).

### Aspect 68

The method of ASPECT 67, wherein the material is a sample of a geologic material obtained from a geologic unit.

### Aspect 69

The method of ASPECT 68, wherein the material comprises a rock material comprising ferrous oxide.

### Aspect 70

The method of ASPECT 69, wherein the method further comprises (1) obtaining a plurality of compositionally similar samples of the material and (b) contacting each sample of the plurality of compositionally similar samples with an aqueous substance selected from at least two different aqueous substances, wherein the at least two different aqueous substances differ in one or more physiochemical properties, and wherein the method is used to further evaluate the impact of using the different aqueous substances on the generation of hydrogen from the material.

### Aspect 71

The method of ASPECT 70, wherein the at least two different aqueous substances differ in salinity, dissolved solids, pH, Eh (oxidation-reduction potential), or a combination of any or all thereof.

### Aspect 72

The method of ASPECT 69, wherein the method further comprises (1) obtaining a plurality of compositionally similar samples of the material and (b) contacting each sample of the plurality of compositionally similar samples with an aqueous substance under different environmental conditions, and wherein the method is used to further evaluate the impact of the different environmental conditions on the generation of hydrogen from the material.

### Aspect 73

The method of ASPECT 72, wherein the different conditions comprise application of different mechanical stresses, different temperature conditions, different pressure conditions, or a combination of any or all thereof.

### Aspect 74

The method of ASPECT 69, wherein the method further comprises evaluating the material's ability to efficiently sequester carbon, sulfur, or both.

### Aspect 75

The method of ASPECT 74, wherein the evaluation of the material's ability to effectively sequester carbon, sulfur, or both comprises application of x-ray diffraction (XRD), x-ray fluorescence (XRF), or equivalent means for identifying material chemistry, and using the results thereof to compare against empirical data in evaluating the material's ability to effectively sequester carbon, sulfur, or both.

### Aspect 76

A method of generating hydrogen from a material comprising (1) contacting a material identified as an effective hydrogen-generating material using the method of any one or more of aspects ASPECT 1-ASPECT 75 under conditions that are effective for the generation of hydrogen from the material and (2) collecting at least a portion of the generated hydrogen.

### Aspect 77

A device for the rapid analysis of hydrogen in a solid or semisolid mineral aggregate material, comprising
(1) a movable container comprising a plurality of compartments, each compartment occupying a position among a plurality of positions, each compartment (a) comprising a separate inlet, a separate outlet, or both, and (b) being configured to receive an analyzable sample of a solid or semisolid mineral aggregate material at a first position among the plurality of positions and to deliver the sample to one or more other positions of the plurality of positions,
(2) an extraction component positioned in effective proximity to at least one of the plurality of positions and that is configured to selectively apply one or more gentle vacuum equivalent extraction forces to extract one or more aliquots of easily extractable volatile substances from the sample of solid or semisolid mineral aggregate material received by the movable container,
(3) a container movement component that causes the movable container to move and thereby causes the different compartments of the plurality of the compartments to be located at the plurality of positions at different times during operation of the device, the plurality of positions comprising a first position and a second position, wherein a first compartment when located in the first position is configured to receive a first sample delivered to the device at the same time that a second compartment in the second position is oriented to expose a second sample contained therein to the extraction component,
(4) a trap component that selectively removes water from at least a first portion of each of the one or more aliquots,
(5) a collection component that selectively collects a second portion of each of the one or more aliquots,
(6) an analytical component that analyzes the easily released volatile content of each collected second portion of the one or more aliquots and measures the amount of the at least one hydrogen proxy therein, and
(7) an output component for relaying the analysis of the analytical component to a user, a different system, or both, wherein
(8) the device is configured to cause the container movement component to move the movable container to cause the first compartment to be in the first position and the second compartment to be in the second position and to thereafter move each of the first compartment and second compartment to different positions.

### Aspect 78

The device of ASPECT 77, wherein the moveable (or “movable”) container is a rotational container that is configured to gravitationally receive the first sample when delivered to the device and to gravitationally deposit the second sample in a location where the second sample can be subjected to the extraction component.

### Aspect 79

The device of any one or more of the preceding applicable aspects, wherein the device comprises a disposal component that is configured to remove a sample from the device and dispose of such sample after the sample has been exposed to the extraction component, and wherein the disposal component is configured to automatically discard the sample after the extraction component ceases operating on the sample, and, in operation, the disposal of a third sample occurs within 0-60 seconds from the time that the second sample is delivered to second position.

### Aspect 80

The device of ASPECT 79, wherein the disposal of the third sample occurs within 0-15 seconds (e.g., 0-10 seconds, 0-8 seconds, 0-6 seconds, 0-4 seconds, 0.001-5 seconds, 0.01-3.5 seconds, etc.) from the time that the second sample is delivered to the second position.

### Aspect 81

The device of any one or more of the preceding applicable aspects, wherein the trap component comprises a cryogenic trap (e.g., a liquid nitrogen cryogenic trap).

### Aspect 82

The device of ASPECT 81, wherein the cryogenic trap is not associated with any heating element, the device comprises a trapped gas disposal component, or both. Related method aspects of this technology can comprise discarding a cryogenically trapped portion of extracted easily released volatile substances prior to the collection and analysis of the other portion.

### Aspect 83

The device of any one or more of the preceding applicable aspects, wherein (1) the device comprises a flow path between the extraction component and the analytical component and is configured such that each of the one or more aliquots flow from the extraction component to the trap component, the second portion flows from the area of the device comprising the trap component to the collection component, and subsequently to the analytical component and (2) the device comprises a plurality of valves or valve equivalents that permit selective isolation, conditional automatic isolation, or both, of the extraction component, the trap component, the collection component, the analytical component, or a combination of some or all thereof.

### Aspect 84

The device of any one or more of the preceding applicable aspects, wherein the device comprises a selectively operable, conditionally automatically operable, or selectively and conditionally automatically operable vacuum system that is positioned downstream of the analytical component, such that the vacuum system applies a vacuum force that draws an aliquot or portion of the aliquot through the flow path from the direction of the extraction component to the analytical component.

### Aspect 85

The device of any one or more of the preceding applicable aspects, wherein the collection component is adapted such that the second portion comprises at least one hydrogen proxy if present in the sample.

### Aspect 86

A method of reducing hydrogen-material interactions comprising (1) contacting a hydrogen-reactive material with an effective amount of helium, (2) allowing the helium to develop a protective association with the hydrogen-reactive material, and (3) removing excess helium from the material.

### Aspect 87

The method of ASPECT 86, wherein the step of developing a protective association between the helium and the hydrogen-reactive material comprises placing the material in a pressure chamber and exposing the material to a pressure that significantly increases the efficacy of the association, speed of the association, or efficacy and speed of the association of the helium and the hydrogen-reactive material.

### Aspect 88

The method of ASPECT 87, wherein the material comprises iron, steel, or both.

### Aspect 89

A method of evaluating the ability of helium to block hydrogen-material interactions comprising (1) contacting a hydrogen-reactive material with an effective amount of helium, (2) allowing the helium to remain in contact with the hydrogen-reactive material under contact conditions for a contact period, (3) removing excess helium from the material, (4) contacting the helium-treated material with hydrogen, and (5) evaluating the ability of the material to resist/block hydrogen interactions.

### Aspect 90

The method of ASPECT 89, wherein the method comprises testing a number of contact conditions, a number of contact periods, or both, for any material or samples of any material, and selecting the contact conditions, contact periods, or both, which result in a desired amount of hydrogen blocking in the material/sample.

### DETAILED DESCRIPTION OF THE INVENTION

For convenience, both combinations of elements/steps and individual elements/steps may be described in this section of this disclosure. Despite the inclusion of passages focused on specific elements/steps, any aspect, facet, embodiment, or other description of particular step(s) or element(s) can be applied to any general description of the compositions/methods of the invention, or any other recited element(s)/step(s) thereof, which are provided in any part of this disclosure.

### Limited General/Common Concepts and Elements

This disclosure introduces a number of related novel concepts. In an attempt to aid the reader, some concepts are presented in section(s). However, it should be understood that uncontradicted, one, some, most, generally all, substantially all, essentially all, or all element(s) of such concept(s), e.g., step(s) of method(s), material(s), volatile(s), equipment, teaching(s), interpretation(s), and the like can be applied to other related concepts. In aspects, disclosure in any one section, uncontradicted, can, in aspects, be applied to and incorporated with the disclosure in one, some, most, generally all, substantially all, essentially all, or all other section(s).

Typically, herein, a material sample is a measurable amount of a sample that occupies a unit of volume. The sample typically contains mostly solids. Exemplary material samples can be, e.g., core samples, drill cuttings samples, or combinations thereof. However, it may also contain liquids, for example, drilling mud, or gas, e.g., hydrogen or helium or both liquids and gases that are associated with the solid in any amount or volumetric percentage of the total volume of the sample. The sample may be homogeneous or heterogeneous.

Rock materials can be interpreted to encompass disclosure of other solid material sample(s) as suitable for use in the methods described herein. In aspects, a solid material can be, e.g., a rock material. In aspects, a rock material can be drill cuttings. Herein, specific reference to cuttings can be, uncontradicted, may be interpreted to encompass disclosure of other rock material(s) (such as, e.g., core sample(s), or, e.g., other solid materials as suitable for use in methods described herein.

In aspects material samples, such as, e.g., as a geologic material, such as a drill cuttings, can be, e.g., a collection of drill cuttings, such as at least 20, at least 30, at least 50, at least 75, or at least 100 cuttings, such as about 25-500, about 50-500, about 75-600, about 80-400, about 75-300, about 50-350, about 40-400, about 40-800, about 60-360, about 100-500, about 100-1000, about 50-1000, about 35-700, or about 35-350 cuttings.

Technology (ies) previously developed and previously patented (in the Prior Smith Patents) provides the capability to analyze volatiles in drill cuttings, core, or any other solids or fluids that are hermetically sealed at the well as soon as possible to when the cuttings or other solids or fluids of interest arrive at the surface on a drill rig.

Some materials, e.g., geologic materials, e.g., drill cuttings, core samples, other solid materials, or drilling muds, contain hydrogen. Upon sealing in the sample container, hydrogen evolving from the geologic materials can react with the nitrogen in the air to make ammonia, and hydrogen evolved from the sample can also react with oxygen to make water. In certain aspects, materials, e.g., material samples, can be, e.g., drilling muds.

In aspects, technology(ies) herein describe the differential storage of hydrogen and helium, identifying that hydrogen and helium may be differentially stored within different geological locations in close physical proximity to each other. Alternatively, hydrogen and helium may be differentially stored within the same material within the same geologic location. Such differential storage may be accomplished by the hydrogen and helium selectively being associated with different structures within the material, e.g., crystal lattice(s), pores, cracks, fissures, inclusions, liquids, e.g., water or hydrocarbons or the hydrogen and helium may be differentially chemically-associated with different minerals or compounds within a material. As such, in aspects, materials herein comprise hydrogen and helium wherein each of the hydrogen and helium is differentially stored and can be selectively removed from the rock material by application of different extraction states. Extraction states are described elsewhere herein but are characterized as, e.g., an extraction force, a strength of the extraction force, and a time of application of the extraction force.

In aspects, material samples herein are hydrogen-rich. In aspects, a hydrogen-rich material is a material having a concentration of hydrogen that is greater than the concentration of hydrogen in the Earth's atmosphere or having a purity of hydrogen that is greater than the purity of hydrogen in the atmosphere, or both the concentration and purity of the hydrogen are greater than the concentration and purity in the Earth's atmosphere. Such concentration of hydrogen may be greater than previously believed achievable based on the pressure, temperature and volume of cracks, inclusions or liquids in the location. Additionally, the purity of the hydrogen as a percentage of other gases from the same entity may be greater than 50%, greater than 75%, greater than 90%, greater than 95%, greater than 99%, greater than 99.8% or almost 100% when measured at standard temperature, 30 degrees Celsius, and pressure, 1 atmosphere.

In aspects, material samples herein are hydrogen-poor. In aspects, a hydrogen-poor material is a material having a concentration of hydrogen that is less than the concentration of hydrogen in the Earth's atmosphere or having a purity of hydrogen that is less than the purity of hydrogen in the atmosphere or both the concentration and purity of the hydrogen are less than the concentration and purity in the Earth's atmosphere.

Typically herein, solid or semi-solid material samples, e.g., cuttings, (or, e.g., fluid sample(s) as applicable) can be hermetically sealed, usually promptly following collection, e.g., within less than about one day, less than about 4 hours, less than about 1 hour, less than about 20 minutes, less than about 10 minutes, less than about 5 minutes, less than about 1 minute, or less than 15 seconds, to avoid loss of compounds.

Cuttings, core samples, other solids or fluids, each sealed at the well or borehole, for example, can be hermetically sealed in specially designed sample containers, with open head space above the cuttings, core samples, other solids or fluids in the sealed container. The open head space above the cuttings, core samples, other solids, or fluids allows the introduction of a needle through a sealing cap to extract volatiles from the cuttings, core samples, other solids, or fluids for analyses using our previously patented base technology. When a cuttings sample, core samples, other solids or fluids are hermetically sealed in a sample tube, the headspace normally contains ambient air. However, the headspace (head space) may optionally be purged with an inert heavier-than-air gas, such as argon, krypton, or xenon, prior to the sample being sealed hermetically.

In certain aspects, The method of any one or more or all of the preceding aspects, wherein the method comprises applying at least two different forces (resulting in measurably different substances) to generate two different analyzed aliquots for the materials, such as an aliquot produced by applying a pressure of about 20 millibars and a second aliquot produced by applying a pressure of about 2 millibars, each to the same material(s)/sample(s), e.g., in accordance with the teachings of the Prior Smith Patents.

Volatiles referred to herein are gases that can be removed, e.g., selectively removed by application of one or more extraction forces, from material samples. Volatiles may exist as gases residing within the material sample at standard atmospheric pressure and temperature. Volatiles may exist as liquids or solids at standard atmospheric temperature and pressure, but can be converted to a gas by application of a vacuum to the material sample or by application of increased temperature to the material sample or both application of a vacuum and increased temperature. On occasions, volatiles may be created by both an increase in temperature and increase in pressure applied to a material sample. In the present invention, volatiles may be extracted from the material sample to be eliminated, such as water vapor or certain hydrocarbons, or to be analyzed. Volatile(s) can be condensable or non-condensable.

According to aspects, volatile(s) herein are characterized as easily released volatile substances. In aspects, easily released volatile substances are substances capable of being extracted at gentle vacuum extraction pressure(s). Easily released volatile substances do not require any specific gentle vacuum pressure for their release.

In aspects, the results of analyses herein, alone or in combination with other results from one or more additional methods, e.g., gas chromatography, flame ionization detection, Raman spectrometry, capacitance manometry, material strength or a combination of any two or more of such methods can provide information about whether a material or a location within the Earth contains a target resource, e.g., hydrogen, and, e.g., if so, how much hydrogen and at what level of purity. In aspects, the results of such analyses can be used to determine whether or not hydrogen can be produced, extracted, in a cost-effective manner to provide an energy source that does not result in harmful waste products, its only waste product being water.

In aspects, a material/sample is isolated from potentially interfering elements (e.g., by placing the material in a container), but in a manner that provides for volatile substances associated with the material to react in the isolated environment (e.g., by placing sample(s) in a container that contains “headspace” or an open volume/void space that can contain volatile substances and associated gas (e.g., atmospheric air from the environment) permitting any reactive volatile substances, particularly hydrogen compounds, to chemically react with other reactive compounds if present, e.g., oxygen and nitrogen present in ambient atmospheric air). In aspects, the materials/sample or the headspace/open volume/void space or both the materials/sample and the headspace/open volume/void space are isolated from interfering elements by a sealing component in direct connection with an opening in the container. In aspects, the sealing component is a plug, cap, stopper, or similar component. In other aspects, the sealing component is achieved by the direct connection with an opening in the container and a device/system where the analysis is to be performed. The method comprises the collection of gas or one or more volatile substance(s) from the isolated environment/container and an analysis thereof. The analysis can comprise a comparison of the gas or one or more volatile substances to another gas/other gases, e.g., the atmospheric air or one or more volatile substances. The analysis can comprise analyzing a change in one or more substances in the gas.

### Quantification of Hydrogen in Materials

### Generally

According to aspects, technology(ies) herein are directed to the quantification of hydrogen in material(s), such as, e.g., geologic material(s), such as, e.g., subterranean geologic material(s). Geologic hydrogen is an important part of the ongoing global energy transition and, e.g., is relevant to the reduction of greenhouse gases. The accurate estimation of total original hydrogen in geologic materials is of great importance as it relates to the estimation of the size of a location of geologic hydrogen resource(s) in the subsurface. Accordingly, while the quantification of hydrogen in material(s) is relevant to any hydrogen-containing material(s), method(s) of hydrogen quantification provided herein, and, e.g., application of the same, may be particularly useful in geological resource-related endeavors.

In aspects technology(ies) herein provide sufficiently accurate quantification of hydrogen in material(s), by way of effective analysis of sample(s) thereof, such that technology(ies) herein provide actionable data related to the hydrogen content of analyzed material(s). In aspects, actionable data include data relevant to the identification of hydrogen-rich location(s), hydrogen generation, hydrogen production, hydrogen quantification, and hydrogen purity. In specific aspects, this disclosure provides method(s) for the quantification of hydrogen in geologic material(s) comprising consideration of at least one or more substance(s) in addition to hydrogen itself, and which provide detectably or significantly more accurate hydrogen quantification result(s) than the direct measurement of hydrogen alone. In specific aspects, this disclosure provides method(s) for the quantification of hydrogen in geologic material(s) comprising consideration of at least one or more substance(s) in addition to hydrogen itself and which provide data for use in directing resource production-related endeavor(s), such as, e.g., hydrogen production-related endeavor(s).

In one specific aspect, technology(ies) herein provide method(s) of identifying the amount of hydrogen present in a material (e.g., a sample thereof), the amount of hydrogen identified being the amount of hydrogen present in the material sample at the time of its collection. Herein, uncontradicted, use of the word “sample(s)” or “material” or “material sample(s)” can be used interchangeably, such as, e.g., when discussing method(s) comprising analysis of “samples” of a material or “material sample(s)”, such method(s) can be also or alternatively applied to method(s) comprising analysis of a “material” directly rather than a “sample” thereof.

In aspects, the quantity of hydrogen identified by method(s) herein sufficiently represents the true amount of hydrogen in a material such that the quantitation of hydrogen by method(s) herein can be relied upon in hydrogen-related endeavor(s) such as, e.g., hydrogen exploration, location, generation, and production.

In aspects, method(s) comprise sealing material sample(s) upon their collection; applying rock volatile stratigraphy method(s) to the material sample(s) comprising the measurement of a plurality of substances released from the material sample wherein the plurality of substances comprises one or more of hydrogen, oxygen, argon, nitrogen, ammonia, or water; and performing one or more calculation(s) utilizing the results of the measurement of one or more non-hydrogen substances, wherein the result(s) of calculation(s) can be used in quantifying the hydrogen whereby the accuracy of the quantitation of hydrogen is DOS increased compared to the quantitation of hydrogen obtained when such calculation(s) are not applied.

In certain respects, as discussed in greater detail elsewhere herein, the quantification of hydrogen of this disclosure comprises, e.g., directly measuring the amount of hydrogen associated with a sample as analyzed hydrogen, in combination with calculated amount(s) of hydrogen lost due to ammonia production, hydrogen lost due to water production, or both.

### Material(s) & Sample Collection

According to aspect(s), the quantification of hydrogen described herein can be applied to any hydrogen-containing material. In certain aspects, the hydrogen-containing material is a material in which hydrogen at least detectably or significantly present with or associated with the material under typical circumstances but which may DOS separate from, e.g., leave, the material upon contact with atmospheric air. In certain respects, method(s) herein comprise analysis of such material(s) directly, while in common aspects, method(s) herein comprise analysis of sample(s) of such material(s). In aspects, materials which can be the target of hydrogen quantification as described herein are material(s) which are typically, e.g., which are at least mostly, at least generally, at least substantially, at least essentially, or is protected from detectable or significant contact with atmospheric air under typical circumstances.

In aspects, the material is a sample of a geologic material, e.g., a subterranean geologic material, e.g., a rock material (such as a subterranean rock material). In certain aspects, materials are associated with geologic resource exploration, such as, e.g., drilling operation(s). In aspects, material(s) are core sample(s), drill cutting(s), drilling mud(s), or combination(s) thereof.

In aspects, a material sample is a material comprising a solid, a liquid, a gas, or a combination of any two or more thereof. In aspects, the material sample comprises a liquid that has been in contact with a subterranean surface. In aspects, the liquid is a drilling mud. In aspects, the material sample comprises a gas associated with the material sample. According to aspects, the gas is chemically associated with the material sample. In aspects, the gas comprises hydrogen, helium, or both hydrogen and helium. In aspects, the gas is physically associated with the material sample. In aspects, the gas resides in cracks, pores, crystal lattice, or the like of the material sample. In aspects, gas(es) analyzed for this or other embodiment(s) of technology(ies) disclosed herein comprise hydrogen, helium, or both.

In certain aspects, material(s) herein can comprise water. In aspects, some, most, generally all, substantially all, essentially all, or all detectable or significant amount(s) of water present in material(s) is removed prior to the analysis of substance(s) described herein associated with hydrogen quantitation. According to aspects, hydrogen quantitation herein comprises the analysis of a water-free or substantially water-free or essentially water-free non-condensable gas (e.g., that does not condense to a cryogenic trap when applying methods according to the Prior Smith Patents) that is analyzable by providing that water-free gas directly into an analyzer, such as, for example, a mass spectrometer.

According to aspects, method(s) of determining the amount of hydrogen in a material described herein is applied to a collection of multiple samples from different parts of a geologic unit and separately subjecting each of the samples to step(s) of the method(s) to generate a map of hydrogen amounts present in the different parts of the geologic unit. In aspects, the multiple samples comprise a plurality of a drill cutting, a mud sample, a core sample, or a combination of any or all thereof.

### Sealed Samples

It is well appreciated that hydrogen is a very light, volatile element and is both at risk of simply escaping a material or also or alternatively reacting with one or more other element(s) or compound(s) to form new compound(s). As such, in aspects, quantitation of hydrogen herein comprises sealing sample(s) for hydrogen quantitation in an airtight container, e.g., hermetically sealing, sample(s), upon their collection and prior to their analysis.

If the material sample, e.g., a geologic material sample, subject to hydrogen quantitation is not hermetically sealed upon collection, e.g., is not sealed at the well from which it is collected, estimation of the amounts of hydrogen lost from the geologic material sample through reactions with nitrogen and oxygen in air that produces ammonia and water is somewhat futile and thus inaccurate hydrogen quantitation results, as ammonia and water are volatile substances and as such will to a great extent dissipate away from the unsealed geologic material sample. Further, many sources of water exist in nature and many sources of water are utilized or are otherwise involved with geologic material handling, making direct water analyses on unsealed geologic materials of little or no use in estimating original hydrogen in geologic material samples at the time of their capture. Still further, hydrogen is extremely volatile and will dissipate away from unsealed geologic material samples.

In aspects, sample(s) upon collection are sealed in suitable airtight container(s). In aspects, container(s) are any suitable hermetically sealed containers. In aspects, container(s) can comprise, one, some, most, generally all, substantially all, essentially all, all, or can be the containers described in prior U.S. patent application Ser. No. 18/433,409 to Smith (the inventor of the present Application), and patent application(s) related thereto. According to aspects, a suitable container comprises one or more components that are collapsible. In aspects the container is not chemically reactive with one or more gases, such as, e.g., hydrogen, oxygen, ammonia, nitrogen, or argon. In aspects, the container comprises one or more rubber materials, one or more plastic materials, one or more metal materials, or a combination of any or all thereof. In certain aspects, a suitable container can be a bag. Also or alternatively, in aspects a container comprises copper alloys, aluminum, or aluminum alloys such as, e.g., brass. In aspects, the container is a hollow cylinder, wherein one end of the hollow cylinder is enclosed prior to the addition of the material sample. In aspects, sealing of the container affixes a sealing component onto the container. In aspects, a sealing component is not reactive with one or more gases such as, e.g., hydrogen, oxygen, argon, nitrogen, and ammonia

In one example, suitable sample container(s) comprise, for example, a hollow brass cylinder. In aspects, the hollow brass cylinder is sealed at one end before the sample or material is introduced into the cylinder. In aspects, sealing is achieved by a sealing component, for example, a silicone plug. In aspects container(s) and, e.g., sealing component(s) thereof are not chemically reactive with one or more substances, e.g., hydrogen or other volatile substance(s) discussed herein, that contact the container and sealing component. In certain facets, the collection of the material sample, the sealing of the material sample, or both the collection and the sealing of the material sample does/do not require human intervention. In particular aspects, the collection of the material sample, the sealing of the material sample, or the collection and the sealing of the material sample is/are executed by a programmable system.

According to aspects, measuring the amount of hydrogen in a material as provided by method(s) here comprises isolating the sample by placing the sample in an enclosed environment prior to subjecting the sample to the one or more extraction forces, such as, e.g., gentle vacuum equivalent forces, such as, e.g., a gentle vacuum pressure. In aspects, the sample is isolated in the enclosed environment with a gas, wherein, e.g., the gas consists essentially of air.

In aspects, collected sample(s) are placed in sample containers, and the analysis of substance(s) described herein is performed without the removal of the material sample from the container(s). In aspects, volatile substance(s) extracted from a material sample is the only portion of a material sample that exits a sample container during analysis once the sample is collected therein.

According to aspects, sample(s) are sealed within container(s) comprising a detectable or significant amount of atmospheric air, but that isolate the material sample from atmospheric air outside of the container. In aspects, the material/sample is isolated from potentially interfering elements (e.g., by placing the material in a container), but in a manner that provides for volatile substances associated with the material to react in the isolated environment (e.g., by placing sample(s) in a container that contains “headspace” or an open volume/void space that can contain volatile substances and associated gas (e.g., atmospheric air from the environment) permitting any reactive volatile substances, particularly hydrogen compounds, to chemically react with other reactive compounds if present, e.g., oxygen and nitrogen present in ambient atmospheric air).

According to aspects, an amount of headspace present in container(s) represents less than about 10%, such as, e.g., <~9%, <~8%, <~7%, <~6%, <~5%, <~4%, <~3%, <~2%, <~1%, or, e.g., ~0% of the total volume of the container. In certain respects, a headspace present in the container is of sufficient volume to allow the introduction of a needle through a/the sealing component of the container in order to capture one or more of the one or more substances for analysis using a rock volatile stratigraphic method.

In aspects, the materials/sample or the headspace/open volume/void space or both the materials/sample and the headspace/open volume/void space are isolated from interfering elements by a sealing component in direct connection with an opening in the container. In aspects, the sealing component is a plug, cap, stopper, or similar component. In other aspects, the sealing component is achieved by the direct connection with an opening in the container and a device/system where the analysis is to be performed.

Discussion of elements related to atmospheric air and its presence in headspace within container(s) is further provided elsewhere herein.

In respects, hydrogen quantitation method(s) described herein aim to obtain from the testing of a material sample the most accurate quantitation or estimation of the quantity (ies) of hydrogen present in the material in its native state, e.g., in a subterranean location within a geologic unit, as possible. The amount of hydrogen analyzed in a geologic material sample can be appreciably lower than the initial amounts of hydrogen present in the geologic material sample when collected at the Earth's surface, e.g., at the surface of the drilling rig (and, similarly, can be appreciably lower than the actual amount(s) of hydrogen present in the geologic material itself in its original location) by way of hydrogen associated with the material sample reacting with nitrogen in air to make ammonia, and reacting with oxygen in air to make water.

In aspects, the composition of the gases, type and amount, when the sample is collected in the container, may be different from those gases in the atmospheric air which were originally present in the headspace. Such different gases or amounts thereof may be related to hydrogen gas, a reduction in the amount of oxygen gas or nitrogen gas, or an increase in the amount of ammonia or water vapor. If changes occur in the type or amount of gases, such changes can be attributed to hydrogen gas in the sample being released into the headspace and reacting with oxygen to create water or reacting with nitrogen to create ammonia or both reacting with oxygen and nitrogen.

Material sample(s) sealed immediately upon collection, e.g., “sealed at the well” geologic material samples, do not detectably or significantly suffer from extreme volatiles loss and are therefore useful in measuring hydrogen or hydrogen proxies, such as but not limited to nitrogen, ammonia, oxygen, and water. In certain aspects, unsealed geologic material samples are of limited if any use for evaluating subsurface geologic hydrogen resources because volatile components, e.g., hydrogen, helium, argon, nitrogen, ammonia, and oxygen, can dissipate away from the geologic material sample. Hydrogen can in aspects remain in tight cracks and pores in material samples, such as, e.g., the tight cracks and pores of sealed at the well drill cuttings samples; however, reaction products of hydrogen and air, such as ammonia and water (and corresponding decreases in nitrogen and oxygen), and, further, argon present in the air of a sample container headspace, remain sealed in the sealed geologic material sample container.

Accordingly, the estimation of hydrogen amount(s) disclosed herein, when, e.g., applied to geologic materials initially captured and sealed at the well as soon as possible once the geologic materials are at the Earth's surface, comprises the analyses of hydrogen plus the analysis of compounds created by the consumption of hydrogen reacting with another substance or the decrease in the amount of one or more substance(s) from atmospheric air sealed in the container that react with hydrogen from the geologic material. In aspects, method(s) of hydrogen quantitation disclosed herein do not consider directly measured hydrogen alone, but comprise an analysis of the reaction products ammonia and water (and/or the corresponding decrease in nitrogen and oxygen respectively) from the exact same physically sealed at the well geologic material sample, and together such data reflect the inventive quantitation of hydrogen as described herein.

In aspects, sealing samples to be subjected to hydrogen quantitation comprises storing the collected sample(s) in airtight container(s) as quickly upon their collection as possible, such as, e.g., within at least about 10 minutes after their collection, e.g., within at least about 9 minutes, ~8 minutes, ~7 minutes, ~6 minutes, ~5 minutes, ~4 minutes, ~3 minutes, ~2 minutes, ~1 minute, ~45 seconds, ~30 seconds, ~15 seconds, ~10 seconds, or within ~5 seconds or ~2 seconds from their collection. In aspects, collection occurs when sample(s) are first exposed to atmospheric air, such as, e.g., occurring when samples first reach the Earth's surface from a subterranean/subsurface atmospheric-air-protected environment. As such, in aspects, sealing can occur within such time periods of the sample(s)'s first exposure to atmospheric air.

Accordingly, in aspects, method(s) of hydrogen quantification described herein comprise(s) containing material(s)/sample(s) subject to hydrogen quantitation in sealed container(s) upon their collection and prior to performing most or all of the method (e.g., prior to the application of force to release volatile substances from the material(s)/sample(s)).

### Purging

As provided herein, interaction between hydrogen-containing material sample(s) sealed in airtight container(s) and with atmospheric air present in the sealed headspace of such container(s) can yield hydrogen loss. The amount of hydrogen loss can be DOS minimized, mostly eliminated, or eliminated by the purging of air out of the sample container(s) before sealing, or, e.g., through seal(s) of the sample container(s). In aspects, purging can comprise use of hydrogen-inert gas(es) such as, e.g., krypton, argon, or, e.g., xenon. In aspects, argon can be used. Minimizing the headspace inside of sample containers can DOS reduce the nitrogen and oxygen available for hydrogen consumption. Stated another way, reducing the amount of nitrogen and oxygen present as component(s) of atmospheric air within sealed containers, e.g., by purging, can DOS reduce the amount of hydrogen (which would otherwise be measured as material-associated hydrogen and directly quantified) consumed in the formation of other compound(s) (such as, e.g., ammonia and water).

As an alternative to purging, reducing the amount of nitrogen and oxygen present in atmospheric air with which hydrogen associated with a material sample can interact can be minimized by maximizing the volume of the material sample within sample container(s). Reducing the amount of nitrogen and oxygen present for material sample hydrogen to interact can also or alternatively be minimized by minimizing the headspace comprising atmospheric air present in such containers. Maximizing the volume of, e.g., drill cuttings samples in sealed sample container(s), minimizing the headspace within container(s), or both, can DOS reduce the percentage of total drill cuttings hydrogen consumed by reaction with nitrogen and oxygen from air in the sealed tubes headspace.

According to aspects, method(s) of hydrogen quantitation can comprise, e.g., purging air from a sample container, reducing or eliminating airspace in a container, maximizing sample volume within a sample container, or combination(s) thereof, as part of the method prior to the analysis of compound(s) or element(s) indicative of hydrogen content of the evaluated material(s).

### Overview of Hydrogen Quantitation: Hydrogen Analysis & Proxy-Supplemented Analysis

### Formula Overview

According to aspects, hydrogen quantitation provided herein comprises the estimation of the amount of hydrogen present in a material sample when sealed at the Earth's surface after collection from a sub-surface location, e.g., a location typically protected from DOS exposure to atmospheric air.

Because of potential losses of hydrogen within sealed tubes discussed herein, the inventive hydrogen estimation/quantification provided herein comprises the analysis and subsequent calculation of hydrogen lost, and adding the amount of hydrogen lost to the amount of still preserved hydrogen. The result of that calculation represents original material, e.g., original drill cuttings hydrogen. Such a value is, in aspects, very valuable for use in the estimation of the size and purity of subsurface hydrogen reservoirs.

To effectively estimate the original hydrogen amounts present in a material sample (and accurately reflect the amount of hydrogen present in the material itself in its original location) measurement of hydrogen alone is supplemented by the analysis of the reaction products of ammonia and water, and the results of the assessment of hydrogen consumed in making those compounds are added to any molecular hydrogen also analyzed in the exact same physical sealed at well cuttings sample. Again, the analysis of all substance(s) discussed herein relative to the inventive method(s) of quantifying hydrogen is conducted within the same container; that is, the same container in which the sample is collected is utilized for the analysis of compound(s) discussed here.

According to aspects, quantitation of hydrogen disclosed herein can comprise use of three values: (1) hydrogen analyzed, (2) hydrogen lost making ammonia upon its interaction with atmospheric air (e.g., atmospheric air sealed with the sample inside of the sample container), and (3) hydrogen lost making water upon its interaction with atmospheric air (e.g., again, atmospheric air sealed with the sample inside of the sample container). According to aspects, such three values are utilized to estimate the amount of original material sample (e.g., drill cuttings) hydrogen (H2) present at the time of capturing the sample(s), e.g., cuttings, at the surface and sealing in the sealed cuttings container.

As an equation, the total original sample (original cuttings) hydrogen in moles is calculated as:

H2_{Original cuttings}=H2_{Analyzed}+(Ammonia_{produced})*1.5+ (Water_{produced})

In aspects, values in such an equation can be obtained using rock volatile stratigraphy system(s) disclosed herein or, e.g., in Prior Smith Patents.

According to certain aspects, a DOS amount of hydrogen present in a subsurface location protected from DOS exposure to atmospheric air can be lost from a sample thereof while the sample is transported from the sampling location to the point of collection, e.g., at the Earth's surface. That is, in aspects, a DOS amount of hydrogen can be lost from a material sample as it travels up a borehole from the point of its collection to the Earth's surface. In aspects, the amount of hydrogen lost increases as the distance the sample must travel between its source and its point of collection, e.g., the Earth's surface. Accordingly, in aspects, calculation(s) provided herein can comprise, e.g., use of a collection factor which accounts for an estimated amount of hydrogen lost based upon, e.g., a number of factor(s), including, e.g., but not limited to, the distance a sample travels between its source location and its point of collection, the time between when a sample is first taken to when it is hermetically sealed, the environment to which the sample is exposed between the time the sample is first taken to when it is collected/hermetically sealed, and the like.

### Volatile(s)

According to aspects, quantitation of hydrogen as disclosed herein comprises the analysis of volatile substance(s), including, e.g., hydrogen and, e.g., at least one other volatile compound, as described herein.

The estimation of hydrogen amounts when cuttings are initially captured and sealed at the well as soon as possible, once the cuttings are at the surface, requires the analyses of hydrogen plus the consideration, e.g., the analysis, of compounds created by the consumption of hydrogen reacting with another compound.

In certain aspects, volatile(s) utilized in determining the amount of hydrogen present in a material sample are volatiles that are easily extracted volatile substances. In aspects, easily extracted volatile substances are substances that can be captured or otherwise isolated by way of application of a gentle vacuum force or a gentle vacuum force equivalent. In aspects, typically a sufficiently gentle vacuum force herein is a vacuum force which is no stronger than about 2 mbar, such as, e.g., no stronger than about 4 mbar, ~6 mbar, ~8 mbar, ~10 mbar, ~12 mbar, ~14 mbar, ~16 mbar, ~18 mbar, ~20 mbar, ~22 mbar, or, e.g., ~24 mbar. In aspects, one or more volatile substances are removed prior to analysis, e.g., one or more easily extracted volatile substances are removed prior to analysis.

### Hydrogen-Direct Measurement

In aspects, hydrogen quantitation method(s) provided herein comprise the direct measurement of hydrogen. As previously provided, in aspects all analysis of hydrogen quantitation method(s) provided herein are performed on the same sample within the same collection container. Thus, the quantitation of hydrogen can be performed by any suitable method or technology, so long as the method or technology is capable of analyzing each of the additional substance(s) relevant to the hydrogen quantitation method(s) described herein. In one aspect, hydrogen is directly quantified using mass spectrometry.

In facets, the amount of hydrogen directly analyzed in a material sample, e.g., a cuttings sample, can be appreciably lower than initial (original) amounts of hydrogen present in the cuttings sample (e.g., amounts of hydrogen present in the material sample at the time of its collection and more accurately reflecting the hydrogen content of the source material from which the sample was taken).

### Hydrogen Proxy(ies)

As discussed, hydrogen is highly subject to environmental loss, e.g., by dissipation or, e.g., by interaction with other substances, e.g., other substances in atmospheric air, and can be consumed by the formation of new compounds such as, e.g., ammonia and water. Accordingly, method(s) of hydrogen quantitation disclosed herein comprise the assessment of one or more other compounds which DOS reflect such lost hydrogen.

In certain respects, the amount of hydrogen consumption in some sealed geologic material samples could be so high as to totally or nearly totally eliminate all the hydrogen in a sealed at well geologic material sample. Even in samples still containing appreciable molecular hydrogen itself, much or even most of the original geologic material hydrogen may be lost by the time the sample undergoes analysis, having been consumed in the production of ammonia and water in the sealed sample tube.

Due to these potential losses of hydrogen within sealed tubes, in one aspect, a preferred manner in which an estimate of original geologic material hydrogen can be made is through analyses and subsequent calculation of hydrogen lost, and the addition of the amount of hydrogen lost to the amount of still preserved hydrogen. The result of such calculation represents original geologic material hydrogen, a very important number for the estimation of the size and purity of subsurface hydrogen reservoirs.

In aspects, method(s) of evaluating/determining hydrogen quantity comprises analyzing one or more “proxy compounds” that are generated by the reaction with hydrogen in the isolated environment/container. In aspects, calculations are performed to convert the presence or absence of proxy compounds into an estimation of the amount of hydrogen in the sample at the time of its collection into a sample container.

According to aspects, hydrogen quantitation provided herein comprises analyzing the amount of hydrogen in a sample and using both the amount of hydrogen and the amount of the at least one hydrogen proxy to quantify the hydrogen in the material. In aspects, the at least one hydrogen proxy comprises ammonia, an ammonia proxy, or both. In aspects, the at least one hydrogen proxy is nitrogen. In aspects, the at least one hydrogen proxy is a water proxy. In aspects, the at least one hydrogen proxy comprises oxygen.

According to particular embodiments, use of hydrogen proxy (ies) is dependent upon knowing the amount of air present in the sealed sample container. In aspects, samples are isolated in the enclosed environment with a gas, wherein the gas is air, and the method comprises measuring the amount of argon in the air to determine the amount of air in the enclosed environment.

In certain respects, method(s) of hydrogen quantitation comprises/comprise removing water from the sample prior to or concurrently with collecting a portion of extracted easily extracted volatile substances comprising the at least one hydrogen proxy.

In certain further aspects, the determination of the amount of hydrogen comprises using an atom compound presence factor to assess the amount of hydrogen originally contained in the sample.

### Nitrogen/Ammonia

In one aspect, technology(ies) provided herein are applicable for use in resource exploration, and, accordingly, method(s) herein can be used in resource exploration, e.g., the quantification of hydrogen stored in a hydrogen reservoir. As described, upon exposure to air, or, e.g., upon sampling, material(s) can lose a DOS amount of hydrogen, e.g., in one aspect by dissipation or, e.g., by interaction with one or more other substances. In one aspect, the loss of hydrogen from, e.g., drill cuttings sample(s) could, at least in part, be supplemented by further measuring the amount of hydrogen present in the mud(s) associated with the drill cutting(s). In aspects, the analysis of both cuttings and muds provides DOS improved accuracy of the estimated quantity of hydrogen present in the source material.

### Ammonia

In another aspect, upon sealing a material sample, e.g., a drill cuttings sample, in a sample container, a DOS amount of hydrogen evolving from the sample, e.g., cuttings sample, can react with the nitrogen in air present in the sealed container to generate ammonia. Such a loss of material hydrogen to ammonia production, left unaddressed, reduces the accuracy of material hydrogen quantification.

Ammonia is not a common component of sub-surface volatiles. Accordingly, the analyses of ammonia in sealed cuttings samples mostly, generally, substantially, or at least essentially reflect the analyses of the ammonia formed in the sample container from the reaction of hydrogen evolved from the samples, e.g., cuttings, with nitrogen in the air in the sealed sample container headspace.

According to certain aspects, ammonia is measured directly as a hydrogen proxy. In aspects, measurement of ammonia can be used to assess and account for material hydrogen lost, e.g., consumed by, the production of ammonia. 1.5 molecules of H2 are consumed for each molecule of NH3 produced. As the amount of ammonia in samples derived from the Earth is negligible, any ammonia generated and measured can be directly equated with an amount of hydrogen consumed to produce such ammonia.

Ammonia can, in aspects, be difficult to analyze using particular technology(ies), such as, e.g., mass spectrometry, because the typically small amount(s) of ammonia in a material shares its main analytical peaks with the almost always extremely abundant amounts of water. The main mass spectrometer peaks for ammonia are Mass/charge values 17 (NH3+), 16 (NH2+), 15 (NH+), and 14 (N+). Of these peaks, 15 is difficult in the presence of methane from methane's contribution on 15 from (CH3+). Ammonia on the 14 peak is difficult to analyze because of the very large N+peak on 14 from N2 in air, and, further, if methane is present, ammonia also competes with the methane CH2+ peak on 14. The peak on 16 is also difficult due to O+ on 16 from CO2, water, and O2 in the air. The only common ammonia interference on the 17 peak is from water (H₂O), which is typically present in relatively high amounts, e.g., DOS higher amounts than ammonia, in geologic samples. H2O has its major peak on mass/charge peak 18 for H2O+. However, H2O also has a very major 17 peak (OH+) at about 20% of the intensity as H2O's 18 peak. Accordingly, without the removal of most of the water from the sample, it is difficult to analyze ammonia on the 17 peak.

According to aspects, it is trace ammonia can be analyzed in the presence of large amounts of water by subtracting out the appropriate amount from the 17 peak based on the size of water's 18 peak. In such cases, the amounts of ammonia can, in aspects, be lower than the imprecision inherent in the analyses of the 18 and 17 water peaks. As such, direct ammonia mass spectrometer analyses using the 17 peak for ammonia in the presence of normally abundant water is typically impractical.

Prior Smith Patents reflect that establishing a volume of water-free, non-condensable gas for volatile assessment can be beneficial. In aspects, condensing some, e.g., most or at least a substantial amount of volatiles, including water, on a cryotrap, such as, e.g., a liquid nitrogen trap, can create a volume of water-free non-condensable gas analyzable by providing such water-free gas directly into a mass spectrometer. Ammonia is a component of the non-condensable gas fraction in prior disclosed cryogenic mass spectrometer system(s), systems which, in aspects, are associated with rock volatile stratigraphy method(s) previously disclosed and which are further expanded upon herein. While some background water in the system may still be present, ammonia can be directly analyzed from this water-free gas fraction using mass 17 corrected for any residual water by subtracting the appropriate percentage of the water's main 18 peak, about 20%.

According to certain specific aspects, method(s) of hydrogen quantification provided herein comprise the measurement of ammonia, nitrogen and argon, or each of ammonia, nitrogen, and argon. In certain aspects, method(s) comprise analyzing ammonia generated in a sealed sample container which may be from the reaction of geologic hydrogen gas released from the sample with nitrogen in the air in the headspace in the sealed container. In aspects the assessment of nitrogen comprises consideration of the reduction in an expected amount of nitrogen to be present if no nitrogen is consumed my material hydrogen to form ammonia. In aspects, method(s) comprises measuring the amount of argon associated with a sample, in a sample container (e.g., in the gas/headspace in a container), or both, and optionally using such data to evaluate nitrogen loss from a container. In aspects, method(s) comprise analyzing the amount of ammonia associated with the material/sample/cutting from a water-free gas fraction using mass 17, optionally wherein the method comprises correcting the measurement/analysis for any residual water by subtracting the appropriate percentage of the water's main mass spectrometry mass 18 peak (H2O+).

In aspects, analyzing ammonia directly provides a realization of the amount of hydrogen consumed to make such ammonia. In aspects, such hydrogen lost is in addition to and can be added to analyzed hydrogen still in the sample container to aid in the quantification of material hydrogen.

### Nitrogen+Argon

In aspects, ammonia production in a sample container as described herein can also be estimated by analyzing nitrogen in the sample container and from those data the amount of nitrogen consumed to make ammonia can be determined. In aspects, the amount of air sealed into the sealed cuttings container at the well is approximately the same for all samples. In aspects, simply graphing variation in total nitrogen in a series of sealed cuttings samples can identify samples that have lost nitrogen from the air in the sealed at well cuttings containers from ammonia production. In other aspects, the amount of air trapped into the sealed at well cuttings sample container can be determined using analyses of argon, whereby argon makes up, e.g., about 1% of air. The amount of nitrogen, present in a concentration of about 77% in air, can be compared to the amount of argon analyzed to determine if there has been nitrogen loss from the air sealed with the cuttings at the well caused by ammonia production. Argon is a non-reactive noble gas and is uncommon in the earth's subsurface except in only trace amounts.

Alternatively, one can measure the amount of Nitrogen in the air within the container (identifying the amount of Nitrogen consumed) to estimate the amount of hydrogen that was present when the sample was sealed with ambient air but consumed in the production of ammonia. According to certain aspects, nitrogen is measured directly as a hydrogen proxy. When nitrogen is measured directly, it is accompanied by the direct measurement of argon. The amount of nitrogen in the air (~77%), as provided above, is known. In aspects, the amount of air present in the sample container can be determined, e.g., by further measuring argon, and thus any reduction in the expected amount of nitrogen to be present based upon the amount of air present can be interpreted as having been lost to ammonia production, from which the amount of material hydrogen lost to ammonia production can be determined.

Specifically, for each molecule of nitrogen (N₂) consumed, three molecules of hydrogen (H₂) are consumed to produce 2 molecules of NH3. Thus, if the concentration of N2 in the air is expected to be, e.g., was originally 77% when the sample was sealed in the sample container with such air, and the concentration of nitrogen is determined to be 74% when the sample is analyzed, then a 3% reduction in the concentration of nitrogen by virtue of it reacting with hydrogen in the sample at the time it was sealed is identified. Such a percentage reduction, e.g., 3%, can be multiplied by the volume of ambient air that was sealed in the tube (which can be determined by way of measuring argon) to get the absolute amount of nitrogen consumed.

As with the case for oxygen consumed (discussed below), the amount of nitrogen in the ambient air sealed in the sample container with the material sample can be estimated by measuring the amount of a gas, such as argon, which is present when the sample is analyzed, since the amount of argon in samples derived from the Earth is negligible. Thus, the amount of argon measured can be divided by the typical concentration of argon in ambient air, about 1%, to estimate the volume of ambient air sealed in the tube.

Upon calculating the absolute amount of nitrogen consumed, the amount of hydrogen that was in the sample when sealed but consumed to produce ammonia can be calculated by multiplying the amount of nitrogen consumed by 3 to estimate the amount of hydrogen consumed to form ammonia. As the relationship between the amount of nitrogen consumed in reacting with hydrogen is directly related to the amount of ammonia produced, that is 1 molecule of N2 is consumed to produce 2 molecules of ammonia (NH3), both the amount of ammonia produced and the amount of nitrogen consumed can be determined to generate a more accurate estimation of the amount of material hydrogen consumed to form ammonia.

Notably, it is not necessary to measure both nitrogen consumed and ammonia generated to generate an estimate of hydrogen consumed in the production of ammonia. Estimated hydrogen lost due to ammonia production will, in aspects, e.g., can typically generate a positive estimate of hydrogen present when sealed, but consumed when there is an amount of directly measured hydrogen is present. In certain aspects, such a relationship may not always exist.

Measurement of nitrogen, assessing the reduction in the amount of nitrogen expected to be present based on the known amount of air in the container (as determined, at least in part, by the amount of argon present), provides an indirect assessment of the amount of ammonia produced and, e.g., accordingly, can be used as a proxy to determine how much hydrogen was lost due to ammonia production. In certain respects, accordingly, nitrogen can be referred to as a hydrogen proxy, an ammonia proxy, or both.

### Nitrogen+Argon & Ammonia

In particular aspects, it can be beneficial to measure ammonia directly as well as to measure nitrogen and argon directly to indirectly assess the amount of ammonia produced (as described above). In aspects, having each such hydrogen proxy can aid in validating the other. In particular aspects, ammonia is measured directly as is nitrogen (and argon); and, e.g., if the amount of ammonia measured is more than the amount of nitrogen calculated to have been consumed in the production of ammonia, this demonstrates that ammonia was generated prior to sealing tube, and in such case the directly measured ammonia value can be used in further calculations related to the establishment of the amount of material hydrogen.

The chemical reaction for nitrogen loss for ammonia production by reaction with nitrogen is: N2ₐᵢᵣ+3H2_{cuttings}=2NH3 sealed container

The calculations for hydrogen loss by ammonia production from estimating N2 loss are:

Nitrogenₗₒₛₜ=(77*Argon)−(Nitrogen_{analyzed})

and

NH3_{generated}=(Nitrogenₗₒₛₜ)/2

and

H2 Lost Making Ammonia=(Nitrogenₗₒₛₜ)*3

Alternatively, using analyzed ammonia, the equation for H2 lost is: H2 Lost=(Ammonia_{analyzed})*1.5

The chemical reaction for nitrogen loss for ammonia production by reaction with nitrogen is:

N2ₐᵢᵣ+3H2_{cuttings}→2NH3_{sealed container}

The calculations of hydrogen loss by ammonia production from estimating N2 loss are:

Nitrogen Lost=(77*Argon)−(Nitrogen_{analyzed})

and

NH3_{generated}=(Nitrogenₗₒₛₜ)*2

and

H2 Lost Making Ammonia=(N2ₗₒₛₜ)*3

Alternatively, using analyzed ammonia, the equation for H2 lost is:

H2 Lost=(Ammonia_{analyzed})*1.5

### Oxygen/Water

Upon sealing in the sample tube Hydrogen evolving from the cuttings sample can react with the Nitrogen in the air to make Ammonia, and Hydrogen evolved from the sample can also react with Oxygen to make Water.

### Oxygen+Argon

In aspects, just as nitrogen loss from air sealed in the sample tube can be used to estimate ammonia production from nitrogen reacting with hydrogen, so too can water production be estimated from oxygen loss from the air that is trapped as a head space gas in the sealed at the well cuttings container.

Air is known to contain about 22% oxygen. The amount of air sealed in the sealed-at-well-cuttings containers varies little from sample to sample. As such, in aspects, graphing the amount of oxygen in a series of samples allows the ready identification of such samples that have experienced significant oxygen loss through reaction with cuttings hydrogen to produce water. Like with nitrogen, the analysis of argon, wherein argon is present at a concentration of about 1% in air, can be used to estimate the amount of trapped air in the sealed at well cuttings container, from which an original amount of oxygen in that sample container can be calculated, and, e.g., further, the loss of oxygen can be calculated by subtracting the amount of analyzed oxygen from the amount of oxygen calculated to be present based upon the amount of argon. Such data can be used to determine the amount of material hydrogen lost due to the production of water. The calculation in moles is approximately:

Oxygenₗₒₛₜ=(22*Argon_{air in tube})−(Oxygen_{analyzed})

and

H2O_{generated}=Oxygenₗₒₛₜ*2

and

H2ₗₒₛₜ making water=(O2ₗₒₛₜ)/2

The chemical reaction where O2 and H2 are lost to produce H₂O is

O2ₐᵢᵣ+2H2_{cuttings}→2H2O_{sealed container}

According to aspects, quantitation of hydrogen comprises measuring the amount of oxygen in a sample/container, e.g., in a series of samples or sample containers, e.g., to allow the identification of such samples that have experienced significant oxygen loss through reaction with cuttings hydrogen to produce water.

### Water

In aspects, it is impractical to estimate the amount of hydrogen consumed in a sealed at well cuttings sample container directly using the analysis of water, as, e.g., there is typically a significant amount of water in such samples, e.g., water from subsurface fluid(s), drilling mud(s), etc., and such amount(s) of water can vary unpredictably from sample to sample. This makes the estimation of hydrogen consumed by water production from the analyses of water in the sealed at well cuttings sample challenging. However, just as nitrogen loss from air sealed in the sample tube can be used to estimate ammonia production from nitrogen reacting with hydrogen, so too can water production be estimated from oxygen loss from the air that is trapped as a head space gas in the sealed at the well cuttings container.

In aspects, analyzing the amount of oxygen (and, e.g., argon) to obtain an estimation of the amount of hydrogen consumed to make water can be added to analyzed hydrogen still in the sample container to aid in the quantification of material hydrogen.

In aspects, removal of water from the sample is performed by subjecting the easily extracted volatile substances to a media that selectively traps a portion of the easily extracted volatile substances that (1) comprises water, (2) does not comprise all of the very small volatile substances in the easily extracted volatile substances, and (3) does not comprise, e.g., at least one hydrogen proxy.

In aspects, removal of water from the sample is performed by contacting the easily extracted volatile substances with a cryogenic trap such that the trapped portion of the easily extracted volatile substances condenses to the cryogenic trap and the collected portion of the easily extracted volatile substances comprising the at least one hydrogen proxy does not condense to the cryogenic trap.

### Quantitation: RVS Method(s) & Related Calculation(s)

In aspects, the analysis of ammonia directly, alone or in combination with the analysis of nitrogen in combination with argon; the analysis of oxygen in combination with argon; and the direct measurement of hydrogen, can be used together in the quantification of hydrogen, e.g., the amount of hydrogen expected to be present in a material source when such data is collected from a sample of such material. According to aspects, rock volatile stratigraphy method(s) disclosed herein and in Prior Smith Patents can be used to analyze the quantity (ies) of substances described here relative to method(s) of hydrogen quantification, e.g., method(s) of hydrogen quantification comprising use of one or more hydrogen proxy (ies).

According to aspects, an estimate of the amount of hydrogen present in sample(s) at the time of sealing, the amount of hydrogen that is measured directly in the sealed sample, the amount of hydrogen that was in a sample when sealed but consumed to produce water, and the amount hydrogen that was in the sample when sealed but consumed to produce ammonia, can be added together. This sum provides an estimate of the original cuttings H2 before water & ammonia production.

According to aspects, hydrogen quantitation herein comprises the analysis of a water-free or substantially water-free or essentially water-free non-condensable gas (e.g., that does not condense to a cryogenic trap when applying methods according to the Prior Smith Patents) that is analyzable by providing that water-free gas directly into an analyzer, such as, for example, a mass spectrometer. In certain further respects, method(s) of measuring hydrogen or hydrogen proxy (ies) are indifferent to what technology(ies) are used to measure them. Mass spectrometry is one example of an analytical method that may be used to measure one or more of the aforementioned gaseous molecules. However, other analytical methods may be substituted or combined with mass spectrometry to measure one or more of the aforementioned gaseous molecules. In aspects, any method suitable for quantifying, measuring, or otherwise obtaining relative amount(s) of the gaseous molecules, compound(s), or combination(s) thereof may be suitable for use in aspects of the disclosure described herein.

In aspects, such quantitation is performed by method(s) comprising applying a rock volatile stratigraphy method to the material sample, the rock volatile stratigraphy method comprising (1) application of a first force to the material sample to release at least a first aliquot of one or more substances from the material sample; (2) optionally applying a second force to the material sample to release at least a second aliquot of one or more substances which differ from the one or more substances released by the application of the first force; (3) concentrating or otherwise detectably or significantly enriching at least one of the one more substances released by the application of the first force and optional second force by applying a technique capable of capturing one or more of the other substances released by the application of the first force and optional second force; (4) measuring a plurality of substances released from the material sample by an analytical method such as mass spectrometry or a suitable equivalent thereof, wherein the plurality of substances comprises hydrogen, oxygen, argon, nitrogen, ammonia or combination(s) thereof; (5) and estimating the amount of hydrogen present in the material sample at the time of its collection and prior to its exposure to the atmospheric air within the sealed container

In aspects, the calculation comprises (1) calculating the amount of hydrogen present in the material sample consumed in generating ammonia upon the interaction of the material sample with atmospheric air; and (2) calculating the amount of hydrogen present in the material sample consumed in generating water upon the interaction of the material sample with atmospheric air; and adding the calculated amounts of hydrogen from preceding steps to the amount of hydrogen measured directly.

In aspects, method(s) of quantifying hydrogen comprise applying at least two different forces (resulting in measurably different substances) to generate two different analyzed aliquots for the materials, such as an aliquot produced by applying a pressure of about 20 millibars and a second aliquot produced by applying a pressure of about 2 millibars, each to the same material(s)/sample(s), e.g., in accordance with the teachings of the Prior Smith Patents. In certain aspects, method(s) comprise condensing measurably or significantly less types, concentrations, or amounts of any one, some, many, or most of any of the specific volatile substances analyzed in methods described in the Prior Smith Patents, to a trap (e.g., a cryogenic trap) used in performing the method. In particular aspects, any trapped substance(s) are discarded. In other particular aspects, a cryogenic trap lacks any heating element capable of releasing trapped substances.

In certain aspects, method(s) of hydrogen quantitation comprise the collection of gas or one or more volatile substance(s) from an isolated environment, e.g., a sealed sample container, and the analysis thereof. The analysis can comprise a comparison of the gas or one or more volatile substances to another gas/other gases, e.g., the atmospheric air, or, e.g., one or more other volatile substances. In aspects, the analysis can comprise analyzing a change in one or more substances in the gas in the isolated environment/container indicative of the amount/concentration/purity of hydrogen in the sample/material (e.g., a relative change in the amount of oxygen, nitrogen, ammonia, water vapor, hydrogen, or a combination of some or all thereto or other compound that may react with hydrogen in the isolated environment open space/container headspace).

It may be that in certain scenarios, an amount of H2 is obtained from analysis(es) which represents an amount too high for it to be present as gas in the sample material. In some aspects, such hydrogen can be present as a condensed phase. In aspects, hydrogen can be present chemically bonded to the rock as a hydride. According to such aspects, the amount of H2 stored at, e.g., the subsurface location from which material sample(s) may be collected can be far higher, e.g., orders of magnitude higher, than the quantification method(s) herein may indicate. According to particular method(s), provided herein are method(s) of identifying hydrogen stored as a condensed phase. Hydrogen quantification method(s) described herein can indicate amount(s) of hydrogen which are at least about 1, ~2, ~3, ~4, ~5, orders of magnitude or more higher than amount(s) actually measured.

### SPECIFIC EXEMPLARY EMBODIMENT(S)

In one particular embodiment, the invention provides a method for measuring the amount of hydrogen in a material comprising (1) obtaining an analyzable amount of a material as a sample, (2) subjecting the sample to one or more gentle vacuum equivalent forces that extract one or more easily released volatile substances from the sample, if present, the one or more easily released volatile substances comprising at least one hydrogen proxy, collecting at least a portion of the extracted easily released volatile substances, and measuring the amount of the at least one hydrogen proxy in the collected portion of the easily released volatile substances and using the measurement in a determination of the amount of hydrogen in the material.

In another particular aspect, the invention provides a method of identifying the amount of hydrogen present in a material sample, the amount of hydrogen identified being the amount of hydrogen present in the material sample at the time of its collection, the method comprising (1) collecting a sample of a material from a location protected from exposure to atmospheric air until the time the sample material is collected; (2) sealing the material sample upon collection, wherein the material sample is sealed within a container wherein the container contains an amount of atmospheric air, to isolate the material sample from atmospheric air outside of the container; (3) applying a rock volatile stratigraphy method to the material sample, the rock volatile stratigraphy method comprising (a) applying a first force to the material sample to release at least a first aliquot of one or more substances from the material sample; (b) optionally applying a second force to the material sample to release at least a second aliquot of one or more substances which differ from the one or more substances released by the application of the first force; (c) concentrating or otherwise detectably or significantly enriching at least one of the one more substances released by the application of the first force and optional second force by applying a technique capable of capturing one or more of the other substances released by the application of the first force and optional second force; and (d) measuring a plurality of substances released from the material sample by an analytical method such as mass spectrometry or a suitable equivalent thereof, wherein the plurality of substances comprises one or more of hydrogen, oxygen, argon, nitrogen, and ammonia; and (4) estimating the amount of hydrogen present in the material sample at the time of its collection and prior to its exposure to the atmospheric air within the sealed container, wherein the calculation comprises (a) calculating the amount of hydrogen present in the material sample consumed in generating ammonia upon the interaction of the material sample with atmospheric air; (b) calculating the amount of hydrogen present in the material sample consumed in generating water upon the interaction of the material sample with atmospheric air; and (c) (A) adding the calculated amounts of hydrogen from steps (4) (a) and (4) (b) to the amount of hydrogen measured directly in step (3) (d); (B) calculating the amount of ammonia produced based upon the amount of nitrogen lost to due ammonia production, calculating the amount of water produced based upon the amount of oxygen lost due to water production, and adding the two calculated amounts to the amount of hydrogen measured directly in step (3) (d); or (C) both (A) and (B).

In further respects, the method concentration step of step (3) (c) comprises application of a technology capable of capturing one or more condensable substances released by the application of the first force and optional second force, such as the use of a cryogenic trap or condensable material trap, the use of Drierite™, the use of desiccant, or, e.g., the use of Teflon with sulfonyl channels. In certain respects, the one or more substances that are concentrated or otherwise enriched comprise one or more of hydrogen, helium, argon, nitrogen, oxygen, and ammonia. In certain aspects, the step of measuring a plurality of substances released from the material sample by an analytical method comprises measuring substances by mass spectrometry.

According to aspects, calculating the amount of hydrogen present in the material sample consumed in generating ammonia is performed according to one or both of the following sets of equations:
1. From estimating nitrogen lost to ammonia production:

N2 lost to ammonia production=(77*argon measured)-N2 measured, and

H2 lost to ammonia production=(N2 lost to ammonia production)*3; or
2. Using presence of ammonia:

NH3 generated with H2 from sample=(N2 lost to ammonia production)*2 or

NH3 measured, and

H2 lost to ammonia production=(Ammonia measured)*1.5

According to aspects, calculating the amount of hydrogen present in the material sample consumed in generating water is calculated based on the following equations: O2 lost to H2O production=(22*argon measured)-O2 measured, wherein

H2O generated with H2 from sample=(O2 lost to H2O production)*2, and

H2 lost to water production=(O2 lost to water production)*2.

In aspects, calculating the total amount of hydrogen present in the material sample at the time it was collected is calculated according to the following equation:

H2 (material sample total)=Amount of H2 measured directly (H2 measured)+Amount of H2 lost to ammonia production (which is 1.5*NH3 measured or 1.5*NH3 estimated from N2 lost)+Amount of H2 lost to water production (which is 2*O2 lost to water production).

In certain additional aspects, one or more additional mathematical factors can be applied to calculation(s) herein to refine the amount of hydrogen estimated to be associated with the material in its source location, wherein the application of the one or more additional mathematical factors renders an amount of hydrogen estimated to be present in the material sample at the time of its collection, prior to its exposure to the atmospheric air within the sealed container, or both, which is detectably or significantly closer to the actual amount of hydrogen present in the material sample prior to it being collected from the location protected from exposure to atmospheric air. Such a factor can aid or partially aid in accounting for time between collection and analysis; distance between source and collection point, or both time and distance.

According to particular embodiments, method(s) of determining the amount of hydrogen in a material disclosed herein comprise(s) comparing the quantity of hydrogen measured in the one or more easily released volatile substances to the quantity of helium measured in the one or more easily released volatile substances. In aspects, the method(s) comprise analyzing a plurality of samples from different parts of a geologic unit, determining the amount of hydrogen and the amount of helium in the plurality of samples, and identifying one or more areas of high hydrogen content; one or more areas of high helium content; one or more areas of high hydrogen content and low helium content; one or more areas of high helium content and low hydrogen content; or any combination thereof in the geologic unit. In aspects, the method(s) comprise subjecting one or more samples to different volatile extraction conditions, wherein one of the different volatile extraction conditions results in the extraction of a significantly greater amount of hydrogen than helium, and a different one of the different volatile extraction conditions results in the extraction of a significantly greater amount of helium than hydrogen.

Aspects related to the quantification of hydrogen in materials are further described by the Examples and associated figures provided herewith.

### Comparative Analysis of Hydrogen and Other Substances (e.g., Helium)

In one aspect of method(s) disclosed herein, at least two samples are collected and analyzed separately to determine if there is a difference between the samples in the amount of hydrogen, the purity of the hydrogen, etc., or the amount and purity of the hydrogen in the sample at the time it was collected from a subterranean location to assess where to further explore for or produce hydrogen.

According to aspects, method(s) and device(s) described herein are capable of identifying detectably or significantly stratified versus co-mingled helium and hydrogen deposits, wherein such deposits may be useful hydrogen extraction target(s). Understanding in the art, at the time of this Application is that the two gases, helium and hydrogen, are found together in geologic locations. However, novel technology(ies) and application(s) thereof described herein demonstrates that this is not the case.

In aspects, method(s) herein identify different geologic locations where the amount of hydrogen, the purity of hydrogen, or both the amount and purity of hydrogen may differ or where hydrogen can be differentially extracted from a sample containing other volatile substances, e.g., helium, in order to provide higher purity hydrogen, e.g., greater than about 99.95%, greater than about 99.99% or greater than about 99.995% pure on a molar basis.

In aspects, method(s) herein are capable of distinguishing helium-rich zone(s) which are in very close proximity to hydrogen-rich zone(s); such as, e.g., distinguishable zones which are adjacent to one another or, e.g., are within less than 500 ft, <400 ft, <300 ft, <200 ft, <100 ft, <50 ft, <40 ft, <30 ft, <20 ft, <10 ft, <5 ft, <4 ft, <3 ft, <2 ft, <1 ft, or, e.g., within inches of one another.

In one variation of method(s) herein, the analysis of samples provides a means to identify target locations within, e.g., a geologic unit, where production of hydrogen may be undertaken. In aspects, the methods provide information about the purity of hydrogen, amount of hydrogen, or both the purity and amount of hydrogen for hydrogen prospectors. In aspects, the purity of the hydrogen is influenced by the presence of other gases, e.g., helium or liquids, e.g., water or both gases and liquids.

In one aspect of method(s) herein, more than one sample is analyzed to determine differences in the purity of the hydrogen across samples. In aspects, the method is used to test more than one sample across different geographic locations or different locations within a well or borehole, for example, or both different geographic locations and locations within a well. In aspects, the method provides information about where to consider production of geologic hydrogen in one or more geographic locations or one or more locations within a well or both production in one or more geographic locations and locations within a well.

In one aspect, the invention provides a method of identifying the difference in hydrogen content between two or more material samples, the method comprising collecting a plurality of material samples from one or more locations wherein each of the one or more materials are protected from exposure to atmospheric air until the time each of the material samples are collected; sealing each material sample upon its collection, wherein each material sample is sealed within its own container wherein each container contains an amount of atmospheric air, to isolate each material sample from atmospheric air outside of each container; applying a rock volatile stratigraphy method to each material sample, the rock volatile stratigraphy method comprising applying a first force to each material sample to release at least a first aliquot of one or more substances from the material sample; optionally applying a second force to the material sample to release at least a second aliquot of one or more substances which differ from the one or more substances released by the application of the first force; concentrating or otherwise detectably or significantly enriching at least one of the one more substances released by the application of the first force and optional second force by applying a technique capable of capturing one or more of the other substances released by the application of the first force and optional second force. In one aspect, the invention provides a method of identifying the relative purity of hydrogen present in a material, wherein the purity of hydrogen is the amount of hydrogen present relative to the amount of helium present, the method comprising (a) collecting a sample of a material from a location protected from exposure to atmospheric air until the time the sample material is collected; (b) sealing each material sample upon collection, wherein the material sample is sealed within a container wherein the container contains an amount of atmospheric air, to isolate the material sample from atmospheric air outside of the container; (c) applying a rock volatile stratigraphy method to the material sample, the rock volatile stratigraphy method comprising (i) applying a force to the material sample to release one or more volatile substances, wherein the one or more volatile substances comprise hydrogen, helium, or both hydrogen and helium, and (ii) a means for concentrating the hydrogen, helium or both which removes one or more volatile substance(s) other than hydrogen, helium, or both; (d) measuring the amount hydrogen and the amount of helium released by the application of the force by an analytical method such as mass spectrometry or a suitable equivalent thereof; (e) comparing the ratio of the amount of hydrogen to the amount of helium measured in step (d) to a known value, wherein the known value is a value indicating an expected ratio of hydrogen to helium in such a material under typical or known conditions; (f) determining if the ratio of hydrogen to helium in the material sample is higher than the known value.

In aspects, in such an exemplary method, after performing one or more steps (c), (d), (e) or (f), step (c) is repeated and a second force is applied to release one or more additional volatile substances and steps (d)-(f) are performed on the additional volatile substances. In aspects, the means for concentrating is accomplished by a cryogenic trap or condensable material trap. In aspects, estimating the amount of hydrogen present in the material sample at the time of its collection and prior to its exposure to the atmospheric air within the sealed container comprises one or more of the hydrogen quantification method(s) described herein.

According to aspects, the amount of hydrogen, purity of hydrogen, or both the amount and purity of hydrogen inform hydrogen prospectors of suitable target locations for hydrogen production.

In certain respects, method(s) and device(s) described herein identify strata wherein helium forms a layer which detectably or significantly prevents, e.g., blocks hydrogen from entering or otherwise associating with rock. Not to be bound by hypothesis, one theory is that this may be due to how each element interacts with rock; e.g., helium, as a Noble gas, may not interact with rock but rather may simply be present and physically block other interactions with the rock from occurring. On the other hand, hydrogen is known to bond with mineral surface(s) of rock, e.g., in aspects, in the form of hydrides. According to aspects, the invention provides, method(s) for the identification of strata of helium-rich rock, wherein the top of such a helium-rich zone may represent a geologic “seal” wherein permeable rocks exist below such seal and impermeable rock exist above such seal. Accordingly, in aspects, the technology described herein identifies a geologic seal formed by helium.

Aspects related to the comparative analysis of hydrogen and other substances are further described by the Examples and associated figures provided herewith.

### Identification and Production of Pure Substances/Purity Evaluation & Production of Pure Hydrogen/Helium by Differential Extraction

The technology described herein provides methods for identifying, in aspects, locations containing exclusively pure hydrogen, or locations that contain hydrogen comingled with other volatile substances, e.g., helium, but wherein one or more conditions may be applied to such locations to extract pure hydrogen.

The analyses of multiple volatile aliquots extracted at different pressures in aspects provides insight as to whether or not pure hydrogen may be contained in portions of a reservoir that would not be realized by a bulk analysis of hydrogen from the extraction of all volatiles at a single aggressively low pressure. This may be especially a problem in cuttings where rock grains from a number of depths are collected at the surface at the same time. A much greater level of hydrogen purity in under certain pressure extractions, versus less pure hydrogen extraction at other pressure extractions indicates certain layers or fractures or other features of the earth's subsurface somewhere in the vicinity that the cuttings sample was caught may contain very pure hydrogen. Other more expensive and more depth definitive technologies, such as conventional coring and analyses, closely spaced side wall coring and analyses, closely spaced drill stem tests (DSTs) and analyses, or hydrogen sensitive wireline logs including fiber optics analyses technologies such as Raman Spectroscopy, can be used to pinpoint any discreet zones of very pure hydrogen resource that can then be targeted for production of very pure hydrogen.

Hydrogen must be of extremely high purity to be used as a fuel, for example, greater than about 99.95, greater than about 99.99%, or greater than about 99.995% pure on a molar basis.

Helium is commonly considered a likely contaminant of hydrogen in-as-much-as hydrogen and helium are both very small and both hydrogen and helium are very light. These shared properties of size and mass are considered conducive to hydrogen and helium co-mingling in the subsurface. However, described herein are data demonstrating that hydrogen and helium can be identified as DOS segregated from one another. Such segregation of hydrogen from helium has been observed uniquely in data generated on rock volatile stratigraphy method analyzer(s) described herein, and, e.g., in certain respects, in Prior Smith Patents.

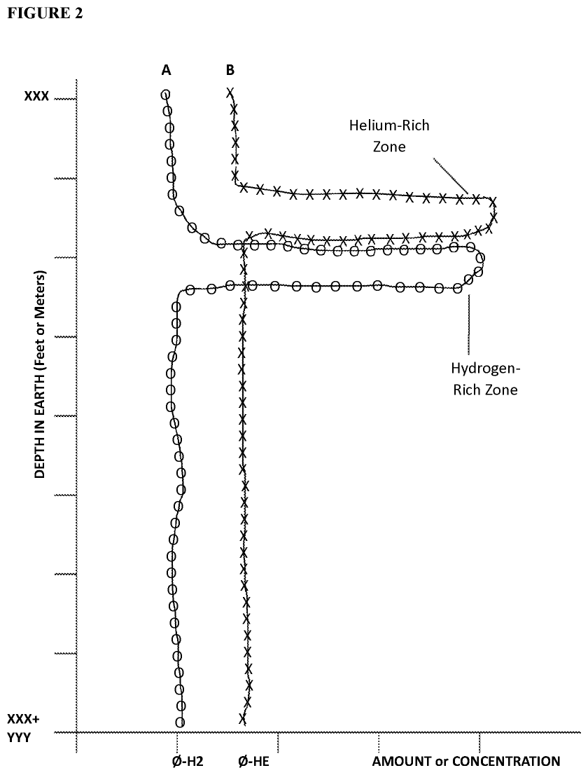

The first aspect is the stratigraphic separation of helium and hydrogen. In aspects, disclosure herein provides observations of rock zones rich in helium directly adjacent to rock zones rich in hydrogen. See, for example, FIG. 2 exemplifying such data. In aspects, such zones may occur at a variety of thicknesses of these rock zones. Pinpointing the hydrogen-rich and helium-poor zones allows for the production of purer hydrogen with lower helium contents. In aspects, observations can allow for pinpointing zones for the production of purer helium from the helium-rich hydrogen-poor zones.

The stratigraphic separation of helium from hydrogen can, in aspects, be due to a seal, such as rock with very low permeability being above the helium and not allowing the helium to be transmitted towards the surface. The helium in itself may then create a seal which prevents the hydrogen from migrating further towards the surface, as the helium may occupy crevices, pores, etc. that would otherwise be available for the hydrogen to occupy.

The stratigraphic separation of helium and hydrogen may allow for the extraction of both highly pure helium and highly pure hydrogen or both. In aspects, the identification of such circumstances described here represents a novel element of this technology.

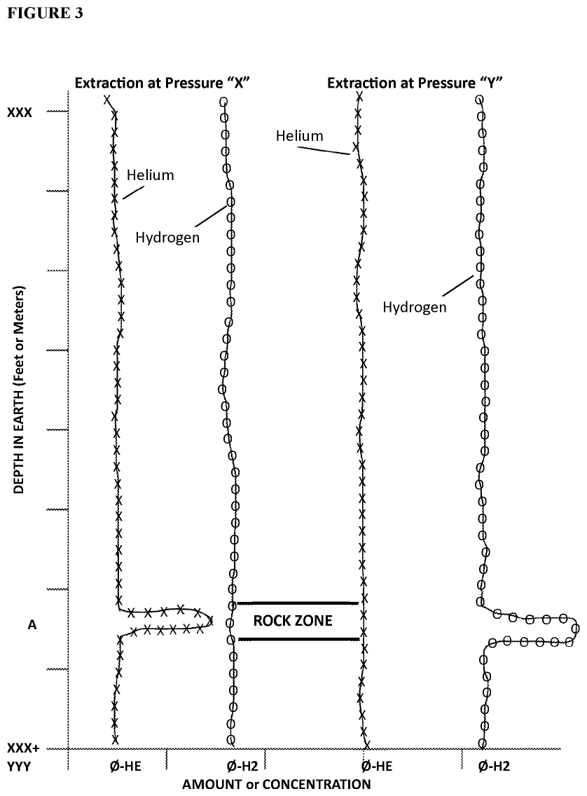

In another aspect, single zones containing both hydrogen and helium are identified, but in which hydrogen and helium each show higher and lower abundances in different volatiles aliquots from analyses of rock volatiles extracted at different pressures (see, e.g., FIG. 3). This suggests perhaps that the same rock is holding hydrogen and helium in different pore spaces in the same rock, or within pores within different but spatially close aspects of the same rock, within different pore spaces closely spaced rocks maybe in layers, or that the helium is not chemically associated with the rock whereas the hydrogen is chemically associated with the same rock, making it easier to extract the helium from the rock with a less strong vacuum, perhaps about 20 mbar of residual pressure versus hydrogen being more difficult to extract, perhaps requiring a stronger vacuum with a residual pressure of about 2 mbar. . . . Other more expensive and more depth definitive technologies, such as conventional coring and analyses, closely spaced side wall coring and analyses, closely spaced drill stem tests (DSTs) and analyses, or hydrogen sensitive wireline logs including fiber optics analyses technologies such as Raman spectroscopy, can be used to pin point any discreet zones of very pure hydrogen or very pure helium resources that can then be targeted for production of either or both very pure hydrogen and/or very pure helium.

Such single zones containing both hydrogen and helium, thus each unpure, may be each extracted in pure form, by the application of one or more forces, e.g., a weaker force (such as a weaker differential in pressure between the zone in the subsurface and the surface) to extract pure helium, e.g., >95%, >99%, or even >99.9%.

In such single zones, having extracted the helium, a second force may then be applied to the zone to extract hydrogen in pure form, e.g., >95%, >99%, >99.9%, or even greater than 99.95%.

In one aspect, the invention provides a method of comparing the relative purity of hydrogen between two or more material samples, wherein the purity of hydrogen is the amount of hydrogen present relative to the amount of helium present, the method comprising: (a) collecting at least two material samples; (b) applying a rock volatile stratigraphy method to each material sample, the rock volatile stratigraphy method comprising (i) applying a force to the material sample to release one or more volatile substances, wherein the one or more volatile substances comprise hydrogen, helium, or both hydrogen and helium, and (ii) a means for concentrating the hydrogen, helium or both which removes one or more volatile substance(s) other than hydrogen, helium, or both; (c) measuring the amount hydrogen and the amount of helium in each material sample released by the application of the force by an analytical method such as mass spectrometry or a suitable equivalent thereof, to establish a ratio of the amount of hydrogen to the amount of helium in each material sample; and d) comparing the ratio of the amount of hydrogen to the amount of helium established in step (c) of at least one material sample, to the ratio of the amount of hydrogen to the amount of helium established in step (c) of at least one other material sample, to determine which material sample has a higher ratio of hydrogen to helium.

In aspects, the method may provide for identifying zones of a single well wherein in one or more zones there are subzones that each contain both highly pure hydrogen overlaid by highly pure helium and in one or more other zones helium and hydrogen are comingled but can each be extracted from one another in highly pure form, i.e., highly pure helium and highly pure hydrogen by the application of at least two different conditions, e.g., a weaker pressure differential from the surface and a stronger pressure differential from the surface.

According to one aspect of the technology(ies) herein, single zones containing both hydrogen and helium, but in which hydrogen and helium each show higher and lower abundances in different volatiles aliquots from analyses of rock volatiles extracted at different pressures (see FIG. 3) are identified and exploited. Such findings, in aspects, indicate that the same rock is holding hydrogen and helium in different pore spaces in the same rock, or within pores within different but spatially close aspects of the same rock, or with different pore spaces closely spaced rocks may be in layers. Other more expensive and more depth definitive technologies, such as conventional coring and analyses, closely spaced side wall coring and analyses, closely spaced drill stem tests (DSTs) and analyses, or hydrogen sensitive wireline logs including fiber optics analyses technologies such as Raman Spectroscopy, can be used to pin point any discreet zones of very pure hydrogen or very pure helium resources that can then be targeted for production of either or both very pure hydrogen and/or very pure helium.

The analyses of multiple volatile aliquots extracted at different pressures disclosed herein, in aspects, provides insight as to whether or not pure hydrogen may be contained in portions of a reservoir that would not be realized by a bulk analysis of hydrogen from the extraction of all volatiles at a single aggressively low pressure. This is especially a problem in cuttings where rock grains from a number of depths are collected at the surface at the same time. A much greater level of hydrogen purity in under certain pressure extractions, versus less pure hydrogen extraction at other pressure extractions, indicates certain layers or fractures or other features of the earth's subsurface somewhere in the vicinity that the cuttings sample was caught may contain very pure hydrogen. Other more expensive and more depth definitive technologies, such as conventional coring and analyses, closely spaced side wall coring and analyses, closely spaced drill stem tests (DSTs) and analyses, or hydrogen sensitive wireline logs including fiber optics analyses technologies such as Raman Spectroscopy, can be used to pinpoint any discreet zones of very pure hydrogen resource that can then be targeted for production of very pure hydrogen.

In certain aspects of method(s) herein, the volatile(s) analysis provides information about the purity of the hydrogen relative to helium that may also be present in the same material. In aspects, the methods provide information that suggests the hydrogen may not exist solely as a gas in the material by virtue of the porosity of the material. In aspects, the method provides information that the hydrogen may exist in the material in an amount that far exceeds the amount that would be expected based on the porosity of the material. In aspects, the method indicates that the hydrogen in the material must be present in a form other than a gas, e.g., a condensed phase or chemically associated with the material, e.g., in the form of a hydride. In aspects, the method provides information that indicates the hydrogen is of a purity to be usable as a fuel. In aspects, the method demonstrates that the purity of the hydrogen meets or exceeds one or more government standards. In aspects, the methods demonstrate that the hydrogen is at least 95%, at least 99.5%, at least 99.9% or at least 99.995% pure relative to other gases in the material.

In aspects, the invention provides method(s) of applying two or more different force(s), e.g., differing in their type or degree, to a single sample, resulting in the extraction of helium, hydrogen, or both, wherein the force(s) applied is/are selected according to the target element. In aspects, a force can be, e.g., a temperature (e.g., the application of heat energy), a pressure force such as a vacuum, or any such force capable of detectably or significantly providing for the selective extraction of helium versus hydrogen or vice-versa. In aspects, the varying degree in a force can be, e.g., the application of two or more detectably or significantly different pressures, e.g., two or more pressures selected from 1 millibar, 2 millibars, 3 millibars, 4 millibars, 5 millibars, 6 millibars, 7 millibars, 8 millibars, 9 millibars, 10 millibars, 15 millibars, 20 millibars, 30 millibars, 40 millibars, 50 millibars, 75 millibars, 100 millibars, or higher, or, e.g., any pressure between such pressure(s) exemplified here. In one particular example, e.g., a first pressure of 20 millibars and a second pressure of 2 millibars may be used as the two different applied forces.

According to certain aspects, the technology herein provides a method of identifying a geologic seal formed by helium within a geologic unit, the geologic seal being a layer of material within the geologic unit comprising an amount of helium which is detectably or significantly greater than the amount of hydrogen, and wherein the layer is positioned adjacent to a layer of material within the geologic unit comprising an amount of hydrogen which is detectably or significantly greater than the amount of helium, the method comprising (a) collecting a plurality of material samples representing different locations within a geologic unit; (b) applying a rock volatile stratigraphy method to each material sample, the rock volatile stratigraphy method comprising (i) applying a force to the material sample to release one or more volatile substances, wherein the one or more volatile substances comprise hydrogen, helium, or both hydrogen and helium, and (ii) a means for concentrating the hydrogen, helium or both which removes one or more volatile substance(s) other than hydrogen, helium, or both; (c) measuring the amount hydrogen and the amount of helium in each material sample released by the application of the force by an analytical method such as mass spectrometry or a suitable equivalent thereof, to establish a ratio of the amount of hydrogen to the amount of helium in each material sample; (d) comparing the amount of hydrogen to the amount of helium established in step (c) in the material samples to identify one or more locations within the geologic unit comprising an amount of helium which is detectably or significantly greater than the amount of hydrogen, and wherein the location is positioned adjacent to a layer of material within the geologic unit comprising an amount of hydrogen which is detectably or significantly greater than the amount of helium.

In one aspect, the invention provides a method of identifying one or more strata within a geological unit demonstrating a higher level of hydrogen purity than one or more other strata within the same geologic unit, wherein the purity of hydrogen is the amount of hydrogen present relative to the amount of helium present, the method comprising (a) collecting a plurality of material samples representing different strata within a geologic unit; (b) applying a rock volatile stratigraphy method to each material sample, the rock volatile stratigraphy method comprising (i) applying a first force to each material sample to release a first aliquot of one or more volatile substances, wherein the one or more volatile substances comprise hydrogen, helium, or both hydrogen and helium; (ii) applying a second force to each material sample to release a second aliquot of one or more volatile substances, wherein the one or more volatile substances comprise hydrogen, helium, or both hydrogen and helium, and wherein the one or more volatile substances released upon the application of the second force were not released upon application of the first force; (iii) applying or otherwise utilizing a means for concentrating the hydrogen, helium or both the hydrogen and the helium released from the application of the first force, the second force, or both, which removes one or more volatile substance(s) other than hydrogen, helium, or both; (iv) measuring the amount hydrogen, if present, and the amount of helium, if present, in the first aliquot of volatile substances released from each material sample by an analytical method such as mass spectrometry or a suitable equivalent thereof, (v) measuring the amount of hydrogen, if present, and the amount of helium, if present, in the second aliquot of volatile substances released from each material sample by an analytical method such as mass spectrometry or a suitable equivalent thereof; (c) comparing the amounts of hydrogen and helium measured in the first aliquot of each material sample in step (b) (iv) to the amounts of hydrogen and helium measured in the second aliquot of each material sample in step (b) (v); (d) identifying one or more material samples wherein the amount of hydrogen, the amount of helium, or both the amount of hydrogen and the amount of helium is/are different between the first aliquot and second aliquot; and (e) associating the material samples identified in (d) with strata within the geological unit having a higher hydrogen purity.

In one aspect, the invention provides a method of identifying one or more locations within a geological unit demonstrating a higher level of hydrogen purity than one or more other locations within the same geologic unit, wherein the purity of hydrogen is the amount of hydrogen present relative to the amount of helium present, the method comprising (a) collecting a plurality of material samples representing different locations within a geologic unit; (b) applying a rock volatile stratigraphy method to each material sample, the rock volatile stratigraphy method comprising (i) applying a first force to each material sample to release a first aliquot of one or more volatile substances, wherein the one or more volatile substances comprise hydrogen, helium, or both hydrogen and helium; (ii) applying a second force to each material sample to release a second aliquot of one or more volatile substances, wherein the one or more volatile substances comprise hydrogen, helium, or both hydrogen and helium, and wherein the one or more volatile substances released upon the application of the second force were not released upon the application of the first force; (iii) applying or otherwise utilizing a means for concentrating the hydrogen, helium or both the hydrogen and the helium released from the application of the first force, the second force, or both, which removes one or more volatile substance(s) other than hydrogen, helium, or both; (iv) applying or otherwise utilizing a means for concentrating the hydrogen, helium or both the hydrogen and the helium released from the application of the first force, the second force, or both, which removes one or more volatile substance(s) other than hydrogen, helium, or both; (v) measuring the amount hydrogen, if present, and the amount of helium, if present, in the first aliquot of volatile substances released from each material sample by an analytical method such as mass spectrometry or a suitable equivalent thereof, (vi) measuring the amount of hydrogen, if present, and the amount of helium, if present, in the second aliquot of volatile substances released from each material sample by an analytical method such as mass spectrometry or a suitable equivalent thereof; (c) comparing the amounts of hydrogen and helium measured in the first aliquot of each material sample in step (b) (iv) to the amounts of hydrogen and helium measured in the second aliquot of each material sample in step (b) (v); (d) identifying one or more material samples wherein the amount of hydrogen, the amount of helium, or both the amount of hydrogen and the amount of helium is/are different between the first aliquot and second aliquot; and (e) associating the material samples identified in (d) with strata within the geological unit having a higher hydrogen purity.

In certain aspects, a hydrogen payzone is identifiable as a zone where hydrogen is primarily present in a first, weak-extraction aliquot, e.g., aliquot Al described herein. In certain aspects, a hydrogen payzone is identifiable as a zone where hydrogen is primarily found in stronger-extraction aliquot(s).

In one aspect, the invention provides a method of identifying a geologic seal within a geologic unit, the geologic seal being a first layer of material within the geologic unit comprising an amount of hydrogen which is detectably or significantly greater than the amount of hydrogen within a second layer of material more proximal to the surface of the Earth than the first layer of material, and wherein the first layer is positioned adjacent to and more proximal to the surface of the Earth than a third layer of material within the geologic unit comprising hydrogen, the method comprising (a) collecting a plurality of material samples representing different locations within a geologic unit; (b) applying a rock volatile stratigraphy method to each material sample, the rock volatile stratigraphy method comprising (i) applying a first vacuum force of about 20 mbar for about 1 minute to the material sample to release a first aliquot of one or more volatile substances, wherein the one or more volatile substances comprise hydrogen, helium, or both hydrogen and helium, (ii) a means for concentrating from the first aliquot the hydrogen, helium or both which means of concentrating removes one or more volatile substance(s) other than hydrogen, helium, or both, (iii) applying the first vacuum force for about 8 additional minutes to release a second aliquot of one or more volatile substances, wherein the one or more volatile substances comprise hydrogen, helium, or both hydrogen and helium, (iv) applying the means for concentrating from the second aliquot the hydrogen, helium or both, (v) applying a second vacuum force of about 2 mbar for about 1 minute to the material sample to release a third aliquot of one or more volatile substances, wherein the one or more volatile substances comprise hydrogen, helium, or both hydrogen and helium, (vi) applying the means for concentrating from the third aliquot the hydrogen, helium or both, (vii) applying the second vacuum force for about 8 additional minutes to the material sample to release a fourth aliquot of one or more volatile substances, wherein the one or more volatile substances comprise hydrogen, helium, or both hydrogen and helium, and (viii) applying the means for concentrating from the fourth aliquot the hydrogen, helium or both, (c) measuring the amount hydrogen in each aliquot; (d) comparing the amount of hydrogen measured in each aliquot established in step (c) in each material sample to identify the first layer wherein the amount of hydrogen in the first aliquot is detectable or significantly more than the amount of hydrogen from the second aliquot, the third aliquot and the fourth aliquot from the same material sample and wherein the amount of hydrogen in the first aliquot from the first layer is detectably or significantly more than the first aliquot from the second layer and the first aliquot from the third layer and to identify the third layer wherein the amount of hydrogen from the fourth aliquot from the third layer is detectably or significantly more than the hydrogen from the first aliquot, the second aliquot and the third aliquot from the third layer.

Aspects related to pure hydrogen and pure helium zones, production, and evaluation of purity are further described by the Examples and associated figures provided herewith.

### Generation of Hydrogen in Materials and Analysis of Hydrogen-Generation Capacity

### Producing and Analyzing Hydrogen Through Fluid-Rock Interactions in Sealed Tubes and Subsequent Analyses of the Volatiles in the Same Sealed Tube

Much, perhaps most, hydrogen is believed to be generated by oxidation of reduced, divalent, ferrous iron (Fe²⁺) to oxidized, trivalent, ferric iron (Fe³⁺) through the reaction of ferrous iron with water in the subsurface via the reaction:

2Fe²⁺O+2H₂O->Fe⁺³₂O₃+2H₂

This hydrogen-producing reaction is often thought to occur in mafic and ultramafic igneous rocks such as peridotites, eclogites, amphibolites, dunites, etc., that are relatively rich in ferrous iron, and are often derived from the Earth's upper mantle, or the Earth's lower crustal zones in mafic volcanic provinces. The hydrogen production reaction is thought to often occur as part of the serpentinization process of converting ultra-mafic igneous rocks to serpentine.

It has been proposed by others that given the appropriate rock types that certain fluids may be able to be injected into such rocks through an appropriate well or array of wells to artificially produce synthetic hydrogen in the subsurface, and then to recover that hydrogen through the production of hydrogen via the same or associated wells.

The sealed at well containers used in our patented base technology for analyses of drill cuttings can be used to study the natural production of hydrogen in the subsurface, as well as studying potential rocks and fluids for synthetic hydrogen production so as to maximize and determine the best hydrogen-producing rocks and hydrogen-producing fluids, and the best combination of rocks and fluids for synthetic hydrogen production.

Atmospheric air, which normally occupies the headspace in our sealed samples, can be purged and replaced with an appropriate nonreactive gas, such as argon, krypton, xenon, or another nonreactive gas. A more reactive gas, such as CO2, CO, or another reactive gas, might be used to purge and fill the headspace to evaluate the effects of various reactive gases on hydrogen production.

If necessary, the tubes can be modified to minimize any chemical reactions of the fluid and rocks with the tube itself during these experiments. Also, the polymer seals can be modified or replaced, either using some other more temperature/pressure resistant polymer, such as Kevlar, or an appropriate metal seal, or some optimum combination of polymer(s) and metal(s) for the seal. Direct connection to the analytical apparatus might be made using stainless steel valves and fittings, or valves and fittings made of other appropriate materials such as PEEK. Or for some experiments our normal method of joining the sample tubes inner space to the instrument by passing a stainless-steel needle through a nitrile cap may prove the best and most efficient connection from the sample to the inlet of the apparatus.

Using these appropriate modifications a multitude of experiments can be performed using a variety ferrous-iron bearing rocks, or mixture of rocks, introduced as small cuttings-like particles, or as an intact micro-core that can be snuggly or loosely fit into the container, and using a variety of waters having differing salinities, dissolved solid compositions, and Ph and Eh, that can be loaded together and sealed into the tubes and subjected to elevated heat and pressure and a variety of mechanical, including vibrational and impact stresses, to determine the optimum rocks, temperature and pressure conditions, and possibly information for optimizing completion procedures to optimize the formation and recovery of synthetically produced hydrogen.

Following each experiment, the sample container can be appropriately interfaced with the analytical apparatus, previously patented, and the volatiles extracted, analyzed, and evaluated for amounts of produced hydrogen. A higher level of classification can be performed by maintaining the sealed sample at higher temperatures, perhaps optimally at the estimated anticipated producing reservoir's temperature, during extraction.

The data from these experiments will facilitate determining the optimum rocks and fluids for targeting and optimized drilling and completing, the optimum fluid to be injected into these selected rocks for optimum hydrogen generation and production.

Others have also suggested that the production of synthetic hydrogen can also be combined with the sequestration of carbon and sulfur compounds as solid residue in the produced zones. The rocks used to generate hydrogen in the experimental sample tubes can be removed from the tubes after the volatiles analysis process, and the efficiency of carbon and sulfur sequestration can be evaluated using XRD, XRF, or other appropriate technology for identifying solid chemistries and mineralogic contents.

While data collected in these manners will have significant value in evaluating how best to produce synthetic hydrogen from subsurface formations, the methods described herein also have much positive potential in exploring for naturally occurring geologic hydrogen. Even though much is known from the geologic study of natural geologic hydrogen, the additional experimentally derived knowledge of optimum rock and fluid compositions and conditions for generating and naturally storing hydrogen realized by these procedures and experiments is directly applicable to designing, guiding, and realizing successful exploration for and production of geologic hydrogen.

In one aspect of the technology, methods are provided to identify rock that is high in ferrous oxide, e.g., >50%, >75%, >90%, or even greater than 99% w/w. Having identified such rock, an aqueous fluid may be injected into the rock to produce hydrogen which can be extracted in high purity, >95%, >99%, >99.9%, or even >99.95%.

Method(s) of the technology herein (in this section and elsewhere) are often provided in a single paragraph, disclosing multiple step(s) of such method(s). The reader should note that in certain respects, each step of the method should be interpreted as independently present and method(s) may not require each and every step disclosed, even if step(s) are disclosed together.

In one aspect, the technology provides a method of synthetically producing and analyzing one or more target volatile substances through one or more fluid-rock interactions within a sealed container, the method comprising (a) collecting a plurality of samples of rock material, wherein the plurality of rock material samples represent a plurality of rock types; (b) placing each of the plurality of rock material samples in a selectively sealable container wherein the container (i) isolates the sample and any volatile substances associated with the sample or gas therein from the environment; (ii) comprises a portion that is selectively accessible by a device suitable for extracting a substance comprising one or more volatile substances therefrom; and (iii) is optionally adapted to be compressed without release of gases contained therein; to establish a plurality of containers each containing a rock material sample; (c) adding to each of the plurality of containers containing a rock material sample a fluid, wherein the fluid added is different for a plurality of containers containing a rock sample and wherein at least one of the fluids added to at least one of the plurality of containers containing a rock sample causes the generation of one or more target volatile substances, the generation of one or more target volatile substances to establish an amount of one or more target volatile substances consisting of (i) any amount of each of the target volatile substances which was present in the rock material sample prior to the addition of the fluid in combination with (ii) the amount of each target volatile compound generated upon the addition of the fluid; (d) immediately sealing each container to establish a plurality of sealed containers each containing a rock-fluid combination sample; (e) applying a rock volatile stratigraphy method to each rock-fluid combination sample, the rock volatile stratigraphy method comprising (i) applying a force to one or more of the plurality of containers containing a rock-fluid combination sample, wherein the application of the force results in the release of one or more volatile substances from the rock material sample, wherein the one or more volatile substances may comprise one or more of the target volatile substances, but wherein the one or more volatile substances remains within the container containing the rock-fluid combination sample until actively withdrawn for analysis; (ii) optionally applying an additional one or more forces to one or more of the plurality of containers containing a rock-fluid combination sample, wherein each of the additional one or more forces is detectably or significantly different from the force of (e) (i) and the application of each additional force results in the release of one or more volatile substances which are detectably or significantly different from one another and from the one or more volatile substances released in (e) (i) and which may comprise one or more of the one or more target volatile substances, but which also remain within the container containing the rock-fluid combination sample until actively withdrawn for analysis; wherein (e) (i) and (e) (iii) establish a force profile applied to each container containing the rock-fluid combination sample; (iii) optionally concentrating one or more of the one or more volatile substances released in one or both of (e) (i) and (e) (ii) by applying or otherwise utilizing a collection means to collect one or more of the volatile substances released by the application of the force profile, wherein one or more of the volatile substances collected by the collection means may comprise one or more of the target volatile substances; (iv) optionally applying a force to the collection means, if applicable, to cause the release of one or more volatile substances captured by the collection means, wherein one or more of the volatile substances released may be one or more of the target volatile substances; and (v) actively withdrawing from the sealed container and measuring the one or more target volatile substances in each container containing a rock-fluid combination sample and further optionally measuring one or more additional volatile substances in each container which are not target volatile substances; and (f) characterizing the amount of each target volatile compound resulting from each rock-fluid combination sample and any associated applied force profile and determining which rock-fluid combination and associated force profile results in yielding the highest amount of each target volatile compound.

In one aspect, the technology provides a method of synthetically producing and analyzing one or more target volatile substances through one or more fluid-rock interactions within a sealed container, the method comprising (a) collecting a plurality of samples of rock material, wherein the plurality of rock material samples represent a plurality of rock types; (b) placing each of the plurality of rock material samples in a selectively sealable container wherein the container (i) isolates the sample and any volatile substances associated with the sample or gas therein from the environment; (ii) comprises a portion that is selectively accessible by a device suitable for extracting a substance comprising one or more volatile substances therefrom; and (iii) is optionally adapted to be compressed without release of gases contained therein; to establish a plurality of containers each containing a rock material sample; (c) adding to each of the plurality of containers containing a rock material sample a fluid, wherein the fluid added is different for a plurality of containers containing a rock sample and wherein at least one of the fluids added to at least one of the plurality of containers containing a rock sample causes the generation of one or more target volatile substances, the generation of one or more target volatile substances to establish an amount of one or more target volatile substances consisting of (i) any amount of each of the target volatile substances which was present in the rock material sample prior to the addition of the fluid in combination with (ii) the amount of each target volatile compound generated upon the addition of the fluid, and wherein each of the target volatile substances has a purity level relative to one or more other volatile substances; (d) immediately sealing each container to establish a plurality of sealed containers each containing a rock-fluid combination sample; (e) applying a rock volatile stratigraphy method to each rock-fluid combination sample, the rock volatile stratigraphy method comprising (i) applying a force to one or more of the plurality of containers containing a rock-fluid combination sample, wherein the application of the force results in the release of one or more volatile substances from the rock material sample, wherein the one or more volatile substances may comprise one or more of the target volatile substances, but wherein the one or more volatile substances remains within the container containing the rock-fluid combination sample until actively withdrawn for analysis; (ii) optionally applying an additional one or more forces to one or more of the plurality of containers containing a rock-fluid combination sample, wherein each of the additional one or more forces is detectably or significantly different from the force of (e) (i) and the application of each additional force results in the release of one or more volatile substances which are detectably or significantly different from one another and from the one or more volatile substances released in (e) (i) and which may comprise one or more of the one or more target volatile substances, but which also remain within the container containing the rock-fluid combination sample until actively withdrawn for analysis; wherein (e) (i) and (e) (iii) establish a force profile applied to each container containing the rock-fluid combination sample; (iii) optionally concentrating one or more of the one or more volatile substances released in one or both of (e) (i) and (e) (ii) by applying or otherwise utilizing a collection means to collect one or more of the volatile substances released by the application of the force profile, wherein one or more of the volatile substances collected by the collection means may comprise one or more of the target volatile substances; (iv) optionally applying a force to the collection means, if applicable, to cause the release of one or more volatile substances captured by the collection means, wherein one or more of the volatile substances released may be one or more of the target volatile substances; and (v) actively withdrawing from the sealed container and measuring the one or more target volatile substances in each container containing a rock-fluid combination sample and further optionally measuring one or more additional volatile substances in each container which are not target volatile substances; (f) characterizing the amount of each target volatile compound resulting from each rock-fluid combination sample and any associated applied force profile and determining which rock-fluid combination and associated force profile results in yielding the highest amount of each target volatile compound; and (g) characterizing the purity of each target volatile compound resulting from each rock-fluid combination sample and any associated force profile and determining which rock-fluid combination and associated force profile results in the highest purity of each target volatile compound.

In one aspect, the technology provides a method of producing and analyzing hydrogen through one or more fluid-rock interactions, the method comprising (a) collecting a plurality of samples of rock material, wherein the plurality of rock material samples represent a plurality of rock types; (b) placing each of the plurality of rock material samples in a selectively sealable container wherein the container (i) isolates the sample and any volatile substances associated with the sample or gas therein from the environment; (ii) comprises a portion that is selectively accessible by a device suitable for extracting a substance comprising one or more volatile substances therefrom; and (iii) is optionally adapted to be compressed without release of gases contained therein; to establish a plurality of containers each containing a rock material sample; (c) adding to each of the plurality of containers containing a rock material sample a fluid, wherein the fluid added is different for a plurality of containers containing a rock sample and wherein at least one of the fluids added to at least one of the plurality of containers containing a rock sample causes the generation of one or more target volatile substances, the generation of one or more target volatile substances to establish an amount of one or more target volatile substances consisting of (i) any amount of each of the target volatile substances which was present in the rock material sample prior to the addition of the fluid in combination with (ii) the amount of each target volatile compound generated upon the addition of the fluid; (d) immediately sealing each container to establish a plurality of sealed containers each containing a rock-fluid combination sample; (e) applying a rock volatile stratigraphy method to each rock-fluid combination sample, the rock volatile stratigraphy method comprising (i) applying a force to one or more of the plurality of containers containing a rock-fluid combination sample, wherein the application of the force results in the release of one or more volatile substances from the rock material sample, wherein the one or more volatile substances may comprise one or more of the target volatile substances, but wherein the one or more volatile substances remains within the container containing the rock-fluid combination sample until actively withdrawn for analysis; (ii) optionally applying an additional one or more forces to one or more of the plurality of containers containing a rock-fluid combination sample, wherein each of the additional one or more forces is detectably or significantly different from the force of (e) (i) and the application of each additional force results in the release of one or more volatile substances which are detectably or significantly different from one another and from the one or more volatile substances released in (e) (i) and which may comprise one or more of the one or more target volatile substances, but which also remain within the container containing the rock-fluid combination sample until actively withdrawn for analysis; wherein (e) (i) and (e) (iii) establish a force profile applied to each container containing the rock-fluid combination sample; (iii) optionally concentrating one or more of the one or more volatile substances released in one or both of (e) (i) and (e) (ii) by applying or otherwise utilizing a collection means to collect one or more of the volatile substances released by the application of the force profile, wherein one or more of the volatile substances collected by the collection means may comprise one or more of the target volatile substances; (iv) optionally applying a force to the collection means, if applicable, to cause the release of one or more volatile substances captured by the collection means, wherein one or more of the volatile substances released may be one or more of the target volatile substances; and (v) actively withdrawing from the sealed container and measuring the one or more target volatile substances in each container containing a rock-fluid combination sample and further optionally measuring one or more additional volatile substances in each container which are not target volatile substances; and (f) characterizing the amount of each target volatile compound resulting from each rock-fluid combination sample and any associated applied force profile and determining which rock-fluid combination and associated force profile results in yielding the highest amount of each target volatile compound.

In aspects, the technology provides a method of synthetic hydrogen production from a geologic unit, the method comprising (a) collecting one or more samples of rock material, wherein the one or more rock material samples represent a plurality of rock types collected from one or more locations of the geologic unit; (b) placing each of the plurality of rock material samples in a selectively sealable container wherein the container (i) isolates the sample and any volatile substances associated with the sample or gas therein from the environment; (ii) comprises a portion that is selectively accessible by a device suitable for extracting a substance comprising one or more volatile substances therefrom; and (iii) is optionally adapted to be compressed without release of gases contained therein; to establish a plurality of containers each containing a rock material sample; (c) adding to each of the plurality of containers containing a rock material sample a fluid, wherein the fluid added is different for a plurality of containers containing a rock sample and wherein at least one of the fluids added to at least one of the plurality of containers containing a rock sample causes the generation of hydrogen, the generation of hydrogen establishing an amount of hydrogen consisting of (i) any amount of hydrogen which was present in the rock material sample prior to the addition of the fluid in combination with (ii) the amount of hydrogen generated upon the addition of the fluid; (d) immediately sealing each container to establish a plurality of sealed containers each containing a rock-fluid combination sample; (e) applying a rock volatile stratigraphy method to each rock-fluid combination sample, the rock volatile stratigraphy method comprising (i) applying a force to one or more of the plurality of containers containing a rock-fluid combination sample, wherein the application of the force results in the release of one or more volatile substances from the rock material sample, wherein the one or more volatile substances may comprise hydrogen, but wherein the one or more volatile substances remains within the container containing the rock-fluid combination sample until actively withdrawn for analysis; (ii) optionally applying an additional one or more forces to one or more of the plurality of containers containing a rock-fluid combination sample, wherein each of the additional one or more forces is detectably or significantly different from the force of (e) (i) and the application of each additional force results in the release of one or more volatile substances which are detectably or significantly different from one another and from the one or more volatile substances released in (e) (i) and which may comprise hydrogen, but which also remain within the container containing the rock-fluid combination sample until actively withdrawn for analysis; wherein (e) (i) and (e) (ii) (if performed) establish a force profile applied to each container containing the rock-fluid combination sample; (iii) optionally concentrating one or more of the one or more volatile substances released in one or both of (e) (i) and (e) (ii) by applying or otherwise utilizing a collection means to collect one or more of the volatile substances released by the application of the force profile; and (iv) actively withdrawing from the sealed container and measuring hydrogen in each container containing a rock-fluid combination sample; (f) characterizing the amount of hydrogen resulting from each rock-fluid combination sample and any associated applied force profile and determining which rock-fluid combination and associated force profile results in yielding the highest amount of hydrogen.

In aspects, the technology provides a method for evaluating the hydrogen generation capacity of a material comprising, (a) obtaining a solid or semisolid mineral aggregate material; (b) contacting the material with an aqueous liquid under conditions that will result in hydrogen generation if the material is an effective hydrogen-generating material, to form a water-treated material; (c) subjecting the water-treated material to one or more gentle vacuum equivalent forces that extract one or more easily released volatile substances from the sample, if present, the one or more easily released volatile substances comprising hydrogen, at least one hydrogen proxy, or both; (d) collecting at least a portion of the extracted easily released volatile substances; (e) measuring the hydrogen content, hydrogen proxy content, or both the hydrogen and hydrogen proxy content of the portion in the portion; and (f) evaluating the ability of the material to generate hydrogen from the measurement or measurements obtained from step (e).

In aspects, the technology provides a method of synthetic hydrogen production from a geologic unit, wherein the material is a sample of a geologic material obtained from a geologic unit.

In aspects, the technology provides a method of synthetic hydrogen production from a geologic unit, wherein the material comprises a rock material comprising ferrous oxide.

In aspects, the technology provides a method of synthetic hydrogen production from a geologic unit, wherein the method further comprises (1) obtaining a plurality of compositionally similar samples of the material and (b) contacting each sample of the plurality of compositionally similar samples with an aqueous substance selected from at least two different aqueous substances, wherein the at least two different aqueous substances differ in one or more physiochemical properties, and wherein the method is used to further evaluate the impact of using the different aqueous substances on the generation of hydrogen from the material.

In aspects, the technology provides a method of synthetic hydrogen production from a geologic unit, wherein the at least two different aqueous substances differ in salinity, dissolved solids, pH, Eh (oxidation-reduction potential), or a combination of any or all thereof.

In aspects, the technology provides a method of synthetic hydrogen production from a geologic unit, wherein the method further comprises (1) obtaining a plurality of compositionally similar samples of the material and (b) contacting each sample of the plurality of compositionally similar samples with an aqueous substance under different environmental conditions, and wherein the method is used to further evaluate the impact of the different environmental conditions on the generation of hydrogen from the material.

In aspects, the technology provides a method of synthetic hydrogen production from a geologic unit, wherein the different conditions comprise application of different mechanical stresses, different temperature conditions, different pressure conditions, or a combination of any or all thereof.

In aspects, the technology provides a method of synthetic hydrogen production from a geologic unit, wherein the method further comprises evaluating the material's ability to efficiently sequester carbon, sulfur, or both.

In aspects, the technology provides a method of synthetic hydrogen production from a geologic unit, wherein the evaluation of the material's ability to effectively sequester carbon, sulfur, or both comprises application of x-ray diffraction (XRD), x-ray fluorescence (XRF), or equivalent means for identifying material chemistry, and using the results thereof to compare against empirical data in evaluating the material's ability to effectively sequester carbon, sulfur, or both.

In aspects, the technology provides a method of synthetic hydrogen production from a geologic unit, comprising (1) contacting a material identified as an effective hydrogen-generating material and (2) collecting at least a portion of the generated hydrogen.

Aspects related to the generation of hydrogen in materials and the analysis of hydrogen are further described by the Examples and associated figures provided herewith.

### Time-Differentiated Extraction of Substances

According to aspects, inventive technology(ies) provided herein are directed to obtaining and analyzing a plurality of aliquots of volatile substance(s) extracted from material sample(s), wherein each of the plurality of aliquots are obtained under different extraction states, wherein an extraction state is defined at least in part by the type of extraction force, the strength of the extraction force, the time the extraction force is applied, and combination(s) thereof. In aspects, application of such a plurality of extraction states, e.g., at least about 3 or at least about 4 extraction states, provides actionable insight(s) regarding the presence or absence of commercially relevant resource(s), such as, e.g., hydrogen, and the location(s) within a geologic unit where such resource(s) are located. In certain aspects, step(s) of method(s) related to the use of multi-aliquot rock volatile stratigraphy method(s) can be applied to any suitable material, not just rock materials. In aspects, analysis can be of any volatile substance, e.g., hydrogen and other volatile substances such as, e.g., helium and others, including but not limited to ammonia, nitrogen, oxygen, and argon.

According to aspects, technology(ies) provided herein are directed to multi-aliquot extraction method(s), wherein, at least 2, at least 3, or at least 4 or more aliquots are obtained. In aspects, aliquots referenced here are aliquots of non-condensable volatile(s) obtained by the application of different extraction states.

In summary, testing rock across different depths of the well can yield useful data about the presence of a given resource, e.g., H2. However, as discussed here, limited extraction state(s) are applied, e.g., a single extraction state is applied or two extraction states are applied, no hydrogen at one or more locations may be observed or one may be misled as to how tightly the H2 is held. For example, if it is tight rock, H2 may be present but weak extraction states (e.g., A1 or A2 exemplified herein) are note sufficient to get it out; or, similarly if only a strong extraction state (e.g., B1 or B2 exemplified herein) is used, such state(s) may be strong enough to release any H2 present, regardless of how tightly it is held. Obtaining the multiple aliquots (different pressures, different times) provides granularity as to (a) where H2 is located and (b) what type of rock is present at each location (loose vs. tight). Further, the relationship(s) of the various aliquot profiles to one another across depths can tell you something about where pay zones of H2 may be located. In certain aspects, easy to extract (high Al aliquot, low other aliquots) located above hard to extract (high B2 aliquot, low other aliquots) may indicate that a seal exists-H2 is caught below a seal. The seal is over rock which has held onto lots of H2 (given the size of a B2 peak) due to the high pressure caused by the seal-it is saturated rock. That H2 is still there and hasn't been lost, whereas elsewhere, H2 gets lost because the rock is unable to hold onto it and there is no seal keeping it from escaping.

In certain aspects, extraction states vary by the type of extraction force applied in the generation of each of a multitude of aliquots, e.g., crushing, vacuum pressure, temperature, etc. In aspects, extraction states vary by the strength of force applied in the generation of each of a multitude of aliquots, e.g., the strength of vacuum pressure applied. In aspects, the type of extraction force applied in the generation of each of a multitude of aliquots vary by the length of time an extraction force is applied, such as, e.g., a time ranging from, e.g., about 10 sec to about 15 min, e.g., ~30 sec-~15 min, ~45 sec-~15 min, 10 sec to about 10 min, e.g., ~30 sec-~10 min, ~45 sec-~10 min, 1 min to about 10 min, e.g., ~1 min-~10 min, ~1 min-~9 min, or, e.g., e.g., ~30 sec-~2 min, ~6 min-~10 min, or, e.g., ~1 min-~8 min.

In certain specific aspects, disclosed herein are multi-aliquot RVS methods wherein at least 3, e.g., at least 4 aliquots are obtained, wherein at least two aliquots vary by the strength of a single extraction force, e.g., a vacuum force; at least two aliquots vary by the time such a single extraction force is applied, or both are true.

In one specific example, a first extraction state yielding a first aliquot of volatile(s) extracted from a material sample comprises a relatively weak vacuum pressure applied for a relatively short period of time, e.g., a vacuum pressure of about 20 mbar applied for between about 10 sec and about 2 min, such as, e.g., for about 1 min. In aspects, such an aliquot, e.g., “A1” represents the gentlest extraction, trapping non-condensable gas (NCG) within, e.g., in less than about 1 minute after sample volatiles are inlet into the static-vacuum inlet system. These volatiles are captured with the least energy of the 4 NCG extracts, and as such are the most easily extracted volatiles.

In another specific example, a second extraction state yielding a second aliquot of volatile(s) extracted from a material sample comprises a relatively weak vacuum pressure, e.g., the same relatively weak vacuum pressure, applied for a relatively longer period of time, e.g., a vacuum pressure of about 20 mbar applied for between about 5 min and about 15 min, e.g., for about 8 min. In aspects, such an aliquot, e.g., “A2” is also part of a first aliquot of volatiles to be analyzed, but these gas samples are trapped at the end of the approximately 8 minute extraction of volatiles from the sample under the more gentle vacuum conditions, starting at about, e.g., 20 millibars. These volatiles are extracted from deeper in the fabric of the rock cuttings than the A1 data.

In aspects, a third extraction state yielding a third aliquot of volatile(s) extracted from a material sample comprises a relatively stronger vacuum pressure (but, e.g., still a vacuum pressure characterizable as a gentle vacuum), applied for a relatively short period of time, e.g., a vacuum pressure of about 2 mbar applied for between about 10 sec and about 2 min, such as, e.g., for about 1 min. In aspects, such an aliquot, e.g., “B1” is the gentler aliquot 2 volatiles extraction, captured in less than 1 minute after the extraction chamber vacuum has been dropped to about 2 millibars pressure (representing a vacuum pressure increase). These volatiles are extracted from deeper in the fabric of the rock cuttings than the A1 and Al data.

In aspects, a fourth extraction state yielding a fourth aliquot of volatile(s) extracted from a material sample comprises a relatively stronger vacuum pressure, e.g., the same relatively stronger vacuum pressure, applied for a relatively longer period of time, e.g., a vacuum pressure of about 2 mbar applied for between about 5 min and about 15 min, e.g., for about 8 min. In aspects, such an aliquot, e.g., “B2” data are the last volatiles to be trapped at the end of the B2 volatiles extraction for about 8 minutes. These are the volatiles extracted from the deepest parts of the cuttings samples.

According to aspects, samples utilized in obtaining such aliquots are sealed at the well samples and are not physically crushed prior to volatiles extraction. In alternative aspects, such samples could further be extracted to crushing as part of an extraction step.

In certain aspects, the amount of one or more volatiles extracted varies by aliquot, e.g., varies by extraction state applied. In certain aspects, one or more volatiles can be present in DOS quantity (ies) in one aliquot but not another. For example, in certain aspects, hydrogen may be present in one aliquot in DOS quantities but not other aliquots. If a target analyte, e.g., hydrogen, were to be absent from a single or multiple aliquots collected according to particular extraction states, but present in at least one aliquot obtainable by a different extraction state which is not applied and thus such an aliquot is not collected and analyzed, it would appear that such a target analyte, e.g., hydrogen, is not present in the material sample, e.g., is not present at the location from which the material sample is collected.

In a more specific example, in certain aspects, if only a single extraction force e.g., a vacuum pressure, is used to obtain volatile(s), e.g., hydrogen, from material sample(s), e.g., drill cuttings in methods of identifying target location(s) for resource such as hydrogen production; if a single extraction force strength, e.g., a relatively weak gentle vacuum extraction force, e.g., a force of about 20 mbar, is used to obtain volatile(s), e.g., hydrogen, from material sample(s), e.g., drill cuttings in methods of identifying target location(s) for resource such as hydrogen production; or, e.g., if a single extraction force application time, e.g., a relatively short extraction time, e.g., an extraction time of less than about 5, ≤~4, ≤~3, ≤~2 or ≤1 minute, is used to obtain volatile(s), e.g., hydrogen, from material sample(s), e.g., drill cuttings, in methods of identifying targets for resource (hydrogen) production, but the target resource (hydrogen) is only detectable under a different extraction force, a different extraction force strength, or when exposed to a/the extraction force for a different period of time, then it would appear that such a resource is not present in the material, e.g., material sample collected from a location under consideration for potential resource production.

This can be misleading, and result in potential targets for resource production being missed. In aspects, it is now demonstrated that how easy volatile(s) are released from material(s) is useful/insightful information.

In aspects, provided herein are multi-aliquot analytical method(s), e.g., multi-aliquot rock volatile stratigraphy method(s) wherein a plurality of combination(s) of extraction force(s), extraction force strength(s), and extraction force application time(s) are used to identify possible location(s) for resource production. In aspects, provided herein are multi-aliquot analytical method(s), e.g., multi-aliquot rock volatile stratigraphy method(s) wherein a plurality of combination(s) of extraction force(s), extraction force strength(s), and extraction force application time(s) are used to identify possible location(s) for resource production wherein if one or more aliquots of such a multi-aliquot analysis were not obtained or were not available for analysis, one would not identify a likely resource payzone which is otherwise identifiable in the presence of such aliquot(s).

According to aspects, extraction force(s) applied herein are gentle vacuum forces or gentle vacuum equivalent forces. In aspects, sample(s) are subjected to a single gentle vacuum force or single gentle vacuum equivalent force, however for at least two different periods of time. In certain respects, such extraction state(s) yield different amount(s) of one or more target volatile(s), e.g., hydrogen. According to aspects, sample(s) are subjected to at least two different gentle vacuum forces or gentle vacuum equivalent forces, however for a single (the same) period of time. In certain respects, such extraction state(s) yield different amount(s) of one or more target volatile(s), e.g., hydrogen. According to aspects, sample(s) are subjected to at least two different gentle vacuum forces or gentle vacuum equivalent forces and are further subjected to such different gentle vacuum forces or gentle vacuum equivalent forces for different periods of time. In certain respects, such extraction state(s) yield different amount(s) of one or more target volatile(s), e.g., hydrogen.

In aspects, method(s) of target resource production location(s) (or, e.g., also or alternatively target resource production location(s) comprising step(s) described above identify locations likely to be payzones for the target resource, e.g., hydrogen.

In certain specific embodiments, sample(s) are subjected to a first gentle vacuum extraction force, e.g., a force of between about 10 mbar and about 30 mbar, such as, e.g., ~15 mbar-~25 mbar, e.g., ~20 mbar, for a first time period, e.g., a time of about 30 sec to about 3 min, e.g., ~45 sec-~2 min, or, e.g., ~1 min. In aspects, a first aliquot is obtained under such conditions. Samples are then subjected to the same gentle vacuum extraction force, but for a second time period, e.g., a time of about 5 min to about 10 min, e.g., ~7 min-~9 min, or, e.g., ~8 min. In aspects, a second aliquot is obtained under such conditions. Samples are then subjected to a different, e.g., stronger gentle vacuum extraction force, e.g., a force of between about 0.5 mbar and about 5 mbar, e.g., ~1 mbar-~3 mbar, e.g., ~2 mbar, for a first time period, e.g., a time period of about 30 sec to about 3 min, e.g., ~45 sec-~2 min, or, e.g., ~1 min. A third aliquot is obtained under such conditions. Samples are then subjected to the same stronger gentle vacuum force but for a second time period, e.g., a time of about 5 min to about 10 min, e.g., ~7 min-~9 min, or, e.g., ~8 min. In aspects, a fourth aliquot is obtained under such conditions. Upon the collection of all aliquots, data from each aliquot for each analyte measured can be plotted. Differences in the amount(s) of the analyte relative to one another at different depths from which samples were collected can be considered.

In certain aspects, the identification of locations comprising a high amount of relatively easily extractable target substance indicates a potential payzone, or target location for resource production.

In certain alternative aspects, the identification of locations comprising a high amount of target resource that is DOS more extractable under the stronger extraction conditions, located in locations below, deeper to, locations at which high amounts of more loosely held resource (hydrogen) indicate a potential payzone or target location for resource production. In aspects, such scenarios may indicate that a cap exists. In aspects a cap may be a helium cap. In aspects, samples at a depth indicating a possible depth can be tested for, e.g., helium, to validate such a prediction. In aspects, the identification of such scenarios indicate that the resource (hydrogen) is so loosely held and so readily accessible that the majority of it is lost before reaching the Earth's surface across most depths. Above the cap, hydrogen is not identifiable in weaker extraction aliquots because it has escaped. Below the cap the hydrogen is trapped; it is or may be abundant and is identifiable as readily releasable just under the cap. However, further below the cap, the resource (hydrogen) again does not appear in the weaker extraction aliquots, because it has migrated up to the cap. Deeper to such an abundance of readily releasable resource (hydrogen) an increase in tightly held resource is observed while aliquots measuring loser-held hydrogen at such depths demonstrate little to no hydrogen. This can indicate that the hydrogen is abundant at this location. The seal is preventing hydrogen from migrating and a possible payzone exists in tightly held H2 form.

According to aspects, aliquot(s) comprise only non-condensable gases. In certain aspects, the disclosure here can be applied to all rock volatile stratigraphy (RVS) methods disclosed here and further disclosed in Prior Smith Patents, wherein in aspects such RVS method(s) comprise volatile trapping (e.g., the presence of a cryotrap) and wherein alternative aspects such RVS method(s) do not comprise volatile trapping (e.g., no cryotrap is present) and further in methods wherein sample crushing is applied as one extraction force and alternatively no sample crushing is applied. In aspects, applicable RVS method(s) can comprise use of any suitable analytical instrument such as, e.g., a mass spectrophotometer. In aspects where a trapping device is used, trapped volatiles may or may not be released and may or may not be analyzed. In aspects, trapped volatile(s) can be intermittently released, intermittently analyzed, or both. In aspects, speed and sampling density are relevant to such an analysis, as speed and sampling density can DOS improve the resolution of where potential payzone(s) exist. In aspects, device(s) provided herein can be used to DOS improve such resolution by, e.g., increasing the speed and sampling density but still yielding multi-aliquot analyses.

In certain aspects, method(s) disclosed here can be applied to petroleum exploration, hydrogen exploration, helium exploration, carbon sequestration endeavors, carbon storage endeavors, and other geological exploration and exploitation-related endeavors.

Aspects related to the multi-aliquot and extraction-state differentiated analysis of hydrogen and other substances are further described by the Examples and associated figures provided herewith.

### Multiple Non-Condensable Extractions

Another aspect of the technology described herein provides the initially exclusive, primary, or exclusive use of non-condensable gas portion(s) of easily extracted volatile substance(s) from sample(s) (hereinafter “the portion(s)”) for analysis. The portion(s) may be the primary or only source of non-condensable gas(es).

In aspects, the portion(s) disclosed here are obtained by the application of force(s), e.g., vacuum force(s), to sample(s) and passing easily extracted volatile substance(s) from the sample(s) through location(s) to condense condensable gas(es) from the easily extracted volatile substance(s). In aspects, such location(s) comprise a cryotrap with a temperature of about −100 degrees Celsius. The easily extracted volatile substance(s) having been passed through location(s) in aspects then comprise primarily, or exclusively all non-condensable gas(es), e.g., hydrogen, helium, argon, nitrogen, ammonia, oxygen, non-condensable hydrocarbon(s), e.g. hydrocarbon(s) other than ethane and methane, and other gas(es) that are not condensable when passed through, e.g. a cryotrap.

In aspects, more than one portion described here is generated from a sample by subjecting the sample to more than one condition, e.g., a vacuum, an amount of time, or both a vacuum and amount of time, and passing easily extracted volatile substance(s) from the sample(s) through location(s) to condense condensable gas(es) from the easily extracted volatile substance(s). Such location(s) may comprise a cryotrap with a temperature of about −100 degrees Celsius. In aspects, the easily extracted volatile substance(s), having been passed through location(s), now comprise primarily, or exclusively all non-condensable gas(es), e.g., hydrogen, helium, argon, nitrogen, ammonia, oxygen, non-condensable hydrocarbon(s), e.g. hydrocarbon(s) other than ethane and methane, and other gas(es) that are not condensable when passed through, e.g. a cryotrap. In aspects, the composition of the non-condensable gas(es) may be different than those obtained in a first portion, i.e., different volatile substances, fewer or greater, different amounts of the same volatile substances in the first portion, or both.

The portion(s) described here, in aspects, are analyzed to assist in hydrogen/helium exploration and production, e.g., for the amount of hydrogen, helium and hydrogen proxies, e.g., ammonia, water, nitrogen, and oxygen, wherein nitrogen and oxygen are analyzed in conjunction with argon.

The portion(s) described here may be additionally analyzed to assist in hydrocarbon exploration and production.

In aspects, portion(s) herein may be analyzed to assist in carbon sequestration, sulfur sequestration, both carbon and sulfur sequestration, or non-carbon, non-sulfur compound(s) sequestration.

Aspects related to the formation of multiple, non-condensable extractions, e.g., portions, are further described relative to the disclosure of Examples and associated figures provided herewith.

### Analytical Devices

According to embodiment(s), disclosed herein are device(s) for the rapid analysis of one or more volatile substances, e.g., easily extracted volatile substance(s), e.g., hydrogen and helium, among others.

In aspects, device(s) provided herein are designed for use in conjunction with solid or semi-solid material samples, e.g., rock material samples, e.g., solid or semisolid mineral aggregate material(s) from which at least two easily extracted volatile substances are obtained.

However, as discussed above, successful geologic hydrogen exploration and production is not simply a matter of producing very large volumes of hydrogen, but often very much requires the production of very pure hydrogen if that hydrogen is to be used for energy production.

Work in the field of resource exploration has shown that hydrogen purity varies stratigraphically, with hydrogen-rich zones occurring juxtaposed to Helium-rich zones. It is not known to what scale such zoning occurs. For example, large oil fields in the Mexico portions of the Gulf of Mexico found large oil deposits disseminated in a multitude of fine layers, layers too thin to be realized by state-of-the-art wireline assessment. The resource was only realized by extensive coring. Without taking such expensive cores, this resource may not have been realized.

What is typically required for fine-scale stratigraphic identification of pure hydrogen zones is a nearly as continuous as possible method of analyzing hydrogen and related volatiles in cuttings at a well site as quickly as practical after the cuttings arrival at the surface, and to dispose of those cuttings from the apparatus.

Technology (ies) disclosed herein comprise a device capable of providing nearly continuous sampling and analysis of drill cuttings collected from an active drilling operation, wherein the device further disposes such cuttings from the device upon completion of analysis.

Device(s) herein comprise a chamber that quickly inlets bulk cuttings samples into the vacuum inlet, quickly extracts hydrogen and other volatiles, if desired moves these volatiles though or near a cryogenic trap to remove other hydrogen bearing species such as water and hydrocarbons, passes that gas through appropriate inlets into an analytical device, e.g., the high vacuum mass spectrometer chamber, where those volatiles are rapidly analyzed while the previous cuttings sample is discarded and the following cuttings sample is being extracted.

In one aspect, the inlet design of the device is similar to a horizontal revolving door. In aspects, material samples, e.g., cuttings samples (“cuttings”) are loaded through an open port at the top of the device, moved, e.g., rotated, to a horizontal volatiles extraction position, then moved, e.g., rotated, to a bottom section where the cuttings are discarded using gravity. In aspects, the process is repeated as quickly as possible to provide the densest cuttings hydrogen and other volatiles log possible. These data generated, e.g., generated at a well site, are used to aid in directing drilling decisions, select zones for other types of testing and analyses, monitor for rig hazards, and to define depths for other testing of cuttings for chemical and physical characterization.

### Characteristics

### Location

In aspects, device(s) can be located at a resource exploration, e.g., a resource production site. In aspects, device(s) can be located at an active drilling site.

In aspects, device(s) are located a sufficient distance from the possum belly to DOS reduce the risk of sparking, e.g., to reduce fire risk. In aspects, devices are located in a mud shack. In aspects, device(s) are located near the slew of an active drilling site.

Due to its potential location at an active drilling site, device(s) herein are characterizable as well-site analyzer(s).

According to aspects, device(s) herein are compact enough such that it can be deployed at a well site to enable highly refined data to indicate where resources are located that would not otherwise be located. According to aspects, device(s) herein are fast enough that it can be deployed at a well site to enable highly refined data to indicate where resources are located that would not otherwise be located. According to aspects, device(s) herein are both sufficiently compact and fast such that they are suitable for deployment at a well site.

### Speed

According to aspects, well site analyzer(s) operate at speed of sample collection and analysis of between about 1 inch (in, or”) and about 5 feet (ft, or ‘) per sample, e.g., ~1 in-~4 ft, ~1 in-~3 ft, ~1 in-~2 ft, or, e.g., ~1 in-~1 ft per sample, such as, e.g., ~2 in-~5 ft, ~4 in ~5 ft, ~6 in-~5 ft, ~8 in-~5 ft, ~10 in-~5 ft, or, e.g., ~1 ft-~5 ft, as in, e.g., ~2 in-~4 ft, ~4 in-~3 ft, ~6 in-~2 ft, ~1 in-~18 in, ~8 in-~16 in, or ~10 in-~14 in, such as, e.g., about 1 foot per sample.

In aspects, well site analyzer(s) herein operate at a speed of between about 10 ft and about 2000 ft per hour (hr), such as, e.g., ~10 ft-~1800 ft, ~10 ft-~1600 ft, ~10 ft-~1400 ft, ~10 ft-~1200 ft, ~10 ft-~1000 ft, or ~10 ft-~900 ft per hour, as in, e.g., ~50 ft-~2000 ft, ~100 ft-~2000 ft, ~150 ft-~2000 ft, ~200 ft-~2000 ft, ~250 ft-~2000 ft, ~300 ft-~2000 ft, ~350 ft-~2000 ft, ~400 ft-~2000 ft, ~450 ft-~2000 ft, ~500 ft-~2000 ft, ~550 ft-~2000 ft, ~600 ft-~2000 ft, ~650 ft-~2000 ft, ~700 ft-~2000 ft, ~750 ft-~2000 ft, ~800 ft-~2000 ft, ~850 ft-~2000 ft, or, e.g., ~900 ft-~2000 ft per hour, as in, e.g., ~100 ft-~1800 ft, ~200 ft-~1600 ft, ~300 ft-~1400 ft, ~400 ft-~1200 ft, ~500 ft-~1150 ft, ~600 ft-~1100 ft, ~700 ft-~1050 ft, ~800 ft-~1000 ft, or, e.g., ~900 feet per hour.

In aspects, well site analyzer(s) herein operate at such a speed that drill cuttings samples can be analyzed at between about every 1 foot and about 50 feet of well depth, such as, e.g., ~1 ft-~45 ft, ~1 ft-~40 ft, ~1 ft-~35 ft, ~1 ft-~30 ft, ~1 ft-~25 ft, ~1 ft-~20 ft, or every ~1 ft-~15 ft of well depth, such as, for example, every ~2 ft-~50 ft, ~4 ft-~50 ft, ~6 ft-~50 ft, ~8 ft-~50 ft, ~10 ft-~50 ft, ~12 ft-~50 ft, ~14 ft-~50 ft, ~16 ft-~50 ft, or every ~18 ft-~50 ft of well depth, as in, for example, every ~2 ft-~45 ft, ~4 ft-~40 ft, ~6 ft-~35 ft, ~8 ft-~30 ft, ~10 ft-~25 ft, ~12 ft-~20 ft, ~14 ft-~18 ft, or, e.g., every ~15 feet.

In aspects, between about 10 and about 3000 samples can be analyzed per day by analyzers herein, such as, e.g., ~10 samples/day-~2800 samples/day, ~10 samples/day-~2800 samples/day, ~10 samples/day-~2600 samples/day, ~10 samples/day-~2400 samples/day, ~10 samples/day-~2200 samples/day, ~10 samples/day-~2000 samples/day, ~10 samples/day-~1800 samples/day, ~10 samples/day-~1600 samples/day, or ~10 samples/day-~1500 samples/day, as in, e.g., ~100 samples/day-~3000 samples/day, ~200 samples/day-~3000 samples/day, ~400 samples/day-~3000 samples/day, ~600 samples/day-~3000 samples/day, ~800 samples/day-~3000 samples/day, ~1000 samples/day-~3000 samples/day, ~1200 samples/day-~3000 samples/day, or ~1400 samples/day-~3000 samples/day, as in, e.g., ~100 samples/day-~2800 samples/day, ~200 samples/day-~2600 samples/day, ~400 samples/day-~2400 samples/day, ~600 samples/day-~2200 samples/day, ~800 samples/day-~2000 samples/day, ~1000 samples/day-~1800 samples/day, ~1200 samples/day-~1600 samples/day, or, e.g., ~1400 samples/day-~1500 samples/day, as in, e.g., about 1440 samples/day can be analyzed by analyzer(s)/device(s) herein.

In certain specific aspects, well site analyzer(s) herein operate at a speed of about one foot per sample which drilling at 900 feet/hour. In aspects, well site analyzer(s) herein operate at a speed allowing for drill cutting(s) samples to be analyzed at about every 15 feet of depth. In aspects, about 1440 samples per day can be tested.

Device(s) herein are suitable for use with one, some, most, generally all, substantially all, essentially all, or all rock volatile stratigraphy method(s) described herein, described some, most, generally all, substantially all, essentially, all, or all Prior Smith Patents, or combinations thereof. In certain aspects, one or more steps of rock volatile stratigraphy method(s), or, e.g., the time allocated for the completion of one or more steps of the rock volatile stratigraphy method, can be varied to accommodate for the amount of time required to collect and seal one or more material samples by device(s) herein. In aspects, a variation in the time allocated to one or more steps of rock volatile stratigraphy method(s) is established in coordination with the frequency of collection of multiple material samples such that the analysis of each of material sample is completed in coordination with when the next material sample becomes available for analysis. In some respects, one or more steps of rock volatile stratigraphy method(s) herein or the time allocated for the completion of one or more steps of the rock volatile stratigraphy method, can vary between the rock volatile stratigraphy method applied to one material sample and that applied to another material sample.

### Samples

According to aspects, device(s) herein are suitable for analyzing solid or semi-solid material samples, e.g., geological samples, e.g., rock material samples, e.g., drill cutting(s) samples, core sample(s), drilling muds, etc. In aspects, rock material samples are solid or semisolid mineral aggregate material(s). In aspects, sample(s) are any type of rock material sample(s).

In aspects, samples are suitable for the use in the analysis of hydrogen, helium, and, e.g., other non-condensable gases, which can be, for example, applied at or near a well site and applied to geologic materials including, materials which are mostly, materials which consist substantially of, or materials consisting of, drill cuttings samples, core samples, or drilling muds.

According to aspects samples comprise a known amount, e.g., a known volume, a known weight of material (e.g., cuttings and herein simply referred to as cuttings or simply “sample” for sake of illustration, as is the case with all the examples herein), or both a known volume and known weight of sample.

In aspects, sample(s) are delivered to the analyzer(s) herein, e.g., more specifically, to a movable container component, also referred to herein as the collection and transfer component (CTC) either, for example, automatically or, e.g., by person. In aspects, sample(s) are delivered as a stream of material and, e.g., the stream of material is sampled at set interval(s), e.g., analyzer(s) are adapted to capture sample(s) from the stream of material at defined interval(s). In aspects, such intervals are, e.g., between about every 0.25 min and about every 3 min, such as, e.g., ~0.25 min-~2.75 min, ~0.25 min-~2.5 min, ~0.25 min-~2.25 min, ~0.25 min-~2 min, ~0.25 min-~1.75 min, ~0.25 min-~1.5 min, ~0.25 min-~1.25 min, or ~0.25 min-~1 min, as in, e.g., ~0.5 min-~3 min, ~0.75 min-~3 min, or ~1 min-~3 min, as in, e.g., ~0.35 min-~2 min, ~0.45 min-~1.5 min, or, e.g., about every 0.5 min-~every 1 min.

In aspects, the amount of sample that is captured in the location can vary in volume such as to ensure there is little to no open headspace or a lot of headspace in the compartment of a movable container component into which sample is collected.

According to aspects, sample(s) can be collected from a stream of material delivered to the analyzer. Whereby a known volume, weight, or both known volume and weight, of sample is collected. Here, a “known” volume, weight, or both, of material can be, e.g., an at least relatively uniform and consistent or approximated volume, weight, or both, which is established by the timing of the rotation of the movable container component/CTC, whereby the sample entry point of the device is alternatingly exposed and not exposed to the stream of material for consistent periods of time and wherein the stream of material is provided at a relatively consistent volume and rate. In aspects, a “known” volume, weight, or both, is a volume, weight, or both which varies from a target or expected volume, weight, or each of an expected volume and weight by no more than about 50%, such as, e.g., ≤~45%, ≤~40%, ≤~35%, ≤~30%, ≤~25%, ≤~20%, ≤~15%, ≤~10%, ≤~5%, ≤~4%, ≤~3%, ≤~2%, or ≤~1%.

### Extraction

In certain aspects, samples are not crushed prior to the extraction of volatile substance(s) therefrom. In aspects, device(s) herein extract one or more volatile substance(s), e.g., rock volatile substances, extracted therefrom. In aspects, sample(s) captured and analyzed by device(s) herein comprise an amount of volatile(s) extracted, wherein the volatile(s) extracted comprise a condensable portion and a non-condensable portion. In aspects, the non-condensable portion is larger than the condensable portion. In aspects, condensable volatile(s) extracted are removed prior to the analysis of the non-condensable portion by, e.g., one or more component(s) of device(s), e.g., a trap component, such as, e.g., a cryotrap.

According to aspects, volatile(s) trapped by a trap component are not analyzed. According to certain aspects, volatile(s) trapped by a trap component are discarded. In certain other aspects, trapped volatile(s) can be analyzed.

### Purging

According to aspects, sample(s) are collected in chamber(s), e.g., compartment(s), of a movable container component of device(s) herein. In aspects, such compartment(s) are at least at certain points of operation of the device exposed to the environment, e.g., to the open air. In aspects, device(s) herein comprise a location, e.g., compartment(s), to capture sample(s) that is/are open to the external environment whereby upon capture of sample(s), the compartment(s) comprise a headspace; e.g., sample(s) do not completely fill the compartment(s) such that an amount of ambient air exists in the compartment. The amount of sample that is captured in the location can vary in volume, such as to ensure there is little to no open headspace or a lot of headspace.

According to certain aspects, such headspace can be purged. In aspects, the location of device(s) herein where sample(s) are captured can be overlaid with a heavier gas than air. In aspects, a purging gas can be supplied through the same device opening through which sample(s) is/are collected. In aspects, a purging gas can be supplied through one or more other opening(s) of the device, such as, e.g., from a position opposite to, adjacent to, lateral to, etc. the location where sample(s) enter the device.

According to aspects, suitable purging gases can be any inert gas heavier than air, such as, e.g., xenon, krypton, or argon. In aspects, argon is used as a purging gas. In aspects, the purging gas is DOS recoverable. In aspects, some, most, generally all, substantially all, essentially all, or all gas overlaying the sample can be extracted at one or more pressures, e.g., one or more vacuum pressures. In certain aspects, a purging gas overlaying sample(s) can be passed over a cryogenic chamber.

### Exemplary Device Components

According to aspects, device(s) (well site analyzer(s)/analyzer(s)) provided herein comprise a number of components, the components being suitable for rendering the device capable of performing the function(s)/activity (ies) described herein. Exemplary device component(s) are described here. Such device component(s) can be arranged in any suitable configuration which render(s) the device(s) operable and capable of performing their designed function, e.g., rapidly collecting sample(s), extracting volatile(s) therefrom, analyzing extracted volatile(s), and discarding sample(s), at such a speed so as to provide content analysis of material samples collected at very fine intervals, e.g., very fine depth intervals, such as intervals of less than about 50 ft, e.g., ≤~45 ft, ≤~40 ft, ≤~35 ft, ≤~30 ft, ≤~25 ft, ≤~20 ft, or, e.g., ≤~15 ft.

### Movable Container Component

In aspects, device(s) comprise a movable container component, which may be referred to as a collection and transfer component (CTC). Herein, movable container component and collection and transfer component or CTC can be used interchangeably. In aspects, a movable container component is a relatively rapid moving component, e.g., capable of rapidly collecting sample(s) from a first device location and transferring the sample(s) to a second device location at intervals of no more than, e.g., about 2 min, e.g., no more than about 1.5 min or, e.g., in less than or equal to about 1 min, 50 seconds (50 sec), 40 sec, or, e.g., in less than or equal to about 30 sec. In aspects, a movable container component can be any component, in any shape, size, or orientation, capable of collecting, e.g., receiving, sample(s) and moving the sample(s) from the point of their receipt by the device to one or more other position(s) within the device.

In aspects, a movable container component comprises a plurality of compartments.

According to embodiments, the moveable container component is a rotational container. In aspects, the rotational container is configured to gravitationally receive a second sample when delivered to the device while depositing a first, prior-collected sample to a location where such a sample can be subjected to an extraction component present in the device. Further, the movable container component is, in aspects, capable of moving collected sample(s) to a position for discarding the sample(s) in order of their collection. In aspects, a movable container component (or CTC) is capable of sequentially moving one or more compartments thereof from a first location for sample collection, to a second location for sample extraction, to a third location for sample discard, and, e.g., returning the compartment to the first location of collection of another sample. As depicted in exemplary figures of such device component(s) in figures provided herewith, the presence of a plurality of compartments of movable container components (aka CTCs) allows for ongoing and cyclical sample collection, analysis, and discard.

In aspects, a device location containing a sample can be moved, e.g., rotated, to move the samples through the device.

In certain aspects, a movable container component/CTC operates to (a) collect material sample(s) and (b) transfer collected sample(s) to one or more different positions. In aspects, a CTC serves to collect geologic material or drilling mud samples from a larger source of material, such as, e.g., a stream of geologic material or, e.g., drilling mud, provided to the analyzer. In aspects, a CTC can be characterized as a rock volatiles sampling device.

According to aspects, device(s) can comprise one or more vacuum seal(s). In aspects, vacuum seal(s) can serve to participate in the sealing of each of the compartments of a movable container component when such compartments are in one or more positions.

According to aspects, movable container components (CTCs) comprise an entry point at which a sample is dropped, conveyed, or collected. A movable container component can move such that, e.g., after collection, the CTC is moved, e.g., rotated 90 degrees, such that collected samples are in position for volatile(s) extraction. In aspects, after extraction, a movable container component can move such that extracted samples are positioned at or near a device sample exit whereby extracted samples (samples having had some, most, generally all, substantially all, essentially all, or all, volatile(s) extracted), exit the analyzer.

Discarded samples can be collected upon discard as collected discarded samples in, e.g., one or more collection unit(s) or, e.g., a system of collection unit(s) can be present for collecting discarded sample(s) for further use (e.g., testing, storage, etc.).

### Compartments

A movable container component/CTC can in aspects comprise a plurality of partitioned sections, e.g., any suitable and practical number of partitioned sections, e.g., between about 1 and about 10 partitioned sections, e.g., ~1-~9, ~1-~8, ~1-~7, ~1-~6, ~1-~5, ~1-~4, ~1-~3, or, e.g., ~2-~10,~3-~10, ~4-~10,~5-~10, ~6-~10, or ~7-~10, as in, e.g., ~2-~9, ~2-~7, or ~2-~5, e.g., ~3 or ~4 partitioned sections or compartments. In aspects, CTCs comprise at least 3 partitioned compartments. In aspects, CTCs comprise about 3 or about 4 partitioned compartments. As the movable container component can be moved, e.g., rotated, the position of each of each of any present compartments is modifiable. Each of the compartments (also referred to herein as “chambers”) can be positioned in each of any number of positions, wherein, in aspects, the present number of positions is the same as the number of present compartments.

In certain aspects, each compartment of a movable container component can occupy each position among a plurality of positions, each compartment (a) comprising a separate inlet, a separate outlet, or both. In aspects, each compartment is capable of both receiving and discarding sample(s). In aspects, each compartment is configured to receive an analyzable sample of a solid or semisolid mineral aggregate material at a first position among the plurality of positions and to deliver the sample to one or more other positions of the plurality of positions of a movable container component.

In aspects, compartments of a CTC/movable container component can comprise one or more vacuum seal(s). In aspects, vacuum seal(s) operate to, at least for a period of time during device operation, and at least in part, seal compartments from the external environment.

### Extraction Component

According to aspects, device(s) herein comprise an extraction component. In aspects, an extraction component can be any component, feature, or, e.g., system, capable of extracting one or more volatile substance(s) from sample(s) collected by the device, e.g., by application of one or more force(s).

In aspects, an extraction component is positioned in effective proximity to at least one of the plurality of positions of a movable container component such that samples present in a compartment located at such position can be at least selectively exposed to the extraction component.

In aspects, an extraction component is configured to selectively apply one or more gentle vacuum equivalent extraction forces to extract one or more aliquots of easily extractable volatile substances from sample(s) of material received by the movable container. In aspects, an extraction component is associated with, directly or indirectly, e.g., is operationally associated with (e.g., depends upon the presence of) one or more other device component(s) to aid in its operation, such as, e.g., one or more vacuum pump(s).

### Vacuum System

In aspects, device(s) herein can comprise a vacuum system. In aspects, such a vacuum system can be any vacuum system suitable for the application of a vacuum, e.g., a vacuum extraction force. In aspects, a vacuum system is a selectively operable, conditionally automatically operable, or selectively and conditionally automatically operable vacuum system.

In aspects, a vacuum system of device(s) herein is positioned downstream of an extraction component, analytical component, or both. In aspects, a vacuum system applies a vacuum force that draws an aliquot or portion of the aliquot of extracted volatile(s) through a flow path from the material sample located in a compartment of a movable container component, to an extraction component, and to the analytical component.

In aspects, a vacuum system is operationally associated with an extraction component, such that, e.g., the vacuum system applies the vacuum to an extraction component which extracts volatiles by way of the application of vacuum pressure. In aspects, pressure(s) applied to samples as extraction force(s) to release volatile substances for analysis can be some level of vacuum pressure, such as two or more different pressures that result in measurably different, substantially different, or significantly different aliquots with respect to one or more substances. In aspects, vacuum pressures are gentle vacuum pressures, e.g., pressures no stronger than about 2 mbar, e.g., ≤~4 mbar, ≤~6 mbar, ≤~8 mbar, ≤~10 mbar, ≤~12 mbar, ≤~14 mbar, ≤~16 mbar, ≤~18 mbar, or ≤~20 mbar in strength (wherein the lower the mbar value, the stronger the vacuum strength).

According to particular aspects, vacuum pressures are programmed or are present as part of default setting(s) of a device.

Analyzer(s) can comprise one or more vacuum valves. In aspects, vacuum valve(s) can be present in any suitable location within the device(s) to participate in the control of vacuum forces and where and to what such vacuum force(s) are applied. In aspects, vacuum valves can operate to control the flow of fluid through portion(s) of the analyzer wherein such portion(s) of the analyzer may be subject to a vacuum pressure.

In certain respects, an inlet chamber is positioned between 2 vacuum valves. In operation, an inlet chamber can be brought to detectable or significant vacuum using one or more vacuum pumps e.g., more specifically a high vacuum pump and a roughing pump which can be present as element(s) of a vacuum system. In aspects, a vacuum system can be sealed off to an inlet chamber by vacuum valve(s).

In certain aspects, pressure in a cuttings volatiles extraction chamber associated with the extraction component is controlled the vacuum system. In aspects, the vacuum system can establish the vacuum of an inlet chamber at any suitable value, such as, e.g., a value between about 40 millibars and about 1 mbar, e.g., ~30 mbar-about 1.5 mbar, or, e.g., ~20 mbar-~2 mbar.

In aspects, analyzing cuttings volatiles extracted from samples at 2 or more different pressures has been demonstrated herein to reveal zones from which high-purity hydrogen may be extracted, and as such, multiple-pressure analyses by analyzer(s) described herein can be desirable or otherwise advantageous and can be provided by device(s) herein.

In certain aspects, analyzers provided herein can comprise an exhaust associated with vacuum pump(s) of a vacuum system.

### Container Movement Component

In aspects, device(s) herein comprise a container movement component. In aspects, a container movement component is any component capable of causing the movement of a movable container component, such as, e.g., a motor and the like. In aspects, a container movement component at least in part relies upon gravity. In aspects, a container movement component causes the movable container to move and thereby causes the different compartments of the plurality of compartments of a movable container component to be located at a/the plurality of positions at different times during operation of the device.

According to aspects, device(s) are configured to cause the container movement component to move the movable container in such a manner facilitating a first compartment of a CTC to be in a first position (for sample collection) and a second compartment to be in a second position (for sample extraction) and to, thereafter move each of the first compartment and second compartment to different positions, such as, e.g., the first compartment to the second position whereby the sample therein is in position for sample extraction and the second compartment to a third position whereby the sample therein is in position for discard.

### Trap Component

In certain respects, device(s) herein comprise the ability to remove one or more volatiles(s) extracted by material samples. In aspects, such removal facilitates a DOS improvement in the ability of the device(s) to accurately measure the amount of one or more other volatile compound(s)/substance(s). In aspects, such a trap component can be any component or comprise any technology suitable for selectively removing one or more target volatile substance(s). Herein, a trap component may also or alternatively be referred to as a volatile compound capture component (“VCCC”).

In aspects, a trap component is a component adapted to selectively remove condensable volatiles. In aspects, a VCCC is a component that selectively captures certain volatile compound(s) by way of condensation. In aspects, device(s) can comprise trap component(s) for capturing released volatile substances (released by application of force(s), e.g., pressure(s)), such as, e.g., cryogenic trap(s), such as a material cooled with liquid nitrogen applied to a suitable surface, wherein such a cryochamber/cryogenic trap can remove a measurable or significant amount, concentration, or number of condensable gases, such as, e.g., water and, e.g., hydrocarbons other than ethane and methane.

In aspects, a VCCC (trap component), e.g., a cryogenic trap, can be held at, e.g., a temperature of minus 100 degrees C (-100 degrees C) or colder.

Device(s) herein are capable of extracting one or more aliquots of volatile(s) from samples. In aspects, a collection component, e.g., a trap component, e.g., a cryogenic trap of device(s) selectively collects a portion of each of the one or more aliquots. In certain respects, a trap component is not associated with any heating element. In aspects, device(s) comprise a trapped gas disposal component. In aspects, volatile(s) captured by a trap component are not released from the trap component and, e.g., are not measured by device(s). In alternative aspects, trapped volatiles can be released from the trap, e.g., by the presence and application of a heating element, and some, most, generally all, substantially all, essentially all, or all released volatile(s) are measured by the device.

As provided elsewhere herein, an analyzer can comprise vacuum valve(s) positioned between a trap component/VCCC (e.g., cryotrap) and an inlet chamber, whereby, in operation, a vacuum valve positioned between the VCCC and the inlet chamber can be opened, allowing volatile(s), e.g., volatile(s) extracted from material sample(s) to enter a trap component/VCCC, e.g., a cryotrap, through a trap component/VCCC inlet, when such a trap component/VCCC component is present.

### Collection Component

According to aspects, device(s) can comprise a collection component. In aspects, a collection component is any component capable of collecting volatile(s) which, having passed through a trap component if present, collect non-trapped volatile(s). In aspects, a collection component collects volatile(s) extracted from material samples prior to their analysis by an analytical component, wherein the collection component collects at least a portion of any aliquot of volatile(s) extracted from sample(s), wherein the at least portion of aliquots collected comprises volatile(s) not trapped by any present trapping component.

In certain respects, a collection component is adapted such that volatile(s) collected thereby comprise at least one hydrogen proxy if present in the sample.

### Analytical Component

According to aspects, device(s) herein comprise an analytical component. In aspects, an analytical component is any component suitable for and capable of analyzing volatile substances, e.g., substance(s) of each collected aliquot collected by a collection component. In aspects, analytical component(s) can be any suitable analytical component described herein, such as, e.g., any analytical component associated with rock volatile stratigraphy method(s) described herein or in Prior Smith Patents. In certain common aspects, an analytical component is a mass spectrometer.

In aspects, analyzers herein comprise an analytical device wherein, after a sufficient period of time, vacuum valve(s) located between a VCCC, e.g., cryogenic trap, and an inlet chamber are closed, and vacuum valve(s) positioned at a VCCC exit the entry of the analytical device (e.g., mass spectrometer) are opened, either simultaneously or one at a time, to facilitate to delivery of volatile substances to the analytical component within the device. According to aspects, hydrogen, helium, and, e.g., other non-condensable volatiles are allowed to enter the analytical device via an analytical device inlet and are subsequently analyzed by the analytical device. Analyzed gases can, in aspects, be pumped out of the analytical device and exit the vacuum system.

### Output Component

In aspects, device(s) herein can comprise an output component, also referred to herein as a control system, for relaying the analysis of the analytical component to a user, a different system, or both, wherein the output component can comprise one or more computer-controlled feature(s) and function(s), such as, e.g., the ability to control the device(s), to collect, analyze, and transmit data, receive device control input(s), and the like.

In aspects, a control system can comprise one or more computer-related or data-related components, such as, e.g., data interface and control component(s), computer component(s), or both.

### Disposal Component

According to aspects, device(s) herein comprise a device disposal component. In aspects, a disposal component can be any component suitable for the removal of analyzed samples. In aspects, a disposal component is configured to remove a sample from the device and dispose of such a sample after the sample has been exposed to the extraction component. In aspects, a disposal component is configured to automatically discard the sample after the extraction component ceases operating on the sample.

In aspects, a disposal component is adapted to dispose of a sample within about 0-60 seconds, e.g., ~15-60 seconds, ~30-60 seconds, ~45-60 seconds, ~0-45 seconds, ~0-30 or, e.g., ~0-15 seconds from the time that a first other sample is collected, a second other sample is delivered for extraction, or both.

### Use

According to particular aspects, in use, device(s) disclosed herein (1) acquire sample(s) such as, e.g., solid or semi-solid samples such as, e.g., drill cuttings, or fluid(s); (2) establish such sample(s) in a selectively sealable container or compartment; (3) optionally replace any headspace comprising ambient air in the container or compartment with a purging gas such as argon; (4) seal the sample from exposure to the outside environment (such as, e.g., by way of repositioning the container or compartment; (5) extract gas(es), e.g., volatile(s), from the headspace of sealed container(s) compartments under one or more extraction conditions, such as, e.g., under one or more vacuum pressures; (6) optionally pass extracted gas(es) over an optionally present trap component such as, e.g., a cryotrap to, e.g., selectively remove volatile(s), such as, e.g., condensable gas(es); (7) analyze at least a portion of extracted volatile(s), e.g., non-condensable gases, in acquired samples; (8) optionally estimate hydrogen content (e.g., by way of participating in, guiding, performing, or controlling hydrogen quantitation method(s) described herein) of material sample(s) provided to the device; (9) optionally collecting purge gas, if utilized, (10) dispose of analyzed samples, wherein disposition can comprise, e.g., collecting analyzed samples for future use, or, e.g., any combination(s) of any or all thereof. In aspects, all such function(s) are performed at a rate of, e.g., about no less than about 1 sample per every 5 minutes, e.g., at a rate of no less than ~1 sample/4.75 min, ~1 sample/4.5 min, ~1 sample/4.25 min, ~1 sample/4 min, ~1 sample/3.75 min, ~1 sample/3.5 min, ~1 sample/3.25 min, ~1 sample/3 min, ~1 sample/2.75 min, ~1 sample/2.5 min, ~1 sample/2.25 min, ~1 sample/2 min, ~1 sample/1.75 min, ~1 sample/1.5 min, ~1 sample/1.25 min, or, e.g., at a rate of no less than about ~1 sample/1 min.

Aspects related to analytical devices are further described by the Examples and associated figures provided herewith.

### Hydrogen Blocking Applications

Another aspect of the technology is to provide methods for protecting materials from hydrogen and for providing materials that have been protected from hydrogen.

Hydrogen is known to be a corrosive substance that can detectably or significantly reduce the performance of materials that have been exposed to hydrogen.

Materials may be naturally exposed to hydrogen or, e.g., may be exposed to hydrogen during one or more processes.

In certain aspects, provided herein are methods of utilizing an inert gas, e.g., helium, for example, to block hydrogen from harming materials. The application of helium to materials may be done on an industrial scale. Examples of materials that may be treated with helium to detectable or significantly reduce the corrosive effects of hydrogen include, for example, any hydrogen-sensitive material, e.g., any material which, either immediately or over time, can be detrimentally affected (in terms of appearance, strength, flexibility, longevity, and the like) by exposure to hydrogen, such as, e.g., steel, and the many forms in may take, e.g., pipes, valves, plumbing fixtures, and building materials.

In certain aspects, hydrogen-sensitive materials are treated with helium in, for example, a chamber that contains helium. In aspects, the chamber air tight. In aspects, materials in the chamber are exposed to a one or more vacuums, e.g., about 100 mbar, about 10 mbar, or less for one or more periods of time, e.g., about 1 minute, about 10 minutes, or longer to remove loosely associated volatile substances from the material, for example, water vapor.

According to aspects, hydrogen-sensitive materials are bathed in helium at less than, equal to or greater than atmospheric pressure inside the container for one or more periods of time, e.g., about 1 minute to about 10 minutes, or longer at one or more other conditions, e.g., less than or greater than room temperature, e.g., about 22 degrees Celsius.

The helium, thus applied, in aspects coats or penetrates the materials to prevent any exposure of the material to hydrogen thereafter from detectably or significantly reducing the performance of the materials.

The helium may, in aspects, occupy voids within in the materials thereby preventing any hydrogen from occupying such voids wherein the hydrogen would otherwise be able to react with the material and detectably or significantly reduce the performance of the material, for example, reduce the material's strength or make the material more brittle.

In certain respects, helium-protected materials may be used for example in the exploration, production and analysis of hydrogen and helium.

In aspects the technology provides method(s) of reducing hydrogen-material interactions comprising (1) contacting a hydrogen-reactive material with an effective amount of helium, (2) allowing the helium to develop a protective association with the hydrogen-reactive material, and (3) removing excess helium from the material. In aspects, method(s) provide a protective association between the helium and the hydrogen-reactive material comprising placing the material in a pressure chamber and exposing the material to a pressure that significantly increases the efficacy of the association, speed of the association, or efficacy and speed of the association of the helium and the hydrogen-reactive material, e.g., iron, steel or both.

Aspects related to hydrogen blocking and hydrogen blocking applications may be further described by the Examples and associated figures provided herewith.

### Computer-Implemented Applications and AI Applications

The technology includes/provides new computer devices/systems (e.g., comprising (1) a computer processor, (2) memory, (3) an input component, and (4) an output component), wherein the computer processor comprises a computer program/engine that is programmed to analyze data regarding materials relating to any one or more above-described aspects of the invention (comprises engine(s) that perform functions related to such aspects). As such aspects of computer technology are well known, they are only briefly discussed here.

In general, methods or steps of methods, described herein, can be performed by one or more devices that are computerized or comprise a computer. Uncontradicted, any “method” (uncontradicted, meaning a “method of the technology”) described here or any step(s) of any method(s) can be adapted to provide a corresponding device/system and vice versa. Thus, disclosure of any method including steps simultaneously implicitly discloses a corresponding device/system comprising components (e.g., programs ARTA engines) that can perform functions corresponding to the steps of the described method (and vice versa).

Terms such as “computing unit,” “computer component,” “computer,” and the like, typically mean a device or device component or system component comprising physical/persistent computer-readable media or other suitable memory media (e.g., PTRCRM as described below) and a processor that processes (“reads”) information in such media. Computer readable media (CRM) can comprise informative data and also functional information (modifiable/programmable computer executable instructions (CEI)). Memory can comprise, mostly comprise, physical, transferrable, and reproducible computer-readable media (PTRCRM) containing stored instructions (engine(s)) and non-functional data (input, analytical data, in-process data, stored record data, output, etc.). Given the nature of the technology, and the specialized functions such devices are programmed/configured to carry out, the computerized devices or systems described herein can be considered special purpose computing devices or systems as such devices or systems typically require specialized engine(s) as exemplified below.

A computer will comprise a memory component or memory system (uncontradicted, such terms being substitutable for one another here) for storing data and for storing instructions (code, engines, an operating system, aspects of user interfaces, and the like). The memory component will typically store computer executable instructions (CEI) (code) (e.g., engines). Memory may also store data used by these and other programs and applications. Data storage/memory unit(s) of a system or other network device can comprise/be in data storage arrays that can include drive array controllers configured to manage read and write access to groups of hard disk drives, solid state drives, etc. Drive array controllers, alone or in conjunction with system component(s) (server device(s)), also can be configured to manage backup or redundant copies of data stored in data repository (ies) (DR(s)) of a system (also sometimes called memory unit(s)) to protect against data failures, etc., e.g., failures that prevent system component(s) from accessing parts/units of memory/data storage. DR(s) can include any suitable form of data repository (ies), including any suitable database(s), such as a structured query language (SQL) database, no-SQL databases, data lakes, and the like. Various types of data structures can store information (e.g., analytical data) in such a database, including but not limited to tables, arrays, lists, trees, and tuples. Furthermore, databases or other DR(s) in data storage can be monolithic or distributed across multiple physical devices. In specific facets, DR(s) are stored in whole or in part and processor function(s) are employed via a cloud platform such as Microsoft Azure, AWS, or Google Cloud, or other cloud platforms exhibiting some, most, or generally all of the capabilities of one or of such systems/networks. Distributed system(s)/component(s) that can make up some, most, generally all, or all parts of a system processor can use distributed memory, whereby, e.g., processors can be endowed with “chunks” of the memory space and communicate via message-passing or similar methods. Each processor can in such aspects have direct access to its local memory and indirect access to nonlocal or remote chunks of memory. In aspects, some, most, generally all or all of system memory is based on a cloud computing platform/paradigm (e.g., DR/data store as a Service/Storage (DaaS) Platform or an Infrastructure as a Service (IaaS) or Platform as a Service (PaaS) Platform, comprising processing and possibly other functions in addition to memory and memory-supportive functions only). In aspects, cloud-based system memory element(s) of a system is or are based on a distributed system, scalable on demand (or automatically) system, or both. In aspects, distributed file systems that form some, most, generally all, or all of the system memory store data across many separate servers. In some facets, most, generally all, or substantially all the system memory is not distributed. Examples of cloud-based storage systems having these, and other features discussed here include Amazon Simple Storage Service (S3) and Windows Azure Binary Large Object (Blob) storage.

The memory component will store data—e.g., data regarding the analysis of material(s) or sample(s), data regarding the generation of hydrogen, data regarding the presence of other volatiles, etc. Terms such as “data” and “information” are used interchangeably here and are only limited to a specific form when so indicated by explicit definition/statement or clear context. Interrelated data, as recognized by the system or a system-component (through programming or otherwise), can be described as a “Record” or “record.” Records can include, e.g., attributes (characteristics) and values (measurements/attributes). In connection with this technology, relevant records comprise, mostly comprise, or at least generally consist of (directly or when translated) natural language messages. E.g., a record can include the body of some, most, or all of an email or other message that is suitable for use with the systems/methods provided here. Records and other data can be stored in memory of the system or a device, etc. In cases, components/systems for collection of large amounts of data may be referred to as a “data repository” (examples of which include data lakes, data warehouses, databases, and the like). Systems of the technology can include such memory storage devices/systems and methods can include storing data generated by the systems/methods, e.g., method outcome data (described elsewhere) in such storage systems for later application. A record can include, e.g., data concerning a particular sample (e.g., including the volatiles extracted from such sample and analyzed by method(s)).

The memory component will also comprise instructions (code, engines, programs) for carrying out function(s), which typically correspond to steps of the above-described methods or for controlling a device of the technology. E.g., the analysis of volatiles in an extracted volatile aliquot can be performed according to a computer program/engine that controls the analysis of analytical data (e.g., peaks in a mass spectrophotometry spectrogram) to provide a quantification of other evaluation of measured volatiles. Thus, the computer/software units/components of devices/systems can be characterized on the basis of “function(s)” that it/they perform. In aspects, a “function” is a computer-implemented action performed by a system component based on both preprogrammed computer readable and executed instruction(s), e.g., in response to input(s). A function also can describe the result of step(s) of a method. The step(s) of such methods/elements of such function(s) can comprise algorithms, methods, and the like described below. In aspects, an “engine” (sometimes also referred to as an “Engine” or “data engine”) refers to computer/processor-executable software program/application (code, algorithm, etc.) or other components of or executed by a computer system which is configured to perform specified function(s), based on computer-executable/readable instructions (“CEI”) contained in a memory (such CEI typically make up much, most, generally all, or all of the engine), or an engine can refer to any other component(s)/system(s) that perform similar functions on a recurring basis (either automatically, conditionally/situationally, on command/selectably, or a combination thereof). Typically, an engine processes input(s) and generates output(s). An engine typically can be implemented on computer(s) located in one or more locations. In aspects, multiple components of an engine are installed and running on one or more computer(s); multiple instances of an engine are installed and running one or more computers; or both. The operation of an engine typically performs function(s), which can represent step(s) in method(s). Such corresponding aspects are implicitly described by any explicit description of an engine or a function (e.g., description of a system/component comprising an engine for performing function(s) implicitly discloses a method including performance of the function as step(s)). An engine that receives user input and provides output, i.a., to an end user can be characterized as an “application.” Engines can make up part of, or also be described as, or can comprise “programs,” code,” “algorithms,” or similar elements known in the art for the control of electronic computer systems.

Engines/programs typically can encoded/written in any form of programming language (code), including compiled or interpreted languages, or declarative or procedural languages; and it can be deployed in any form, including as a standalone program or as a module, component, subroutine, or other unit suitable for use in a computing environment (e.g., Python, Java, C++, Ruby, C#, Go, JavaScript, R, Swift, and the like, which are known in the art). A program may, but need not, correspond to a file in a file system. A program/Engine can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program/Engine can be deployed to be executed on one computer or on multiple computers that are located at one site or distributed across multiple sites and interconnected by a data communication network. Programs/engines also can be described as “instructions” or “computer-implemented instructions,” “processor-implemented instructions,” “computer-readable instructions,” “computer implemented data engines,” etc. Features/characteristics of engines are further described below. Functions can also be performed by more advanced systems/components in the context of methods/systems, such as neural network(s).

Examples of functions carried out by engines (AKA, operations, processes, or protocols) that can be employed as S(s)/FM(s) include data/data set analysis, search, and modification functions, such as art-recognized regular expression (“Regex”) function(s) (e.g., Count, Extract, or Replace function); data cleaning functions (such as Clean, Count, Remove, TRIM, and Extract, and functions for finding/fixing double encodings, encoding error identification/fixing functions, etc.); data/data set field (DSF) comparison functions (e.g., string comparison functions, e.g., string functions focused on comparison of longest common subsequence, Levenshtein distance, and other comparison functions such as optimal string alignment functions, etc. “Engines” are components of computerized devices/systems, having a structure that comprises computer readable/executable media (CRM) (e.g., encoded in PTRCRM covered elsewhere) that carry out function(s) when acted on by associated processor(s). The structure of an engine is usually provided primarily through computer-readable instructions (code). As such, uncontradicted terms such as “program,” “code,” “module,” etc. can be used in place of “engine” and vice versa. The term “module” is often used here to refer to one or more engines that perform a function. The structure/encoding of an engine will vary depending on the features of the computer/system that executes an engine, as can human readable instructions that are provided to the system/computer. Typically, an engine processes input(s) and generates output(s). In aspects, multiple components of an engine are installed and running on one or more computer(s) in a system; multiple instances of an engine are installed and running one or more computers of a system; or both. A program/engine can be stored in a portion of a file that holds other programs or data, e.g., one or more scripts stored in a markup language document, in a single file dedicated to the program in question, or in multiple coordinated files, e.g., files that store one or more modules, sub programs, or portions of code. An engine can be deployed to be executed on one computer or on multiple computers that are located at one site or distributed across multiple sites and interconnected by a data communication network. Uncontradicted, any description of an engine can be performed by a neural network, other artificial intelligence (AI) component/model (e.g., a Random Forest machine learning element, an SVM, etc.), or a non-AI/non-neural network engine (e.g., a typical program that carries out algorithm(s)). Typically, an “engine” also is recognized as a type of electronic/computerized component/device comprising physical media comprising instructions in computer readable media (CRM) executed (read and carried out) by processor component(s) to perform function(s).

Functions can comprise other smaller units such as routines, analyses, and the like. Readers will understand that there can be considerable overlap between such terms based on context and, accordingly, at least in aspects, such overlaps are acceptable, and each term provides support for the other in terms of what the explicit content of this document implicitly discloses. Such small units of a function (e.g., commands, statements, function calls, etc.) can comprise any suitable type and number of algorithmic statements for performing the function, e.g., INITIATLIZATION statements for variable generation, IF/THEN or IF/THEN/ELSE statements and the like (e.g., ISEMPTY/ISNULL), FOR/WHILE statements and other looping statements/conditional statements (e.g., SEQUENCE statements, REPEAT-UNTIL statements, e.g., comprising CASE statements, etc.), INCREMENT statements, e.g., related to looping, GET/READ or CALL statements, etc. (for data retrieval), DISPLAY/SAVE/PRINT commands, DO statements, mathematical functions (e.g., SUM, DIFFERENCE, etc.), statistical, and other quantitative comparisons or other computational analysis statements (“COMPUTE”), LIKE statements for comparing similar data elements, which can be, e.g., contained in part in nested constructs, sub-procedures, etc. (see, e.g., users.csc.calpoly.edu/~jdalbey/SWE/pdl_std.html and geeksforgeeks.org/what-is-pseudocode-a-complete-tutorial/, geeksforgeeks.org/functions-programming/and other suitable guides to pseudocode and coding elements).

Computerized devices in communication with each other over distance, and the data connections between such devices, can be said to form a “network” or a “system.” In aspects, some, most, or all of a system can also be considered a network. System/network components typically interact/communicate on a recurring basis (typically a regular or continuous basis), usually using common communication protocols over digital interconnections for the purpose of sharing data, functions, or other resources. Networks can comprise other physical components, e.g., routers, switches, and the like, described elsewhere or known in the art, or suitable virtual counterparts thereof. Some computer device components of networks are sometimes described as “clients” and “servers.” E.g., in embodiments, a server transmits data, e.g., an HTML page, to a user device, e.g., for purposes of displaying data to and receiving user input from a user interacting with the device, which acts as a client. Data generated in a user device, e.g., a result of user interaction, can likewise be relayed to a server. Elements of network systems provided herein that can be alternatively described as clients and servers will be clear to readers. The presence of other network elements described herein is understood as implicitly described in aspects relating to network systems. Additional network elements can include firewalls, authentication protocols, virtual private network elements, etc.

Also or alternatively to algorithmic engines (software based on algorithm(s)/programming), engines can comprise or can represent artificial intelligence models (AIMs) or AI systems. Artificial intelligence systems are well known and accordingly only briefly described herein. In aspects, function(s) are carried out by AIM(s). In aspects, some, most, generally all, or all of the AIM(s) of a device/method are trainable AIMs (e.g., AIMs that perform machine learning (ML)). E.g., computerized devices can comprise AIMs that are trained with the analyses and associated results/outcomes from performing methods of the technology (e.g., an AIM can be trained with analysis of material samples from various geologic units along with associated outcomes relating to, e.g., the production of hydrogen from such geologic units).

A variety of known ML algorithms/models are known that can be employed in such approaches (executed in/by AIMs), such as data classification methods, Naïve Bayes classification (or Bayesian network methods), decision tree methods, decision rule methods, regression methods (e.g., logistic regression, lasso regression, SVM regression, ridge regression, or linear regression), random forest methods, support vector machine methods, and neural network methods, which are often employed in supervised ML methods. In aspects, ML models comprise method(s) often used in unsupervised or reinforced ML methods such as k-means (or variants thereof, such as K density-based neighbor analytical models, such as k-nearest neighbor analysis; other clustering methods (e.g., partitional clustering, mean shift clustering, density based clustering (e.g., DBSCAN methods), or hierarchical clustering (such as agglomerative clustering)); and multi-dimensional mapping methods, such as self-organizing mapping methods; and affinity mapping (e.g., for detection of events or prediction of events). In aspects, reinforced learning methods are applied such as artificial neural network methods. In aspects, ML methods include ML methods for decomplicating data such as decomposition methods, such as single value decomposition methods, dimensionality reduction methods (e.g., principal component analysis (PCA), Singular value decomposition (SVD), or TSNE), etc. In aspects, ML methods employ model-free methods, such as in the context of reinforced learning, such as a Q-Leaning method. In aspects, MLIFs comprise model-agnostic methods, such as Partial Dependence Plot (PDP) methods, ICE methods, ALE plot methods, LIME methods, and the like. Other ML models that can be employed include partial dependence plot methods, Generalized Linear Models (GLMs) and Generalized Additive Models (GAMs), and the like. MLIFs can comprise deep learning methods, shallow learning methods, or a combination of these or any other suitable “ML” (machine learning) methods alone or in combination with other AI methods.

In aspects, engine(s) of a system can be implemented, in part, by operation of one or more neural network(s), either by direct operation of the neural network or by indirect operation of the neural network operating in coordination with other system elements. E.g., analytical neural networks are often referred to as “inferencing engines” or “neural network inferencing engines.” Thus, in general, any general description of an engine herein, can be performed by any type of computer component, including a classic computer program, object, library, or more advanced system such as neural networks, and uncontradicted, the general use of the term “engine” implicitly discloses corresponding aspects of each such type of engine and for neural network and non-neural network engines, in general. Neural network methods and systems for understanding and applying user personality types and for operation of other functions/steps are known, which may be useful in the performance of some methods of the technology. Relevant principles, methods, and the like are described in, e.g., US20210288926; U.S. Pat. No. 10,991,142; US20190205382; US20200395008; U.S. Pat. No. 11,275,895; US20210182663; U.S. Pat. No. 10,049,103; WO2021185998; US20180075483; US20210390876; U.S. Pat. No. 11,257,496; WO2021258000; and U.S. Pat. No. 11,516,158. Neural networks can be referred to as a “model” or in respect of a “model.” The term “model” is sometimes applied to NNs, in general (i.e., in the sense that a NN can be considered an analytical model, computational model, machine learning model, etc.). In another sense, the term “model” in the art refers to either the function or type of function performed by a neural network (e.g., semantic element prediction) or a neural network characterized as being trained to perform a function. Neural networks and other AIMs typically are “intelligent” systems and can “learn” or “evolve” with training/learning, including learning/training that occurs through use that increases the overall training set available to the NN, feedback, or both. Methods of learning/training NNs are known in the art. Uncontradicted, training of an NN herein comprises performance of various techniques that typically result, usually indirectly, in detectable or significant changes in weight(s) of neuron(s) of an NN, usually also or alternatively leading to a detectably or significantly change in output, in terms of output of the NN or of the system, in respect of a given analysis/function, input, or both. Typically, such changes in weights occur from further training of the neural network or changing the parameters of such training (e.g., applying different rewards to an NN for complying with a standard, such as output of another NN). Skilled readers will understand that in this and several other respects, systems/methods of the technology embody “deep learning” approaches to information processing/analysis. Because NN(s) “learn” and “evolve,” NN(s) (or models) can be described as either immature or mature. While such terms are relative, they will be generally understood in the art at least in clear cases (e.g., immature NN(s)/model(s) meaning models in the first number of iterations of a method/use of a system and mature NN(s)/model(s) meaning when an NN approaches convergence (e.g., generally or substantially producing substantially identical results in each use/iteration).

In one such aspect, the technology includes a computer that is configured to evaluate hydrogen proxy measurements or hydrogen proxy+hydrogen measurements in material(s), e.g., in a plurality of samples of material(s), and from such data to aid in the identification of areas of likely high hydrogen content/purity. Such functions/engines can comprise, e.g., comparison of data against standards, comparison of data to each other (performing comparative analyses), or both. Such functions can comprise, e.g., data regarding patterns of data from materials, e.g., from geologic units, with relation to hydrogen location, production, etc. E.g., such a function may identify, e.g., a zone within a geologic unit that is expected to contain highly purified hydrogen, e.g., by the identification of a helium cap or other feature that appears to be trapping such hydrogen.

In aspects, computer technology can be used in providing recommendations to a user of a device or analytical system for carrying out any one or more of the above-described methods of the technology. For example, a recommendation function/engine can recommend the use of different extraction conditions (e.g., time, amount of force, or both). A recommendation engine might also or alternative recommend analysis of samples from a nearby location. A recommendation engine might also or alternatively recommend analyzing other sources of data from those currently input into an analysis, or removing certain data (e.g., outlier data, confusing data, etc.). A computer system can comprise rules/algorithms for eliminating data, for optimizing data relied upon in analyses, etc.

Computer technology also can be used in automating any of the above-described steps of methods of the technology. E.g., computer technology can be used to control the operation of an analytical device of the technology or used to perform the technology. E.g., the computer technology can either allow a user to selectively control operation of some, most, generally all, or all components or devices used to perform steps of methods, to control the operation of some, most, generally all, or all such devices/components based on preprogrammed conditions (which can be hard programmed, conditionally triggered (e.g., by if/then or while engine structures/algorithms), or both).

The term “training” in connection with an AIM, such as a neural network, is understood in the art as comprising determining better/optimal operating parameters/conditions for a neural network to perform function(s). Typically, training comprises determining and applying the best set of weights for maximizing accuracy of an NN (adjusting weights in iteration(s) of use/performance) or other AIM.

AIMs are computer implemented models of analysis, functioning, or both, can mimic human learning, e.g., by regularly, frequently, or continuously learning with further uses/iterations/applications. AIMs can, in aspects, such as in neural networks, also may undergo some amount of training by human system administrators or by operation of other device/system components as described herein (e.g., a secondary AIM). E.g., a device may utilize an AIM in determining a user's PSP, in selecting/generating operational control instructions, in determining trigger event criteria, optimizing stimulation, etc.

### REPRESENTATIVE EXPERIMENTS/EMBODIMENTS (“EXAMPLES”)

The following detailed exemplary expository descriptions or experiments involving embodiments, applications, or related principles, of or otherwise related to the invention (“Examples”) are provided to assist readers in further understanding aspects of the invention or principles related to the invention or practice of aspects of the invention.

Any particular materials, methods, steps, and conditions employed/described in the following Examples, and any results thereof, are merely intended to further illustrate aspects of the invention. These Examples reflect exemplary embodiments of the invention, and the specific methods, findings, principles of such Examples, and the general implications thereof, can be combined with any other part of this disclosure. However, readers should understand that the invention is not limited by these Examples or any part thereof.

The following exemplary embodiments are shown in the Figures provided with this disclosure.

### DESCRIPTION OF THE EXEMPLARY EMBODIMENTS SHOWN IN THE FIGURES

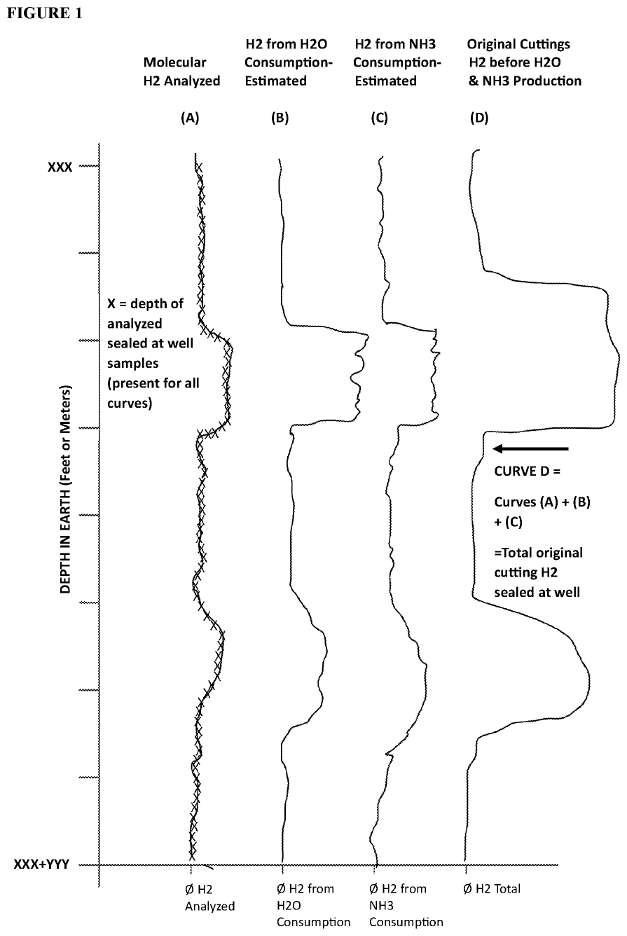

### Example 1 (FIG. 1)

Example 1 demonstrates an aspect of the invention directed to determining the total amount of hydrogen in a drill cutting sample collected from a petroleum well which is sealed at the well upon its collection. FIG. 1 provides an example demonstrating the determination of the amount of hydrogen present in a sample, e.g., a geologic sample, e.g., drill cuttings, core samples, or other solid samples or drilling muds, at the time the sample was collected, such as, e.g., at the time, e.g., drill cutting(s) sample(s) arrived at the surface from their original sub-surface location.

In a first aspect, FIG. 1 illustrates the determination of the total amount of hydrogen originally present in a material prior to its collection, wherein the material is sampled, e.g., in this example collected as a drill cutting sample and sealed upon collection from a petroleum well by way of (1) measuring the amount of molecular hydrogen in the sample; and (2) adding the measured amount of molecular hydrogen to (a) an originally present, but now consumed, amount of hydrogen used in (consumed by) water (H2O) production, wherein water is produced in the sealed cuttings tube from the reaction of the drill cuttings hydrogen with oxygen present in the collection tube headspace and wherein the amount of water produced is estimated by the reduction in the amount of oxygen present, and (b) an originally present, but now consumed, amount of hydrogen used in (consumed by) ammonia (NH3) production, wherein ammonia is produced in the sealed cuttings tube from the reaction of the drill cuttings hydrogen with nitrogen present in the collection tube headspace and wherein the amount of ammonia produced is estimated by the reduction in the amount of nitrogen present.

As shown in FIG. 1, the amount of original material-associated hydrogen (e.g., rock material associated hydrogen), e.g., present in a rock material sample at the moment such a rock material sample, e.g., drill cutting sample, is hermetically sealed in a container (such as a cuttings sample tube) can be reconstructed. Specifically, as illustrated in FIG. 1, the amount of directly measured (analyzed) molecular hydrogen associated with a material sample, e.g., drill cuttings sample, or, e.g., as shown a plurality of samples collected from varying depths of a petroleum well, is obtained (Line (A)). An estimated amount of hydrogen originally present in the material but which is consumed in the production of water within the sealed sample container by way of the sample being in contact with air within the sample container headspace upon sample collection is further obtained for each sample (Line (B)). Water is produced by the reaction of material sample-associated molecular hydrogen with oxygen trapped in the sealed container headspace. An estimated amount of hydrogen originally present in the material but which is consumed in the production of ammonia within the sealed sample container by way of the sample being in contact with air within the sample container headspace upon sample collection is also obtained for each sample (Line (C)). Ammonia is produced by the reaction of material sample-associated molecular hydrogen with nitrogen trapped in the sealed container headspace.

The sum of analyzed molecular hydrogen (H2) (reflected in Line (A)), the amount of hydrogen in the water (H2O) produced in the sealed tube from cuttings hydrogen and head space oxygen (reflected in Line (B)), and the hydrogen in the ammonia (NH3) produced in the sealed tube from cuttings hydrogen and head space nitrogen (reflected in Line (C)) is obtained and is illustrated as Line D in FIG. 1. Line D represents the original material/cuttings hydrogen at the moment the cuttings were hermetically sealed in the cutting's container. Line D equals the sum of lines A, B, and C.

Plots of depth on the vertical axis (y-axis) (with more shallow depths on the top and deeper depths on the bottom), versus geologically relevant/geologically significant value(s) on the horizontal axis (x-axis), is a common depiction of bore hole data and is referred to as logs, or well logs. Lines A to D reflect material/cuttings well logs molecular hydrogen (Line A), hydrogen in produced water (Line B), hydrogen in produced ammonia (Line C), and total hydrogen at time the sample was trapped and hermetically sealed in a material/drill cuttings container (Line D).

In an additional or alternative aspect, an element of this disclosure provided in or relating to the disclosure of FIG. 1 is as follows.

As described elsewhere herein, a detectable or significant amount of hydrogen present in a material sample, e.g., a geologic sample, e.g., drill cutting(s) sample at the time the sample is first collected may be converted to ammonia. Further, a detectable or significant amount of hydrogen present in a material sample, e.g., a geologic sample, e.g., drill cutting(s) sample at the time the sample is first collected may be converted to water. Such hydrogen conversion can occur upon the exposure of the material sample to air, e.g., air present in the headspace of a sample collection container.

In one sense, FIG. 1 provides a graph much like a traditional well log (or graphs presented in the Prior Smith Patents), showing depth of the geologic location from which samples were collected (e.g., the depth of a well from which cutting samples were collected) on the Y-axis and the amount of analyte measured in the sample collected from each depth on the X-axis. Line A of FIG. 1 illustrates the analyzed hydrogen log of the collected and analyzed samples.

This line can indicate the amount of molecular hydrogen directly measured in each sample. The line is a zero line, indicated by the “Ø” at the bottom of the graph. “X” indictors indicate where, along the depth of the geologic location, e.g., oil well, the sample was collected. Note that specific “X” indicators are provided for Line A but for the sake of simplicity are not reflected in each of Lines B-D. Line B of FIG. 1 illustrates the amount of hydrogen calculated to be needed to make water in each of the analyzed samples. Line B indicates the amount of hydrogen consumed by the formation of water. This line is also a zero line, as indicated by the “0” at the bottom of the graph. Line C of FIG. 1 illustrates the amount of hydrogen needed to make ammonia in each of the analyzed samples. Line C indicates the amount of hydrogen consumed by the formation of ammonia. This line is also a zero line, as indicated by the “0” at the bottom of the graph. Line D of FIG. 1 illustrates the log created by adding all three of the values associated with each of lines A, B, and C together. Line D represents the amount of hydrogen in the material sample, e.g., drill cutting(s) sample(s), at the time they arrived at the Earth's surface and were hermetically sealed in collection container(s). Line D indicates the amount of hydrogen in the original sample prior to water and ammonia production.

As shown in FIG. 1, the amount of hydrogen in the original material sampled at different depths of an, e.g., petroleum well, can be determined and compared. As illustrated in FIG. 1, the invention provided herein is able to detect variation(s) in the amount(s) of hydrogen present across varying depths of a geologic location such as a petroleum well.

FIG. 1 can be in one sense considered to provide an illustration of adding analyzed molecular hydrogen, plus original but now consumed hydrogen calculated from estimated ammonia production, e.g., in a sealed cuttings tube from reaction of cuttings hydrogen with nitrogen in the tubes head space air., plus original but now consumed hydrogen calculated from, e.g., estimated water production in the sealed cuttings tube from reaction of cuttings hydrogen with oxygen in the tubes head space air, to obtain a calculated amount of hydrogen estimated to be present in the material located at the location from which the material sample was collected.

In yet an additional/alternative description of FIG. 1, FIG. 1 illustrates how hydrogen, originally present in samples collected from various depths below the surface of the Earth and sealed in the presence of ambient air prior to analysis, can be determined.

The graph of FIG. 1 shows the depth from the surface of the Earth on the y-axis with the top of the y-axis indicated as, e.g., 0, that is the surface of the earth. The y-axis is also labeled with xxx, where xxx is the depth at which a sample is collected. For example, if a sample was collected at a location identified as being 50 feet below the surface, such sample would be identified as “50” or “−50.” If another sample was collected from a location identified as 100 feet below the surface, it would be identified as “100” or “−100,” respectively.

The depth at which the sample is collected is typically measured in feet or meters. However, miles or kilometers (or fractions thereof) may be appropriate units of measure.

The lower part of the y-axis which intersects the x-axis is labeled as “xxx+yyy.” In the case that the top of the graph equals the surface of the earth, then xxx would equal 0 feet or meters, for example (as appropriate), and a sample taken from 1,543 feet below the surface for example would be labeled as 1543, −1543, 01543, −01543, as appropriate. However, the top of the y-axis does not need to be the surface of the Earth. For example, it may be a certain depth within a known formation which is identified as being a certain depth from the surface in a well or borehole, for example, where such samples were collected. In such case, if a reference point within the formation was 540 feet below the surface in the well or borehole where samples were being collected, then xxx would be 540 or 0540 (for example) and a sample collected at 1,680 feet below such reference point would have a yyy of 1680 and xxx+yyy would be labeled as 2220.

Alternatively, a particular reference point in a formation may be given an xxx value of 0, 00, 000, or 0000 for example. In this case a sample collected 1,000 feet below the reference point would be given a value of 1000 or 01000, for example which equals xxx+yyy, that is yyy equals 1000, for example. In the event that a sample was collected from a location closer the surface than the reference point of 0, then it would be given an negative value, i.e., a sample collected at 100 ft above the reference point would be given a value of −100, −0100, or −00100 for example, that is xxx equals 0 and yyy equal-100.

The x-axis of FIG. 1, Example 1, shows four (4) separate zero-lines. Each line provides values for the amount of hydrogen gas, or proxy for hydrogen gas, in a sample collected at a given depth, wherein the x on the left most curve indicates a sample collected at given location (xxx+yyy). For a given line, any deviation to the right of the zero reference marked on the x-axis for such curve is indicative that hydrogen was measured using the methodology specified for such curve, to be discussed. In this case, the amount of deviation to the right does not indicate an absolute amount of hydrogen. Rather the deviation is relative to another sample evaluated using the same methodology. That is, a point for a sample further to the right has more hydrogen than a point further to the left, and a point associated with a sample which is positioned further to the left has less hydrogen than a point associated with a sample which is positioned further to the right as measured by the same methodology. Such a scale may be linear, logarithmic, or some other function but will be the same function for a given curve. The same function does not need to be used for each curve; that is, e.g., one curve may be a linear function, and one or more other curves may be logarithmic.

As previously described, the samples identified by the x points on the left most curve of FIG. 1 were sealed upon collection in the presence of ambient air. At some point after sealing the samples, they were analyzed for the amount of hydrogen or hydrogen proxy in the sample.

The leftmost line (also referred to herein as a curve) identified as “(A) Molecular H2 analyzed” is the actual amount of hydrogen gas measured in the analyzed sealed sample. As is illustrated, there are numerous samples which contain little to no hydrogen as suggested by large portions of the line being aligned with the zero point associated with line A. However, there are 2 distinct locations, as indicated by portions of the line, where a distinctly identifiable amount of hydrogen was measured in the location-associated samples; one at a relatively shallower depth and another at a relatively deeper depth.

The line/curve to the right of the left most line/curve, labeled “(B) H2 from water consumption estimate” provides an estimate of the amount of hydrogen that was in the sample at the time it was sealed but which has reacted with oxygen, O2, from the ambient air in the sealed sample to produce water (H₂O). For every molecule of water that is produced, a single molecule of H2 is consumed. Alternately, for every molecule of oxygen, O2, consumed, 2 molecules of hydrogen (H₂) are consumed. Thus, to better account for the amount of hydrogen in the sample present immediately upon its collection, when it was sealed, the amount of hydrogen that was consumed by reacting with oxygen is added to the amount of hydrogen reflected in Line A (directly measured hydrogen). Line B reflects an estimate of the amount of hydrogen consumed by its reaction with oxygen in the ambient air present in the headspace of the sample container comprising the material sample.

It should be noted that such an estimate can be made based on the amount of oxygen that remains in the tube when the sample was analyzed for hydrogen directly, i.e., when curve A was generated. Typically, ambient air is about 21-22% O2; as such, if it is observed that the amount of O2 was 22% when the sample was sealed and 20% when the sample was analyzed, then the amount of oxygen consumed can be estimated to be 2% multiplied by the volume of the ambient air in the sample when sealed, which can be estimated, e.g., by measuring, the amount of argon in the sealed sample when analyzed since the amount of argon in samples derived from the Earth is typically close to zero, and, further, argon does not detectably or significantly react with hydrogen. As the amount of hydrogen consumed is twice the amount oxygen consumed in the production of water, the amount of hydrogen consumed in the production of water would be twice the amount of oxygen consumed.

Alternatively, the amount of water generated to yield curve B can be measured. For each molecule of water generated, one molecule of hydrogen is consumed.

Still further, as an additional alternative, as the amount of oxygen consumed in reacting with hydrogen is directly proportional to the amount of water produced, 1 molecule of oxygen consumed for each 2 molecules of water produced, one could measure both the amount of oxygen consumed and the amount of water produced to obtain an estimate of the amount of hydrogen consumed by the sealed sample reacting with the oxygen in the volume of trapped ambient air within the container. However, the measurement of both oxygen consumed and water produced is not necessary to generate line B.

It should be noted that line/curve B typically only provides an estimate of hydrogen in the original sample when curve A also indicates that hydrogen is present in the sample. In aspects, exceptions are (1) when all of the hydrogen in the sample is consumed, then curve A may provide indicate zero or essentially zero value for the amount of hydrogen in the sample and curve B may give a positive value, or (2) no ambient air is sealed with the sample; e.g., the sample occupies the entire space in the tube or the ambient air is displace with an inert gas such as argon such that there is no oxygen to react with the hydrogen. If an insufficient amount of time is allowed for the oxygen present in the sealed sample to react, line/curve B may not give a positive estimate, or may provide a diminished estimate, of the amount of hydrogen in the sample, whereby line/curve A can indicate that hydrogen was present at the time the sample was sealed.

The curve to the right of curve B, labeled “(C) H2 from ammonia consumption estimate” is similar to curve B in that it provides an estimate for the amount of hydrogen in the sample when it was sealed, with ambient air, immediately upon its collection, except in the case of line/curve C it reflects the amount of hydrogen that was consumed by the hydrogen of the sample reacting with Nitrogen in the ambient air, typically present in ambient air at a concentration of about 77%, to produce ammonia. 1.5 molecules of H2 are consumed for each molecule of NH3 produced. As the amount of ammonia in samples derived from the Earth is negligible, any ammonia generated and measured can be directly equated with an amount of hydrogen consumed to produce line/curve C.

Alternatively, one can measure the amount of Nitrogen in the air within the container (identifying the amount of Nitrogen consumed) to estimate the amount of hydrogen that was present when the sample was sealed with ambient air but consumed in the production of ammonia. For each molecule of Nitrogen (N₂) consumed, three molecules of hydrogen (H₂) are consumed to produce 2 molecules of NH3. Thus, if the concentration of N2 in the air was originally 77% when the sample was sealed and is determined to be 74% when the sample is analyzed, then a 3% reduction in the concentration of nitrogen by virtue of it reacting with hydrogen in the sample at the time it was sealed is identified. Such percentage reduction, 3%, can be multiplied by the volume of ambient air that was sealed in the tube to get the absolute amount of nitrogen consumed. As with the case for oxygen consumed, the amount of nitrogen in the ambient air which was sealed with the sample can be estimated by measuring the amount of a gas, such as argon, which is present when the sample is analyzed since the amount of argon in samples derived from the Earth is negligible. Thus, the amount of argon measured can be divided by the typical concentration of argon in ambient air, about 1%, to estimate the volume of ambient air sealed in the tube.

Upon calculating the absolute amount of nitrogen consumed (N2), the amount of hydrogen (H₂) that was in the sample when sealed but consumed to produce ammonia can be calculated by multiplying the amount of nitrogen consumed by 3 to generate line curve C. As the relationship between the amount of nitrogen consumed in reacting with hydrogen is directly related to the amount of ammonia produced, that is 1 molecule of N2 is consumed to produce 2 molecules of ammonia (NH3) both the amount of ammonia produced and the amount of nitrogen consumed can be determined to generate a more accurate curve C. However, it is not necessary to measure both nitrogen consumed and ammonia generated to generate an estimate of hydrogen consumed in the production of ammonia. For the same reasons that were discussed relative to line/curve B, typically line/curve C will generate a positive estimate of hydrogen present when sealed but consumed when there is a positive value for curve A for the same sample. For the same reasons that were discussed for curve B, such a relationship may not always exist.

To obtain a more accurate estimate of the amount of hydrogen present in sample(s) at the time of sealing, the amount of hydrogen that is measured directly in the sealed sample (curve A), the amount of hydrogen that was in sample when sealed but consumed to produce water (curve B) and the amount hydrogen that was in the sample when sealed but consumed to produce ammonia (curve C) can be added together. This sum is reflected in curve D labeled “original cuttings H2 before water & ammonia production;” where curve D is the addition of the amount of hydrogen present for a given sample as indicated in lines/curves A, B, and C. If, for example, there was no hydrogen in a sample when sealed, the values of lines/curves A, B, and C would be 0 respectively, and curve D would also be 0. Alternatively, if there was a measurable amount of hydrogen in the sample when it was sealed with ambient air immediately upon its collection, and not all, but some, of the hydrogen reacted with oxygen and nitrogen to form water and ammonia respectively, then lines/curves A, B and C may give values of, e.g., 1, 2, and 3 respectively (ignoring the stoichiometry of the reaction between hydrogen, oxygen and nitrogen) to generate a value of 6 for curve D for such sample when analyzed. To be clear, the value provided for a given sample, collected from a location at a given depth, represented in line/curve D is always the summation of the values of hydrogen (measured directly or estimated) indicated in lines/curves A, B, and C.

As the FIG. 1 graph indicates, this non-limiting example provides results for drill cutting samples that were collected and sealed immediately with ambient air, e.g., in a container with headspace containing ambient air. However, other material sample(s) could be, e.g., other types of geological samples, such as, e.g., core samples. This representative sample provides an estimate of the amount of hydrogen that was in the sample when sealed at the surface. In aspects, one may also perform this procedure to measure the amount of hydrogen that is in the drilling mud derived from the same location as the cutting or core sample to account for any hydrogen that escapes from the sample when collected at a particular location in the Earth into the drilling mud. As with the drill cutting(s) or core sample(s), drilling mud sample(s) can be sealed with ambient air, and any hydrogen in the drilling mud when the sample is sealed may react with oxygen and nitrogen to produce water and ammonia. As such, complementary lines/curves A, B, C and D can be generated for the drilling mud. In certain aspects, to obtain an estimate of the amount of hydrogen in a sample at the time of its collection from a location below the surface of the Earth, results from drilling mud obtained as described above can be combined with the results obtained as described above from drill cutting(s) sample(s), core sample(s), or both, for location(s) from which sample(s) are/were collected. In aspects, such an estimate can assume that no hydrogen is lost from the cuttings/core samples and drilling mud or gained into cuttings/core samples and drilling muds into/from the rest of the rock in the well/borehole. However, if such a loss or gain were to occur, then adjustment(s) can be made to account for such gain(s) or loss(es) to better (detectably or significantly more accurately) estimate of the amount of hydrogen present in a material sample collected from a location, e.g., from a location within a well/borehole.

Combining lines/curves A, B, and C generates an estimate of the amount of hydrogen in a sample when sealed with ambient air (line/curve D). In the event that one or more of lines/curves A, B, or C (or any combination thereof) cannot be generated, an alternative estimate of the amount of hydrogen in sample when sealed with ambient air can generated by combining the any available, remaining curves, to generate a different line/curve D (e.g., a modified line/curve D). Such a modified line/curve D is detectably or significantly different than that formed by the combination of lines/curves A, B, and C when all such lines/curves are available. While such a modified line/curve D may suggest less hydrogen being present in the sample when sealed with ambient air, such a modified line/curve D can still be useful for identifying locations with relatively more or less hydrogen.

FIG. 1 is indifferent to how the hydrogen, water, oxygen, nitrogen, ammonia, or argon (for example) (or any combination thereof) are measured. Mass spectrometry is one example of an analytical method that may be used to measure one or more of the aforementioned gaseous molecules. However, other analytical methods may be substituted or combined with mass spectrometry to measure one or more of the aforementioned gaseous molecules. In aspects, any method suitable for quantifying, measuring, or otherwise obtaining relative amount(s) of the gaseous molecules, compound(s), or combination(s) thereof may be suitable for use in aspects of the disclosure described herein.

In the case that mass spectrometry is used, analytical method(s) comprising mass spectrometry can be combined with the application of one or more vacuums, at one or more temperatures, for one or more time periods, to the sealed sample containing ambient air to extract the gaseous molecules from the sample and to supply such gaseous molecules into the mass spectrometer for measurement, e.g., for absolute or relative measurement. Suitable analytical method(s) or element(s) of suitable analytical method(s) (such as, e.g., systems/devices/instrumentation) are described in, e.g., the Prior Smith Patents and, e.g., in new disclosure provided herein.

In certain embodiments, the sealed sample is subjected to a less strong vacuum to extract and measure gaseous molecules that are easily extractable, such as gases that are in the headspace of the sealed sample or easily dissociated from the material sample (e.g., drill cuttings/core/mud), followed by the application of one or more stronger vacuums to extract gases that are more tightly bound to or associated with the material sample. Further, in the case that, e.g., mass spectrometry is used, it is possible for the analytical method to be conducted with or without the presence of a cryotrap. In aspects, a cryotrap can condense condensable gases such as hydrocarbons other than ethane and methane and water. In aspects, analytical method(s) can be conducted with a desiccant or, e.g., in alternative aspects can be conducted without a desiccant, to remove water.

In the non-limiting example of Example 1, an estimate of the amount of hydrogen present in a material sample sealed with an amount of ambient air in a sample collection container immediately upon the collection of the material sample is obtained by combining the amount of hydrogen still contained in the sample and the amount of hydrogen consumed by reacting with oxygen and nitrogen. In embodiments, headspace of a sample container can be purged of ambient air with a non-reactive, gas, e.g., a gas which is more dense than air, such as, e.g., argon, krypton, or xenon. Upon purging, the sample can be sealed. In such an example, there is no detectable or significant air (e.g., there is no DOS nitrogen or oxygen) with which the hydrogen from the material sample can react to form ammonia, water, or both. In such embodiments, one would expect the amount of hydrogen measured directly (represented in/by line/curve A) to be detectably or significantly greater than that for the same sample when allowed to react with ambient air if/when the headspace of the sample collection container is not purged. Similarly, the amount of hydrogen reflected by lines/curves B and C would be expected to be detectably or significantly less than those for the same sample when allowed to react with ambient air. In an idealized scenario, it may be expected that the amount of hydrogen in the sample sealed with ambient air or purged with an inert gas and sealed to produce similar line/curve Ds; however, there may be reasons why such curves may be different, such as, e.g., in the case of mass spectrometry, different times, temperatures, and vacuums applied.

### Example 2 (FIG. 2)

Example 2 provides an example of the inventive technique(s) provided herein can reveal specific stratigraphy (ies) more or less suitable for resource production, such as, e.g., hydrogen production. FIG. 2 is provided as an example of the application of technology(ies) provided herein to identify geologic location(s) comprising a high purity resource, e.g., hydrogen, wherein such identification can be used, in aspects, to guide production of the same.

FIG. 2 shows drill cuttings well logs of hydrogen (H2) and helium (He) in a petroleum well bore hole. The analysis of materials/drill cuttings-associated hydrogen measures are shown as open circles. The line connecting the open circles is a hydrogen trend line versus depth at which the material sample was collected. The analyses of materials/drill cuttings-associated helium measures are shown as X's. The line connecting the X's is a helium trend line versus depth at which the material sample was collected.

This illustrative “log” reveals a shallow helium-rich, hydrogen-poor zone overlying a hydrogen-rich, helium-poor zone. That is, within a shallower location, a zone exists where material(s) comprise little to no hydrogen but contain a detectable or significant amount of helium, and within a deeper location, a zone exists where the same material(s) comprise little to no helium but contain a detectable or significant amount of hydrogen.

Geologic hydrogen typically must be of significantly high purity to be useful as a fuel. The inventive methods here provide, in aspects, methods for identifying very high purity hydrogen deposits in geologic sites or in materials. In aspects, such high purity hydrogen deposits comprise, e.g., at least about 95%, ≥~96%, 2~97%, ≥~98%, ≥~99%, ≥~99.5%, ≥~99.9%, ≥~99.95%, ≥~99.99%, or, e.g., ≥~99.995% pure hydrogen content, (e.g., in gas form, condensed form, or a combination thereof).

In this respect, the inventive method reflected/embodied in FIG. 2 reveals that there can exist, and method(s) herein can detect, discrete stratigraphic zones of relatively high, e.g., commercially relevantly high, hydrogen and relatively low helium content(s), indicating that in at least some cases, hydrogen and helium can have an opposing relationship (e.g., where one is high, the other is low). In such respects, the inventive method demonstrates that hydrogen and helium may not always be associated with one another in terms of amount(s) and, e.g., that method(s) herein can identify stratigraphic zones wherein one is present in significant amount(s) while the other is effectively absent.

Identifying and logging zones, e.g., zones in, e.g., a bore hole, that are hydrogen-rich and helium-poor identifies zones of potential hydrogen production that not only contain significant amounts of hydrogen, but also can indicate that the hydrogen in such a zone is of higher purity, and that these zones may produce hydrogen of sufficient purity as to be acceptable for use as a fuel.

An additional/alternative description of aspects of the invention in or relating to the disclosure of FIG. 2 follows.

FIG. 2 can demonstrate an aspect of the invention wherein the difference in the level (amount of or presence of) helium versus the level (amount of or presence of) hydrogen can be identified across a plurality of locations within or across a geologic test site, such as, e.g., across varying depths of a well, using method(s) and device(s) described herein.

FIG. 2 can demonstrate, e.g., that helium and hydrogen can be identified as being present in distinguishably different strata of the Earth. FIG. 2 demonstrates that helium can reside directly above hydrogen. FIG. 2 demonstrates that both a helium layer and the hydrogen layer can be extremely pure, e.g., can be highly pure relative to the presence of the other. FIG. 2 demonstrates that stratigraphic separation of helium and hydrogen are identifiable using the method(s) and device(s) described herein.

FIG. 2 can, in one sense, be characterized as a graph of depth versus amount of analyte measured using method(s) and device(s) described herein. FIG. 2 illustrates a first line, line (A), showing the amount of hydrogen measured in samples collected from a plurality of depths of a geologic location, and a second line, line (B), showing the amount of helium measured in samples collected from the plurality of depths of the same geologic location, as measured from, in aspects, the same samples, or, e.g., in alternative aspects, different samples collected from the same general geologic location, e.g., the same well. As shown in FIG. 2, distinct helium-rich and hydrogen-rich zones are distinguishable. In aspects, a more hydrogen-rich zone identifiable using method(s) and device(s) herein indicates areas of higher hydrogen purity and thus provides an opportunity for targeted hydrogen extraction effort(s).

According to aspects, method(s) and device(s) described herein are capable of identifying detectably or significantly stratified versus co-mingled helium and hydrogen deposits, wherein such deposits may be useful hydrogen extraction target(s). Understanding in the art, at the time of this disclosure, is that the two gases, helium and hydrogen, are found together in geologic locations. However, FIG. 2 demonstrates that this is not the case. According to aspects, the pattern provided in FIG. 2 is a pattern that can be sought in a material sample, e.g., drill cutting, analysis/analyses using the techniques described herein to identify helium-rich and hydrogen-rich zones, providing insight into locating detectably or significantly higher purity hydrogen provided by one or more other identified geologic locations. In aspects, the identification of distinct strata detectably or significantly reduces the extraction of hydrogen from locations of a geologic site which would otherwise yield less pure hydrogen as, e.g., it is mixed with a higher amount of helium.

According to aspects, method(s) and device(s) described herein identify strata wherein helium forms a layer which detectably or significantly prevents, e.g., blocks hydrogen from entering or otherwise associating with rock. According to aspects, the invention provides, as illustrated by FIG. 2, method(s) for the identification of strata of helium-rich rock, wherein the top of such a helium-rich zone may represent a geologic “seal” wherein permeable rocks exist below such seal and impermeable rock exists above such seal. Accordingly, in aspects, this Example identifies a geologic seal formed by helium.

As illustrated by FIG. 2, method(s) and device(s) described herein can identify stratified helium and hydrogen zones within a geologic location, wherein both are present but which are distinguishable from one another. If necessary or otherwise helpful, if distinguishable strata of hydrogen-rich rock are identified, other technology(ies) known in the art can be deployed to more accurately locate the distinct demarcation(s) of such zone(s).

Another, additional or alternative description of FIG. 2 here provides an illustration of how hydrogen and helium may segregate into different locations, e.g., layers or stratigraphies, within the Earth.

On the y-axis of FIG. 2, depth is shown relative to a reference point, such as, e.g., a specific location in a formation in a well or borehole, or relative to the Earth's surface, where the closer to the x-axis, the deeper relative to the reference point or Earth's surface. The depth may, e.g., be typically expressed in feet or meters. The scale of the y-axis may be linear, logarithmic, or may reflect another suitable function.

The x-axis indicates an amount or concentration in a given unit. The axis may be linear, logarithmic, or may reflect another suitable function.

The graph of FIG. 2 illustrates the amount or concentration of hydrogen and helium measured in samples collected from one or more locations below the surface of the Earth. The amount of helium and hydrogen collected from the same or different samples are shown on (plotted on) 2 separate curves, where a zero amount of helium in a sample is indicated where the line/curve indicated by X's (indicators of where samples were taken and measured) labeled “helium-rich zone” crosses the x-axis, and where a zero amount of hydrogen in a sample is indicated where the line/curve indicated by circles crosses the x-axis. The amount of hydrogen and the amount of helium can be measured in the same or different samples. Helium and hydrogen can, in aspects, be measured in the same sample collected from a single location. In such case, the Xs and Os would be present in positions on the graph reflecting the same depth(s).

In aspects, samples across a distance or span at intervals which are at least mostly, at least generally, at least substantially, at least essentially, which are essentially, or which are equal, for example, samples being collected at about every 50 feet. In aspects, interval(s) of sample collection across a span or distance are not equal and reflect different collection interval(s), e.g., about 50 feet in certain sections of the well or borehole and about 10-foot intervals in other sections, and perhaps about 200-foot or greater intervals in other sections of the well or borehole. In certain aspects, one or more sections of a distance or span, such as a well or borehole, are not sampled.

In this example, the line(s)/curve(s) of FIG. 2 indicate that there are 2 separate zones for helium and hydrogen. Each zone is discrete and separate, e.g., detectably or significantly distinguishable, from the other zone. In this example, a relatively helium-rich zone resides in a location that is closer to the Earth's surface than a relatively hydrogen-rich zone. Both zones are extremely pure for either hydrogen or helium, as is reflected by the relative absence of the other molecule within each zone. That is, the zone containing helium contains very little or almost no hydrogen (hydrogen is detectably or significantly absent), and the zone containing hydrogen contains almost exclusively hydrogen with little or no helium (helium is detectably or significantly absent).

Hydrogen- or helium-rich zone(s) can be present in any “thickness” or can exist across a span of any distance. For example, such a zone may be quite narrow or thin, such as, e.g., representing a span or distance of about 5 feet (ft), ~10 ft, ~20 ft, ~50 ft, ~100 ft, or, e.g., ~200 ft, or, e.g., a distance of greater than ~200 ft. In aspects, each zone can be about the same thickness, as suggested by the exemplary graphs (lines/curves) of FIG. 2, or in aspects can be quite different in their thicknesses, such as, e.g., a helium zone having a thickness of about 10 ft and a hydrogen zone having a thickness of about 75 feet, or vice versa.

While these graphs illustrate that hydrogen- and helium-rich zones are almost exclusive of one another, in aspects, two such zones may detectably or significantly overlap. In certain aspects, analyses provided herein may yield the identification of a zone where hydrogen and helium comingle (are independently present in detectably or significant amounts), wherein adjacent to such a zone is a zone where helium is detectably or significantly pure, and further adjacent to but on the opposite side of such a zone is a zone where hydrogen is detectably or significantly pure.

The exemplified graphs (lines/curves) of FIG. 2 indicate that the highest identified amount of helium (in the helium-rich zone) is detectably or significantly similar to the highest amount of hydrogen (in the hydrogen-rich zone) (presuming, e.g., that the scale of each curve is the same/shared). In certain aspects, the highest amount of helium and the highest amount of hydrogen identified in each of helium- and hydrogen-rich zones, respectively, is at least mostly, generally, substantially, essentially, or is at least about the same. In alternative aspects, the amount or concentration of helium in an identified helium-rich zone is detectably or significantly different than the amount or concentration of hydrogen in an identified hydrogen-rich zone, e.g., within a single geographic unit, e.g., within a single well/borehole.

In certain aspects, the distribution of helium or hydrogen across respective helium- or hydrogen-rich zones is a somewhat normal distribution. However, in one or both such zone(s), other distributions of molecule(s) can be present.

FIG. 2 of Example 2 reflects the presence of a single helium-rich zone and a single hydrogen-rich zone within an analyzed span of, e.g., a well/borehole. In certain aspects, a plurality of helium-rich zones may be present; a plurality of hydrogen-rich zones may be present; or a plurality of both helium- and hydrogen-rich zones can be present within a given analyzed span of, e.g., a well/borehole. In certain aspects, where such a plurality of zone(s) exist, at least one helium-rich zone is positioned closer to the surface of the Earth than any one or more hydrogen-rich zone(s).

While not wishing to be bound by any particular theory, one hypothesis for such phenomenon of helium overlaying hydrogen within geologic location(s) is that there exists a cap above (closer to the Earth's surface) a helium zone through which the helium cannot migrate, wherein such a cap is, e.g., chemically repulsive to helium or does not contain the porosity through which helium can migrate. As such, as helium migrates towards the surface, it encounters such a cap, its migration is interrupted, and accordingly the helium is forced to remain in such a location and to, e.g., fill in (or otherwise occupy or become associated with) spaces, pores, cracks, etc. of such a location. Further, when any hydrogen migrates up towards the surface, it is subjected to conditions where it is relegated to its own zone below the helium zone, as helium may act, as discussed elsewhere herein, as a cap for, sealant against, or other blockage to, hydrogen migration or association.

The observations and teachings presented in this disclosure, that helium and hydrogen can occupy different zones within, e.g., a well or borehole, goes against the prevailing understanding in the art that helium and hydrogen travel through geographic strata together, wherein wherever helium is identified, typically the presence of hydrogen is also identified. However, this disclosure demonstrates that each of He and H2 can be identified and quantified, e.g., directly or relatively quantified immediately or at a later date, in sealed-at-the-well samples, and the results of such analysis can identify geologic locations where each of hydrogen and helium exist, but they can exist in different amounts within different zones/layers of the geologic location.

One of many practical implications of this discovery is the ability to identify location(s) that contain nearly exclusively helium or nearly exclusively hydrogen. Such an ability can have significant positive economic impacts on the production of highly pure helium or the production of highly pure hydrogen, perhaps, e.g., purity of one or both at levels greater than about 95%, ≥~96%, ≥~97%, ≥~98%, ≥~99%, ≥~99.5%, ≥~99.9% or, e.g., about 100% purity.

The exemplary findings presented in FIG. 2 of Example 2 do not require a specific material sample. In aspects, the material sample is a geologic material, such as, e.g., a rock material, e.g., drill cutting(s), core sample(s), drilling mud(s), or a combination thereof. Many types of sample(s) can be useful, alone or in combination, provided that they can be subject to the analysis of present hydrogen and helium. Further, the exemplary findings presented in FIG. 2 do not require a specific method for analyzing the amount(s) of hydrogen, helium or both. In aspects, the presence and amount(s) of each of the two molecules can be detected using the same method. In aspects, the presence and amount(s) of each of the two molecules can be detected using different methods. In certain aspects, mass spectrometry can be used to measure helium, hydrogen or both. In aspects, analytical method(s) for determining the amount(s) of helium, hydrogen, or both may comprise use of a cryotrap to remove condensable gases from sample(s), such as, e.g., water and hydrocarbons other than methane and ethane, prior to introducing the sample(s) onto the analytical instrument, e.g., mass spectrometer, to reduce background or interfering signal(s). In aspects, such as, e.g., aspects wherein mass spectrometry is used to quantify (directly or relatively) the amount(s) of hydrogen, helium, or both, the application of one or more different strength vacuum(s) can be used to participate in the extraction of one or more volatile(s). In aspects, vacuum strength(s) such as vacuum pressure of about 20 mbar as a lesser vacuum strength and vacuum pressure of about 2 mbar as a stronger vacuum strength can be applied to extract volatiles from sample(s) which are more or less tightly bound to sample(s). Method(s) involving such approach(es) are described in the Prior Smith Patents. In aspects, in case(s) of measuring both hydrogen and helium in sample(s), it can be desirable to protect the sample from exposure to ambient air, such as sealing sample(s) in a hermetically sealed container, to prevent helium and hydrogen from diffusing out of the sample and thereby leading to a potential underestimation in the amount of one or both present, under- or over-estimating the purity of hydrogen in an apparent hydrogen-rich zone, under- or over-estimating the purity of helium in an apparent helium-rich zone, or, e.g., missing or misidentifying helium-rich or hydrogen-rich zone(s).

FIG. 2 further represents another, distinct but related, aspect of the invention, whereby helium is identifiable as, e.g., helium is characterizable as, helium is capable of operating as, helium is capable of providing, or helium is otherwise capable of establishing a “coating,” “sealant,” or “block” of material(s), preventing other molecule(s), compound(s), or both, from detectably or significantly interacting with the material(s). This is described in more detail elsewhere herein.

### Example 3 (FIG. 3)

Example 3 illustrates an aspect of the invention wherein method(s) herein utilize a plurality of volatile compound extraction pressures to extract volatile substance(s) such as hydrogen and helium (e.g., different vacuum pressures are utilized to obtain hydrogen and helium from material sample(s)) and the use of a plurality of extraction pressures yield insight(s) which are less obvious or which are not observable at all if single extraction pressure(s) are used in similar or the same method(s).

FIG. 3 of Example 3 illustrates two (2) sets of hydrogen-helium drill cuttings well logs for the exact same physical samples from the same bore hole, but the two sets of samples have had the hydrogen and helium extracted from their samples at different pressures (set 1 at one extraction pressure, set 2 at a different extraction pressure). In each of the 2 logs, amount(s) of helium in each sample is indicated by an X and amount(s) of hydrogen in each sample is indicated by open circles.

The left helium-hydrogen log pair provides the results of the analyses of a first-pressure extraction, at pressure “X”, e.g., at about 20 millibars (this reflects an aliquot which can, in aspects, have any of the characteristics of aliquots described in any of the Prior Smith Patents in terms of force/pressure applied, etc.). The right helium-hydrogen log pair provides the results of the analyses of a second pressure extraction, at pressure “Y”, e.g., at about 2 millibars (or, again, at any of the higher force/pressure conditions described with such aliquots in any of the Prior Smith Patents).

Both the pressure “X” and the pressure “Y” extractions are performed on the exact same physical cuttings sample without any physical movement of the joining between the sample tube and the vacuum inlet system of/within device(s) or system(s) used in such an extraction (and, e.g., analysis), such as, e.g., device(s)/system(s) described herein or in any of the Prior Smith Patents.

The pressure “X” extraction (the 2 lines/curves on the left) data reveal a “ROCK ZONE” of high helium and low hydrogen. The pressure “Y” extraction (the 2 lines/curves on the right) data reveal the same ROCK ZONE as low helium and high hydrogen. Again, the pressure “X” and pressure “Y” respectively identified ROCK ZONE is established by analysis of the exact same physical material samples (drill cuttings samples), collected from the exact same physical depth zones, from the exact same bore hole. Yet application of different pressures yields differing amounts of each of hydrogen and helium.

These data demonstrate that the rock within a given ROCK ZONE contain hydrogen within them which can be produced under different conditions from that of helium. For example, pressure X can be a lower vacuum pressure, e.g., about 20 mbar. Upon its application, helium is released from the material sample. Pressure Y can be a higher vacuum pressure, e.g., about 2 mbar. Upon its application to the same material (samples thereof), hydrogen is released from the material sample. In aspects, these data demonstrate that hydrogen of higher purity can be obtained from specific zones of, e.g., a well, if, for example, a different or specific, e.g., a higher pressure is utilized for such hydrogen collection. In certain aspects, detectable or significant amount(s) of helium can be removed from a zone by application of a low pressure, followed by the application of a high pressure for the collection of hydrogen.

The fact that the exact same rocks can be analyzed for the identification of hydrogen-poor helium zones, and helium-poor hydrogen zones by varying the extraction pressure suggests that a detailed study of, e.g., a given reservoir may allow for different production streams of both hydrogen-depleted helium as well as helium-depleted hydrogen. The production stream of helium-poor hydrogen may be of sufficient hydrogen purity so as to be used as a fuel, such as for example, greater than about 95%, ≥~96%, ≥~97%, ≥~98%, ≥~99%, ≥~99.5%, ≥~99.9%, or, e.g., ≥~99.95% or greater purity on a molar basis.

An additional or alternative description of aspects of the technology in or relating to the disclosure of FIG. 3 follows.

FIG. 3 can be additionally or alternatively viewed as illustrating that method(s) and device(s) herein can in aspects provide for the selective extraction of helium, the selective extraction of hydrogen, or the selective extraction of each of helium and hydrogen by varying the condition(s) to which a single material sample, or, e.g., a single location comprising the material, is exposed.

In aspects, the invention provides method(s) of applying two or more different force(s), e.g., differing in their type or degree, to a single sample, resulting in the extraction of helium, hydrogen, or both, wherein the force(s) applied is/are selected according to the target element. In aspects, a force can be, e.g., a temperature (e.g., the application of heat energy), a pressure force such as a vacuum, or any such force capable of detectably or significantly providing for the selective extraction of helium versus hydrogen or vice-versa. In aspects, the varying degree in a force can be, e.g., the application of two or more detectably or significantly different pressures, e.g., two or more pressures selected from between about 1 millibar and about 100 mbar (or more). In one particular example, e.g., a first pressure of 20 millibars and a second pressure of 2 millibars may be used as the two different applied forces.

FIG. 3 provides an example of the extraction of helium and hydrogen from the same set of samples, e.g., drill cuttings collected across various depths of a geologic location, e.g., a well, at a first extraction pressure “X” (left) and a second extraction pressure “Y” (right). “X” and “O” indicators indicate the samples as collected from each depth (Y-axis) and the respective amount of each element measured at such depth (X-axis). “X” indicators are used for helium results and “O” indicators are used for hydrogen results. Stated alternatively, FIG. 3 shows samples, e.g., drill cuttings, from a geologic location; each “X” and “O” demarcation indicates the location from which the sample was taken. Samples were extracted at a first pressure (left, pressure “X”) and a second pressure (right, pressure “Y”), with the resulting helium and hydrogen results at each pressure for each sample provided. Samples are the same on the right and the left of FIG. 3.

To exemplify performance of such a method, samples, e.g., drill cuttings samples, representing different depths of a well are collected. Each sample is first analyzed at a first pressure, wherein the quantity of released analytes of interest (e.g., hydrogen and helium) is analyzed. This may be referred to as a first aliquot. Subsequently, the sample is run at a second pressure, wherein the quantity of released analytes of interest (e.g., hydrogen and helium) is analyzed. This may be referred to as a second aliquot.

FIG. 3 illustrates that at given depth “A,” the extraction at extraction force “X” results in the extraction of helium only; no detectable or significant hydrogen (or, e.g., at least a significantly different amount of hydrogen) is detected; whereas, the subsequent extraction at extraction force “Y” results in the extraction of hydrogen only; no detectable or significant helium (or, e.g., at least a significantly different amount of helium) is detected.

In aspects, results shown in FIG. 3 demonstrate that helium and hydrogen may not comingle within rock in the same way(s) as the presently held belief in the art at the time of this filing. Rather, the present invention demonstrates that (a) helium and hydrogen may be stored separately within, but spatially close to one another, within rock, such that they can be extracted separately from a single sample by application of different force(s), (b) helium and hydrogen may be stored differently within rock, such as, for example, one being chemically bound to rock while another is not (e.g., with hydrogen being chemically bound to rock while helium is not), or, e.g., both (a) and (b) are true.

In aspects, results illustrated in FIG. 3 demonstrate that helium is present in a form which can be extracted at lower (er) vacuum pressure, e.g., 20 millibars, while hydrogen is present in a form which cannot be extracted at such pressure and requires application of a higher vacuum pressure, e.g., 2 millibars, to be released. This may indicate that hydrogen is present in, e.g., condensed form or associated with the rock in (e.g., perhaps in aspects in the form of a hydride) versus gaseous form. In aspects, it may be required to apply a sufficiently high vacuum pressure to get hydrogen past its boiling point to release it from the rock, e.g., to break its bonds with the mineral(s) of the rock, etc.

Data provided by FIG. 3 demonstrates that hydrogen and helium are not always co-located in the subsurface. While FIG. 2 shows their separation can exist stratigraphically, FIG. 3 demonstrates that even within the same strata, e.g., a single sample collected from a single location, hydrogen and helium can be stored differently within the same, e.g., rock-such as, e.g., in different pore structure(s).

In aspects, method(s) described herein can be applied to the extraction of hydrogen. For example, as shown in FIG. 2, method(s) and technology(ies) provided herein can identify zones of a geologic location comprising specific hydrogen-rich rock. Thus, such data can be used to target such strata or location(s) for exploration or extraction of hydrogen resources. As shown in FIG. 3, method(s) and technology(ies) provided herein can identify areas of geologic location(s) which should be produced at low vacuum pressure (higher mbar values) to facilitate the removal of helium, then revisited at a high vacuum pressure (lower mbar values) to facilitate obtaining hydrogen.

Accordingly, provided herein are method(s) of informing completion strategy (ies) of well(s) utilizing any one or more method(s) described herein. In embodiments, method(s) provided herein identify zone(s) or specific area(s) within a geologic location having specific characteristic(s) as described herein regarding helium and hydrogen content. In aspects, the invention further provides method(s) wherein differential extraction techniques can be applied to areas identified as having specific helium and hydrogen characteristic(s) to facilitate the removal of hydrogen. In aspects, method(s) herein identify areas to which differential extraction technique(s) (such as a variation in pressure) can be applied to facilitate the extraction of a detectably or significantly pure resource, such as, e.g., ≥~95%, ≥~96%, ≥~97%, ≥~98%, ≥~95%, 99% pure, ≥~99.5%, or ≥~99.9%, e.g., purity approaching or reaching 100% hydrogen (from, e.g., a geologic location).

In certain aspects, method(s) described herein, such as, e.g., method(s) of this Example, can be applied to helium-treated materials, the treatment of material(s) with helium to, e.g., protect the material(s) from hydrogen exposure, being described elsewhere herein.

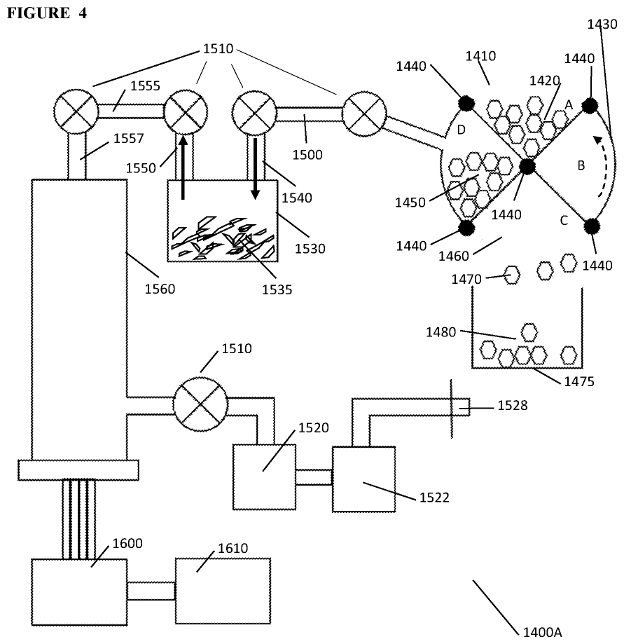

### Example 4 (FIGS. 4-8)

Example 4 illustrates exemplary configurations of a novel material collection and analysis system (which may herein be simply referred to as an “analyzer”), the system being capable of relatively rapidly analyzing hydrogen, helium, and other non-condensable gases, which can be, e.g., used at or near a well site and applied to geologic material(s) collected therefrom. Exemplary configurations of the analyzer of the technology described herein are provided as FIGS. 4-8 accompanying this disclosure. In certain aspects, benefit(s) of such analysis system(s) provided herein include the rapid collection of material samples and transfer of the same to an environment for volatile compound extraction, e.g., a vacuum chamber, wherein volatile substance(s) can be rapidly extracted and measured. In typical embodiments, analyzer system(s) provided herein comprise a component for removing select hydrogen-bearing substances (such as, e.g., water and hydrocarbons), which may be beneficial for particular method(s) in which such analyzer system(s) may be used.

FIGS. 4-8 illustrate various embodiments of novel, relatively rapid, and, e.g., in aspects, relatively portable material analyzers (1400A), (1400B), (1400C), (1400D), and (1400E), each suitable for the use in the analysis of hydrogen, helium, and, e.g., other non-condensable gases, which can be, for example, applied at or near a well site and applied to geologic materials including, materials which are mostly, materials which consist substantially of, or materials consisting of, drill cuttings samples, core samples, or drilling muds. The analyzers of FIGS. 4-8 differ in the presence of various component(s). Descriptions of components are not repeated for each separate Figure. The various exemplified embodiments of analyzers described here are intended to illustrate that one, some, most, generally all, substantially all, essentially all, or all components described herein can be present or absent in analyzers of this disclosure, can be present in any suitable combination in analyzers of this disclosure, and, e.g., uncontradicted, can be present in any suitable orientation relative to one another. A suitable orientation is any orientation wherein the relationship between component(s) renders the analyzer functional and capable of performing the activity (ies) described herein.

The exemplified analyzers of FIGS. 4-8 comprise a collection and transfer component (“CTC”) (1430) which, in the exemplary embodiments, looks like a revolving door on its side (description provided for the sake of orienting the reader to the Figures but which shape/appearance is non-limiting). The CTC (1430) operates to (a) collect material sample(s) and (b) transfer collected sample(s) to one or more different positions. In aspects, the CTC (1430) serves to collect geologic material or drilling mud samples from a larger source of material, such as, e.g., a stream of geologic material or, e.g., drilling mud, provided to the analyzer. In aspects, the CTC (1430) can be characterized as a rock volatiles sampling device.

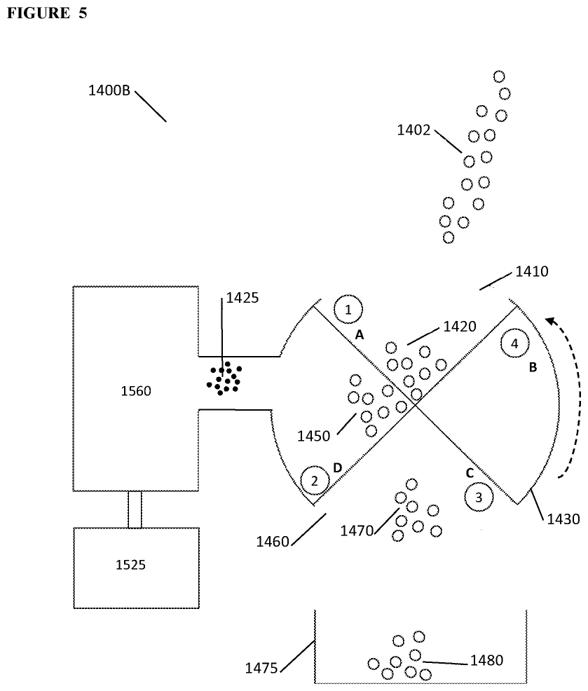

The CTC (1430) comprises a plurality of partitioned sections. As exemplified, the CTC (1430) is partitioned into 4 compartments, shown as A, B, C, and D in each of FIGS. 4-8. As will be described further elsewhere herein, the CTC (1430) can be rotated, e.g., in a clockwise or, e.g., a counterclockwise direction (as illustrated) such that the positions of each of the compartments A, B, C, and D is modifiable. Each of the compartments (also referred to herein as “chambers”) can be positioned in each of positions 1, 2, 3, and 4 (as shown in FIGS. 5-8).

The CTC (1430) comprises a compartment, illustrated in each of FIGS. 4-8 as compartment A, which comprises a sample entry point (1410) through which sample (sampled) material (“sample”) (1420) enters the analyzer; e.g., through which the sample (1420) enters the CTC (1430). Sample entry point (1410) is positioned within a portion of a wall defining a boundary of the compartment, defining a boundary of the CTC, or both.

The CTC (1430) can comprise one or more vacuum seal(s) (1440). Vacuum seal(s) can, in aspects, serve to participate in the sealing of each of the compartments A-D when such compartments are in one or more positions.

A known amount, e.g., a known volume, a known weight of material (e.g., cuttings and hereinafter just referred to as cuttings or simply “sample” for sake of illustration, as is the case with all the examples herein), or both a known volume and known weight of sample (1420) is delivered to the analyzer, e.g., more specifically the CTC (1430), either, for example, automatically or, e.g., by person. Sample (1420) is deposited into the CTC (1430) via the sample entry point (1410), shown as positioned on the top of the CTC (1430). Sample (1420) can be collected from a stream of material (1402) (depicted in FIGS. 5-8) delivered to the analyzer. Here, a “known” volume, weight, or both, of material can be, e.g., an at least relatively uniform and consistent or approximated volume, weight, or both, which is established by the timing of the rotation of the CTC (1430) whereby the sample entry point (1410) is alternatingly exposed and not exposed to the stream of material (1402) for consistent periods of time, and wherein the stream of material (1402) is provided at a relatively consistent volume and rate. In aspects, a “known” volume, weight, or both, is a volume, weight, or both which varies from a target or expected volume, weight, or each of an expected volume and weight by no more than about 50%, such as, e.g., ≤~45%, ≤~40%, ≤~35%, ≤~30%, ≤~25%, ≤~20%, ≤~15%, ≤~10%, ≤~5%, ≤~4%, ≤~3%, ~2%, or ~1%.

Rotating the CTC (1430), as illustrated, 90 degrees moves the sample (1420) into the cuttings volatiles extraction chamber, indicated as D. Samples in this position are indicated as extraction-ready samples (1450).

Analyzer(s) can comprise one or more vacuum valves (1510). Vacuum valves (1510) are illustrated as component(s) of the analyzer embodied in FIG. 4 but, as with any other component(s) of analyzer(s) provided herein, such component(s) illustrated in one Figure (e.g., as being present in one embodiment) can be present in other embodiment(s) even if not illustrated in such embodiments. For example, vacuum valve(s) (1510) can be present in the analyzer(s) embodied in any one, some, or all of FIGS. 5-8. Vacuum valves (1510) can operate to control the flow of fluid through portion(s) of the analyzer, wherein such portion(s) of the analyzer may be subject to a vacuum pressure. An inlet chamber (1500), illustrated in the embodiment shown in FIG. 4, is positioned between 2 vacuum valves (1510) closest to and on either side of the inlet chamber (1500). In operation, inlet chamber (1500) can be brought to detectable or significant vacuum using one or more vacuum pumps (one or more vacuum pumps being depicted as vacuum pump(s) (1525) of FIGS. 5, 7, and 8) such as, e.g., more specifically a high vacuum pump (1520) and a roughing pump (1522) as exemplified in FIG. 4. As depicted in FIG. 4, vacuum pumps (1520) and (1522) can be sealed off to the inlet chamber (1500) by a vacuum valve (1510). Both vacuum valves (1510) at the ends of the inlet chamber (1500) can be sealed. In operation, after the/a vacuum valve (1510) to the vacuum pumps (1520) and (1522) are sealed to the inlet chamber (1500), the vacuum valve (1510) closest to the cuttings volatiles extraction chamber (chamber D) is opened. Opening chamber D reduces the pressure in the cuttings volatiles extraction chamber (chamber D) and the inlet chamber (1500) to “x”, possibly, e.g., about 20 millibars. In operation, the vacuum valve (1510) between the cuttings volatiles extraction chamber (chamber D) and the inlet chamber (1500) is then closed.

According to aspects, analyzer(s) provided herein can comprise a component for selectively capturing particular volatile substance(s) (a volatile compound capture component (“VCCC”) (1530) as is illustrated in FIG. 4. In aspects, a VCCC is any component that selectively captures certain volatile substance(s). In aspects, a VCCC is a component that selectively captures certain volatile substance(s) by way of condensation. In aspects, a VCCC is a component capable of applying very low temperature(s) to condense and capture volatile substance(s) (gases) by freezing them onto a cold surface, such as, e.g., can be accomplished by a cryogenic trap (“cryotrap”). In aspects, analyzers comprise a cryogenic trap (e.g., VCCC (1530) can be a cryogenic trap). A VCCC (1530), exemplified as a cryogenic trap, can in aspects be held at, e.g., a temperature of minus 100 degrees C (−100 degrees C) or colder. In aspects, the purpose of the VCCC is to eliminate condensable volatile substances, wherein upon their capture by the VCCC, such volatile substances are discarded. In aspects, analyzer(s)/system(s) herein do not comprise a heating element suitable for heating a VCCC, such as a cryotrap. In aspects, volatile substances captured by a VCCC are not DOS released from the VCCC unless or until discarded. Volatile substances captured by a VCCC in typical aspects are not analyzed, e.g., are not analyzed by an analytical device associated with the system.

As provided in FIG. 4, an analyzer can comprise a vacuum valve (1510) positioned between the VCCC (1530) (e.g., cryotrap) and the inlet chamber (1500). In operation, a vacuum valve (1510) positioned between the VCCC (1530) and the inlet chamber (1500) is opened, allowing volatile(s) (1425) (as shown in FIGS. 5-8), which can include, e.g., volatile(s) extracted from extraction-ready sample(s) (1450), to, e.g., enter a VCCC (1530), e.g., a cryotrap, through a VCCC inlet (1540), when such a VCCC component is present.

In the embodiment of FIG. 4, condensable gases are frozen out of the volatiles (1425) in the VCCC (1530), e.g., a cryogenic trap. This includes the freezing out of water, which, in aspects, is the most hydrogen-rich compound in the extracted gas, and, e.g., hydrocarbons other than ethane and methane. Frozen volatiles (1535) are depicted in the embodiment of FIG. 4.

In aspects, analyzers herein comprise an analytical device (1560). In aspects, an analytical device can be, e.g., a mass spectrometer. After a sufficient period of time, a vacuum valve (1510) located between the VCCC (1530), e.g., cryogenic trap, and the inlet chamber (1500) is closed, and a vacuum valve (1510) positioned at the VCCC exit (1550) and a second vacuum valve (1510) at the entry of the analytical device (1560) (e.g., mass spectrometer)-that is, more specifically, e.g., vacuum valves (1510) located between the VCCC (1530) (e.g., cryogenic trap) and an analytical device inlet chamber (1555) of an analytical device (1560)—are opened (either simultaneously or one at a time). Vacuum valve(s) (1510) between the analytical device (1560) and the analytical device inlet chamber (1555) remain(s) closed.

In operation, a vacuum valve (1510) positioned between the analytical device inlet chamber (1555) and the analytical device inlet (1557) is then opened.

Hydrogen, helium, and, e.g., other non-condensable volatiles are allowed to enter the analytical device (1560) via the analytical device inlet (1557) and are analyzed by the analytical device (1560). Analyzed gases can, in aspects, be pumped out of the analytical device (1560) and exit the vacuum system.

The vacuum valve between the inlet system and the cuttings volatiles extraction chamber (chamber D), e.g., the vacuum valve between the inlet chamber (1500) and chamber D, can be re-opened, and the vacuum increased, e.g., to a higher vacuum “y”, such as, for example, to about 2 millibars or lower (e.g., wherein the lower the millibar pressure, the higher the vacuum), and the process can be repeated one or more times to analyze additional aliquot(s) at these one or more lower pressures.

The CTC (1430) in the embodiment(s) shown can be rotated another 90 degrees such that the extracted samples are now positioned in chamber C of the CTC (1430). Chamber C of the CTC (1430) comprises a sample exit (1460). Extraction-ready samples (1450), after having been exposed to the extraction chamber (D) and having had some, most, generally all, substantially all, essentially all, or all, volatile(s) extracted, exit the analyzer, e.g., specifically exit the CTC (1430), via sample exit (1460) as discarded samples (1470). Discarded samples (1470) can be collected upon discard as collected discarded samples (1480). One or more collection unit(s) (1475) or a system of collection unit(s) can be present for collecting discarded sample(s) for further use (e.g., testing, storage, etc.)

Analyzing cuttings volatiles at 2 or more different pressures has been demonstrated herein to reveal zones from which high-purity hydrogen may be extracted, and as such, multiple pressure analyses by analyzer(s) described herein can be desirable or otherwise advantageous.

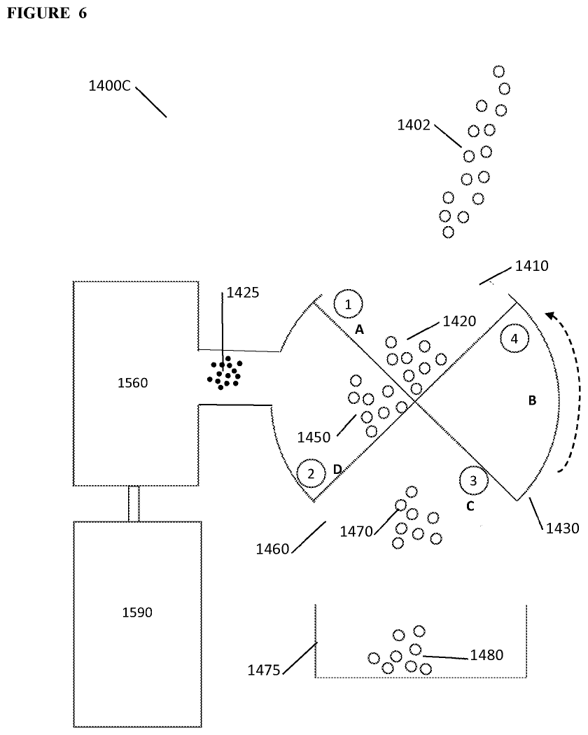

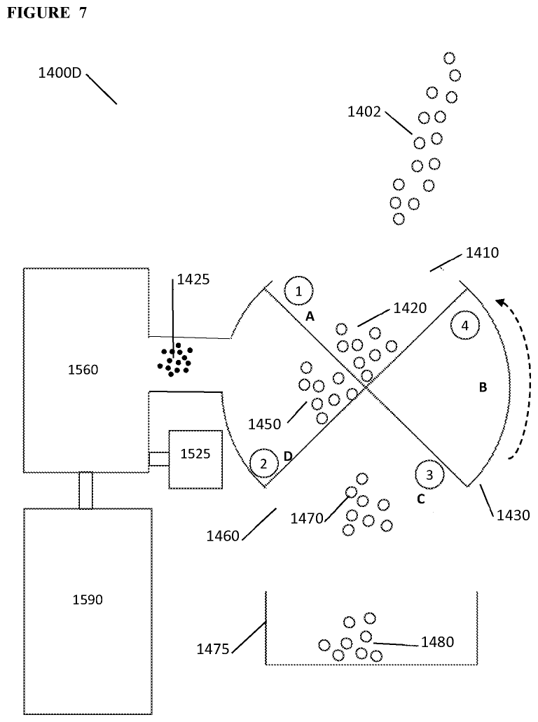

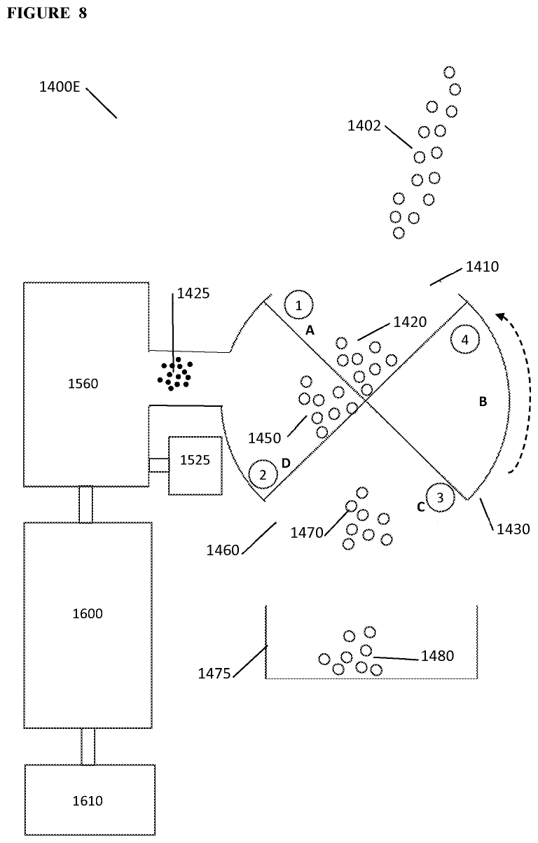

Analyzers provided herein can, in aspects, comprise a control system (1590), as illustrated in FIG. 6 and FIG. 7. A control system (1590) can comprise one or more computer-related or data-related components, such as, e.g., data interface and control component(s) (1600) (as shown in FIG. 4 and FIG. 8), computer component(s) (1610) (as shown in FIG. 4 and FIG. 8), or both.

Further, analyzers provided herein can, in aspects, comprise an exhaust (1528) (as illustrated in FIG. 4), associated with vacuum pumps (1525) or, e.g., specifically high vacuum pump(s) (1520), roughing pump(s) (1522), or both.

The analyzer/system described here can, in aspects, be fully automated. In aspects, analyzer(s)/system(s) disclosed herein can operate continuously while, e.g., a well is being drilled, whereby, for example, drill cutting(s) samples, or, e.g., drilling mud, is delivered to the analyzer/system in a stream and the analyzer/system is continuously sampling such material as described above. System(s) comprising control system(s) (1590), e.g., data interface and control unit(s) (1600), one or more computer(s) (1610), and the like, or combination(s) thereof, can allow operational control setting(s), such as, e.g., speed of sample collection and the like, to be modified. According to aspects, control system(s) can receive and collect data from an analysis component (1560), compile received/collected data, analyze or otherwise interpret such data, store data, store analysis(es) of such data, transmit data or analyses thereof, and the like.

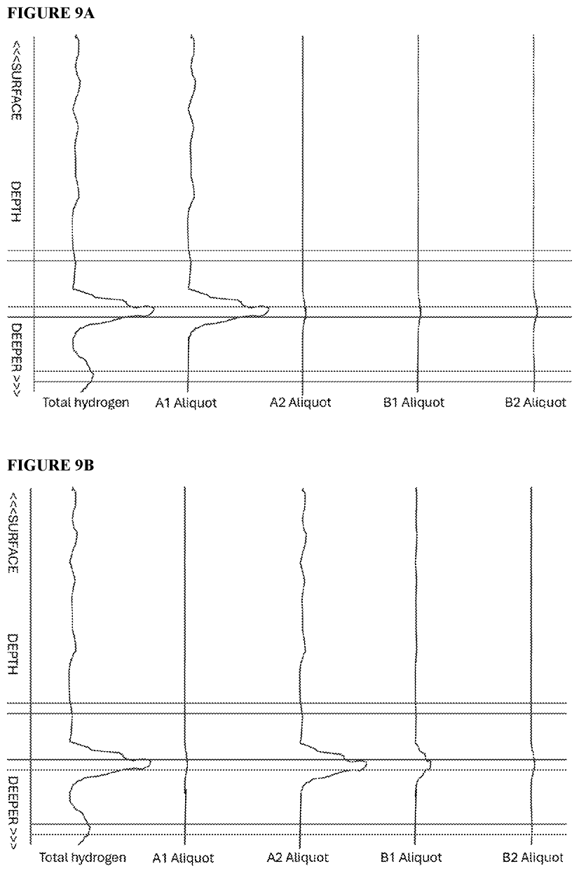

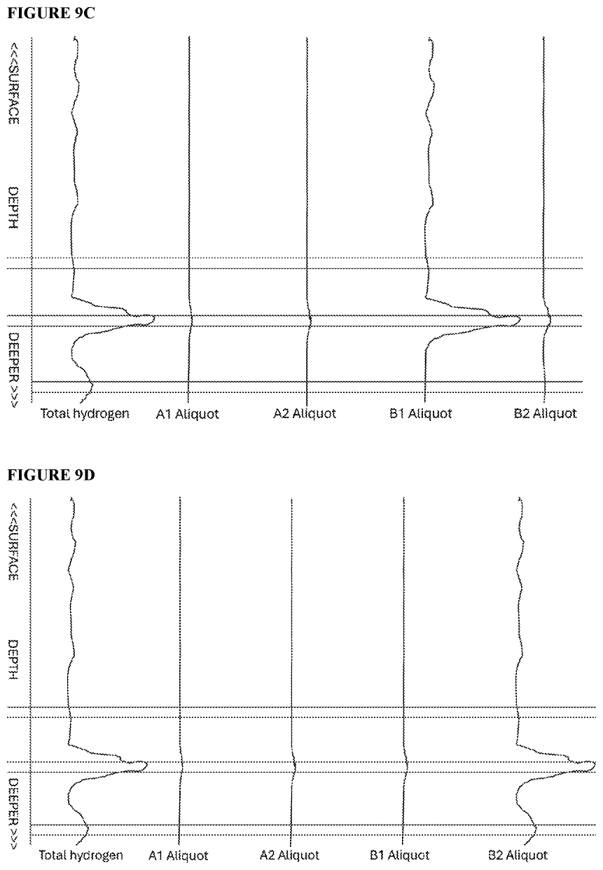

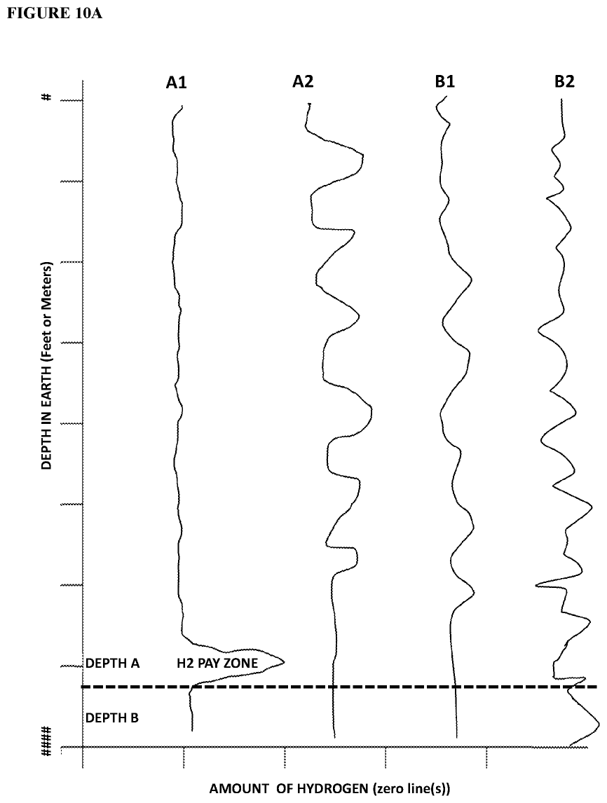

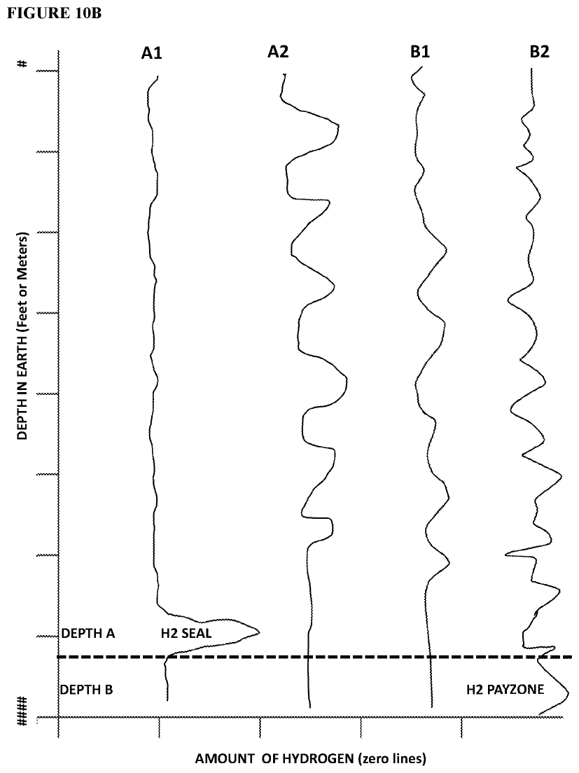

### Example 5 (FIGS. 9A-9D and FIGS. 10A-10B)

Example 5 demonstrates a rock volatiles extraction protocol comprising varied extraction strength and time, wherein such a protocol yields the identification of likely resource payzone(s) when applied to geologic materials.

Rock volatile(s) stratigraphy method(s) described herein and in the Prior Smith Patents, comprise a step for applying a force to a material sample to release one or more volatile substances. In certain aspects, a second force can be applied to the same material sample to release one or more additional and different volatile substances. In certain aspects, a first force, a second force, or both can be a vacuum pressure.

As has been described elsewhere herein, e.g., in Example 3 (and FIG. 3), the analysis of hydrogen and helium after the application of two different vacuum pressures as first and second forces can yield actionable data relative to the identification of possible hydrogen-rich, helium-poor zones as targets for the extraction of highly pure hydrogen; actionable data relative to the identification of possible helium-rich, hydrogen-poor zones as targets for the extraction of highly pure helium; or both.

It has been further discovered that modifying each of the first and second forces, to establish, for example, at least 3 or at least 4 extraction conditions, can provide further insight(s) relative to the identification of possible payzones, e.g., hydrogen payzones. The varied extraction conditions of such method(s) described here are designed to establish a picture of a span of, e.g., a well, from which rock material samples are collected, whereby differences between the presence of weakly held compound(s) and more tightly held compound(s) at locations across the span provides insight(s) into the location(s) or zones of such a span likely to be resource, e.g., hydrogen, payzones.

The concepts of Example 5 are illustrated in FIGS. 9A-9D and FIGS. 10A-10B. The rock volatile stratigraphy method(s) described in this Example and, e.g., method(s) such as those described in Example 3, can be referred to as “multi-aliquot rock volatile stratigraphy methods.” Multi-aliquot rock volatile stratigraphy methods can be applied to a variety of fields of endeavor, including but not limited to geological resource exploration, including but not limited to petroleum exploration and hydrogen exploration.

According to aspects, a rock volatile stratigraphy method can be applied to material sample(s), e.g., rock material sample(s), e.g., drill cutting(s) sample(s), core sample(s), and the like. The concepts illustrated in FIGS. 9A-9D and FIGS. 10A-10B can be applied to any such samples.

FIGS. 10A and 10B provide graphs wherein the depth within the Earth from which a series of rock material samples was collected is represented on the Y-axis, with shallower depths at the top of the Y-axis and deeper depths at the bottom of the Y-axis. The X-axis of the graphs of FIGS. 10A and 10B reflects the amount of hydrogen measured in each of the rock material samples under each extraction condition, identified as A1, A2, B1, and B2.

In support of the exemplified scenario(s) shown in FIGS. 10A and 10B, FIGS. 9A-9D illustrate results of a series of theoretical volatile compound, e.g., hydrogen, analyses. In the theoretical analyses, the location of material sample collection is provided on the Y-axis, e.g., the depth below the Earth's surface of a material sample, e.g., rock material sample, e.g., drill cuttings sample collection is shown on the Y-axis, and zero-line volatile compound, e.g., hydrogen, analyses are shown on the X-axis. Each of the zero line analyses “Al Aliquot,” “A2 Aliquot,” “B1 Aliquot,” and “B2 Aliquot,” illustrates theoretical amounts of hydrogen measured in samples collected from locations at varying depths. The “total hydrogen” line represents the total amount of hydrogen observed.

Each of “A1,” “A2,” “B1,” and “B2” represents different extraction states, wherein an extraction state comprises an extraction force applied for a given period. Extraction states “A1” and “A2” comprise an extraction force being a relatively weaker vacuum force. Extraction state A1 comprises the relatively weaker vacuum force applied to samples for a relatively shorter period of time. Extraction state A2 comprises the relatively weaker vacuum force applied to samples for a relatively longer period of time. Extraction states “B1” and “B2” comprise an extraction force being a relatively stronger vacuum force. Extraction state B1 comprises the relatively stronger vacuum force applied for a relatively shorter period of time. Extraction state B2 comprises the relatively stronger vacuum force applied to samples for a relatively longer period of time.

FIG. 9A illustrates that hydrogen is present at the depth indicated by the peak in A1 Aliquot graph. However, such hydrogen is only detected by the A1 extraction state (is only present in the A1 Aliquot.) This represents hydrogen present in a “loosely” bound state, e.g., it is somewhat easy to extract using the application of a relatively weak vacuum force for a relatively short period of time.

FIG. 9B illustrates the same analysis; however, in this hypothetical scenario, the hydrogen is only detected by the A2 extraction state (is only present in the A2 Aliquot.) This represents hydrogen present in still a somewhat “loosely” bound state; however, it is a bit harder to extract, requiring the application of the relatively weak vacuum force, but for a relatively longer period of time.

FIG. 9C again illustrates the same analysis; however, in this hypothetical scenario, the hydrogen is only detected by the B1 extraction state (is only present in the B1 Aliquot.) This represents more tightly bound hydrogen, requiring the application of a relatively strong vacuum force for a short period of time.

FIG. 9D again illustrates the same analysis; however, in this hypothetical scenario, the hydrogen is only detected by the B2 extraction state (is only present in the B2 Aliquot.) This represents hydrogen present in a very tightly bound form, requiring the application of a relatively strong vacuum force for a relatively longer period of time.

In each scenario illustrated in FIGS. 9A-9D, the total amount of hydrogen remains the same; however, the hydrogen making up such a total is only accessible when, in the different hypothetical scenarios, different extraction states are applied.

FIGS. 9A-9D illustrate that hydrogen can be present in samples collected from a given depth or zone of, e.g., a well. However, if such samples are analyzed under a single condition, e.g., a single extraction state, e.g., comprising a single extraction force (e.g., extraction strength) and for a single period of time, one could be led to believe that, for example, (1) hydrogen is absent from such a location, as would be the case if one utilized extraction state A1 or Al in the scenario of FIG. 9C or 9D, as such hydrogen is not available/detectable when such extraction states are applied; or (2) hydrogen may only present in a tightly bound state at such location, as would be the case if one utilized extraction state B1 or B2 in the scenario of FIG. 9A or 9B, as such hydrogen may be detectable upon application of such stronger extraction state(s) however such hydrogen would have been available/detectable upon application of weaker extraction state(s).

As such, FIGS. 9A-9D illustrate that the application of varied extraction states, e.g., varied extraction force(s) (such as, e.g., varying strength(s) of vacuum force(s)) applied for different times, can yield important data regarding the presence or absence of volatile substance(s) in materials such as, e.g., hydrogen in rock material samples.

Continuing on, the method of Example 5 comprises applying a first force, e.g., a first extraction force, under a first force extraction condition to each material sample; applying the first extraction force under a second force extraction condition to each material sample; applying a second force, e.g., a second extract force, under a second force condition to each material sample; and applying the second extraction force under a second force extraction condition to each material sample. In aspects, the extraction forces are applied to the same samples, in sequence. The amount of rock volatile(s), e.g., hydrogen as exemplified, collected under each condition is measured for each sample and can each be referred to as an “aliquot.” The amount of rock volatile(s) measured under each condition for each material sample is then graphed, and the amount of rock volatile(s), e.g., hydrogen, obtained for each aliquot of each sample is compared.

The four (4) exemplified aliquots of Example 5 illustrated in FIG. 10A and FIG. 10B represent increasing force(s) applied to each sample. The force applied in the rock volatiles stratigraphy method of Example 5 is a vacuum force.

Aliquot “A1” again represents the first and weakest volatiles extraction state applied. Al represents the application of a first vacuum force, e.g., a relatively low vacuum force, under a first condition, e.g., for a relatively short period of time. The extraction state of aliquot Al in this Example is a vacuum force of about 20 mbar applied for a period of about 1 minute.

Aliquot “A2” represents a volatile(s) extraction state which is a bit stronger than that of A1. A2 represents the application of the same first vacuum force, e.g., a relatively low vacuum force, under a second condition, e.g., for a relatively longer period of time. The extraction state of aliquot A2 in this Example is a vacuum force of about 20 mbar applied for a period of about 8 minutes.

Aliquot “B1” represents a volatile(s) extraction state which is still stronger than that of A1 and A2. B2 represents the application of a second vacuum force, e.g., a relatively higher vacuum force, under a first condition, e.g., for a relatively short period of time. The extraction state of aliquot B1 in this Example is a vacuum force of about 2 mbar applied for a period of about 1 minute.

Aliquot “B2” represents a final and strongest volatiles extraction state applied in these Examples. B2 represents the application of the same second vacuum force, e.g., a relatively higher vacuum force, under a second condition, e.g., for a relatively longer period of time. The extraction state of aliquot B2 in this Example is a vacuum force of about 2 mbar for a period of about 8 minutes.

As provided above, the amount of volatile(s) collected upon the application of each volatile(s) extraction state is measured and plotted for each sample. FIGS. 9 and 10 both represent the plot of such data. In each of FIG. 10A and FIG. 10B, it is observed that in aliquot A1, reflecting the gentlest extraction state, little to no hydrogen is collected until Depth A is reached, at which time a spike in the amount of hydrogen is observed. At Depth A, aliquots A2, B1, and B2 demonstrate little to no hydrogen present. Below (deeper to) Depth A, at Depth B, aliquots A2 and B1 demonstrate little to no hydrogen present. Below (deeper to) Depth A, aliquot B2 demonstrates a spike in the amount of hydrogen.

In one aspect, such a pattern can indicate that Depth A represents a potential resource (e.g., hydrogen) payzone target. In one aspect, Depth A represents a location at which high amounts of hydrogen may be present and readily extracted, while depths below that of Depth A, e.g., Depth B, represents a location that perhaps previously represented a hydrogen payzone but which is now drained. This is reflected in FIG. 10A.

In an alternative aspect, such a pattern can indicate that Depth B represents a potential resource (e.g., hydrogen) payzone target. At Depth B, the amount of hydrogen present in aliquots A1, A2, and B1 is/are minimal or absent, while hydrogen is present in detectable or significant amounts in aliquot B2. In one aspect, Depth A represents a “hydrogen seal”, wherein, below such location, the quantity of the resource (hydrogen) is very high and very loosely bound to rock material; sufficiently bound to rock material such that it is lost on its way up the borehole and to its point of collection and sealing as a collected sample; e.g., significant amounts of hydrogen are present at such locations but have been lost prior to its analysis. However, a tightly bound resource, e.g., hydrogen, remains and is identified in aliquot B2.

Accordingly, Example 5 provides method(s) of identifying resource targets, wherein the method comprises the identification of a first zone, Zone 1, within a geologic location where loosely held hydrogen, detectable in an aliquot obtained by the application of a low vacuum pressure for a short period of time (e.g., about 20 mbar for about 1 minute; aliquot A1) is present while more tightly held hydrogen, detectable in aliquots obtained by the application of such a low vacuum pressure for a longer period of time (e.g., about 8 minutes) or detectable in aliquots obtained by the application of a higher vacuum pressure (aliquots A2, B1, and B2) is relatively absent; and wherein such a first zone (Zone 1) exists immediately above (more shallow to) and adjacent to a second zone, Zone 2, where more tightly held hydrogen is relatively abundant, detectable in an aliquot obtained by the application of a higher vacuum pressure for an extended period of time (e.g., about 2 mbar for about 8 minutes; aliquot B2) is present while more loosely held hydrogen, detectable in aliquots obtained by the application of such a higher vacuum pressure for a shorter period of time (e.g., about 1 minute) or detectable in aliquots obtained by the application of a lower vacuum pressure (aliquots A1, A2, and B1) is relatively absent. In aspects, the identification of such a pattern indicates that a hydrogen payzone likely exists in Zone 1; a hydrogen payzone likely exists in Zone 2; or both Zone 1 and Zone 2 represent likely hydrogen payzones. In aspects, Example 5 demonstrates how the use of multi-aliquot rock volatile stratigraphy methods can identify payzones which may otherwise go undetected or which may be erroneous if a multi-aliquot RVS method is not applied.

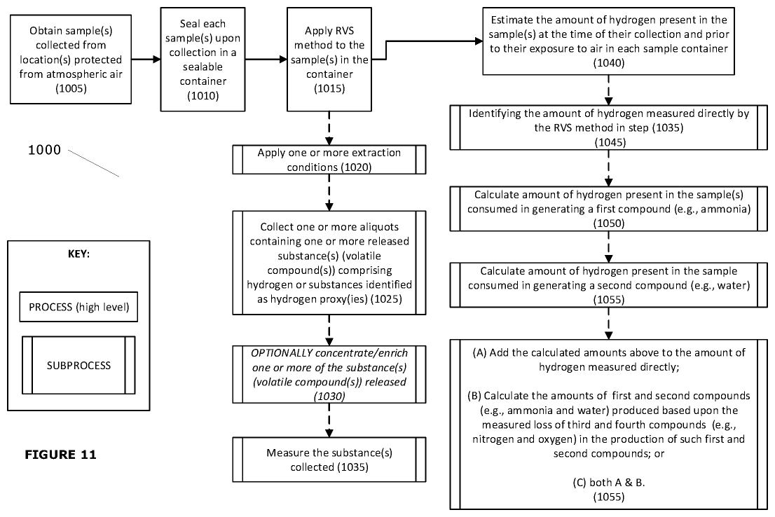

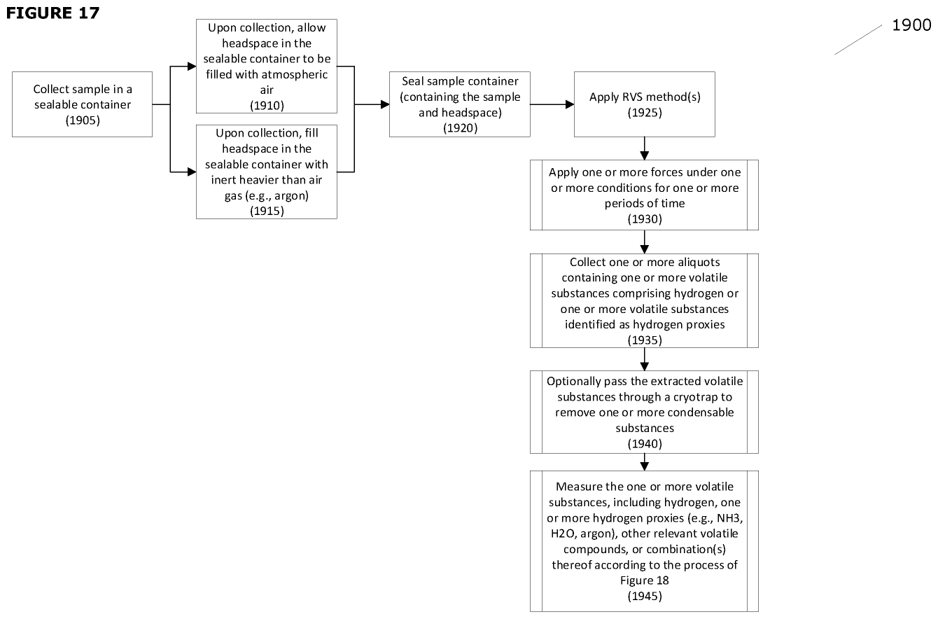

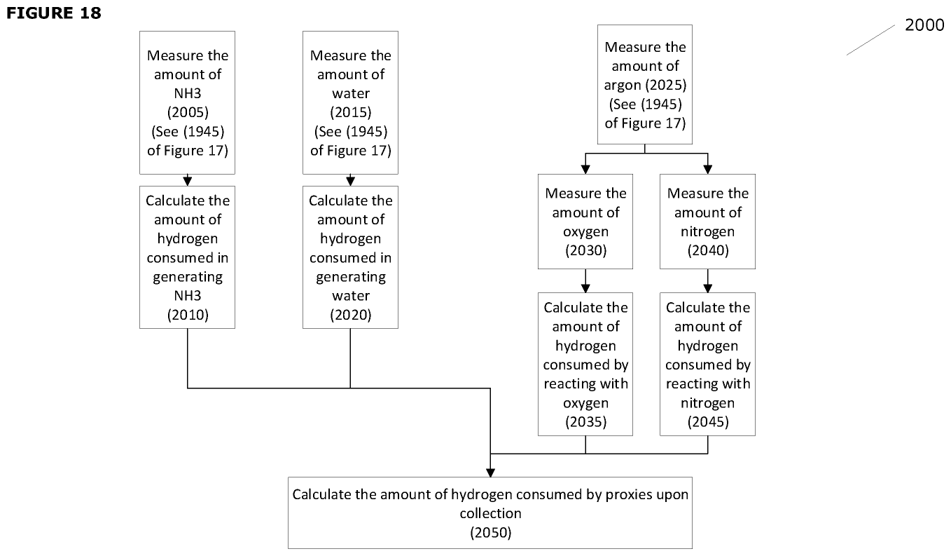

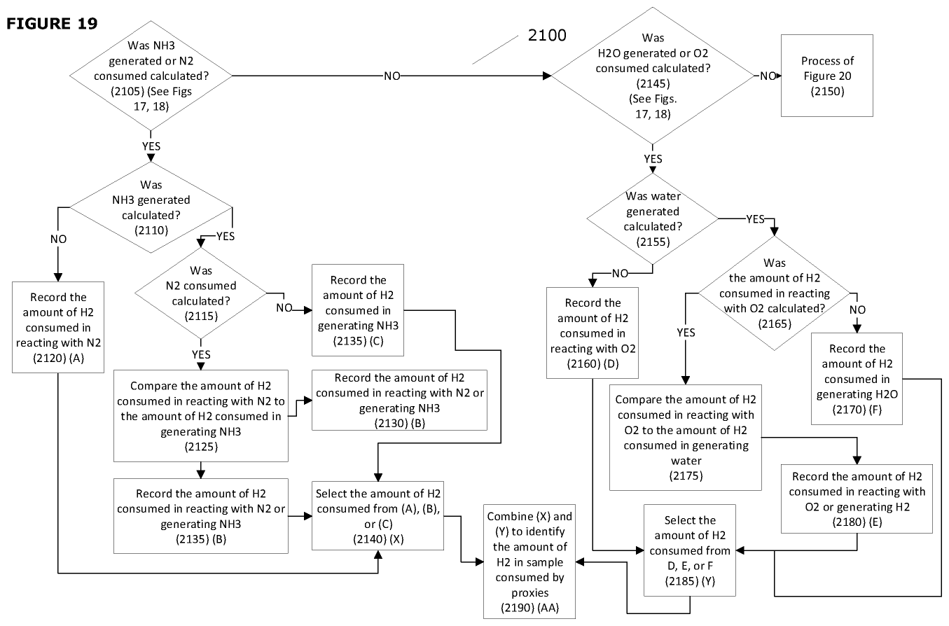

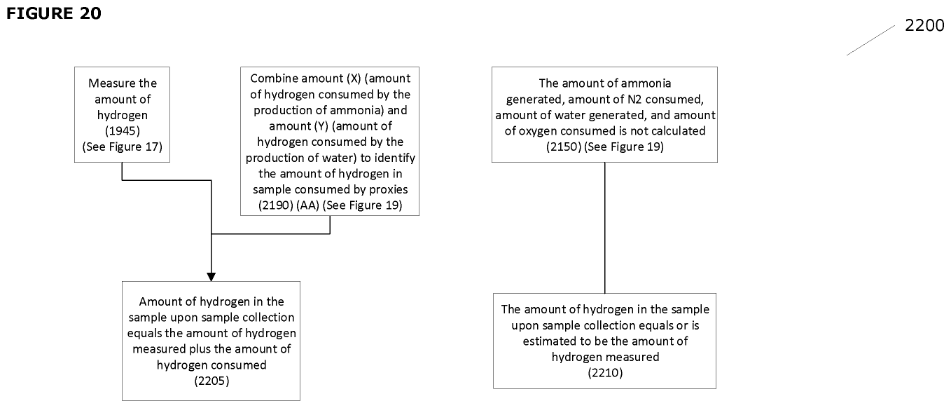

### Example 6 (FIG. 11, FIGS. 17-20)

Example 6 provides an exemplary method (1000) of the technology disclosed herein, wherein the amount of hydrogen present in a material sample at the time of its collection is determined. Such a method is exemplified in FIG. 11 (1000).

Samples, e.g., rock material samples, are obtained (1005) from location(s) which are at least mostly, at least generally, at least substantially, at least essentially, or are protected from atmospheric air. Such locations are typically subterranean locations, e.g., locations associated with a well, e.g., a resource exploration well, such as a petroleum well, gas resource exploration well, and the like.

Samples are sealed (1010) upon collection in a sealable container. Collection can occur as close to the time the sample is first exposed to atmospheric air as possible. For example, samples can be drill cutting(s) or core samples, wherein sealing such collected samples occurs when the samples are first brought to the Earth's surface. Sealable containers can, in aspects, be containers described in Prior Smith Patents.

Samples are subjected to a rock volatile(s) stratigraphy (RVS) method (1015) while in the container. Such RVS method(s) can comprise (as shown as subprocess steps in FIG. 11) a step for applying one or more extraction conditions (1020); a step for collecting one or more aliquots containing hydrogen or substances identified as hydrogen proxy (ies) (1025); optionally concentrating or enriching (1030) one or more of the volatile substances released in the extraction; and measuring the substance(s), e.g., hydrogen, collected (1035). Upon application of the RVS method (1015), the amount of hydrogen present in the samples at the time of their collection and prior to their exposure to air in each sample container is estimated (1040). This estimate comprises: (1) identifying the amount of hydrogen measured directly by the RVS method (1045); (2) calculating the amount of hydrogen present in the sample(s) consumed in the generation of a first compound (e.g., ammonia) (1050); (3) calculating the amount of hydrogen present in the sample(s) consumed in the generation of a second compound (e.g., water) (1055); and (4) (a) adding the calculated amounts of hydrogen present in the sample(s) consumed in the production of first and second compounds (e.g., ammonia and water), (b) calculating the amounts of first and second compounds (e.g., ammonia and water) produced based upon the measured loss of third and fourth compounds (e.g., nitrogen and oxygen) in the production of such first and second compounds, or (c) both (a) and (b) (1055). Therein, consumption of hydrogen can be at least mostly, at least generally, at least substantially, at least essentially, or can be due to the interaction of the sample(s) with atmospheric air, e.g., atmospheric air trapped inside the sample container with the sample(s) when the sample(s) are sealed therein.

Accordingly, Example 6 provides a method of determining the total amount of hydrogen which may be present in or associated with an Earth strata when samples are available from such a location, and wherein such samples are sealed immediately upon their collection. As hydrogen is very light and easily lost, and, further, as hydrogen can be consumed in the production of new compounds upon its interaction with atmospheric air, the method of Example 6 provides a way of accurately estimating the amount of hydrogen which may be present in or associated with geologic location(s), whereby the presence of a significant amount (and, e.g., purity) of hydrogen may indicate commercially relevant target location(s) for resource (hydrogen) extraction.

FIGS. 17-20 further exemplify the method of estimating the amount of hydrogen in sample(s). A general description of the exemplary method(s) is provided here, followed by a more specific description of FIGS. 17-20.

FIGS. 17-20 together provide a description of the overall process that may be performed in measuring the amount of hydrogen in a sample at the time the sample was collected in a sealable container.

The overall process can be broken down into several subprocesses: (1) collecting the sample and sealing in a sealable container, (2) performing an analysis of the sample and any void space to measure one or more volatile substances extracted from the sealed sample, and (3) estimating the amount of hydrogen present in the sample at the time of it being collected in the sealable container by combining the amount of hydrogen measured and any hydrogen consumed as evidenced by the change in one or more hydrogen proxies.

Regarding sample collection and sample sealing in a sealable container, the sample may be a geological material, e.g., drill cuttings or core sample, or e.g., a drilling mud. The sample may be collected by any method known by one of ordinary skill in the art into a sealable container. The sample may occupy a portion of the sealable container, leaving a void above the sample, or the sample may occupy the entire sealable container, not leaving any void above the sample.

Prior to sealing the sample, any void space that exists in the sealable container above the sample may be filled by the ingress of atmospheric air, comprising numerous gases, e.g., nitrogen, oxygen, water vapor, and argon. Alternatively, the void (if any) may be purged using equipment known in the art with a heavier than air gas, e.g., argon, krypton or xenon, thereby mostly eliminating or entirely eliminating any atmospheric air that may have gotten into the void space in the sealable container above the sample.

The sealable container is then sealed by any method known in the art to mostly prevent or entirely prevent any further exchange of any gases in any void space above the sample with atmospheric air that exists outside the sealed container containing sample.

The sample may be analyzed immediately, e.g., in less than about 1 minute after sealing the container by the one or more suitable analysis methods, or analyzed at some point after sealing the container, e.g., greater than 1 minute, hours, days, weeks, months, or, e.g., years.

Regarding the analysis of the sample and any void space which may be present, after the sample is collected and sealed in a sealable container, it may then be analyzed to measure one or more volatile substances present in the sealed container. The volatile substances in the sealed container may be measured by any suitable method known in the art which can provide qualitative or quantitative measurement of volatile substances, e.g. the RVS methodology described herein or in the Prior Smith Patents.

In the analysis process, one or more conditions, e.g., a vacuum force, may be applied to the inside of the sealed container which contains a sample and may contain a void space above the sample which was originally occupied by atmospheric air or a heavier-than-air gas (as applicable) at the time the sample container was sealed.

After the application of a vacuum force, e.g., for a period of time, e.g., a few seconds to several minutes, an aliquot is collected of any volatile substance(s) that are removed during the application of the force.

The aliquot of volatile substances may be passed through a cryotrap, e.g., to condense one or more condensable gases, e.g., water vapor, and hydrocarbons typically with more than 2 carbon atoms.

The aliquot of volatile substances, optionally passed through a cryotrap, is then analyzed using one or more suitable methods that can qualitatively, or more preferably quantitatively measure one or more volatile substances in the aliquot, e.g., hydrogen, helium, argon, nitrogen, ammonia, water vapor, or oxygen.

The sealed sample may be optionally subjected to one or more additional conditions, e.g., a stronger vacuum force, to collect one or more additional aliquots, optionally passed through a cryotrap, wherein the one or more additional aliquots are analyzed to measure one or more volatile substances in the one or more additional aliquots, e.g., hydrogen, helium, argon, nitrogen, ammonia, water vapor, or oxygen.

Having now completed both (1) the sample collection and sample sealing in a sealable container subprocess and (2) the sample and any void space analysis subprocesses, the overall process proceeds to (3) the estimation of the amount of hydrogen in the sample at the time of its collection subprocess. Each of the 3 subprocesses may be completed by the same entity or one or more entities; for example, the estimation subprocess may be completed by one or more entities after having been provided the amounts of one or more volatiles measured in the analysis subprocess.

Regarding the estimation of hydrogen present in the sample at the time of its collection, in order to estimate the amount of hydrogen in a sample at the time of its collection, four steps can be completed, including, e.g.: (1) measuring the amount of hydrogen analyzed in the analysis subprocess, (2) measuring the amount of one or more hydrogen proxies analyzed in the analysis subprocess, (3) determining the amount of hydrogen consumed as evidenced by the change in the amount of one or more hydrogen proxies, and (4) combining the amount of hydrogen measured with the amount of hydrogen consumed as evidenced by a change in the amount hydrogen proxies measured to provide an estimate of the amount of hydrogen in the sample at the time of its collection.

First, the amount of hydrogen remaining in the sample in the sealed container is measured. Second, for measuring changes in hydrogen proxies, either the amount of ammonia, the amount of water vapor, the amount of argon, the amount of oxygen, the amount of nitrogen or any combination thereof is measured.

If the amount of ammonia is measured, then the amount of hydrogen that was consumed to make the ammonia is calculated, wherein the amount of ammonia generated is a proxy for an amount of hydrogen that was in the sample at the time it was originally sealed in the sealable container.

If the amount of water vapor is measured, then the amount of hydrogen that was consumed in making the water vapor is calculated, wherein the amount of water generated is a proxy for an amount of hydrogen that was in the sample at the time it was originally sealed in the sealable container.

If the consumed amount of oxygen, the consumed amount of nitrogen or both are to be used as proxies for the amount of hydrogen that was in the sample at the time the sample was sealed in the sealable container, then the amount of argon that is in the sealed sample container must be measured.

In the event that the amount of oxygen consumed is to be used as a proxy for the amount of hydrogen in sample at the time the sample was sealed, then the amount of oxygen consumed is adjusted by the amount of argon in the sealed container to calculate the absolute amount of oxygen consumed, wherein the absolute amount of oxygen consumed is a proxy for an amount of hydrogen that was in the sample at the time it was originally sealed in the sealable container.

In the event that the amount of nitrogen consumed is to be used as a proxy for the amount of hydrogen in sample at the time the sample was sealed then the amount of nitrogen consumed is adjusted by the amount of argon in the sealed container to calculate the absolute amount of nitrogen consumed, wherein the absolute amount of oxygen consumed is a proxy for an amount of hydrogen that was in the sample at the time it was originally sealed in the sealable container.

The amount of hydrogen measured with the amount of hydrogen consumed as measured by the change in the amount of one or more hydrogen proxies, e.g., the amount of ammonia produced or the absolute amount of nitrogen consumed or a combination thereof, and the amount of water produced or the absolute amount of oxygen consumed or a combination thereof, can then be combined to provide an estimate of the amount of hydrogen that was in the sample at the time the sample was sealed in the sealable container.

A specific description of such steps as illustrated in FIGS. 17-20 follows.

The method of FIG. 17 (1900) begins with the collection of a sample in sealable container (1905). Upon collection in the container, the container may have a headspace, e.g., space not filled by the sample itself. In a first aspect, a headspace may be allowed to be filled with atmospheric air (1910). In an alternative aspect, a headspace may be filled with an inert gas which is heavier than air, such as, e.g., argon (1915). The sample container is then sealed (1920). RVS method(s), such as RVS method(s) described herein or in the Prior Smith Patents are applied to the samples (1925). The RVS method(s) can comprise (1) application of one or more forces under one or more conditions or one or more periods of time (1930); (2) collection of one or more aliquots containing one or more volatile substances comprising hydrogen or one or more volatile substances identified as hydrogen proxies (1935); (3) optionally pass the extracted volatile substances through a cryotrap to remove one or more condensable substances (1940); and (4) measuring the one or more volatile substances, including hydrogen, one or more hydrogen proxies, other relevant volatile substances, or combination(s) thereof (for example, e.g., according to the method/process described in FIG. 18, described below) (1945).

FIG. 18 describes in more detail (2000) step (1945) of FIG. 17, wherein one or more volatile substances, including hydrogen, one or more hydrogen proxies, or other relevant volatile substances, or combination(s) thereof are measured as a step of an RVS method.

In FIG. 18, multiple options are provided for measuring hydrogen proxies which can be performed independently (e.g., without one or more other options being performed) or which can be performed in any combination.

A first option is to measure the amount of ammonia (2005), and from the measured amount of ammonia, the amount of hydrogen consumed in generating ammonia is calculated (2010).

A second option is to measure the amount of water (2015), and from the measured amount of water, the amount of hydrogen consumed in generating water is calculated (2020).

A third option is to measure the amount of argon (2025) in addition to the amount of oxygen (2030), from which the amount of hydrogen consumed by reacting with oxygen (2035) is calculated. Alternatively, an option is to measure the amount of argon (2025) in addition to the amount of nitrogen (2040), from which the amount of hydrogen consumed by reacting with nitrogen is calculated (2045).

From any one of such provided options or from combination(s) thereof, the amount of hydrogen consumed by proxy (ies) upon collection (2050) is determined.

Step (2050) of determining the amount of hydrogen originally present in a sample but which has been consumed by proxies is further described in FIG. 19.

The process of FIG. 19 (2100) begins with determining whether or not the amount of ammonia generated or nitrogen was consumed calculated (2105). If such a calculation was performed, it is determined whether or not ammonia generated was calculated (2110). If it was not calculated, the amount of hydrogen consumed in reacting with nitrogen (2120) is captured as amount A. If it was calculated, it is determined whether or not the amount of nitrogen consumed was calculated (2115). If it was not calculated, the amount of hydrogen consumed in generating ammonia is captured (2135) as amount C. If it was calculated, the amount of hydrogen consumed in reacting with nitrogen is compared to the amount of hydrogen consumed in generating ammonia (2125). The amount of hydrogen consumed in reacting with nitrogen or generating ammonia is captured (2135) as amount (B). The amount of hydrogen consumed is then selected from the amounts (A), (B), or (C) (2140) and is captured as amount X.

If the amount of ammonia generated or nitrogen consumed was not calculated, it is determined whether the amount of water generated or oxygen consumed was calculated (2145). If it was not calculated, the process described in FIG. 20 (2150) is performed.

If it is was calculated, it is determined whether the amount of water generated was calculated (2155). If the amount of water generated was not calculated the amount of hydrogen consumed in reacting with oxygen is recorded (2160) and is captured as amount D. If the amount of water generated was calculated, it is determined whether the amount of hydrogen consumed in reacting with oxygen was calculated (2165).

If the amount of hydrogen consumed in reacting with oxygen was not calculated, the amount of hydrogen consumed in generating water is recorded (2170) and is captured as amount (F). If the amount of hydrogen consumed in reacting with oxygen was calculated, the amount of hydrogen consumed in reacting with oxygen to the amount of hydrogen consumed in generating water is compared (2175). The amount hydrogen consumed in reacting with oxygen or generating hydrogen (2180) captured as amount (E). The amount of hydrogen consumed is then selected form the amounts (D), (E), and (F) (2185) and is captured as amount Y.

The amount (X), representing a first calculation of the amount of hydrogen consumed by proxies, is compared to the amount (Y), representing a second calculation of the amount of hydrogen consumed by proxies, is compared (2190) and is captured as amount (AA).

Moving to the process described in FIG. 20 (2200), upon measuring the amount of hydrogen in step (1945) of FIG. 17, this amount is added to the combined amount of hydrogen consumed by the production of ammonia and the amount of hydrogen consumed by the production of water (2190) and established as amount (AA) to obtain the amount of hydrogen in the sample upon sample collection (2205). However, if the amount of ammonia generated, the amount of nitrogen consumed, the amount of water generated, and the amount of oxygen consumed is not calculated (2150), then the amount of hydrogen in the sample upon sample collection equals or is estimated to be the amount of hydrogen measured (2210).

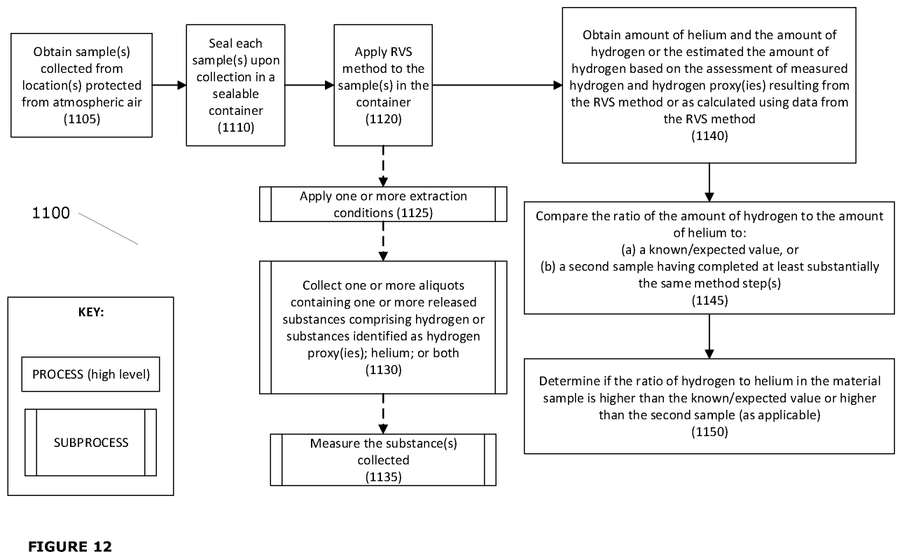

### Example 7 (FIG. 12)

Example 7 exemplifies an embodiment of the technology disclosed herein, wherein the technology provides method(s) of (a) identifying relative analyte purity, (b) identifying separate zones wherein each zone is relatively more pure in one or more analyte(s) than another, (c) identifying target zones for resource production (e.g., hydrogen production), or combination(s) thereof. Such exemplary methods are illustrated in FIG. 12.

FIG. 12 begins with obtaining sample(s) collected from location(s) protected from atmospheric air (1105), typically subterranean locations, e.g., wherein the sample(s) are sample(s) associated with a well, such as drill cutting(s) samples, core sample(s), or the like. Each sample obtained is sealed upon its collection in a sealable container (1110).

Rock volatile stratigraphy (RVS) method(s) are applied to each sample (1120), wherein the RVS method(s) comprise (1) the application of one or more extraction condition(s) to the sample(s) (1125); (2) the collection of one or more aliquots containing one or more released substances comprising hydrogen or substances identified as hydrogen proxy (ies), helium; or both (1130); and (3) the measurement of the substance(s) collected (1135). Upon completion of the application of RVS methods, the amount of helium and the amount of hydrogen or the estimated amount of hydrogen based on the assessment of hydrogen proxy (ies) is obtained (1140). The ratio of the amount of hydrogen to the amount of helium is then compared to (1) a known or expected value, or (2) another/other sample(s) having completed at least substantially the same method step(s) (1145). A determination is made regarding whether the ratio of hydrogen to helium in the material sample is higher than the known/expected value or higher than the second sample (as applicable) (1150).

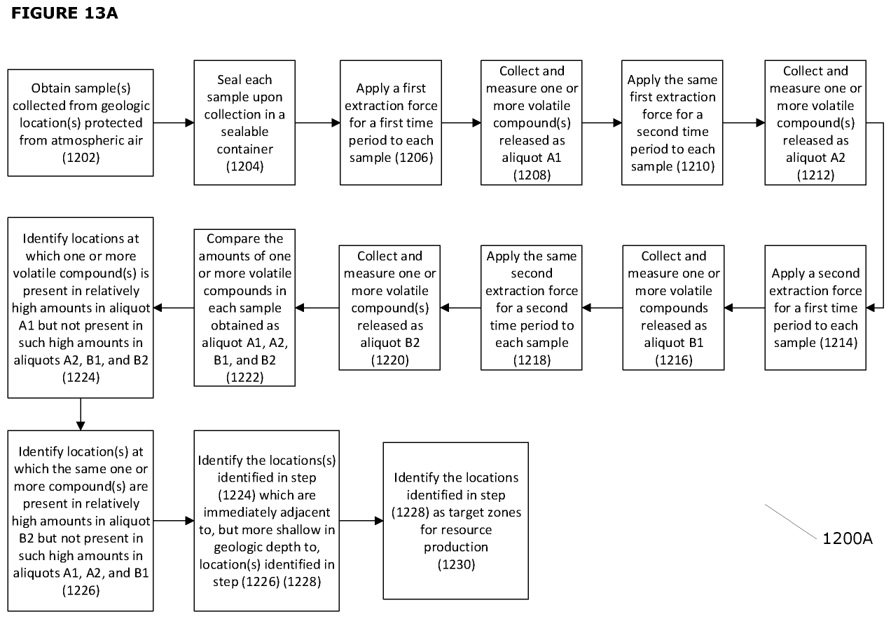

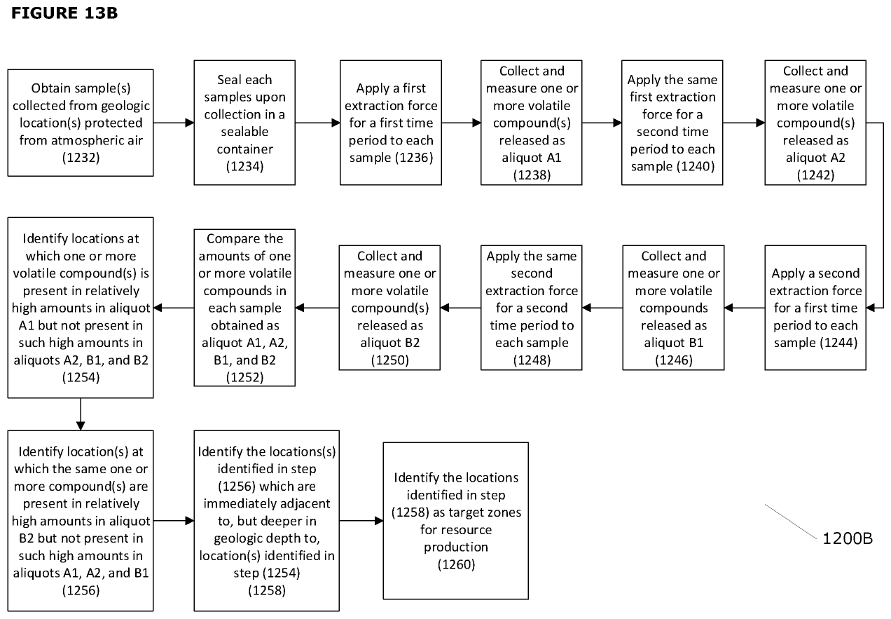

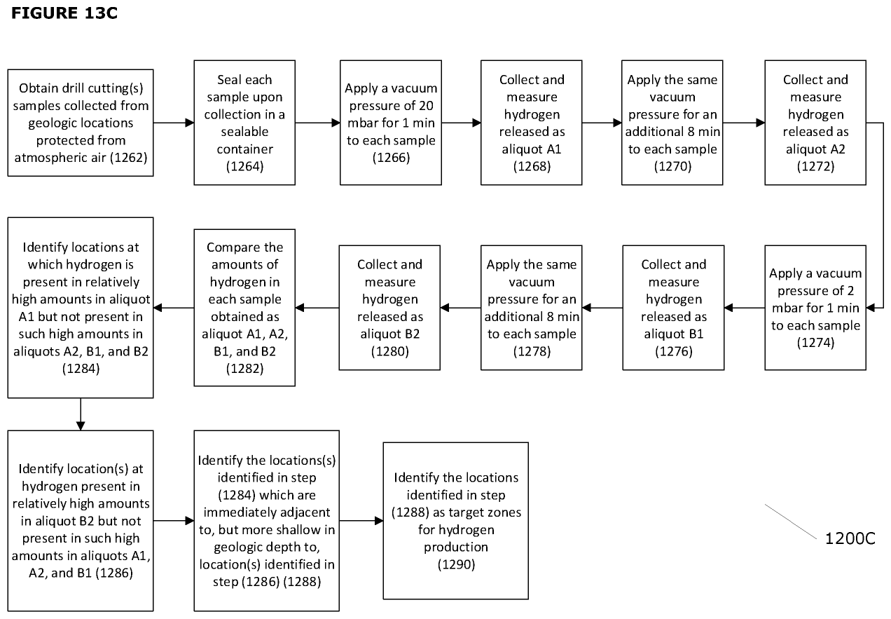

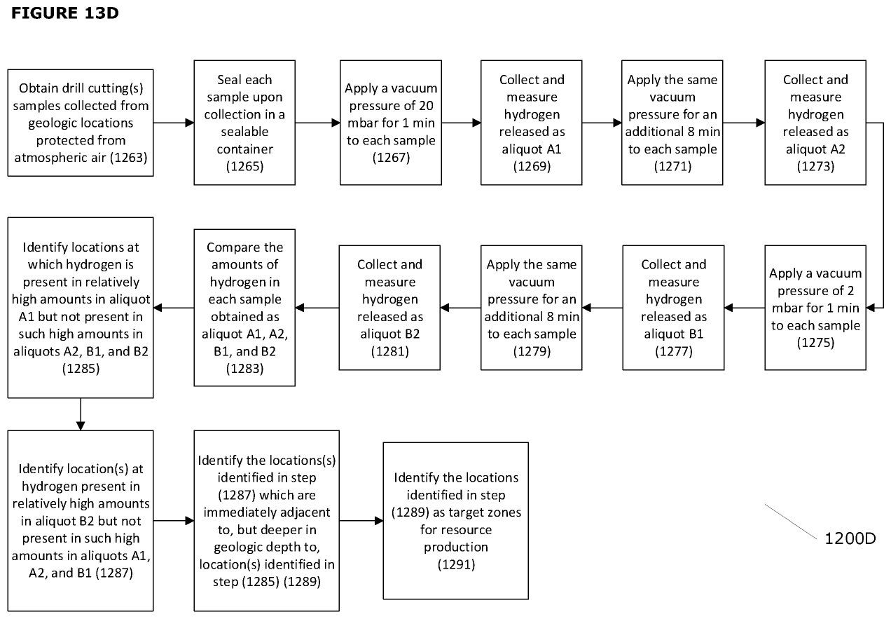

### Example 8 (FIGS. 13A-13D)

Example 8, illustrated in FIGS. 13A-13D, describe method(s) of the technology directed to the capture, analysis, and application of multiple aliquots of extracted volatile substance(s), wherein results from the multiple aliquot analysis can aid in the identification of target resource zones, e.g., target hydrogen-rich production zones.

FIG. 13A provides one such exemplary method (1200A).

FIG. 13A begins with obtaining sample(s) collected from geologic location(s) protected from atmospheric air (1202), typically subterranean location(s), e.g., location(s) associated with a well. Each sample is sealed upon collection in a sealable container (1204). A first extraction force is applied to each sample as part of an RVS method for a first time period (1206). Extracted volatile substances are collected and measured as aliquot A1 (1208). The same first extraction force is again applied to each sample, this time for a second time period (1210). Extracted volatile substances are collected and measured as aliquot A2 (1212).

A second extraction force is then applied to each sample for a first time period (1214). Extracted volatile substances are collected and measured as aliquot B1 (1216). The same second extraction force is again applied to each sample, this time for a second time period (1218). Extracted volatile substances are collected and measured as aliquot B2 (1220).

The amounts of one or more extracted volatile substances in each sample obtained as aliquot A1, A2, B1, and B2 are then compared (1222). Locations at which one or more volatile substance(s) is/are present in relatively high amounts in aliquot A1, but which are not present in such high amounts in aliquots A1, B1, and B2 are identified (1224). Further, locations at which the same one or more compounds are present in relatively high amounts in aliquot B2 but which are not present in such high amounts in aliquots A1, A2, and B1 are identified (1226).

Locations identified in step (1224) which are immediately adjacent to, but shallower in geologic depth to (e.g., exist above) locations identified in step (1226) are identified (1228). Locations identified in step (1228) are identified as target zones for resource production (1230).

In a more specific example of this method is illustrated in FIG. 13C (1200C). Drill cuttings samples are collected from geologic locations protected from atmospheric air (1262). Each sample is sealed upon collection in a sealable container (1264). A vacuum pressure of about 20 mbar is applied to each sample for about 1 minute as part of an RVS method (1266). The amount of hydrogen release as aliquot Al is collected and measured (1268). Notably, this assessment of hydrogen (and any step for measuring hydrogen described herein) can in aspects comprise one or more steps for estimating hydrogen using hydrogen proxies described herein to obtain a more accurate estimate of the amount of hydrogen present in the sample upon its collection, providing, e.g., a more accurate estimate of the amount of hydrogen present in the rock material at the location from which the sample was originally present. The same vacuum pressure is then reapplied for an additional about 8 minutes to each sample (1270). Hydrogen released as aliquot A2 is collected and measured (1272). A vacuum pressure of about 2 mbar is then applied to each sample for a period of about 1 minute (1274). Hydrogen released as aliquot B1 is then collected and measured (1276). The same vacuum pressure is then applied to each sample for an additional about 8 minutes (1278). The amount of hydrogen released is collected and captured as aliquot B2 (1280). The amount of hydrogen in each sample obtained as aliquots A1, A2, B1, and B2 are then compared (1282). Locations at which hydrogen is present in relatively high amounts in aliquot Al but not present in such high amounts in aliquots A2, B1, and B2 are identified (1284). Further, locations at which hydrogen is present in relatively high amounts in aliquot B2 but not present in such high amounts in aliquots A1, A2, and B1 are identified (1286). Locations identified in step (1284) which are immediately adjacent to, but which are shallower than (exist above) locations identified in step (1286) are identified (1288). Locations identified in step (1288) are identified as target zones for hydrogen production (1290).

FIG. 13B provides a second exemplary method (1200B).

FIG. 13B begins with obtaining sample(s) collected from geologic location(s) protected from atmospheric air (1232), typically subterranean location(s), e.g., location(s) associated with a well. Each sample is sealed upon collection in a sealable container (1234). A first extraction force is applied to each sample as part of an RVS method for a first time period (1236). Extracted volatile substances are collected and measured as aliquot A1 (1238). The same first extraction force is again applied to each sample, this time for a second time period (1240). Extracted volatile substances are collected and measured as aliquot A2 (1242).

A second extraction force is then applied to each sample for a first time period (1244). Extracted volatile substances are collected and measured as aliquot B1 (1246). The same second extraction force is again applied to each sample, this time for a second time period (1248). Extracted volatile substances are collected and measured as aliquot B2 (1250).

The amounts of one or more extracted volatile substances in each sample obtained as aliquot A1, A2, B1, and B2 are then compared (1252). Locations at which one or more volatile substance(s) is/are present in relatively high amounts in aliquot A1, but which are not present in such high amounts in aliquots A1, B1, and B2 are identified (1254). Further, locations at which the same one or more compounds are present in relatively high amounts in aliquot B2 but which are not present in such high amounts in aliquots A1, A2, and B1 are identified (1256).

Locations identified in step (1256) which are immediately adjacent to, but deeper in geologic depth to (e.g., exist below) locations identified in step (1254) are identified (1258). Locations identified in step (1258) are identified as target zones for resource production (1260).

In a more specific example of this method is illustrated in FIG. 13D (1200D). Drill cuttings samples are collected from geologic locations protected from atmospheric air (1263). Each sample is sealed upon collection in a sealable container (1265). A vacuum pressure of about 20 mbar is applied to each sample for about 1 minute as part of an RVS method (1267). The amount of hydrogen release as aliquot Al is collected and measured (1269). Notably, this assessment of hydrogen (and any step for measuring hydrogen described herein) can in aspects comprise one or more steps for estimating hydrogen using hydrogen proxies described herein to obtain a more accurate estimate of the amount of hydrogen present in the sample upon its collection, providing, e.g., a more accurate estimate of the amount of hydrogen present in the rock material at the location from which the sample was originally present. The same vacuum pressure is then reapplied for an additional about 8 minutes to each sample (1271). Hydrogen released as aliquot A2 is collected and measured (1273). A vacuum pressure of about 2 mbar is then applied to each sample for a period of about 1 minute (1275). Hydrogen released as aliquot B1 is then collected and measured (1277). The same vacuum pressure is then applied to each sample for an additional about 8 minutes (1279). The amount of hydrogen released is collected and captured as aliquot B2 (1281). The amount of hydrogen in each sample obtained as aliquots A1, A2, B1, and B2 are then compared (1283). Locations at which hydrogen is present in relatively high amounts in aliquot Al but not present in such high amounts in aliquots A2, B1, and B2 are identified (1285). Further, locations at which hydrogen is present in relatively high amounts in aliquot B2 but not present in such high amounts in aliquots A1, A2, and B1 are identified (1287). Locations identified in step (1287) which are immediately adjacent to, but which are deeper than (exist below) locations identified in step (1285) are identified (1289). Locations identified in step (1289) are identified as target zones for hydrogen production (1291).

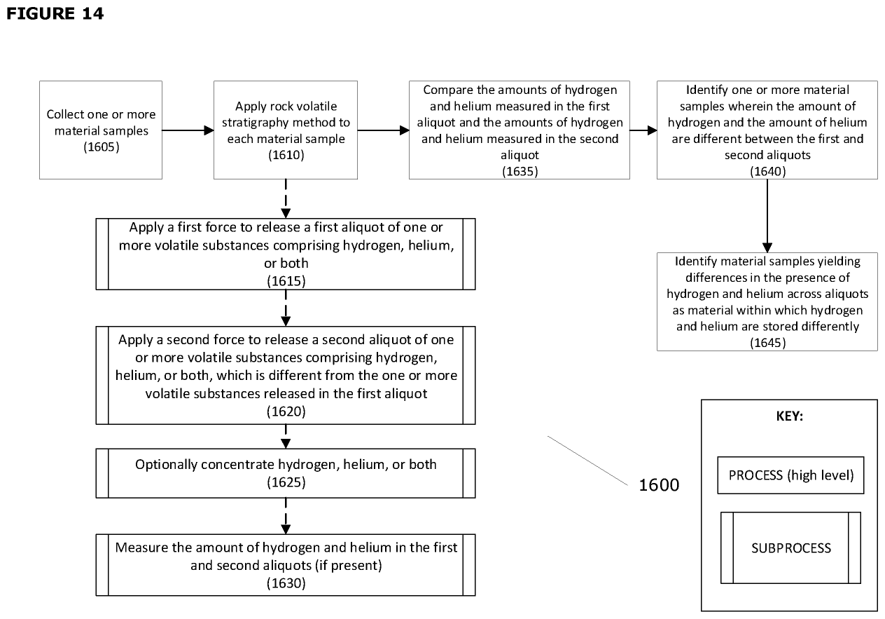

### Example 9 (FIG. 14)

Example 9 provides a method for identifying the difference in storage within a material of hydrogen versus helium (1600). In certain respects, Example 9 demonstrates that while hydrogen and helium can be present together in material(s), they can be separated from one another, e.g., obtained separately, by way of applying different extraction force(s) to the material. In further respects, Example 9 demonstrates that hydrogen and helium are in aspects stored differently within the same material, and use of one extraction technique versus another can allow selective collection of one versus the other. In aspects, Example 9 demonstrates that the technology(ies) disclosed herein are capable of identifying the differentiated storage of helium and hydrogen within the same material.

FIG. 14 illustrates an example (1600) of this/these concepts.

FIG. 14 begins with the collection of one or more material samples (1605). RVS method(s) are then applied to each material sample (1610). Such RVS method(s) first comprise the application of a first extraction force to release a first aliquot of one or more volatile substances, comprising hydrogen, helium, or both (1615). RVS method(s) further comprise the application of a second extraction force to release a second aliquot of one or more volatile substances comprising hydrogen, helium or both, wherein the volatile substance(s) is/are different from the one or more volatile substances released in the first aliquot (1620). The RVS method(s) further optionally comprise removal of condensable volatiles (1625), such as, e.g., by way of a cryotrap, whereby any hydrogen, helium, or both is essentially concentrated or otherwise freed of volatile substances which may DOS interfere with their analysis. The amount of each of hydrogen and helium in the first and second aliquots (if present) is then measured (1630). Again, as noted elsewhere herein, the determination of the amount of hydrogen can, in aspects, comprise the use of methods considering hydrogen proxy (ies) to obtain a more accurate reflection of the amount of hydrogen which may be present in the material at the location from which the sample was collected.

Upon completing the RVS analysis, the amounts of hydrogen and helium measured (obtained) in the first aliquot and the amounts of hydrogen and helium measured (obtained) in the second aliquot are compared (1635). One or more samples are identified wherein the amount of hydrogen and the amount of helium are different between the first and second aliquots (1640). Material samples yielding differences in the presence of hydrogen and helium across aliquots are identified as material within which hydrogen and helium are stored differently (1645).

In aspects, such method(s) described in this Example can be used in the production of a resource, e.g., hydrogen, as such method(s) can identify condition(s) under which hydrogen can be selectively extracted from material (e.g., geologic material), wherein the selective extraction DOS eliminates the co-extraction of helium, yielding a more highly purified collection of hydrogen. In aspects, such method(s) allow for the extraction of more commercially relevant hydrogen, as using such methods can allow hydrogen to be extracted from a geologic location in at a sufficient level of purity to meet purity standards demanded by the commercial marketplace.

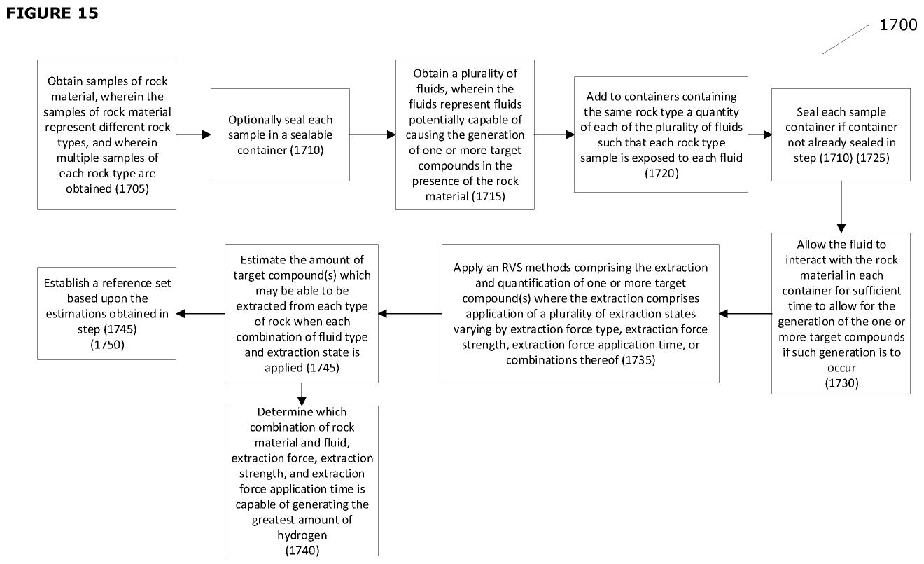

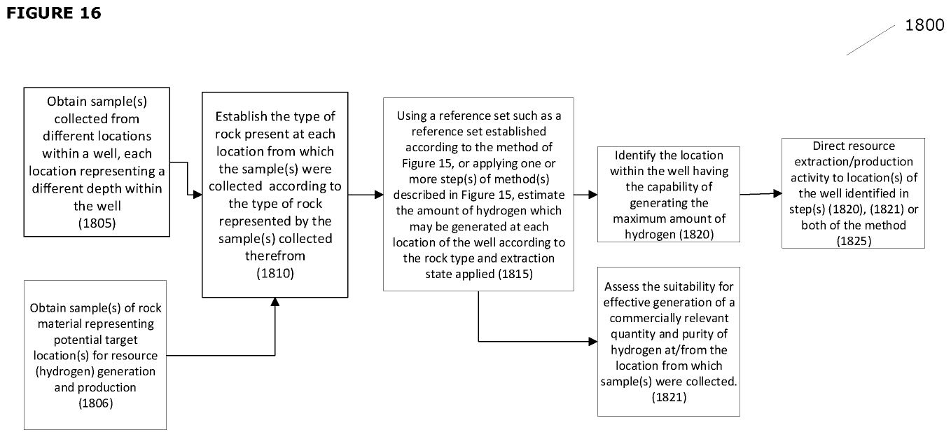

### Example 10 (FIG. 15)

In one aspect, the technology(ies) provided herein are directed to the synthetic production of hydrogen, whereby known rock type(s) are exposed to condition(s) forcing the generation of producible hydrogen. Example 10 provides a method of generating a reference which can be utilized in such endeavors to identify the conditions under which the maximum quantity (ies) of hydrogen can be generated.

FIG. 15 provides an exemplary method (1700) of creating such a reference, or, e.g., stating alternatively, an example method for determining the optimal conditions for synthetic hydrogen production given a particular rock type.

FIG. 15 begins with obtaining samples of rock material, wherein the samples of rock material represent different rock types, and wherein multiple samples of each rock type are obtained (1705). It should be noted that this step can comprise obtaining a single rock type, e.g., a known hydrogen-generating rock when such rock is exposed to one or more conditions, e.g., rock capable of generating hydrogen when exposed to fluid(s) containing water, such as, e.g., ferrous iron-bearing rocks, e.g., ferrous oxide(s) (e.g., high ferrous oxide content rock). Step(s) of the method can be modified accordingly to be directed to identifying the optimal condition(s) for generating hydrogen from such a specific rock type.

Samples can be sealed in a sealable container (1710). A plurality of fluids are then obtained, wherein the fluids represent fluids potentially capable of causing the generation of one or more target compounds, e.g., hydrogen, in the presence of the rock material (1715). Exemplary fluids can be, e.g., water-containing fluids comprising varying salinity, varying pH, varying Eh (oxidation-reduction potential), varying levels of dissolved solids, fluids of varying temperature, etc. A quantity of each of the plurality of fluids is added to the sample containers such that each rock type represented is exposed to each type of fluid (1720). Each sample is sealed in a sample container (if not already completed in step (1710) (1725). The fluid is allowed to interact with the rock material for a sufficient period of time to allow for the generation of the one or more target compounds, e.g., hydrogen, if such generation is to DOS occur (1730). RVS method(s) such as those described herein or described in the Prior Smith Patents are applied, such method(s) comprising the extraction and quantification of one or more target compound(s) where the extraction comprises application of a plurality of extraction states varying by the type of extraction force, the strength of the extraction force, the extraction force application time, or combination(s) and variation(s) thereof (1735). Such variation of extraction state(s) described here can, in further aspects, can comprise other differentiated conditions, such as, e.g., temperature, etc. The amount of target compound(s) which may be able to be extracted from each type of rock when each combination of fluid type and extraction state is applied is then estimated (1745). In one regard, a reference set is then established based upon such estimations (1750). In another regard, the combination of rock material, fluid, and extraction state (e.g., extraction force, extraction strength, extraction force application time, etc.) capable of generating the greatest amount of hydrogen is determined (1740).

### Example 11 (FIG. 16)

Example 11, illustrated by the exemplary method (1800) of FIG. 16, provides a method of generating synthetic hydrogen comprising, e.g., use of knowledge obtained by way of the generation of reference(s)/analysis(es) or variations thereof performed as described in Example 10.

The method of FIG. 16 begins by obtaining sample(s) collected from different locations within a geologic unit, e.g., different locations within a well, each location representing a different depth within the well (1805). Notably, such a method can alternatively begin by obtaining sample(s) of, e.g., rock material, representing potential target location(s) for resource (hydrogen) generation and production (1806). Such potential target location(s) can be known or unknown.

The type of rock present at each location from which the sample(s) were collected is established according to the type of rock represented by the sample(s) collected therefrom (1810). Rock type identification can be performed using any suitable technique (or suitable variation thereof) known in the art.

Using a reference set such as a reference set established according to the method o FIG. 15, or, e.g., by applying one or more step(s) of method(s) described in FIG. 15 (including e.g., the description provided in Example 10), the amount of hydrogen which may be generated at each location from which the sample(s) were collected according to the rock type and extraction state applied is then estimated (1815). The location, e.g., location within the well, having the capability of generating the maximum amount of hydrogen (1820), is then identified. Also or alternatively, the suitability for the effective generation of a commercially relevant quantity, purity, or both quantity and purity of hydrogen at/from the location from which the sample(s) were collected can be assessed. Notably, uncontradicted, analysis performed as a component of such method(s) described herein, e.g., method(s) described in Example 10 and Example 11, can comprise an assessment of substance purity in addition to substance amount. Resource extraction/production activity can then be directed to location(s) identified in steps (1820), (1821), or both (1825).

Example 11 demonstrates that optimized synthetic hydrogen production can be attained by applying technology(ies) described herein.

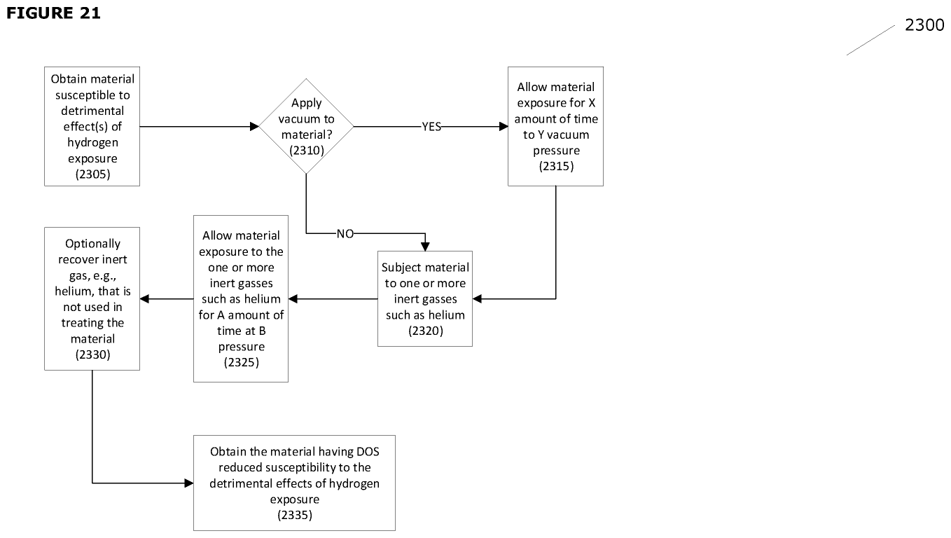

### Example 12 (FIG. 21)

As provided elsewhere in these Examples, the technology described herein provides technique(s) relevant to the identification of helium, establishing a geologic seal. In aspects, such findings have industrial applications.

Hydrogen is known to be corrosive to certain material(s). For example, hydrogen is known to react with metals, e.g., types of steel, and make the metals less durable (weaker), e.g., more brittle. Example 12, illustrated by the exemplary method illustrated in FIG. 21, provides an exemplary method (2300) for protecting material(s) from detrimental effect(s) of hydrogen exposure by way of DOS associating an inert gas such as helium with the material.

The protection method of FIG. 21 (2300) begins with obtaining a material susceptible to detrimental effect(s) of hydrogen exposure, such as, e.g., weakening, etc.

Optionally, one or more conditions, e.g. application of a vacuum (2310), can be applied to the material for a given period of time (2315). Subsequently, the material is treated with one or more inert gases, e.g., helium (2320), for a sufficient period of time to allow for the gas, e.g., helium to DOS associate with or otherwise “coat” the material or to otherwise establish an effective barrier against DOS hydrogen exposure (2325). Such exposure can be performed under a variety of time and pressure condition(s), such as, e.g., condition(s) wherein the pressure of the inert gas, e.g., helium, is below atmospheric pressure or alternatively a pressure greater than atmospheric pressure.

After treatment, the inert gas can optionally be recovered (2330) and, e.g., in aspects be available for treatment of additional material(s).

The inert-gas-treated material, e.g., helium-treated material, now having DOS improved resistance to detrimental effect(s) of hydrogen exposure, is then obtained (2335).

Such material(s) relevant to this method can, in aspects, represent part(s) of composition(s) that is/are used in application(s) where exposure to hydrogen is likely, and wherein such material, if untreated with the inert gas such as helium, would be DOS more likely to be DOS impacted by detrimental effect(s) of hydrogen exposure than the inert-gas (helium)-treated material.

### Construction and Terms

### Construction Principles and Description of Select Terms

This section offers guidelines and resources intended to aid readers in understanding this disclosure.

### General Terms and Principles

The intended audience for this disclosure (“readers”) are persons having at least ordinary skill(s) in the practice of technologies discussed or used herein. Readers may also be called “skilled persons,” and such technologies and related publicly available prior knowledge are collectively referred to as “the art.” Terms such as “understood,” “known,” and “ordinary meaning” refer to the general knowledge of skilled persons.

The purpose of this document (“disclosure”) is to describe a number of new technologies (sometimes just called the “technology” and each discrete embodiment of which sometimes being called “a technology”) and to provide readers with the ability to practice such technologies (e.g., by exemplification, by description of elements thereof and relationship(s) of such elements to each other, and possibly other means). Readers will understand from this disclosure whether the technology/technologies include different forms, e.g., objects (such as systems, compositions, or devices), methods, or both, as will be clear from the disclosure. Disclosed technologies may be associated with surprising, unexpected, or otherwise inventive properties and, accordingly, in many cases, a described technology may reflect an invention.

The term “uncontradicted” means not contradicted by this disclosure, logic, or plausibility, the latter two elements being based on the knowledge of skilled persons.

Disclosed here are several different but related exemplary aspects (variations) of the technology(ies) (also referred to as, e.g., “cases,” “facets,” “respects,” or “embodiments”). The disclosure encompasses all such aspects as described individually and as can be arrived at by any combination of such individual aspects. Thus, uncontradicted, any reference to “aspects” (e.g., “according to aspects” or “in an aspect”) will be understood as referring to according to any of the other suitable aspects of the technology(ies) described herein. In this respect, the breadth and scope of the disclosure should not be limited by any exemplary aspect(s)/embodiment(s) herein. No language in this disclosure should be construed as indicating any element/step is essential to the practice of any technology or group of technologies provided by this disclosure unless such a requirement is explicitly stated. Uncontradicted, any aspect(s) described in any part of this disclosure can be combined with any other aspect(s) in any other part. Readers will discern that the term “aspect” is also sometimes used to refer to portions of this disclosure and, in such respects, is used in a manner similar to “subject” or “topic.”

Uncontradicted, all technical/scientific terms used here should be read, at least in one aspect, to have the same meanings as commonly understood by skilled persons, regardless of any narrower examples or descriptions provided here (including any term introduced initially in quotations). However, readers will also recognize that some aspects can be characterized by the inclusion of elements, steps, features, characteristics, etc., associated with specific descriptions provided here and that such specific disclosures represent distinct embodiments of the disclosure apart from the corresponding aspect that is provided by interpreting the relevant aspect using any broader commonly used terminology or concept. Uncontradicted, disclosure of any aspect using known terms, which terms are narrowed by example or otherwise, implicitly discloses one or more related aspects in which the applicable terms are alternatively interpreted using the broadest reasonable interpretation of skilled persons.

Uncontradicted, the term “or” means “and/or” here, regardless of any inclusion of the actual phrase “and/or” (e.g., phrases such as “A, B, or C” and “A, B, and/or C” each simultaneously discloses aspects including (1) all of A, B, and C; (2) A and C; (3) A and B; (4) B and C; (5) only A; (6) only B; and (7) only C (and also support sub-groupings, such as “A or B,” “A or C,” etc.)). Uncontradicted, the use of a modifier/term such as “or both” in connection with elements (e.g., “element A, element B, or both”) does not mean or imply that elements listed only as “A or B” do not include combinations of A and B.

For conciseness, symbols are used where appropriate. E.g., “&” is used for “and,” & “for “about.” Symbols such as < and > are given their ordinary meaning (e.g., “≤” means “less than or equal to” & “>” means “greater than or equal to”). A slash “/” between terms here can represent “or” (“A/B” means “A or B”) or identify synonyms of an element, depending on context. The inclusion of “(s)” after an element or a step indicates that ≥1 of such an element is present, step performed, and the like. E.g., “element(s)” refers to both 1 element and ≥2 elements, with the understanding that each thereof is an independent aspect of the disclosure.

Uncontradicted, the term “also” means “also or alternatively.” Uncontradicted, the terms “here” & “herein” mean “in this disclosure.” The abbreviation “i.a.” (alternatively “ia” or “ia”) means “inter alia” or “(possibly) among other things.” “Also known as” is abbreviated “aka,” “AKA,” “a.k.a” (and can also or alternatively mean “is otherwise referred to here,” even if the relationship between the terms is not well known). The similar abbreviation “aka/ac” means “also known as or otherwise called” and is sometimes alternatively used to stress this point about the nature of the acronym. Uncontradicted, the term “elsewhere” means “elsewhere herein.”

Use of the abbreviation “etc.” (or “et cetera”) in association with a list of elements/steps means any or all suitable combinations of the recited elements/steps or any known equivalents of such recited elements/steps for achieving the function(s) of such elements/steps known in the art. Readers should interpret phrases like “and the like” similarly.

Uncontradicted, terms such as “and combinations,” “or combinations,” and “combinations thereof,” etc., regarding listed elements/steps, means any or all possible/suitable combinations of the associated elements/steps. Thus, e.g., uncontradicted, a phrase like “combination of any thereof” refers to any or all combinations.

Aspects may be described as suitable for method(s)/use(s) disclosed herein. Uncontradicted, terms such as “suitable” or “suitability” mean acceptable, appropriate, or, in aspects practical for performing a particular function/achieving particular state(s)/outcome(s), and typically means effective, practical, and non-deleterious/harmful to associated valuable subject matter (human health, resource state, etc.). E.g., uncontradicted, the term “suitable” means appropriate, acceptable, or in contexts sufficient, or providing at least generally or substantially all an intended function (of the element or overall whole of the aspect), without causing or imparting significant negative/detrimental impact. Uncontradicted, each method step, component/ingredient, or result element of the technical aspects of this disclosure should be understood to implicitly be mostly, generally only, substantially only, or only of an amount, degree, or character suitable in connection with its intended function, the intended function of the associated whole, or both. In some respects, suitability can be demonstrated through scientific studies and to a degree of significance through suitable tests/measures such as scientific tests, well-controlled and adequate studies (e.g., clinical studies), adequately powered trials, etc.

Steps, elements, compositions, devices, components, and the like also or alternatively can be characterized as being “effective.” Uncontradicted, any disclosed element is to be construed as being effective for its intended purpose and present in an effective amount, and any step performed is to be understood as being performed/applied effectively, such as in an effective amount or an effective number of times, etc. Uncontradicted efficacy can be judged by evaluating the element(s) ability to perform or contribute to the described function(s) or characteristic(s) associated with the component, device, step, etc., the overall aspect, or both in any manner disclosed here, known in the art, or both. For example, when applied to effects in organisms, such as people, efficacy and similar terms uncontradicted should be interpreted to at least implicitly disclose efficacy that can be measured (1) in a treated subject, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects treated. Object elements or steps are, uncontradicted, understood to be implicitly present in “effective amount,” and, uncontradicted, any described class of object or step in connection with a device, system, composition, or method, is understood to be present in the associated whole or performed in association with the associated entire method in an effective amount, effective way, or having effective characteristic(s), which generally means, an amount that the described object/component or step is effective for the described function(s) associated with the element, associated whole, or both. A “step” is not necessarily a general “step for” performing a function.

Uncontradicted, heading(s) (e.g., “Construction and Terms”) and subheadings used here are included for convenience and do not limit the scope of any aspect(s). Uncontradicted, aspect(s), step(s), or element(s) described under one heading can apply to other aspect(s) or step(s)/element(s) here.

Ranges of values here represent each value falling within a range within an order of magnitude of the smallest endpoint of the range, without having to write each value of the range explicitly. E.g., a recited range of 1-2 implicitly discloses each of 1.0, 1.1, 1.2, . . . 1.9, and 2.0, and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593). Uncontradicted, readers will understand that any two values in a range provided as a list herein can be combined as endpoints to form a range defining a more particular aspect of the disclosure (e.g., if a list of values 1, 2, 3, 4, and 5 of element X is provided, readers will understand that the disclosure implicitly discloses an aspect comprising 2-4 X, 3-5 X, and 1-3 X, etc.

Terms of approximation (e.g., “about,” “~,” or “approximately”) can be used here (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding of the art, etc. In the absence of guidance here or in the art for an element, terms such as “about” when used in connection with an element should be interpreted as ±10% of the indicated value(s) and implicitly disclosing ±5%, ±2%, ±1%, and ±0.5%. Two or more values may be characterized as approximately similar if they would be considered to be about the same on such bases.

Aspects may be associated with description of change or difference. In some cases, similarity or difference can be assessed as similar or not (i.e., statistically similar or different). In cases, a difference or change means a “sizable” change or difference, which means a change or difference that is beyond what would be considered substantially the same or approximately the same (approximately or about in such contexts being either defined in the art or +/−10% or being recognized by readers as not having different characteristics or outcomes that are substantially different with respect to intended function) (e.g., a change of ≥12.5%, ≥15%, ≥20%, etc., such as 12.5%-50%, 12.5%-33%, 15-45%, etc., or 15-150%, 20-200%, 30-300%, etc.). In aspects, a change or difference in element(s) can be characterized as a “major” change or difference, which means a change or difference that is an increase or decrease (1) of at least 33% and can be a change of at least 50%, at least 75%, at least 100%, at least 150%, at least 200% (2×), e.g., at least 0.5x-5x, 10x, or 20× or (2) by one or more (e.g., 2 or 3) order(s) of magnitude. Uncontradicted, elements, compositions, outcomes, etc., described as different herein or described as different in any of these ways provide implicit support for corresponding aspects in which the applicable change/difference is characterized as one of the other differences. The modifier “constrained” means that a value, such as a sizable value, a detectable value, or a major value is limited to 50% or less of a whole (e.g., 45% or less, 40% or less, 37.5% or less, or 35% or less of a whole). Uncontradicted, any disclosure of such a term herein provides implicit support for an otherwise corresponding aspect in which the scope of the applicable value can be characterized as a constrained value.

This disclosure includes aspects of the technology that are associated with particular characteristics, such as amounts of components (or ranges thereof), In cases, several such characteristics of varying scope may be provided. Readers will understand that each such characteristic can be associated with particular properties that distinguish such aspects from other aspects, and, accordingly, each such range can be viewed as critical to a particular aspect of the technology, even if the associated results, properties, functions, etc., associated with such aspects are not directly/explicitly communicated in association with any such characteristics.

Lists of aspects, elements, steps, and features are sometimes employed for conciseness. Unless indicated, each member of each list should be viewed as an independent aspect. Each aspect defined by any individual member of a list can have and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses the corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” concerning another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).” The term “measurably” means at a measurable level and, uncontradicted, comprises at a suitable measurable level/amount. The term detectable provides implicit disclosure for aspects that are “measurable,” and the term “measurable” implicitly supports aspects where the measured or measurable element is “detectable.” Uncontradicted, any aspect including an element described as “similar” to another element implicitly discloses, at least as one aspect, where the similarity comprises statistical similarity. Uncontradicted, any reference to a comparison, change, or other relationship between elements (e.g., a result) characterized by similarity or detectability also implicitly discloses changes or comparisons where the difference is approximately/about the same (e.g., within +/−10% of each other).

Uncontradicted, any value provided here that is not accompanied by a unit of measurement (e.g., a weight of 50 or a length of 20), either any previously provided unit for the same element/step or the same type of element/step will apply, or, in cases where no such disclosure exists, the unit most used in association with such an element/step in the art applies.

Uncontradicted, the terms “including,” “containing,” “comprising,” and “having” mean “including, but not limited to,” or “including, without limitation.” Uncontradicted, use of terms such as comprising and including regarding elements/steps means including any detectable number or amount of an element or including any detectable performance of a step/number of steps (with or without other elements/steps). Uncontradicted, “a” means one or more, even when terms such as “one or more” or “at least one” are used in association with the referent “a.”

For conciseness, description of an aspect “comprising” or “including” an element, concerning a collection/whole (e.g., a system, device, or composition), implicitly provides support for any detectable amount/number, such as, e.g., between a detectable or measurable amount and about 33%, such as, e.g., ≥~1%, ≥~5%, ≥~10%, ≥~20%, ≥~25%, or ≥~33%, as in, for example, ~0.00001%-~33%, ~1%-~33%, ~5%-~33%, ~10%-~33%, ~15%-~33%, ~20%-~33%, ~25%-~33%, or, e.g., ~30%-~33%.

In certain respects, description of an aspect “comprising” or “including” an element concerning a collection/whole implicitly provides support for amounts greater than about 33%, such as, e.g., ~50%-~75%, such as, e.g., ≥~50%, ≥~51%, ≥~51%, ≥~66%, or >~70%, such as, e.g., ~55%-~75%, ~60%-~75%, or ~70%-~75%, such as, e.g., ~35%, ~40%, or ~45%.

In still further respects, description of an aspect “comprising” or “including” an element concerning a collection/whole implicitly provides support for amounts greater than about 80%, such as, e.g., ~80%, ~85%, >~90%, ~93%, >~95%, ≥~99%, or ~100% of the whole/collection being made up of the element, as in, e.g., ~77% or more, or essentially all of the whole/collection being made up of the element (i.e., that the collection consists essentially of the referenced element). Similarly, a method described as including a step concerning an effect/outcome implicitly provides support for the referenced step, providing ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the effect/outcome, representing ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the steps/effort performed, or both. Explicit listing of percentages of elements in connection with particular aspects does not limit or contradict such implicit disclosure. Uncontradicted, readers should interpret terms such as “essentially all” or “essentially” consistent with the concept of “consisting essentially of.”

Uncontradicted, terms such as “comprising” when used in connection with a step of a method provide implicit support for performing the step once, ≥2 times, or until an associated function/effect is achieved.

Further, readers will understand that uncontradicted, use of terms such as “comprising” or “including” provides aspects for which the referenced element (or step(s), etc.) is “generally” present, “substantially” present, “essentially” present, or is preset. In certain alternative aspects, “comprising” or “including” can refer to something that is mostly present, about equally present, or is present in another amount, such as about 40%, about 50%, etc. In certain aspects, accordingly, the use of “comprising” and “including” provides support for referenced element(s) to be present in “significant” amounts (e.g., a statistically significant amount) or in DOS amounts, in “some” amount, or, e.g., a “predominate” amount.

Uncontradicted, any disclosure of an object or method (e.g., composition, device, or system) “comprising” or “including” element(s) provides implicit support for an alternative corresponding aspect that is characterized by the object consisting of that element or “consisting essentially of” that element (excluding anything that would “materially affect” the “basic and novel characteristic(s)” of any inventive aspect of this disclosure). Uncontradicted, any specific use of phrases such as “consists of” and “consists essentially of” herein does not modify this construction principle.

Readers will understand that the disclosure of concentrations/amounts of different elements/components acts as a disclosure of compositions characterized by relationships in such amounts formed between them. Accordingly, uncontradicted, any disclosure of amounts/concentrations that reflects a suitable relationship between elements/components provides an implicit disclosure of a composition that varies from the specifically disclosed amounts/concentrations, but which retains the relationship. For example, if the disclosure provides 1 unit of A and 3 units of B, readers will understand that this means that the disclosure provides a corresponding aspect characterized by the inclusion of suitable amounts of A and B, wherein such amounts are present in a ratio of about 1 part A to about 3 parts B.

Readers will understand the “basic and novel characteristic(s)” of an invention provided in this disclosure and the scope of what constitutes a “material effect” (or “material effect”) of such “basic and novel characteristics” will vary with the specific applicable aspect at issue. Uncontradicted, the basic and novel characteristics of any inventive aspect include the specific recited and associated elements of an aspect and exclude any other element that significantly detracts from the intended function(s) of the recited elements, that introduce significant new functions that are unrelated to the intended function(s), that significantly reduce the performance of the function(s), or that significantly negatively change other characteristics of performing such function(s) (e.g., by increasing the cost of performing the functions in energy, money, or both). Uncontradicted, the basic and novel characteristics also include at least significantly retaining the suitability, effectiveness, or both, of recited elements or the overall aspect. Accordingly, a material effect can be an effect that reduces, diminishes, eliminates, counteracts, cancels, or prevents one or more of such functions in one or more respects (e.g., delaying onset, reducing scope, reducing duration, reducing output, reducing the level of applicability, reducing effect, or combinations thereof). In an aspect, a material effect is one that changes such functions by making such functions impractical, difficult to obtain, or materially more expensive or otherwise costly in terms of inputs. From this and the other guidance provided herein, readers can understand the scope of an aspect that is defined by consisting essentially of a collection of elements.

Uncontradicted, the term “one” means a single type, single iteration/copy/thing, of a recited element or step, or both, which will be clear from the context of the relevant disclosure. For example, the referent “one” used with respect to a component of a composition/article or system can refer to one type of element (which may be present in numerous copies, as in the case of an ingredient in a composition) one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a type of component of a composition/system/article).

Uncontradicted, the term “some” means ≥2 copies/instances or ≥5% (e.g., ≥7.5%, ≥12.5%, ≥17.5%, ≥27.5%, or ≥37.5%) of a listed collection/whole is or is made up of an element. Regarding methods, some means ≥5% of an effect, effort, or both is made up of or is attributable to a step (e.g., as in “some of the method is performed by step Y”) or indicates a step is performed ≥2 times (e.g., as in “step X is repeated some number of times”). Terms such as “considerable amount” or “considerable portion” mean at least 1%, 2%, or 2.5%, but less than most of a whole, such as 2.5-25%, e.g., 5-25%, 5-20%, 7.5-22.5%, 10-20%, 2.5-10%, 2.5-12.5%, 5-15%, etc. Terms such as “sizable portion” mean 10-50% and in aspects 15-50%, 20-50%, or 25-50%, or subranges thereof (e.g., 15-45%, 20-40%, 25-45%, etc.). Terms such as “predominately,” “most,” or “mostly” (and “primarily” when not used to refer to an order of events or “mainly”) means detectably >50% (e.g., mostly comprises, predominately includes, etc., mean >50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤~25~% of an element, and terms such as “substantially free” of an element mean comprising ≤~5% of an element. Uncontradicted, any aspect described as “generally comprising” or “generally consisting” of an element implicitly discloses an element that “substantially comprises” the element. The same principle applies to any disclosure where an aspect is described as being “generally free” of an element.

Uncontradicted, phrases such as “substantially identical” or “substantially similar” may be used to refer to element(s)/component(s)/ingredient(s)/thing(s) (e.g., composition, system, device, etc.) or step(s)/method(s) that have the same or about the same characteristic(s) or achieve the same or about the same result(s), typically in a similar way, as a referenced element/thing or step/method or otherwise do not meaningfully differ in intended result and manner of achieving such a result or are otherwise recognized in the art as not differing or not differing substantially in the relevant context (e.g., by being considered equivalents). Uncontradicted, readers will understand that a “substantially identical” or “substantially similar” element/thing or step/method when compared to a comparator thing/element or method/step means that the referenced element/thing or step/method exhibits such a similar function as a comparator at identical, approximately identical, or statistically similar amounts as the comparator thing or method when applied under similar conditions of use. Again, where statistical, approximate, or other measured comparisons are not possible, readers will understand the phrase as encompassing those things known as being identical or substantially identical to the referenced element/step or are described as such herein.

Uncontradicted, any aspect described concerning an optionally present element(s)/step(s) also provides implicit support for corresponding aspect(s) in which one, some, most, generally all, nearly all, essentially all, or all such element(s) are lacking/step(s) not performed, in respect of the relevant aspect. E.g., disclosure of a system comprising element X implicitly also supports a system lacking element X. That is, readers will understand that any element, feature, step, or characteristic of any aspect of the technology recited herein as being present in an aspect also implicitly provides support for the element, feature, step, or characteristic as being excluded from a corresponding/similar aspect implicitly disclosed by the explicit positive disclosure and vice versa. Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments in at least the broadest version of the relevant aspect. Uncontradicted, the use of ordinal numbers such as “first,” “second,” “third,” and so on is primarily, though not exclusively, intended to distinguish respective elements rather than to limit the disclosure to a particular order of those elements, importance, or configuration.

Any elements associated with a function can be alternatively described as “means for” performing a function in a composition/device/system or a “step for” performing a part of a method, and parts of this disclosure refer to “equivalents,” which means known equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation but, rather, describe element(s)/step(s) configured to, designed to, selected to, or adapted to achieve a certain performance, characteristic, property, or the like using teachings provided here or in the art.

As used herein, the word “exemplary” means “serving as an example, instance, or illustration.” Any embodiment described herein as “exemplary,” “representative,” or “illustrative,” etc., should not necessarily be construed as preferred or advantageous over other embodiments. Any embodiment described herein as “exemplary” is not necessarily to be construed as preferred or advantageous over other embodiments.

Except where explicitly indicated or clearly indicated by context, terms such as “improved” or “better” mean DOS increased (e.g., sizably, constrained sizably, majorly, or constrained sizably, increased, etc.). In some respects, as will be clear from context or knowledge, terms such as “improved” or “better” mean DOS “reduced,” such as concerning reducing negative elements of a method or composition. Uncontradicted, terms such as “enhanced,” “improved,” “better,” and the like are used synonymously.

Any reference to a trademark name (product trademark (TM)) incorporates all publicly known information about that product as of the filing date of this Application. Further, reference to a trademarked name is intended to refer to those product(s) as recognizable to PHOSITA as of the date of filing of this Application but also to product(s) that are similar to such product(s), such as product(s) demonstrating that are functionally the same (perform the same function(s)); are operationally the same (operate on the same key principles or by way of the same feature(s)); comprise the same key characteristic(s), component(s), mechanism(s), etc. PHOSITA should recognize, however, that simplified and alternative product(s) from those provided herein could be provided; e.g., modification(s) of the product(s) described herein, which maintain the spirit of this disclosure, are implicitly incorporated herein.

Regarding figures, graphs, and the like disclosed in this Application, such figures, graphs, and the like are exemplary, and their disclosure concurrently provides disclosure of anything statistically significantly close to or which is “about” the same as the disclosure—e.g., as the data, feature(s)/characteristic(s), and demonstrated performance characteristic(s) provided. For example, any/all data points provided in, e.g., graph(s) disclosed in this Application incorporate data point(s) within a range of statistical similarity and points which are about the same (e.g., within a reasonable fraction of an order of magnitude) as those exemplified. Graph(s) or figure(s) provided herein should be understood to include where such feature(s) are present, data providing similar patterns, shapes, curves, peaks, etc.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. Uncontradicted, in the event of any conflict between this disclosure and the teachings of such documents, the content of this disclosure takes precedence regarding interpreting aspects of the disclosure. Numerous references are cited here to concisely incorporate existing knowledge and to aid skilled persons in putting aspects into practice. While efforts have been made to include the most relevant references for such purposes, readers will understand that not every aspect of every cited reference will apply to every aspect of this disclosure or the technology.

While elements disclosed in such incorporated references can be combined with aspects of the disclosure provided herein, readers will understand that the described technology is intended to stand apart from such disclosures and, accordingly, uncontradicted, in aspects, any element(s) of the objects or methods of any such references can be considered to be excluded from the scope of what is presented as new technology here (e.g., if reference A discloses object or element B, any aspect that is not directed to object or element B can be characterized by, as one aspect, the lack of object or element B).

All original claims contained in this disclosure, when filed, are incorporated into this specification as if they were a part of the description.

### TERMS AND PRINCIPLES RELATING TO THE TECHNICAL FIELD

Readers should note that some terms specific to the field or that are specific to this disclosure also or alternatively are provided in the Summary/Detailed Description sections or elsewhere. Uncontradicted, any repetition of a term description is meant to reflect alternative aspects that are characterized by the different meanings or examples of such terms.

### TERMS AND PRINCIPLES SPECIFIC TO THE TECHNOLOGY OR THIS DISCLOSURE

The following description of certain terms and acronyms is provided to assist readers in understanding the technology. Additional acronyms may be only provided in other parts of this disclosure and acronyms that are well known in the art may not be provided here.

### Prior Smith Patents

Examples of prior Smith patents and applications are shown in the table below.

| Filing | | | Publication Number (if | |
|---|---|---|---|---|
| Country | Application Number | Filing Date | available) | Patent Number (if issued) |
| AU | 2017376128 | Dec. 12, 2017 | AU 2017/376128 B2 | 2017376128 |
| BR | BR 11 2019 011944 2 | Dec. 12, 2017 | BR 11 2019/011944 A2 | BR 112019011944-2 B1 |
| CA | 3 046 972 | Dec. 12, 2017 | CA 3046972 A1 | |
| EA | 201991461 | Dec. 12, 2017 | EA 2019/91461 A1 | EA 043306 B1 |
| EP | 17880886.1 | Dec. 12, 2017 | EP 3 555 422 A4 | EP3555422 |
| SA | 519402128 | Dec. 12, 2017 | SA 519402128 A | SA13603B1 |
| US | 15/908,760 | Feb. 28, 2018 | US 2018/0195383 A1 | U.S. Pat. No. 10,190,413 B2 |
| US | 16/019,523 | Jun. 26, 2018 | US 2018/0306031 A1 | U.S. Pat. No. 10,494,919 B2 |
| US | 16/019,529 | Jun. 26, 2018 | US 2018/0355717 A1 | U.S. Pat. No. 10,260,336 B2 |
| IN | 201917021802 | May 31, 2019 | IN 2019/17021802 A | 480793 |
| US | 16/654,018 | Oct. 16, 2019 | US 2021/0207477 A9 | U.S. Pat. No. 11,280,186 B2 |
| US | 17/019,130 | Sep. 11, 2020 | US 2020/0408732 A1 | U.S. Pat. No. 11,567,056 B2 |
| CA | 3 171 326 | Feb. 14, 2021 | CA 3171326 A1 | |
| US | 17/194,194 | Mar. 5, 2021 | US 2021/0215652 A1 | U.S. Pat. No. 12,105,076 B1 |
| US | 17/374,912 | Jul. 13, 2021 | US 2021/0341455 A1 | U.S. Pat. No. 12,055,534 B2 |
| IN | 202118050357 | Nov. 2, 2021 | | 531756 |
| US | 17/548,295 | Dec. 10, 2021 | US 2023/0184645 A1 | U.S. Pat. No. 11,927,571 B2 |
| US | 17/720,217 | Apr. 13, 2022 | US 2022/0283138 A1 | |
| US | 17/887,507 | Aug. 14, 2022 | US 2023/0175369 A1 | |
| US | 17/947,145 | Sep. 18, 2022 | US 2023/0081834 A1 | |
| SA | 522441733 | Dec. 14, 2022 | SA 522441733 A | |
| SA | 522441731 | Dec. 14, 2022 | SA 522441731 A | |
| EA | 202390666 | Mar. 21, 2023 | 202390666 | |
| AU | 2023204267 | Jul. 3, 2023 | AU2023204267 | |
| US | 18/433,409 | Feb. 6, 2024 | US 2024/0255398 | |
| US | 18/751,277 | Jun. 23, 2024 | US 2024/0345059 A1 | |

### Selectable/Controllable

In aspects, the operation of systems are performed selectively or a component of a device can be characterized as controllable. Terms such as “selectively,” “selectable,” “settable,” and “controllable” may be used to describe the functionality or state of one or more part, component, device, element, system, etc. Uncontradicted terms such as “selectively” or “selectable” mean designed to be, able to be, or which otherwise are established/set by a user or other controller (e.g., a support person). Uncontradicted, the term “controllable” (or “adjustable”) or “settable” means settable or adjustable in degree, amount, timing, or other characteristic(s) of operation according to user aim(s), desire(s), target use(s), or other user preference(s). An element described as selectable does not necessarily mean that the selected element is entirely on or off when selected/engaged, but, rather, can, in aspects, mean that it (the condition, characteristic, operation) changed from one state/setting to another when a selection is made. Uncontradicted, any element/component or device described by either type of term provides implicit support for a corresponding aspect in which the explicitly stated term is substituted with the other type (e.g., reference to a selectively controlled component implicitly provides support for/implicit disclosure of a settable controlled component and vice versa).

### Selective, Conditional, and Automatic Operation

Uncontradicted, any operation of any component, element, device, system, or performance of any step described here can be performed on a selective basis, a conditional basis, an automatic basis, or a manual basis. The term automatic means that the operation is performed without a person/user causing the operation/step to be performed. In aspects, automatic operation can be initiated by act(s) of a user/person and, uncontradicted, any automatic operation step(s)/function(s)/action(s) should be construed as, at least in aspects, encompassing user initiation of the automatically performed event(s)/function(s). The term manual means that a person must be involved with the initiation, maintenance, or termination of the state. Uncontradicted, any automatic function can AOA be performed manually, at least in part.

Uncontradicted, qualifiers/characterizations including selectively operable, conditionally automatically operable, or selectively and conditionally operable, etc., also can apply to the performance of any suitable step, function, activity, and the like. A selectively operable event/function/activity is one that is selected to be performed and, in aspects the term can be SWOSB the term controllable, or the like. For example, in aspects, the term selectively operable can be viewed as meaning that the operation is only sometimes performed (e.g., can be “turned on and off”). A conditionally operable function/element or activity is one that is performed only on the occurrence of certain conditions (triggers, predicates, etc.), typically automatically when such trigger(s) are present (e.g., by, i.a., user of IF/THEN or WHILE coding structures in a function, or equivalent means). Any function/event/activity that is described herein as associated in connection with a condition can be, in aspects, performed as a conditionally operable function (e.g., if hydrogen is detected, a conditionally operated automatic function can include quantifying the hydrogen; if hydrogen is detected, a conditionally operated automatic function can include direct drilling activity, hydrogen generation activity, other analytical activities (e.g., evaluation of the environment of the material, confirmation of characteristic(s) of the material, and the like).

### Material/Sample

Uncontradicted, the terms material and sample are SWOSB to each other here. The use of material in aspects can, uncontradicted, be used to refer to analyses that are performed in situ rather than through samples (material samples). The term sample is ARTA as material sample here. A material sample is a composition comprising, generally consisting of, or consisting of a measurable amount of a sample which occupies a unit of volume. The sample typically contains mostly solid. However, it may also contain liquids, for example, drilling mud, or gas, e.g., hydrogen or helium or both liquids and gases that are associated with the solid in any amount or volumetric percentage of the total volume of the sample. The sample may be homogeneous or heterogeneous.

In aspects, the material is, comprises, mostly comprises, or essentially is a geologic material. A geologic material can be any suitable material that comprises solid material, semi-solid material, or both, that is from earth or that is modified by contact with geologic material resources. Uncontradicted, the term earth should be considered to be applicable to any suitable planetary body in our solar system, e.g., the Earth, the Earth's moon, i.e., the Moon, Mars, asteroids, e.g., 1986 DA, comets, e.g., 2P/Encke, and other planets and their respective moons. In other words, aspects can apply to the analysis of extraterrestrial rocks and other similar materials.

In aspects, the material is a mineral aggregate, e.g., a rock. In aspects, the rock comprises features that stably contain easily extractible volatile substances (at least that are stable in the environment in which the material is found prior to analysis). The nature of materials that comprise easily extractible volatile substances is described in the Prior Smith Patents (albeit sometimes using different terminology—e.g., volatiles, volatile substances, and the like). E.g., pores or similar structural features may reside within the material and/or samples that can be associated with the presence of easily extracted volatile substances (ARTA simply as “volatiles”). Other examples of such features include fissures, fractures, pockets, cracks, etc., which contain target materials of interest, such as hydrogen or helium. Such fissures, fractures, etc. will often desirably contain target substance or target substance-relevant material that also in some cases are (1) present in relevant amounts in the material (either in fluid form or are absorbed or adsorbed in the material), rather than material that are artifacts of prior existing conditions, (2) are exposed to the surrounding environment in some amount (such as by being contained in a pore in the material that is exposed to the surrounding environment) (in other words are not completely sealed off from the environment as is the case with fluid inclusions), or (3) can be characterized in satisfying both (1) and (2). The term “pore fluid” sometimes is used to mean a substance that is ordinarily liquid or gas in association with the material, contains one or more volatiles, and satisfies conditions (1), (2), or (3) of the preceding sentence. In some aspects, the technology is characterized by analyzing one or more samples containing an analyzable amount of pore fluid(s) and the use of terms such as materials/samples generally provides support for such elements comprising pore fluid(s), e.g., in amounts that DOS or sizably enhance or promote the amount of extracted volatiles therefrom, change the nature of the volatiles extracted therefrom, or both.

### Tight Rock

Tight rock formations are predominantly composed of shale. Tight rock typically is characterized on the basis of permeability. Typical tight hydrocarbon systems have permeabilities in the range of 1-10 millidarcies, whereas shales may be down to nanodarcies. In one aspect, tight rock can be characterized as having a permeability of about 0.1 milli Darcy (mD) or less.

### Volatile/Substance

Volatiles are gases that can be removed from a material samples. Volatiles may exist as gases residing within the material sample at standard atmospheric pressure and temperature. Volatiles may exist as liquids or solids at standard atmospheric temperature and pressure but be convertible to a gas by application of a vacuum to the material sample or by application of increased temperature to the material sample or both application of a vacuum and increased temperature. On occasions, volatiles may be created by both an increase in temperature and increase in pressure applied to a material sample. In the present invention, volatiles may be extracted from the material sample to be eliminated, such as water vapor or certain hydrocarbons, or to be analyzed.

Atmospheric air is air found within 1000 feet of the surface of the Earth at normal temperature and pressure, typically 30 degrees Celsius and 1 atmosphere, that typically contains by volume 78% nitrogen, 21% oxygen, 1% argon, and variable amounts of other gases such as but not limited to carbon dioxide and water vapor.

Compound can refer to an organic or inorganic compound. An “organic compound” encompassed by this term typically is any compound in which one or more atoms of carbon are covalently linked to atoms of other elements, most commonly hydrogen, oxygen, nitrogen, and often phosphorous or sulfur, with the exclusion of certain carbon-containing compounds that in the art are not characterized as organic compounds (e.g., certain carbides, carbonates, and cyanides). An organic compound used in the methods herein can be a hydrocarbon (containing only carbon and hydrogen) and, in aspects, can be a saturated hydrocarbon (often referred to as an alkane) such as, e.g., butane, pentane, hexane, heptane, octane, nonane, decane, undecane, or dodecane. In aspects, the term compound includes one or more cycloalkanes. In aspects, an inorganic compound encompassed by the use of the term compound indicates compounds which can be but may not necessarily be structurally similar, compositionally similar, or both structurally and compositionally similar to the organic compounds suitable for use in the methods herein. In aspects, the inorganic compounds include a hydrogen; in some aspects the inorganic compounds lack a hydrogen. Inorganic compounds can lack carbon, include carbon-containing compounds not characterized as organic compounds in the art, or both. Non-limiting examples of inorganic compounds suitable for use in the method(s) herein and encompassed by the use of the term “compound” include carbon dioxide (CO2), carbonyl sulfide (COS), carbon disulfide (CS2), sulfur dioxide (SO2), and hydrogen sulfide (H2S). Another example of an inorganic compound is water. In aspects, the term compound encompasses such organic and inorganic compounds and further encompasses what are defined as “related compounds” as defined herein. Uncontradicted, in certain aspects, reference to the comparison of a compound as a step of a method described herein (e.g., the comparison of a compound measured in a first sample to the same or related compound measured in a second or additional sample) can be interpreted as the comparison of a ratio containing that compound. Compounds may exist as solids, liquids, or gases depending on the chemical and physical environment in which they reside in and the temperature and pressure to which the compounds are subjected. Certain compounds, such as but not limited to water and decane, may be condensable in a cryotrap, whereas others, such as but not limited to hydrogen, oxygen and nitrogen, may not be condensable in a cryotrap.

### Very Small Volatiles

Hydrogen gas and helium gas are examples of two very small volatiles.

Hydrogen may exist as two hydrogen atoms chemically bound to each other. It may also exist as one hydrogen atom. Each hydrogen atom may contain zero, one or two neutrons. Hydrogen may exist as a solid, liquid, or gas, depending on the volume the hydrogen occupies, and the temperature and pressure that the hydrogen experiences in such volume. Hydrogen may reside within the crystalline lattice, pores, cracks, inclusions, etc., within a solid, or it may be chemically associated with the solid (such as in the form of a hydride), or both reside within pores, cracks, inclusions, etc. and be chemically associated with the solid. Hydrogen may be dissolved in, suspended in, or chemically associated with a liquid.

Helium exists as an atom with two protons and typically two neutrons, although it may possess only one neutron. Helium may exist as a solid, liquid or gas, depending on the volume the helium occupies, and the temperature and pressure that the helium experiences in such volume. Helium may reside within a crystal lattice, pores, cracks, inclusions, etc., within a solid or it may be very rarely chemically associated with the solid, or both reside within pores, cracks, inclusions, etc., and be very rarely chemically associated with the solid. Helium may be dissolved in, suspended in, or very rarely chemically associated with a liquid. Helium may exist in a geologic unit closer to the surface than hydrogen, preventing the hydrogen from escaping and also allowing for the hydrogen to be concentrated and increased in purity. Helium and hydrogen may coexist within the same geologic unit. Helium and hydrogen may be close to one another but separate in that the helium is highly pure and the hydrogen is highly pure.

Helium may exist in a zone under the surface of the Earth, wherein there is a zone, above the helium zone, which is impermeable to helium and provide a geological seal to helium migration.

### Other Volatiles/Non-VS Volatiles (NVSVs)

Hydrogen is an example of a very small volatile.

Other volatiles may be extracted from a sample that are not VSVs. These are termed non-very-small volatiles (NVSVs).

NVSVs may comprise inorganic volatiles, e.g., sulfur dioxide or nitrous oxide, organic volatiles, such as methane, and C2 and higher hydrocarbons, and may also comprise hydrogen proxies (compounds whose presence or change in amount suggests the presence of hydrogen prior to generating the compound or reacting with the compound to change its amount.

### Proxies (E.g., VSV Proxies)

Proxies are substances which can be analyzed herein in lieu of one or more other (e.g., “primary”) substances, wherein the measured amount of a proxy can be used to either directly or indirectly identify or estimate the amount of a primary substance. Proxies herein can be very small volatile proxies, e.g., proxies of a very small volatile such as hydrogen.

### Indirect Proxies, Direct Proxies

Volatiles may exist in a sample and be extractable from a sample, wherein the presence of the volatile may be indicative of a reaction between hydrogen and a compound (direct proxies) or a change in the amount of a compound may indicate that such a compound reacted with hydrogen to change the amount of the compound (indirect proxies).

Ammonia and water are examples of direct proxies of hydrogen that have previously been present but consumed in generating these compounds. For example, nitrogen (present in air) can react with hydrogen to produce ammonia. Similarly, oxygen (present in air) can react with hydrogen to produce water.

Oxygen and Nitrogen, on the other hand, are examples of indirect hydrogen proxies. A decreased presence of either oxygen, nitrogen or both as compared to the concentration of each of these in ambient air may indicate that hydrogen (now consumed) reacted with such compounds to produce water and ammonia, respectively.

Hydrogen proxies, direct, indirect, or both, are volatile substances which may be used in calculating the amount of hydrogen that was in a sample prior to generating or reacting with such respective direct or indirect proxies.

### Hydrogen Content (Hydrogen-Rich, Hydrogen-Poor)

Hydrogen-rich means a material having a concentration of hydrogen that is greater than the concentration of hydrogen in the Earth's atmosphere or having a purity of hydrogen that is greater than the purity of hydrogen in the atmosphere or both the concentration and purity of the hydrogen are greater than the concentration and purity in the Earth's atmosphere. Such concentration of hydrogen may be greater than previously believed achievable based on the pressure, temperature, and volume of cracks, inclusions, or liquids in the location. Additionally, the purity of the hydrogen as a percentage of other gases from the same entity may be greater than 50%, greater than 75%, greater than 90%, greater than 95%, greater than 99%, greater than 99.8%, or almost 100% when measured at standard temperature, 30 degrees Celsius, and pressure, 1 atmosphere.

Hydrogen-poor means a material having a concentration of hydrogen that is less than the concentration of hydrogen in the Earth's atmosphere or having a purity of hydrogen that is less than the purity of hydrogen in the atmosphere, or both the concentration and purity of the hydrogen are less than the concentration and purity in the Earth's atmosphere.

### Generation

Generation means forceable forming, for example, by way of contacting at least a first substance with a second substance, at least one substance which otherwise would not be present in the same amount(s) or available at the same rate. An example of generation is, e.g., the formation of hydrogen from ferrous oxides by way of contacting ferrous oxide rock with one or more fluids.

### Production

Production means the realization of an existing resource, such as petroleum or hydrogen. Production can comprise the extraction of existing, e.g., petroleum or hydrogen from a geological unit.

### Geologic Units, Zones, and Related Terminology

A geologic unit is any discrete geologic area, e.g., a basin, group, formation, member, area, or site from which suitable samples may be obtained for use in methods, devices, or compositions described herein, such as, e.g., a specific well. The term geologic unit is used to refer to any discrete geologic area from which suitable samples are obtained for use in the methods herein. For example, a geologic unit can comprise a portion of one or more formations. In certain aspects, herein, use of the term geologic unit can refer to or encompass a specific geologic site or any discrete geologic area from which suitable samples are obtained for use in methods herein, such as, e.g., a specific well. In aspects, a geologic unit, e.g., comprising one or more geologic sites, e.g., petroleum well(s), or a single geologic site, comprises many distinct locations that can be characterized based on vertical depth and lateral/lengthwise distance. Typically, samples are provided from (or collected from) more than 10 separate locations, such as at least about 20, at least about 30, at least about 40, at least about 50, at least about 65, at least about 75, or at least about 100 locations. In aspects, samples are provided from more than 100 locations, such as at least about 125, at least about 150, at least about 200, at least about 250, or more different locations in a site. The samples can be of any nature that includes an analyzable amount of rock material for the methods described herein.

Resource plays are typically associated with tight conventional formations where fluid is removed directly from a resource as opposed to removing it from the ground after it has migrated to a new location from its original source, direct quantitative compositional matching (e.g., direct measurement of compounds and comparison of the same) can in some cases be possible, as the quantitative amount of each compound should not be significantly altered.

Formation is understood in the art to mean an identified area of strata having similar lithology. In some cases, a formation also may be defined by other characteristics, such as biostratigraphic characteristics, chemostratigraphic characteristics, or both, and sometimes such characterizations of a formation are used interchangeably. Typically, a formation is a series of strata/beds that is distinct from other beds above and below and is thick enough to be shown on the geological maps that are widely used within the area in question. Formations dominated by a rock typically include the dominant rock in the formation's name (e.g., the “Woodford Shale Formation” found in several parts of Oklahoma). However, formations in some cases can contain a variety of related or interlayered rock types, such as the Summerville Formation of Utah, which consists of thin alternating beds of shale, siltstone, and sandstone. Formations can be divided into sub-formations or “members” based on such characteristics.

Plays can be divided into “regions” or “areas” comprising two or more (often several) sites, potential sites, or both.

A site is typically a well, e.g., a petroleum/hydrogen well or an area of prospective drilling within an area or play. In aspects, related samples can be obtained from multiple sites within a single play.

Well or borehole can sometimes be used in common parlance to distinguish mechanisms of drilling (e.g., a borehole typically drilled by machine and being small in diameter, a well typically being sunk by hand and being relatively larger in diameter), herein the two terms are used interchangeably to describe a vertical or horizontal shaft in the ground, commonly used herein to describe a well for discovery, characterization and/or production of helium, hydrogen, and or other compound(s) that are typically gas(es) or liquid(s) when subjected to standard atmospheric temperature and pressure. The terms well or borehole should be interpreted as being applicable to such wells and/or petroleum wells, whereby hydrogen, helium, or other gas(es) or liquid(s) are discovered, characterized, or produced. Alternatively, a well or borehole may be concurrently or previously used as a carbon capture storage well, or a geothermal well. The term “well” as used herein is inclusive of producing and non-producing wells, online wells, wells not yet brought online, and dry (non-producing) wells (e.g., in aspects the methods herein can aid in the evaluation of, or determine, whether additional exploration/drilling of such a dry well should be considered, which can be indicated by cuttings from such a well sharing characteristics with that of nearby productive wells as determined by the application of the method(s)). The phrase “well/borehole” is used to reflect that either of these terms can apply.

The term reservoir is used to describe a geological formation or portion of a formation that includes sufficient porosity and permeability to store and transmit a gas such as carbon dioxide, helium, or hydrogen, or, in aspects, a fluid such as oil. The phrase “reservoir/compartment” is used to reflect that either of such terms applies.

The term compartment describes a geological area effectively sealed off from another, with little to no fluid communication occurring between two or more compartments. The phrase “reservoir/compartment” is used to reflect that either of such terms applies.

### Data/Information

Uncontradicted, terms such as “data” and “information” herein are intended to implicitly disclose any and all forms of applicable or possibly applicable data, information, etc., of any suitable and applicable kind, form, etc. Readers will understand that the term “data” can be used to refer to a particular item of information (fact, measure, record, etc.) or a collection of such items and 2, uncontradicted, should be interpreted as implicitly meaning both. In aspects, the term “data set” is used to specifically refer to a collection of data items/records.

### Properties of Substances

Geologic material is a material derived from the Earth. Such material may be solid, liquid, gas, or any one or more combination of any of these in any relative amounts by weight from 0% to 100%, such that the total percentage by weight of solid, liquid, and gas is 100%.

What is claimed is:

1. A method for measuring the amount of hydrogen in a material comprising
(1) obtaining an analyzable amount of a material from a source, the material comprising one or more easily released volatile substances,
(2) causing the material to contact media comprising air to form a sample under conditions in which at least some of the easily released volatile substances in the material are transferred to the media and at least some of any present easily released volatile hydrogen in the easily released volatile substances reacts with the media to form at least one hydrogen proxy substance within the media, the material, or both,
(3) subjecting the sample to one or more gentle vacuum forces, one or more gentle vacuum equivalent forces, or both, to form an extract comprising at least some of the media and at least some of the easily released volatile substances that were contained in the material before the application of the one or more gentle vacuum forces, gentle vacuum equivalent forces, or both,
(4) collecting at least a portion of the extract as a collection,
(5) subjecting an aliquot comprising some or all of the collection to an analytical process that measures the amount of the at least one hydrogen proxy substance in the aliquot, and
(6) using the measured amount of the at least one hydrogen proxy substance in the aliquot to determine the amount of hydrogen in the sample, and using the amount of hydrogen in the sample to determine the amount of hydrogen in the source.

2. The method of claim 1, wherein the method comprises isolating the material by a process comprising placing the material in an enclosed environment prior to subjecting the sample to the one or more gentle vacuum forces, the one or more gentle vacuum equivalent forces, or both.

3. The method of claim 2, wherein the sample is isolated in the enclosed environment with the media.

4. The method of claim 3, wherein at least 90% of the media is air.

5. The method of claim 4, wherein the one or more gentle vacuum forces, gentle vacuum equivalent forces, or both comprise applying a force of or equivalent to an application of between 10 mbar and 30 mbar of pressure.

6. The method of claim 1, wherein the method further comprises (7) measuring the amount of easily released volatile hydrogen in the sample and (8) using both the measured amount of volatile hydrogen and the amount of the at least one hydrogen proxy substance measured to quantify the amount of hydrogen in the sample.

7. The method of claim 1, wherein the at least one hydrogen proxy substance comprises ammonia.

8. The method of claim 4, wherein the sample is isolated in the enclosed environment with the media, and the method comprises measuring the amount of argon in the media to determine the amount of air in the enclosed environment.

9. The method of claim 8, wherein the at least one hydrogen proxy substance comprises ammonia, at least one ammonia proxy, or both ammonia and at least one ammonia proxy.

10. The method of claim 9, wherein the at least one hydrogen proxy substance comprises at least one ammonia proxy and the at least one ammonia proxy comprises nitrogen, and the method comprises using the amount of air in the enclosed environment to determine the amount of ammonia produced from the amount of easily released volatile hydrogen in the sample prior to isolating the sample.

11. The method of claim 1, wherein the at least one hydrogen proxy substance comprises at least one water proxy.

12. The method of claim 11, wherein the material is isolated in an enclosed environment with air, and the method comprises measuring the amount of argon in the air to determine the amount of air in the enclosed environment; the at least one water proxy comprises oxygen; and the method comprises using the amount of air in the enclosed environment to determine the amount of water produced from the amount of easily released volatile hydrogen in the material.

13. The method of claim 6, wherein the at least one hydrogen proxy substance comprises (a) ammonia or at least one ammonia proxy and (b) water or at least one water proxy.

14. The method of claim 13, wherein the material is isolated in an enclosed environment with air, and the method further comprises measuring the amount of argon in the air to determine the amount of air in the enclosed environment.

15. The method of claim 14, wherein the method comprises removing the water from the sample, the extract, or both, before or concurrently with collecting the collection.

16. The method of claim 15, wherein the removal of the water from the sample, the extract, or both, is performed at least in part by subjecting the sample, the extract, or both to a media that selectively traps a portion of the easily released volatile substances, wherein the portion (1) comprises the water, (2) does not comprise all of the very small volatiles in the easily released volatile substances, and (3) does not comprise the at least one hydrogen proxy substance.

17. The method of claim 16, wherein the removal of the water from the sample, the extract, or both, is performed by actions comprising contacting the extracted easily released volatile substances with the media wherein the media is a cryogenic trap whereby at least a portion of the extracted easily released volatile substances is trapped such that the trapped portion of the easily released volatile substances condenses to the cryogenic trap, the non-trapped portion of the easily released volatile substances comprises the at least one hydrogen proxy substance, and the method comprises using the non-trapped portion of the easily released volatile substances as the collection.

18. The method of claim 17, wherein the determination of the amount of hydrogen in the material comprises using applying one or more mathematical factors to the amount of hydrogen determined to be in the sample, wherein the one or more mathematical factors are selected based on (a) the time between the collection of the material and the analysis of the aliquot, (b) the distance between the source and the location where the material is obtained, or (c) both the time between the collection of the material and the analysis of the aliquot and the distance between the source and the location where the material is obtained.

19. The method of claim 1, wherein the method comprises collecting multiple samples of material from different parts of a geologic unit and separately subjecting each of the material samples to the steps of claim 1 to determine the amount of hydrogen present in the different parts of the geologic unit.

20. The method of claim 19, wherein the multiple material samples comprise a plurality of drill cuttings, at least one mud sample, at least one core sample, or a combination of any or all thereof.

21. The method of claim 1, wherein the method comprises comparing the amount of easily released volatile hydrogen in the sample to an amount of easily released volatile helium in the sample.

22. The method of claim 21, wherein the method comprises collecting a plurality of material samples collected from different parts of a geologic unit, determining the amount of easily released volatile hydrogen in the plurality of the material samples by the method of claim 1, and measuring the amount of easily released helium in the plurality of material samples to determine the amounts of easily released volatile hydrogen and easily released helium present in the different parts of the geologic unit.

23. The method of claim 21, wherein the method comprises subjecting one or more samples to two or more different volatile extraction conditions, wherein (1) the application of a first volatile extraction condition of the at least two volatile extraction conditions results in the extraction of a significantly greater amount of easily released volatile hydrogen than easily released volatile helium and (2) the application of a second volatile extraction condition of the at least two volatile extraction conditions results in extraction of a significantly greater amount of easily released volatile helium than easily released volatile hydrogen.

24. The method of claim 1, wherein the application of the one or more gentle vacuum forces, gentle vacuum equivalent forces, or both comprises subjecting the sample to at least two separate applications of gentle vacuum pressure, the at least two separate applications of gentle vacuum pressure comprising (1) a first vacuum having a first pressure applied for a first time period and (2) a second vacuum having either the first pressure or a pressure that is substantially the same pressure as the first pressure and which is applied for a second period, wherein the second period is substantially longer than the first period.

25. The method of claim 24, wherein the at least two separate applications of gentle vacuum pressure further comprise (1) the application of a third vacuum having a second pressure applied for a third period and (2) the application of a fourth vacuum having either the second pressure or a pressure that is substantially the same pressure as the second pressure applied for a fourth period, wherein the fourth period is substantially longer than the third period, and, wherein at least one of the first vacuum, second vacuum, third vacuum, and fourth vacuum extracts different amounts of easily released volatile hydrogen, at least one hydrogen proxy substance, or both from the material.

26. A method for identifying conditions for enhanced production of highly purified hydrogen from a source comprising
(1) obtaining an analyzable amount of at least one material from the source comprising one or more easily released volatile substances, wherein the one or more easily released volatile substances comprises an analyzable amount of easily released volatile hydrogen if easily released volatile hydrogen is present, an analyzable amount of easily released volatile helium if easily released volatile helium is present, or both analyzable amounts of easily released volatile hydrogen and easily released volatile helium if both easily released volatile hydrogen and easily released volatile helium are present,
(2) contacting each of the at least one material with media comprising air to form a sample under conditions in which at least some of any easily released volatile hydrogen present in the material reacts with the air to form at least one hydrogen proxy substance within the media, the material, or both,
(3) subjecting each sample to one or more gentle vacuum forces, gentle vacuum equivalent forces, or both, to form an extract of each sample comprising at least some of the media and at least some easily released volatile substances that were contained in the material before the application of the one or more gentle vacuum, gentle vacuum equivalent forces, or both, wherein
(a) each sample comprises
(I) easily released volatile hydrogen if easily released volatile hydrogen is present in the extract,
(II) at least one hydrogen proxy substance if at least one hydrogen proxy substance is present in the extract, or
(III) both easily released volatile hydrogen and at least one hydrogen proxy substance if both easily released volatile hydrogen and at least one hydrogen proxy substance are present in the extract, and helium if helium is present in the extract, and
(IV) easily released helium if helium is present in the extract, and
(b) either
(I) the at least one material comprises multiple materials, wherein a relatively higher amount of easily released volatile hydrogen than the amount of easily released volatile helium is extracted from at least one material of the multiple materials than from at least one other material of the multiple materials,
(II) the method comprises subjecting each of the samples to at least two different gentle vacuum forces, gentle vacuum equivalent forces, or both comprising a first gentle vacuum force or gentle vacuum equivalent force that extracts a relatively higher amount of easily released volatile hydrogen than amount of easily released volatile helium from the at least one material than at least a second gentle vacuum force or gentle vacuum equivalent force of the at least two different gentle vacuum forces or gentle vacuum equivalent forces, or
(III) the at least one material comprises multiple materials, wherein a relatively higher amount of easily released volatile hydrogen than amount of easily released volatile helium is extracted from at least one material of the multiple materials than at least one other material of the multiple materials and the method comprises subjecting each of the materials to at least two different gentle vacuum forces, gentle vacuum equivalent forces, or both comprising a first gentle vacuum force or gentle vacuum equivalent force that extracts a relatively higher amount of easily released volatile hydrogen than amount of easily released volatile helium from at least one of the multiple samples than at least a second gentle vacuum force or gentle vacuum equivalent force of the at least two different gentle vacuum forces or gentle vacuum equivalent forces,
(4) collecting at least a portion of each extract as a collection of each extract, and
(5) subjecting an aliquot comprising some or all of each collection to an analytical process that measures the amounts of (i) the easily released volatile hydrogen, the at least one hydrogen proxy substance, or both easily released volatile hydrogen and the at least one hydrogen proxy substance, and (ii) easily released volatile helium, in each aliquot, and
(6) using the measurements of the amounts of easily released volatile hydrogen, at least one hydrogen proxy substance, or both easily released volatile hydrogen and the at least one hydrogen proxy substance, and easily released volatile helium from each aliquot to identify (a) a gentle vacuum extraction force that selectively extracts easily released volatile hydrogen from the at least one material, (b) the at least one material that is associated with the relatively higher amount of easily released volatile hydrogen extraction than the amount of easily released volatile helium extraction, or (c) both a gentle vacuum extraction force that selectively extracts easily released volatile hydrogen from the at least one material and the at least one material that is associated with the relatively higher amount of easily released volatile hydrogen extraction than the amount of easily released volatile helium extraction.

27. The method of claim 26, wherein the at least one material comprises multiple materials, and wherein a relatively higher amount of easily released volatile hydrogen than amount of easily released volatile helium is extracted from at least one material of the multiple materials than at least one other material of the multiple materials, and the measurements of easily released volatile hydrogen, at least one hydrogen proxy substance, or both easily released volatile hydrogen and the at least one hydrogen proxy substance, and easily released volatile helium are used to identify the at least one material that is associated with the relatively higher amount of easily released volatile hydrogen than amount of easily released volatile helium.

28. The method of claim 27, wherein the at least one material of the multiple materials is obtained from a geologic unit, and the method comprises identifying separated areas of relatively high helium concentration and relatively high easily released volatile hydrogen concentration in the geologic unit.

29. The method of claim 26, wherein the method comprises subjecting each sample to at least two different gentle vacuum forces or gentle vacuum equivalent forces comprising a first gentle vacuum force or gentle vacuum equivalent force that extracts a relatively higher amount of easily released volatile hydrogen than easily released volatile helium from at least one sample than at least a second gentle vacuum force or gentle vacuum equivalent force of the at least two different gentle vacuum forces or gentle vacuum equivalent forces and the measurements of easily released volatile hydrogen, at least one hydrogen proxy substance, or both easily released volatile hydrogen and the at least one hydrogen proxy substance, and easily released volatile helium are used to identify a gentle vacuum equivalent force that selectively extracts easily released volatile hydrogen from at least one material.

30. The method of claim 29, wherein the first gentle vacuum force or gentle vacuum equivalent force and the second gentle vacuum force or gentle vacuum equivalent force each comprise gentle vacuum pressures.

31. The method of claim 30, wherein the first gentle vacuum pressure and the second gentle vacuum pressure differ in the duration of the gentle vacuum pressure applied to each sample.

32. The method of claim 30, wherein the first gentle vacuum pressure and the second gentle vacuum pressure differ in the strength of the gentle vacuum pressure applied to each sample.
